(19) United States
(12) Patent Application Publication
(10) Pub. No.: US 2024/0392365 A1
(43) Pub. Date: Nov. 28, 2024

(54) NUCLEIC ACID CHARACTERISATION

(71) Applicant: CAMBRIDGE ENTERPRISE LIMITED, Cambridge, Cambridgeshire (GB)

(72) Inventors: Ulrich KEYSER, Cambridge, Cambridgeshire (GB); Filip BOSKOVIC, Cambridge, Cambridgeshire (GB)

(21) Appl. No.: 18/696,136

(22) PCT Filed: Sep. 29, 2022
(86) PCT No.: PCT/GB2022/052466

§ 371 Date: Mar. 27, 2024

(30) Foreign Application Priority Data
Sep. 29, 2021 (GB) 2113935.7

(51) Int. Cl. C12Q 1/6869; C12Q 1/689; G01N 33/569; G01N 33/58; G01N 33/68

(52) U.S. Cl. CPC C12Q 1/6869; C12Q 1/689; G01N 33/56983; G01N 33/582; G01N 33/6854

(57) ABSTRACT

The invention relates to methods for nucleic acid characterisation. In particular, the method of the invention relates to methods for characterising target nucleic acids in a sample.

The project leading to this application has received funding from the European Research Council (ERC) under the European Union's Horizon 2020 research and innovation programme (grant agreement No 647144).

### FIELD OF THE INVENTION

This invention relates to methods for characterising target nucleic acids.

### BACKGROUND OF THE INVENTION

Nucleic acid characterisation and quantification are central to a wide variety of scientific techniques and underpins both genomic and transcriptomic studies. Traditional methods for characterising and quantifying nucleic acids typically require laborious sample preparation and often involve enzyme mediated amplification or reverse transcription steps which are inherently susceptible to errors induced by enzymatic biases.

Accurate characterisation and quantification of native RNA transcript isoforms are critical for understanding transcriptome diversity and gene expression networks. Various methods known in the art, e.g. RNA-seq, rely on the reverse transcription of native RNA transcripts to produce complementary DNA (cDNA) which is then amplified and sequenced. These methods suffer from errors associated with enzymatic (e.g. reverse transcriptase and polymerase) biases resulting in low reproducibility and results that do not necessarily reflect innate transcriptome diversity.

Nanopore-based sequencing approaches have been developed which allow the direct sequencing of RNA, e.g. RNA transcripts. However, these methods face challenges associated with nanopore translocase biases, low-quality reads and inconsistent sequencing of the 5′ end of RNA.

There is a need in the art for fast and reliable nucleic acid characterisation and quantification methods which are not reliant on laborious sample preparation and enzymatic processing steps. These needs have been acutely felt during the SARS-CoV-2 pandemic. In particular, there is a need for methods that allow the direct characterisation of native RNA molecules, e.g. RNA transcripts.

### SUMMARY OF THE INVENTION

The inventors have overcome the above problems by identifying a novel method for characterising target nucleic acid(s). In more detail, the inventors discovered that native nucleic acids can be characterised by: (i) contacting the target nucleic acid with linearising unit(s) which provide one or more structural unit(s) interspaced by one or more regions of double-stranded nucleic acid; and (ii) detecting structural unit(s) along the target nucleic acid. Linearising unit(s) comprise docking strand(s) which have a region that is complementary to a distinct region of the target nucleic acid. One or more regions of the double-stranded nucleic acid comprises a docking strand of the linearising unit hybridised to the distinct region(s) of the target nucleic acid. Binding of the docking strand(s) to distinct regions of the target nucleic acid reduces secondary structure in the distinct region of the target nucleic acid, thereby allowing structural units to be detected. Structural units may be provided by linearising units that are complementary to distinct regions of the target nucleic acid; and/or by single-stranded regions of the target nucleic acid which self-assemble into secondary structures.

Advantageously, the method of the invention avoids the need for intensive sample preparation and does not rely on enzymatic processing steps, thereby eliminating problems associated with enzymatic biases. The method of invention also provides a high level of sensitivity and can be used to characterise target nucleic acid(s) that are present at low abundance in complex samples comprising a diverse mixture of non-target nucleic acids. The method of the invention is also rapid and can be readily multiplexed allowing the characterisation of multiple target nucleic acids in a single reaction.

The invention provides a method for characterising a target nucleic acid, the method comprising the steps of:
(a) contacting the target nucleic acid with one or more linearising unit(s) to provide one or more structural unit(s) interspaced by one or more regions of double-stranded nucleic acid; and
(b) detecting structural unit(s) along the target nucleic acid;
   wherein:
(i) each linearising unit comprises a docking strand having a region that is complementary to distinct region(s) of the target nucleic acid;
(ii) one or more regions of said double-stranded nucleic acid comprises a docking strand of said linearising unit hybridised to said distinct region(s) of the target nucleic acid; and
(iii) binding of the docking strand(s) to the target nucleic acid reduces secondary structure in the distinct region(s) of the target nucleic acid.

In one embodiment, one or more of the structural unit(s) is provided by the linearising unit(s). In one embodiment, one or more of the linearising unit(s) comprise: (i) a docking strand having a region that is complementary to distinct region(s) of the target nucleic acid and an overhang region; and (ii) a labelling strand that is complementary to the overhang region of the docking strand and comprises a label. In one embodiment, one or more of the linearising unit(s) comprise a docking strand having a region that is complementary to distinct region(s) of the target nucleic acid and a labelling region.

In one embodiment, one or more of the linearising unit(s) are separated by single-stranded region(s) of the target nucleic acid, and wherein one or more of the structural unit(s) is provided by secondary structures formed by said single-stranded region(s) of the target nucleic acid.

In one embodiment, the linearising units provide one or more structural colour(s) wherein each structural colour comprises: (a) an integer number of adjacent structural units detectable as a single signal; and/or (b) structural unit(s) which provide a signal that is distinct from other structural unit(s) and/or colour(s).

In one embodiment, the method comprises detecting the sequence of structural unit(s) and/or structural colour(s) along the target nucleic acid.

In one embodiment, the target nucleic acid is RNA. In one embodiment, the RNA is selected from single-stranded RNA (ssRNA), pre-mRNA, mRNA, miRNA, and non-coding RNA. In one embodiment, the target nucleic acid is an RNA transcript.

In one embodiment, the method comprises characterising more than one target nucleic acid.

In one embodiment, the labelling strand(s) comprise a structural, chemical and/or fluorescent label. In one embodiment, the labelling strand comprises a ligand label. In one embodiment, the method further comprises contacting the target nucleic acid with a receptor for the ligand, and wherein detecting structural unit(s) and/or structural colour(s) comprises detecting ligand/receptor complexes. In one embodiment, the ligand is biotin and the receptor is selected from streptavidin, neutravidin, traptavidin and avidin. In one embodiment, the ligand is an antigen and the receptor is an antibody. In one embodiment, the labelling strand comprises a fluorescent label. In one embodiment, the labelling strand comprises a DNA nanostructure; optionally wherein the DNA nanostructure is a DNA cuboid. In one embodiment, the labelling region comprises a structural label, optionally wherein the structural label is a nucleic acid nanostructure such as a DNA double hairpin structure.

In one embodiment, structural unit(s) along the target nucleic acid are detected using a nanopore-based detection method.

In one embodiment, structural unit(s) and/or structural colour(s) along the target nucleic acid are detected using a fluorescence-based detection method, optionally wherein the fluorescence-based detection method comprises fluorescence microscopy.

In one embodiment, structural unit(s) and/or structural colour(s) along the target nucleic acid are detected by a size-specific readout method, optionally wherein the size-specific readout method is mass photometry or a size-dependent lateral-flow assay.

In one embodiment, the method further comprises quantifying the amount of target nucleic acid in a sample, optionally wherein the target nucleic acid is quantified relative to an internal or external control.

In one embodiment, the target nucleic acid is derived from a virus, optionally wherein the virus is selected from a coronavirus, Influenza virus, Zika virus, Ebola virus, Dengue virus, Hantavirus, Nairovirus, Orthobunyavirus, Phlebovirus, Flavivirus, and Alphavirus. In one embodiment, the target nucleic acid is a coronavirus genome, optionally the SARS-CoV-2 genome.

In one embodiment, the target nucleic acid is derived from a microorganism, optionally wherein the target nucleic acid is derived from a bacteria or a fungi.

In one embodiment, the target nucleic acid is derived from a pathogen, optionally wherein the pathogen is a viral pathogen, bacterial pathogen, fungal pathogen, protozoan pathogen or pathogenic worm.

In one embodiment, the method comprises characterising one or more RNA transcript isoforms, optionally wherein the method further comprises quantifying each of the one or more transcript isoforms.

In one embodiment, the single-stranded region(s) of the target nucleic acid that provide the structural unit(s) and/or structural colour(s) do not hybridise with linearising units. In one embodiment, the single-stranded region(s) comprise a secondary structure that prevents or reduces hybridisation of the single-stranded region(s) with linearising units. In one embodiment, the presence of a nucleic acid binding molecule prevents or reduces hybridisation of the single-stranded region(s) with linearising units, optionally wherein the nucleic acid binding molecule binds to the single-stranded region or stabilises a secondary structure thereof. In one embodiment, the nucleic acid binding molecule is a drug, a protein, nucleic acid, ligand, small molecule, or an RNA binding protein (RBP). In one embodiment, the method further comprises characterising the presence and/or location of binding between the target nucleic acid and nucleic acid binding molecule.

In one embodiment, the target nucleic acid is an RNA molecule and contacting the RNA molecule with linearising units reshapes the target RNA molecule into a linear RNA comprising structural units and/or structural colour(s) interspaced by double stranded regions of nucleic acid.

In one embodiment, the method further comprises characterising the length of a repeated sequence or the number of repeated sequences present in the target nucleic acid. In one embodiment, the method comprises characterising the length of a poly(adenine) tail.

In one embodiment, the target nucleic acid is present in a sample obtained from a subject, optionally wherein the subject is a human. In one embodiment, the sample is selected from blood, serum, plasma, saliva, sputum, urine, faeces, cerebrospinal fluid, a lung tissue sample, a bronchoalveolar lavage sample, a nose and/or throat swab sample, or a biopsy sample.

In one embodiment, the step of contacting the target nucleic acid with one or more linearising unit(s) comprises: (A) contacting a sample comprising a cell and/or a virus having the target nucleic acid with one or more linearising unit(s); and (B) lysing the cell and/or the virus. In one embodiment, lysing the cell and/or the virus comprises heating the cell and/or the virus.

In one embodiment the virus is selected from a coronavirus, Influenza virus, Zika virus, Ebola virus, Dengue virus, Hantavirus, Nairovirus, Orthobunyavirus, Phlebovirus, Flavivirus, and Alphavirus. In one embodiment, the cell is a microorganism cell, optionally a bacterial cell or a fungal cell. In one embodiment, the cell is a eukaryotic cell, optionally a mammalian cell, optionally a human cell.

### BRIEF DESCRIPTION OF THE DRAWINGS

FIG. 1. Example workflow: (A) RNA isoform-specific identifier (ID) fabrication using structural colours (1, 2 and 3). Three RNA isoforms are tagged with an ‘exon-specific’ sequence of structural colours. In this example, structural colours consist of an integer number of linearising-structural units (typically 0-10) that are placed sequentially along the target nucleic acid and read as one structural colour (e.g. the structural colour 10 corresponds to 10 sequentially placed linearising-structural units). (B) A molecular ruler or ID (scaffold strand) with ten different structural colours is read by passing the scaffold strand through a nanopore microscope. In this example, each structural unit is provided by a linearising unit (referred to herein as a linearising-structural unit) comprising a docking strand having a region that binds to the target nucleic acid and an overhang and a labelling strand that is complementary to the overhang of the docking strand and comprises a detectable label, e.g. a terminal structure (e.g. monovalent streptavidin or DNA cuboid). (C) An exemplary nanopore microscope current trace (also referred to herein as an event) demonstrating detection of 10 structural colours within the same molecular ruler. (D) The correct construction of the structural colours (correct number of linearising-structural units per structural colour) was verified using fluorescently labelled (5′-fluorescein) structural units. In the plot, normalized fluorescence for 1-10 structural colours is shown. Error bars indicate a standard error of three repeats. (E) Single-molecule readout of structural colours and their identity in example nanopore events.

FIG. 2. Overview of an exemplary design of a linearising-structural unit and structural colour. (A) Structural units provided by linearising units (linearising-structural units) typically comprise a detectable label, e.g. a structure detectable by a nanopore microscope. The inventors demonstrated the use of a protein structure (monovalent streptavidin—dark grey) and a DNA nanostructure (DNA cuboid—light grey). Each structural colour is produced by an integer number of adjacent linearising-structural units which are detectable as a single signal. The number of linearising-structural units corresponds to the structural colour, e.g. two adjacent linearising-structural units provide structural colour ‘2’ and a specific signal strength (drop in the ionic current) associated with that colour. (B) Physical characteristics of both monovalent streptavidin (52.8 kDa) and DNA cuboid (64.9 kDa) are delineated. Monovalent streptavidin has a diameter of 5-6 nm. DNA cuboid has a length of 15.6 nm (46 bp) with labelling strand and 8.8 nm (26 bp) without, while the width corresponds to two DNA helixes or 4 nm.

FIG. 3. Design and analysis of 4-colour ruler. (A) Design of 4-colour ruler comprising four sites that have 1, 2, 3, or 4 adjacent linearising-structural units (structural colours 1-4). In this example, each linearising-structural unit comprises: (i) a docking strand having 20 nt complementary to the scaffold strand (grey) and an overhang (dark grey); and (ii) a labelling strand with 3′ biotin label (black). Sequences of both strands are shown in the table including their length. (B) Exemplary protocol for the fabrication of a linearising-structural unit. In the ID fabrication step, docking and labelling strands form a duplex. Monovalent streptavidin (which has femtomolar affinity to biotin with inactivated three out of four biotin-binding sites) is added prior to detection. (C) Example ruler events clearly indicate four downward signals corresponding to structural colours from (A) 1, 2, 3, and 4. (D) Each detected structural colour position is plotted by taking structural colour 4 as zero time point and showing the distance from it for structural colours 3, 2, and 1. The current signal for each colour is calculated as a drop from the first current drop level originating from the ruler itself. The sample size is thirty unfolded ruler events.

FIG. 4. Design and additional example events of the 10-colour ruler. (A) Design of 10-colour ruler indicates ten sites that have 1, 2, 3, 4, 5, 6, 7, 8, 9, and 10 linearising-structural units. (B) Example ruler events indicate ten downward signals corresponding to structural colour from (A) 1, 2, 3, 4, 5, 6, 7, 8, 9, and 10. (C) Scatter plot of current drop and normalized position for each structural colour. The correct structural colours are identified as a distinctive signal on a single-event basis. The sample size is sixty unfolded ruler events.

FIG. 5. Electrophoresis analysis of fabricated 4-colour and 10-colour rulers on the agarose gel. Lanes: 1—linear single-stranded M13; 2—double-stranded M13; 3-4-colour ruler without streptavidin; 4-10-colour ruler without streptavidin; 5-4-colour ruler with streptavidin; 6-10-colour ruler with streptavidin; 7-1 kb DNA ladder (NEB); 8—single-stranded RNA ladder (NEB). Gel: 1% (w/v) agarose, 1×TBE.

FIG. 6. Fluorescence quenching assay for validation of expected structural colour. (A) A specific number of linearising-structural units (1 or 2 . . . or 10) with 5′-fluorescein (6-FAM) were added to a double-stranded molecular ruler. Excess 6-FAM DNA cuboid strands were quenched by binding of Iowa Black quencher. After the quenching, only 6-FAM DNA cuboids in the linearising-structural unit emit a fluorescence signal. (B) Using equimolar concentration of molecular rulers the inventors measured fluorescence. Example fluorescence measurements for each of 10 structural colours separately are provided.

FIG. 7. Exemplary one-pot reaction for multiplex gene expression quantification in a complex human transcriptome. (A) ID fabrication and designs for multiple RNA targets in a complex mixture of human total RNA. Contacting target RNA with linearising units produces an RNA ID comprising structural unit(s) and/or colour(s) interspaced by double-stranded regions of nucleic acid, e.g. 18S rRNA ID ‘1111’ comprises four structural units represented by ‘1’ interspaced by regions of double-stranded nucleic acid. Exemplary events for 18S rRNA ID ‘1111’, 28S rRNA ID ‘11111’, and MS2 RNA ID control are presented in (B), (C), and (D), respectively. (E) and (F) Quantification of 18S rRNA, 28S rRNA, and MS2 RNA in human total universal RNA (E) and human cervical adenocarcinoma total RNA (F). (G) Event charge deficit (ECD) of the identified RNA targets illustrates expected differences between IDs. MS2 RNA ID was employed as an external control with a known concentration.

FIG. 8. RNA ID designs for 18S rRNA and 28S rRNA. (A) 18S rRNA ID ‘1111’ design. (B) 28S rRNA ID ‘11111’ design.

FIG. 9. RNA ID additional event examples for 18S rRNA and 28S rRNA IDs. (A) 18S rRNA ID ‘1111’ examples. (B) 28S rRNA ID ‘11111’ examples.

FIG. 10. Fabrication of RNA ID using a part of long MS2 RNA ^{˜}3.6 kb. (A) ID ‘111’ is designed to be in the middle of the target RNA (using only part of the target RNA for ID fabrication). (B) Detected ID events from nanopore recordings have three visible downward signals and a deep drop originating from a native single-stranded coil outside of the ID region. (C) The translocation time difference between the first two (374 nt) and the last two (488 nt) structural colours. (D) Concentration dependence of capture rate/translocation frequency. The sample size is 4083 events.

FIG. 11. Event frequency and concentration estimation from partially and fully complemented MS2 RNA ID ‘111’. (A) Design and example events of partially complemented RNA ID ‘111’ (‘111’p). (B) Design and example events of fully complemented RNA ID ‘111’ (‘111’f). (C) Translocation frequency for three individual measurements in equimolar concentrations of both ‘111’p and ‘111’f is plotted. The event number for all three individual measurements was 6566 events. Error bars are shown as ±standard error.

FIG. 12. Stability of RNA IDs over time under different storage temperatures. (A) Example events indicate correct ID readout over 8 days for IDs stored at 4° C. and −20° C. (B) RNA IDs do not show significant difference overtime for three times points (1 day, 4 days, and 8 days) at both 4° C. and −20° C. Gel: 1% (w/v) agarose, 1×TBE.

FIG. 13. Effects of temperature and salts on ID fabrication. (A) Example events for MS2 RNA ID ‘111’ using 70° C. for fabrication. (B) Example events for MS2 RNA ID ‘111’ using 85° C. for fabrication. (C) Example events for M13 DNA ID ‘111111’ using 70° C. for fabrication. (D) Example for M13 DNA ID ‘111111’ using 85° C. for fabrication. (E) Agarose gel indicates ID fabrication over various conditions. Lanes: 1—ssM13 DNA; 2-4—samples from (C); 6-9—samples from (D); D—1 kb DNA ladder; R—ssRNA ladder; 10—ssMS2 RNA; 11-14—samples from (A); 15-18—samples from (B). Gel: 1% (w/v) agarose, 1×TBE.

FIG. 14. Effects of salt concentration on ID fabrication. Agarose gel indicates ID fabrication for magnesium and lithium chloride at three concentrations. Lanes: 1-1 kb DNA ladder; 2—ssRNA ladder; 3—ssMS2 RNA; 4-6—MS2 RNA ID ‘111’ (fully complementary) for 2.5, 5, and 10 mM MgCl₂, respectively; 7-9—MS2 RNA ID ‘111’ (fully complementary) for 25, 50, and 100 mM LiCl, respectively. Gel: 1% (w/v) agarose, 1×TBE.

FIG. 15. Multiplex viral nucleic acid identification. (A) ID designs for the MS2 RNA virus and M13 DNA virus having three and six sites with the structural colour ‘1’, respectively. (B) Discrimination of detected IDs from nanopore recordings by mean event current vs event duration. (C) Histogram of event charge deficit for events assigned as MS2 ID ‘111’ and M13 ID ‘111111’. (D) Representative events for MS2 RNA ID ‘111’ and M13 DNA ID ‘111111’ IDs are shown. The sample size was 1341 events from the mix of parallelly fabricated IDs.

FIG. 16. The method of the invention discriminates alternative splicing isoforms resulting from any physical transcript arrangement. (A) Isoform-specific labelling may be achieved by labelling each exon with an asymmetric sequence of structural colours to produce unique IDs. (B) Example events for three RNA isoforms that differ in the order of structural elements (exons). (C) Example events for isoforms of different length demonstrating successful discrimination of length isoforms. (D) Example events for RNA isoforms having identical sequence and length but different conformations (circular and linear). (E) Nanopore discriminates linear and circular populations based on the translocation time (Δt) which is ^{˜}2 times shorter for the circular isoform, and the event current blockage (ΔI) which is ^{˜}2 times higher for the circular than for the linear ID.

FIG. 17. Design of exons with example nanopore events. (A) Design of exon I ID ‘112’ with terminal overhangs A and B′. (B) Design of exon II ID ‘312’ with terminal overhangs A′ and B′. (C) Design of exon Ill ID ‘321’ with terminal overhangs A′ and B. (D) Design of extended RNA ID with terminal overhangs A′.

FIG. 18. Design and example nanopore events for order isoforms. (A) Design of RNA ID ‘211312’. (B) Design of RNA ID ‘123112’. (C) Design of RNA ID ‘312123’

FIG. 19. Length isoform ‘211’ with extended RNA.

FIG. 20. Circular and linear isoforms. (A) Example events for circular ID ‘111’. (B) Example events for linear ID ‘111’. (C) The percentage of identified conformation with and without the oligo interlock. As expected in linearized single-stranded conformation (with and without interlock) almost all events are identified as linear conformation. In the case of circular conformation, the majority of events were identified as circular conformation.

FIG. 21. Mimicking of trans-splicing and backsplicing with T4 RNA ligase 1. (A) Experimental design of ‘alternative splicing’ with T4 RNA ligase 1. Circularization assay promotes intramolecular ligation (circularization) with potential intermolecular ligation. (B) Example events identified after ligation with nanopore measurements. circRNA conformation was fixed with two weak oligo linkers and successfully identified. Both the original RNA with unique RNA ID ‘111’ and the trans-spliced variant were detected.

FIG. 22. The method of the invention discriminates alternative splicing isoforms in a complex human transcriptome mixture. (A) Four identified enolase 1 (ENO1) transcript isoform ID designs and example events. (B) Quantification of each ENO1 transcript variant for three individual nanopore measurements. 18S rRNA ‘1111’ was used as internal control with 107±12 events/h. Total events detected were 39521 for three nanopores. (C) Design of Xist lncRNA length isoforms IDs comprising both linearising-structural units (labelled ‘1’) and native structural units (produced by single-stranded regions of nucleic acid) with their representative events are shown (longer L-isoform and shorter S-isoform).

FIG. 23. Xist lncRNA ID design.

FIG. 24. Enrichment of target RNA ID from a background of short nucleic acid fragments (<100 kDa). (A) Cumulative events for MS2 ID ‘111’p after RNA ID fabrication with and without enrichment. The ionic current trace after enrichment indicates the removal of short nucleic acid background. (B) Agarose gel indicates successful removal of oligos and short RNAs after enrichment. Gel: 1% (w/v) agarose, 1×TBE.

FIG. 25. Self-assembled RNA ID for RNA motif mapping. a) Native target 3D RNA molecule is reshaped to a linear RNA ID by contacting with linearising units comprising docking strands (short complementary oligonucleotides). b) Regions of the target RNA that are not bound by linearising units self-assemble into secondary structures thereby forming native structural unit(s) (labelled ‘I’). c) Target RNA molecule pre-treated with RNA binding molecule (e.g. protein, nucleic acid, ligand, small molecule etc.) is mixed with linearising units to form RNA ID. RNA binding molecules block the interaction between the target RNA and linearising units, thereby preventing the formation of double-stranded regions. These unhybridized sites self-assemble to form native structural units of different sizes (labelled ‘1’, ‘2’, or ‘3’), referred to herein as structural colours. These native structural units/colours can be localized, sized, and quantified with nanopore measurement to characterise the binding site(s) and/or activity of the RNA binding molecule.

FIG. 26. Self-assembled RNA ID for RNA motif mapping. Target 3D RNA molecule is reshaped to a linear RNA ID by contacting with linearising units (black lines) comprising docking strands having a region that is complementary to the target RNA. When the target RNA has been pre-treated with RNA binding molecule(s) (e.g. protein, nucleic acid, ligand, small molecule etc.), sites in the target RNA that are occupied or stabilized by RNA binding molecules are prevented from interacting with the linearising units and remain unhybridized. These unhybridized sites self-assemble to form native structural units (RNA secondary structures). Such structural units can be localized, sized, and quantified with nanopore measurement to characterise the binding site(s) and/or activity of the RNA binding molecule.

FIG. 27. Exemplary design of native (RNA origami) structural units and structural colours. (A) Exemplary RNA origami ID designed to have three native structural units provided by secondary structures formed by single-stranded regions of the target nucleic acid. Linearising units (black lines) are designed to provide native structural units at locations I, U, and Y. Each native structural unit represents a specific structure (structural colour) with a unique current downward signal when detected using a nanopore microscope. (B) The inventors demonstrated that the terminal ends of a target RNA provide native structural units when not complemented with linearising units. (C) The inventors also demonstrated that linearising units that are complementary to only a region of the target nucleic acid (e.g. RNA) is sufficient to provide an ID (provided by one or more structural unit(s) interspaced by one or more regions of double-stranded nucleic acid). As demonstrated, an ID may comprise native structural units (self-assembled RNA terminal structures represented by Q and W in part B); linearising-structural units (represented by structures within square brackets in part C); and double-stranded nucleic acid regions (RNA-DNA hybrid origami (grey-black)). (D) Predicted 2D and 3D structures of designed native structural units (RNA origamis) corresponding to I, U, and Y shown in part A. (E) Heatmap indicating correct identification of I, U, and Y with 99.4%, 99.1%, and 99.2% accuracy, respectively, using nanopore-based detection. (F) Heatmap indicating correct identification of terminal structural units Q and W with 100% accuracy using nanopore-based detection. Identification of terminal structural units can be used to determine the directionality of RNA translocation events.

FIG. 28. Agarose gel analysis of RNA IDs. 0.8% (w/v) agarose gel in 1×TBE (Tris-borate-EDTA) of RNA IDs. Lanes: 1-1 kb ladder (NEB); 2—ssRNA ladder (NEB); 3—E. coli total RNA; 4—RNA ID assembly at 70° C., 5 min; 5—RNA ID assembly at 80° C., 5 min; 6—RNA ID assembly at 90° C., 5 min; 7—RNA ID assembly at 100° C., 5 min. Lanes 4-7 show E. coli 16S rRNA ID ‘1131’.

FIG. 29. RNA ID design of E. coli 16S ribosomal RNA annotated as ‘1131’. Nanopore readout clearly shows an example event for an RNA ID generated at 100° C., 5 min assembly.

FIG. 30. Nanopore events for RNA ID ‘111’ (a) in the absence of linearisation; and (b) in the presence of linearisation indicating that in the absence of linearisation, it is not possible to distinguish between structural units. Illustrative RNA ID ‘111’ was assembled by mixing 3,569 nt MS2 RNA with oligonucleotides forming structural colours in (a) the absence of linearisation and (b) the presence of linearisation. Illustrative RNA ID ‘111’ production is described in Example 2.

### DETAILED DESCRIPTION OF THE INVENTION

Methods for characterising nucleic acids typically rely on enzymatic processing of the nucleic acid prior to detection. For example, methods for characterising RNA (e.g. RNA transcripts) typically require reverse transcription of the RNA to produce cDNA which is then amplified prior to detection. These enzymatic processing steps are problematic because they are susceptible to enzymatic biases which reduce the reproducibility and reliability of results.

Nucleic acid characterisation methods in the art often also involve fragmentation of target nucleic acids prior to characterisation which impedes the ability of these methods to characterise conformational and/or structural variations. In transcriptomic methods such as RNA-seq, RNA and/or cDNA is typically fragmented prior to detection which has the potential to disrupt the structure of transcript variants. Methods which require fragmentation and/or enzymatic processing are also unable to detect and differentiate between conformational variants, e.g. circular and linear variants, because conformational features of the native nucleic acid are lost during fragmentation or enzymatic processing, e.g. when RNA is converted to cDNA.

The inventors have overcome these problems by developing a method for characterising target nucleic acid(s) by contacting the target nucleic acid with linearising units to provide one or more structural unit(s) interspaced by one or more regions of double-stranded nucleic acid. The linearising units comprise a docking strand having a region that is complementary to a distinct region of the target nucleic acid and, when bound to the complementary region of the target nucleic acid, the docking strand reduces the secondary structure thereof. Detection of structural unit(s) along the target nucleic acid allows the target nucleic acid to be characterised.

In some embodiments, the structural unit(s) is provided by one or more linearising unit(s). Structural units provided by the linearising units are referred to herein as linearising-structural units. In this embodiment, detecting the structural unit(s) along the target nucleic acid comprises detecting the linearising unit(s) that provide the structural unit(s). Linearising-structural unit(s) typically comprise a label. In some embodiments, the one or more linearising-structural unit(s) comprises: (i) a docking strand having a region that is complementary to distinct region(s) of the target nucleic acid and an overhang region; and (ii) a labelling strand that is complementary to the overhang region of the docking strand and comprises a label.

In some embodiments, the one or more linearising-structural unit(s) comprises a docking strand having a region that is complementary to distinct region(s) of the target nucleic acid and a labelling region. The labelling region may comprise a structural label, e.g. a nucleic acid nanostructure, or may be conjugated to a label.

In some embodiments, the structural unit(s) is provided by single-stranded region(s) of the target nucleic acid. Said single-stranded region(s) are not bound by linearising unit(s). In some embodiments, one or more of the linearising unit(s) are separated by single-stranded region(s) of the target nucleic acid, and the structural unit(s) is provided by secondary structures formed by said single-stranded region(s) of the target nucleic acid. Structural unit(s) provided by single-stranded regions of the target nucleic acid that are not bound by linearising unit(s) are referred to herein as native structural unit(s). When used in the context of structural units, ‘native’ means that the structural unit is formed by secondary structures within the target nucleic acid. Regions of the target nucleic acid that are bound by linearising units are non-native.

In some embodiments, detecting structural units comprises detecting the sequence of structural units along the target nucleic acid. The sequence of structural units along the target nucleic acid is referred to herein as an identifier (ID). An ID is typically unique to a particular target nucleic acid and can be used to characterise the target nucleic acid. Structural unit sequences (IDs) comprise structural units interspaced by one or more regions of double-stranded nucleic acid provided by linearising units. IDs may comprise linearising-structural units, native structural units or both.

The method of the invention advantageously characterises target nucleic acids in their native form, without requiring enzymatic processing (e.g. reverse transcription or amplification). This allows both the structure and the conformation of the target nucleic acid(s) to be characterised. For example, the methods of the invention may advantageously be used to identify and/or differentiate structural (e.g. isoform) and conformational (e.g. linear and circular) variants.

As demonstrated herein, the methods of the invention can also successfully characterise target nucleic acid(s) in a complex mixture of nucleic acids, e.g. human total RNA. The methods of the invention can also characterise and differentiate several target nucleic acids in a single reaction, even when present at low abundances.

RNA molecules are difficult to characterise directly due to the presence of complex secondary structures which self-assemble within the RNA molecule (e.g. stem and loop structures). Existing methods for characterising RNA typically involve converting RNA to DNA (which is typically thought to be more stable than RNA) to remove RNA secondary structures prior to analysis. Surprisingly, the inventors have found that the methods of the invention may be used to characterise RNA directly (without requiring e.g. enzymatic conversion to DNA, or complete removal of secondary structures). In the methods of the invention, target RNA is contacted with one or more linearising unit(s). Each linearising unit comprises a docking strand having a region that is complementary to a distinct region of the target RNA. Binding of the docking strand to the target RNA reduces the secondary structure of that region of the target RNA which advantageously allows structural units to be readily identified. Advantageously, the inventors have demonstrated herein that RNA molecules bound to linearising units exhibit good stability with minimal degradation under standard storage conditions (e.g. when stored at about 4° C. or about −20° C.).

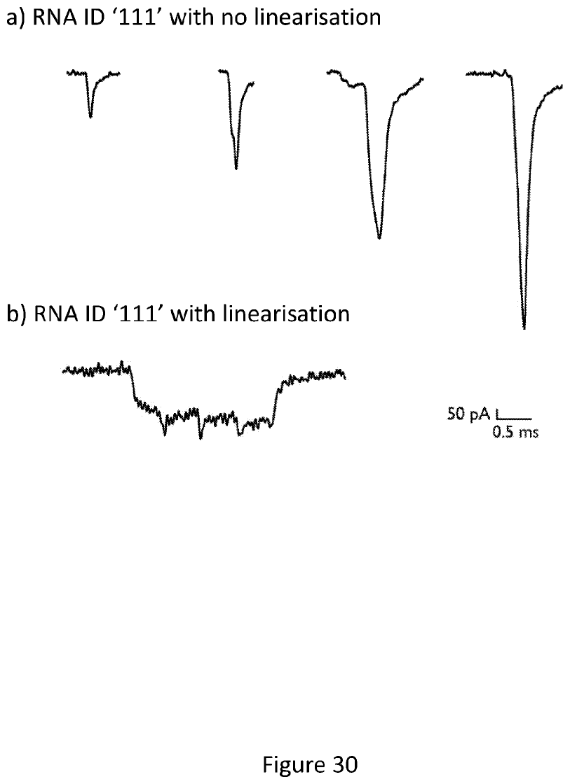

Methods of the invention comprise contacting the target nucleic acid with one or more linearising unit(s) to provide one or more structural unit(s) interspaced by one or more regions of double-stranded nucleic acid. The interspaced double-stranded nucleic acid regions provide linearisation of the target nucleic acid by reducing secondary structure and thereby allow the structural unit(s) to be distinguished. In the absence of linearisation, a single signal is provided by an RNA ID and structural units cannot be identified or distinguished (see FIG. 30(a) which provides exemplary ID events for illustrative RNA ID ‘111’, in the absence of linearisation). Advantageously, linearisation according to the invention enables each structural unit to produce a separate signal that can be identified and distinguished (see FIG. 30(b) which provides exemplary ID events for illustrative RNA ID ‘111’ formed by the method of the invention, in the presence of linearisation).

In some embodiments, the methods of the invention are used to characterise RNA transcript isoform(s) at the single-molecule level. Isoform IDs typically comprise structural units that are specific to distinct regions of the target RNA transcript (e.g. distinct exons). When annealed to the target RNA transcript, the sequence of structural units (ID) that is produced can be used to identify a particular RNA transcript isoform. Isoform IDs may comprise native and/or linearising structural units. The method of the invention advantageously enables simultaneous detection and quantification of multiple distinct transcripts and transcript isoforms, including circular and linear transcript conformations.

### Linearising Units

The method of the invention comprises contacting the target nucleic acid with one or more linearising unit(s) to provide one or more structural unit(s) interspaced by one or more regions of double-stranded nucleic acid. Each linearising unit comprises a docking strand having a region that is complementary to distinct region(s) of the target nucleic acid. The docking strand(s) of the linearising unit(s) bind to complementary regions of the target nucleic acid via specific base pairing interactions to form double-stranded regions (target nucleic acid: linearising unit hybrid regions). Binding of the docking strand(s) to complementary regions of the target nucleic acid disrupts, prevents and/or reduces secondary structures within these regions of the target nucleic acid because intramolecular base pairing interactions are disrupted or prevented from forming.

The sample is contacted with one or more linearising unit(s) under conditions that allow the one or more linearising unit(s) to bind to complementary regions of the target nucleic acid. The linearising unit binding phase may comprise incubating the target nucleic acid with one or more linearising unit(s) at a temperature that is optimal for linearising units to anneal to the target nucleic acid. The temperature may be identified by routine optimisation and will vary depending on the nature of the target nucleic acid and the linearising units used.

The one or more linearising unit(s) may comprise 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500 or more linearising units that anneal to distinct regions of the target nucleic acid. For example, the one or more linearising unit(s) may comprise 2 or more, 3 or more, 4 or more, 5 or more, 6 or more, 7 or more, 8 or more, 9 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 110 or more, 120 or more, 130 or more, 140 or more, 150 or more, 160 or more, 170 or more, 180 or more, 190 or more, 200 or more, 250 or more, 300 or more, 350 or more, 400 or more, 450 or more, 500 or more linearising units that anneal to distinct regions of the target nucleic acid.

In some embodiments, the docking strand is 10-100 nucleotides (nt) in length. In some embodiments, the docking strand is 10-100 nt, 10-90 nt, 10-80 nt, 10-70 nt, 10-60 nt, 10-50 nt, 10-45 nt, 10-40 nt, 10-35 nt, 10-30 nt, 10-25 nt, 10-20 nt, 20-100 nt, 20-90 nt, 20-80 nt, 20-70 nt, 20-60 nt, 20-50 nt, 20-45 nt, 20-40 nt, 20-35 nt, 20-35 nt, 20-30 nt, 20-25 nt, 30-100 nt, 30-90 nt, 30-80 nt, 30-70 nt, 30-60 nt, 30-50 nt, 30-45 nt, 30-40 nt, 30-35 nt, 40-100 nt, 40-90 nt, 40-80 nt, 40-70 nt, 40-60 nt, 40-50 nt, or 40-45 nt in length. In some embodiments, the docking strand is 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt in length.

In some embodiments, the region of the docking strand that is complementary to the target nucleic acid sequence is 10-100 nt, 10-90 nt, 10-80 nt, 10-70 nt, 10-60 nt, 10-50 nt, 10-45 nt, 10-40 nt, 10-35 nt, 10-30 nt, 10-25 nt, 10-20 nt, 20-100 nt, 20-90 nt, 20-80 nt, 20-70 nt, 20-60 nt, 20-50 nt, 20-45 nt, 20-40 nt, 20-35 nt, 20-35 nt, 20-30 nt, 20-25 nt, 30-100 nt, 30-90 nt, 30-80 nt, 30-70 nt, 30-60 nt, 30-50 nt, 30-45 nt, 30-40 nt, 30-35 nt, 40-100 nt, 40-90 nt, 40-80 nt, 40-70 nt, 40-60 nt, 40-50 nt, or 40-45 nt in length. In some embodiments, the region of the docking strand that is complementary to the target nucleic acid sequence is 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt in length.

The docking strand may be formed using any nucleic acid, including but not limited to DNA, RNA, xeno nucleic acid (XNA), and peptide nucleic acid (PNA).

In some embodiments, the target nucleic acid is RNA and the linearising unit docking strand comprises DNA.

In some embodiments, the target nucleic acid is contacted with one or more linearising units that are complementary to the full length of the target nucleic acid. In some embodiments, the target nucleic acid is contacted with one or more linearising units that are complementary to a region of the target nucleic acid.

### Linearising-Structural Unit

In some embodiments, one or more structural unit(s) is provided by linearising unit(s). Structural units provided by linearising units are referred to herein as linearising-structural units.

In some embodiments, one or more linearising unit(s) comprise: (i) docking strand having a region that is complementary to distinct region(s) of the target nucleic acid and an overhang region; and (ii) a labelling strand that is complementary to the overhang region of the docking strand and comprises a label.

In some embodiments, the docking strand comprises an overhang. An overhang comprises at least one unpaired nucleotide. The overhang region of the docking strand comprises nucleotides that are not complementary to the target nucleic acid and thus do not hybridise thereto. In some embodiments, the overhang region of the docking strand is 10-100 nt, 10-90 nt, 10-80 nt, 10-70 nt, 10-60 nt, 10-50 nt, 10-45 nt, 10-40 nt, 10-35 nt, 10-30 nt, 10-25 nt, 10-20 nt, 20-100 nt, 20-90 nt, 20-80 nt, 20-70 nt, 20-60 nt, 20-50 nt, 20-45 nt, 20-40 nt, 20-35 nt, 20-35 nt, 20-30 nt, 20-25 nt, 30-100 nt, 30-90 nt, 30-80 nt, 30-70 nt, 30-60 nt, 30-50 nt, 30-45 nt, 30-40 nt, 30-35 nt, 40-100 nt, 40-90 nt, 40-80 nt, 40-70 nt, 40-60 nt, 40-50 nt, or 40-45 nt in length. In some embodiments, the overhang region of the docking strand is 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt in length.

In some embodiments, the linearising unit comprises a labelling strand. In some embodiments, the labelling strand (which may also be referred to herein as the “imaging strand”) comprises a region that is complementary to the overhang region of the docking strand. In some embodiments, the labelling strand is fully complementary to the overhang region of the docking strand. In some embodiments, the labelling strand is 10-100 nt, 10-90 nt, 10-80 nt, 10-70 nt, 10-60 nt, 10-50 nt, 10-45 nt, 10-40 nt, 10-35 nt, 10-30 nt, 10-25 nt, 10-20 nt, 20-100 nt, 20-90 nt, 20-80 nt, 20-70 nt, 20-60 nt, 20-50 nt, 20-45 nt, 20-40 nt, 20-35 nt, 20-35 nt, 20-30 nt, 20-25 nt, 30-100 nt, 30-90 nt, 30-80 nt, 30-70 nt, 30-60 nt, 30-50 nt, 30-45 nt, 30-40 nt, 30-35 nt, 40-100 nt, 40-90 nt, 40-80 nt, 40-70 nt, 40-60 nt, 40-50 nt, or 40-45 nt in length. In some embodiments, the labelling strand is 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt in length.

The labelling strand may be formed using any nucleic acid, including but not limited to DNA, RNA, xeno nucleic acid (XNA), and peptide nucleic acid (PNA).

In some embodiments, one or more linearising unit(s) comprise a docking strand having a region that is complementary to distinct region(s) of the target nucleic acid and a labelling region that is not complementary to the target nucleic acid. In some embodiments, the labelling region is 10-100 nt, 10-90 nt, 10-80 nt, 10-70 nt, 10-60 nt, 10-50 nt, 10-45 nt, 10-40 nt, 10-35 nt, 10-30 nt, 10-25 nt, 10-20 nt, 20-100 nt, 20-90 nt, 20-80 nt, 20-70 nt, 20-60 nt, 20-50 nt, 20-45 nt, 20-40 nt, 20-35 nt, 20-35 nt, 20-30 nt, 20-25 nt, 30-100 nt, 30-90 nt, 30-80 nt, 30-70 nt, 30-60 nt, 30-50 nt, 30-45 nt, 30-40 nt, 30-35 nt, 40-100 nt, 40-90 nt, 40-80 nt, 40-70 nt, 40-60 nt, 40-50 nt, or 40-45 nt. In some embodiments, the labelling region is 10 nt, 15 nt, 20 nt, 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, or 100 nt in length.

The labelling region may be located at any position within the docking strand, e.g. at a terminal end of the region that is complementary to the target nucleic acid or within the region that is complementary to the target nucleic acid wherein the labelling region is flanked by regions that are complementary to the target nucleic acid.

The labelling strand and/or region comprises a label that can be detected using any suitable method known in the art, e.g. nanopore or fluorescence based detection methods. In some embodiments, the labelling strand and/or region comprises a structural label (e.g. nucleic acid nanostructure). In some embodiments, the labelling strand and/or region comprises a fluorescent label. In some embodiments, the labelling strand and/or region comprises a structural label and a fluorescent label. In some embodiments, the labelling region comprises secondary structures within the labelling region such as loop-stem structures or nucleic acid double hairpin structures. In some embodiments, the labelling region comprises one or more DNA double hairpin structures, e.g. 1, 2, 3, 4, 5, 6, 7, 8, 9, or 10 DNA double hairpin structures. The detectable label may be a label that is attached to the labelling region.

A structural label may be detected by a nanopore-based detection method, wherein the structural label produces an identifiable current change when translocated through the nanopore. In some embodiments, the structural label is selected from a nucleic acid nanostructure (e.g. DNA cuboid, nucleic acid double hairpin structure), biotin, avidin, neutravidin, streptavidin, or traptavidin, or a biotin/avidin, biotin/neutravidin, biotin/streptavidin, or biotin/traptavidin complex. References herein to avidin should be understood to encompass streptavidin, neutravidin, and traptavidin, and vice versa. Avidin, neutravidin, traptavidin and streptavidin for use in the methods of the invention are typically monomeric or monovalent, although multimeric forms (e.g. divalent trivalent or tetravalent) may also be employed.

In some embodiments, the labelling strand and/or region is biotinylated (i.e. the labelling strand and/or region is covalently attached to biotin). In some embodiments, the labelling strand and/or region is biotinylated and the method comprises contacting the target nucleic acid with avidin, neutravidin, traptavidin or streptavidin. In some embodiments, the structural label comprises a nucleic acid nanostructure, e.g. DNA cuboid, or double hairpin structure. In some embodiments, the labelling strand and/or region is conjugated to an antigen and the method comprises contacting the labelling strand and/or region with an antigen binding molecule specific for the antigen, e.g. an antibody.

In some embodiments, structural unit(s) comprising a fluorescent label are detected using a fluorescence-based detection method. A fluorescent label may be detected by fluorescence microscopy. For example, a fluorescent label may be detected by binding activated localisation microscopy (BALM), total internal reflection fluorescence (TIRF) microscopy, stochastic optical reconstruction microscopy (STORM), or stimulated emission depletion (STED) microscopy. In some embodiments, the labelling strand and/or region is conjugated to a fluorophore, e.g. 6-carboxyfluorescein (6-FAM). In some embodiments, the labelling strand and/or region is conjugated to an antigen and the method comprises contacting the labelling strand with an antigen binding molecule specific for the antigen, wherein the antigen binding molecule comprises a fluorescent label, e.g. an antibody conjugated to a fluorescent label.

In some embodiments, each linearising-structural unit comprises a different label. For example, each linearising-structural unit may comprise a label having a different molecular weight and/or different number of fluorophores.

In some embodiments, the docking strand is annealed to the labelling strand prior to contacting the target nucleic acid with linearising-structural unit(s). In some embodiments, the target nucleic acid is contacted with the docking strand of linearising-structural unit(s) and subsequently contacted with the labelling strand of linearising-structural unit(s).

### Native Structural Units

In some embodiments, one or more structural unit(s) is provided by single-stranded regions of the target nucleic acid. Structural units provided by the target nucleic acid are referred to herein as native structural units.

In some embodiments, one or more of the linearising unit(s) are separated by single-stranded region(s) of the target nucleic acid, and one or more of the structural unit(s) is provided by secondary structures formed by said single-stranded region(s) of the target nucleic acid. Said single-stranded region(s) of the target nucleic acid are not bound by linearising unit(s) and self-assemble to form secondary structure(s).

As used herein, a secondary structure refers to a three-dimensional conformation that is formed by interactions between bases of the same single-stranded region of nucleic acid. Exemplary secondary structures include, but are not limited to, nucleic acid coils, hairpin structures, stem-loop structures, internal loops, bulge loops, branched structures, multiple stem loop structures, cloverleaf type structures or any three dimensional structure.

In some embodiments, native structural units are 10 nt or more, 20 nt or more, 30 nt or more, 40 nt or more, 50 nt or more, 60 nt or more, 70 nt or more, 80 nt or more, 90 nt or more, 100 nt or more, 110 nt or more, 120 nt or more, 130 nt or more, 140 nt or more, 150 nt or more, 160 nt or more, 170 nt or more, 180 nt or more, 190 nt or more, 200 nt or more, 250 nt or more, 300 nt or more, 350 nt or more, 400 nt or more, 450 nt or more, 500 nt or more, 550 nt or more, 600 nt or more, 650 nt or more, 700 nt or more, 750 nt or more, 800 nt or more, 850 nt or more, 900 nt or more, 950 nt or more, 1000 nt or more, 1500 nt or more, 2000 nt or more, 2500 nt or more, 3000 nt or more, 3500 nt or more, 4000 nt or more, 4500 nt or more, or 5000 nt or more in length.

Native structural unit(s) may be detected by nanopore-based detection method, wherein native structural unit(s) produces an identifiable current change when translocated through the nanopore.

### Structural Colours

In some embodiments, linearising units provide one or more structural colour(s) interspaced by one or more regions of double-stranded nucleic acid. In some embodiments, structural colour(s) comprise: (a) an integer number of adjacent structural units detectable as a single signal; and/or (b) structural units which provide a distinct signal when detected.

As used herein, the term ‘structural colour’ refers to structural unit(s) that produce a single detectable signal and that can be differentiated from different structural unit(s) and/or colour(s) based on the strength of the signal produced.

In some embodiments, each structural colour comprises an integer number of structural units which are detectable as a single signal. For example, structural colours may comprise an integer number of linearising-structural units designed to ensure that labels associated with each linearising-structural unit are detected as a single signal, e.g. a single fluorescence level or single nanopore current peak.

Advantageously, linearising-structural units comprising the same type of label can be used to produce distinct structural colours which can be detected and differentiated based on the strength of their respective signals. The ability to detect and differentiate multiple signals that are generated by the same type of label is advantageous e.g. because it can simplify experimental design and reduce cost. For example, when a single type of label is used, the same detection method can identify several distinct structural colours without requiring additional calibration (e.g. as would be required to detect several different types of label). The use of the same label also avoids potential errors introduced by labelling and/or detection biases which may exist between different types of labels (e.g. between different sets of ligand-receptor pairs). Furthermore, structural colours can be incorporated into sequence IDs to further improve the multiplexing capabilities of the invention without requiring modification of the method.

In some embodiments, structural colour(s) comprise an integer number of adjacent linearising-structural units that produce a single detectable signal. For example, structural colour ‘1’ may correspond to a single linearising-structural unit; and structural colour ‘2’ may correspond to two adjacent linearising-structural units that produce a single detectable signal. In this embodiment, the signal produced by the structural colour is determined by the number of linearising-structural units that form the structural colour and the type of label present. For example, structural colours produced by adjacent linearising-structural units comprising structural labels will have varying molecular weights, whereas structural colours produced by adjacent linearising-structural units comprising fluorescent labels will produce varying fluorescence levels. The skilled person will understand that in this embodiment, the strength of the signal will correspond to the number of linearising-structural units present, e.g. structural colour ‘10’ comprises ten adjacent linearising-structural units (and therefore ten labels) which will produce a greater signal than structural colour ‘5’ which comprises five adjacent linearising-structural units (and therefore five labels).

As used herein, adjacent linearising-structural units typically means that the linearising-structural units are complementary to sequential regions of the target nucleic acid sequence. In some embodiments, structural colour(s) comprise linearising-structural units that are complementary to regions of the target nucleic acid that are separated by 20 nt, 19 nt, 18 nt, 17 nt, 16 nt, 15 nt, 14 nt, 13 nt, 12 nt, 11 nt, 10 nt, 9 nt, 8 nt, 7 nt, 6 nt, 5 nt, 4 nt, 3 nt, 2 nt, 1 nt, or 0 nt. In some embodiments, structural colour(s) comprises linearising-structural units that are complementary to regions of the target nucleic acid that are separated by 20 nt or fewer, 19 nt or fewer, 18 nt or fewer, 17 nt or fewer, 16 nt or fewer, 15 nt or fewer, 14 nt or fewer, 13 nt or fewer, 12 nt or fewer, 11 nt or fewer, 10 nt or fewer, 9 nt or fewer, 8 nt or fewer, 7 nt or fewer, 6 nt or fewer, 5 nt or fewer, 4 nt or fewer, 3 nt or fewer, 2 nt or fewer, or 1 nt or fewer.

In some embodiments, structural colours comprise between 0 and 50 linearising-structural units. In some embodiments, structural colours comprise between: 0 and 45, 0 and 40, 0 and 35, 0 and 30, 0 and 25, 0 and 20, 0 and 15, 0 and 10, 0 and 9, 0 and 8, 0 and 7, 0 and 6, 0 and 5, 0 and 4, 0 and 3, 0 and 2, 1 and 50, 1 and 45, 1 and 40, 1 and 35, 1 and 30, 1 and 25, 1 and 20, 1 and 15, 1 and 10, 1 and 9, 1 and 8, 1 and 7, 1 and 6, 1 and 5, 1 and 4, 1 and 3, 1 and 2, 2 and 50, 2 and 45, 2 and 40, 2 and 35, 2 and 30, 2 and 25, 2 and 20, 2 and 15, 2 and 10, 2 and 9, 2 and 8, 2 and 7, 2 and 6, 2 and 5, 2 and 4, 2 and 3, 3 and 50, 3 and 45, 3 and 40, 3 and 35, 3 and 30, 3 and 25, 3 and 20, 3 and 15, 3 and 10, 3 and 9, 3 and 8, 3 and 7, 3 and 6, 3 and 5, 3 and 4, 4 and 50, 4 and 45, 4 and 40, 4 and 35, 4 and 30, 4 and 25, 4 and 20, 4 and 15, 4 and 10, 4 and 9, 4 and 8, 4 and 7, 4 and 6, 4 and 5, 5 and 50, 5 and 45, 5 and 40, 5 and 35, 5 and 30, 5 and 25, 5 and 20, 5 and 15, 5 and 10, 5 and 9, 5 and 8, 5 and 7, 5 and 6, 6 and 50, 6 and 45, 6 and 40, 6 and 35, 6 and 30, 6 and 25, 6 and 20, 6 and 15, 6 and 10, 6 and 9, 6 and 8, 6 and 7, 7 and 50, 7 and 45, 7 and 40, 7 and 35, 7 and 30, 7 and 25, 7 and 20, 7 and 15, 7 and 10, 7 and 9, 7 and 8, 8 and 50, 8 and 45, 8 and 40, 8 and 35, 8 and 30, 8 and 25, 8 and 20, 8 and 15, 8 and 10, 8 and 9, 9 and 50, 9 and 45, 9 and 40, 9 and 35, 9 and 30, 9 and 25, 9 and 20, 9 and 15, 9 and 10, 10 and 50, 10 and 45, 10 and 40, 10 and 35, 10 and 30, 10 and 25, 10 and 20, and 10 and 15 linearising-structural units. In some embodiments, structural colours comprise more than 50 linearising-structural units.

In some embodiments, each structural colour comprises structural unit(s) which provide a distinct signal when detected. As used herein, a structural unit which provides a distinct signal means that when detected, the structural unit produces a signal that is different and distinguishable from other structural unit(s)/structural colour(s) used in the method of the invention.

In some embodiments, each structural colour comprises a linearising-structural unit comprising a label of distinct size or a distinct number of labels. In this embodiment, the signal produced by the structural colour is determined by the size and/or number of labels present on the linearising-structural unit.

In some embodiments, each structural colour comprises linearising-structural unit comprising a label that exhibits a different charge to other structural unit(s). In nanopore-based detection methods, the current change produced when structural unit(s)/colour(s) are translocated varies depending on the charge associated with the structural unit/colour. The inventors have found that by making an ID using either DNA nanocuboid structures or monovalent streptavidin as a label, the DNA nanocuboid labelled structural units/colours exhibit increased velocity of ID translocation in nanopore and therefore decreased current blockage relative to streptavidin labelled structural units/colours.

In some embodiments, each structural colour comprises a native structural unit of distinct size. In this embodiment, the signal produced by the structural colour(s) is determined by the length of the single-stranded region which forms the native structural unit, wherein longer single-stranded regions provide larger structural units (with greater molecular weight) than shorter single-stranded regions. In this embodiment, structural colours have varying molecular weights and can be distinguished by the strength of the signal they produce e.g. native structural colours with higher molecular weights will produce a greater reduction in current when translocated through a nanopore than native structural colours with lower molecular weights.

Advantageously, structural colours further enhance the multiplexing capacity of the method. For example, unique IDs can be designed using a distinct structural colour for each target nucleic acid, or using a unique sequence of structural colours for each target nucleic acid. In embodiments wherein the target nucleic acid is an RNA transcript, each exon may be labelled with a distinct structural colour or sequence of structural colours.

### Detecting Structural Units Along the Target Nucleic Acid

The method of the invention comprises detecting structural unit(s) along the target nucleic acid. In some embodiments, the method of the invention comprises determining the sequence of structural units along the target nucleic acid. In some embodiments, the target nucleic acid is characterised by the sequence of structural units along the target nucleic acid.

In some embodiments, unbound linearising units are removed from the mixture prior to detecting structural unit(s) along the target nucleic acid.

In some embodiments, the method of the invention comprises determining the sequence of structural colours along the target nucleic acid and characterising the target nucleic acid by the sequence of structural colours detected. The sequence of structural units and/or structural colours may be determined in the 5′ to 3′ direction or the 3′ to 5′ direction of the target nucleic acid. In some embodiments, excess linearising units are removed prior to detection of structural unit(s) along the target nucleic acid.

In some embodiments, the method of the invention comprises determining the sequence of structural units and/or structural colours by determining the position of structural units and/or structural colours relative to the terminal ends of the target nucleic acid. In some embodiments, one or both terminal end(s) of the target nucleic acid is not bound by linearising units. In this embodiment, the terminal end(s) of the target nucleic acid provide a native structural unit.

Structural units/colours comprising structural label(s) include: native structural units wherein the structural unit/colour is provided by secondary structures formed by single-stranded region(s) of the target nucleic acid; and linearising-structural units comprising a labelling strand and/or region having a structural label. In embodiments wherein structural unit(s)/structural colour(s) comprise structural labels, structural unit(s) and/or structural colour(s) may be detected using e.g. nanopore-based detection methods, also referred to herein as nanopore microscopy. Detecting structural unit(s) and/or structural colour(s) using nanopore-based detection methods provides a rapid, enzyme-free, and low cost alternative to short and long read sequencing. Advantageously, nanopores overcome the technical artifacts of RNA-seq and imperfections of motor proteins used in traditional nanopore sequencing methods.

In nanopore-based detection methods, ions pass through a nanopore due to an applied potential and create an ionic current. When nucleic acids translocate through a nanopore, a current signature or current trace is produced which corresponds to the current level detected over time as the nucleic acid translocates through the nanopore. The current signature (also referred to herein as a ‘nanopore event’ or an ‘event’) may be compared to a negative control (e.g. a current signature produced by the target nucleic acid in the absence of structural unit(s)/structural colour(s)); and/or to a positive control (e.g. a current signature produced by the target nucleic acid in the presence of structural unit(s)/structural colour(s)). Structural labels produce an identifiable current signal (reduction in current), when translocated through a nanopore.

In some embodiments, structural colours are provided by structural units which comprise different structural labels that can be differentiated based on the change in current signal they produce when translocated through a nanopore. For example, structural colours produced by native structural units vary in size, with larger native structural units (produced by longer single-stranded regions of target nucleic acid) producing a larger decrease in current when translocated through the nanopore than smaller native structural units (produced by shorter single-stranded regions of target nucleic acid).

The nanopore may be a solid state or a biological nanopore. In some embodiments, the nanopore is a glass nanopore. In some embodiments, nanopores used to detect structural units along the target nucleic acid comprise a diameter of about 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, or 20 nm. For example, nanopores used to detect structural units along the target nucleic acid comprise a diameter of about 3 nm-about 20 nm, about 3 nm-about 19 nm, about 3 nm-about 18 nm, about 3 nm-about 17 nm, about 3 nm-about 16 nm, about 3 nm-about 15 nm, about 3 nm-about 14 nm, about 3 nm-about 13 nm, about 3 nm-about 12 nm, about 3 nm-about 11 nm, about 3 nm-about 10 nm, about 3 nm-about 9 nm, about 3 nm-about 8 nm, about 3 nm-about 7 nm, about 3 nm-about 6 nm, about 3 nm-about 5 nm, about 3 nm-about 4 nm, 4 nm-about 20 nm, about 4 nm-about 19 nm, about 4 nm-about 18 nm, about 4 nm-about 17 nm, about 4 nm-about 16 nm, about 4 nm-about 15 nm, about 4 nm-about 14 nm, about 4 nm-about 13 nm, about 4 nm-about 12 nm, about 4 nm-about 11 nm, about 4 nm-about 10 nm, about 4 nm-about 9 nm, about 4 nm-about 8 nm, about 4 nm-about 7 nm, about 4 nm-about 6 nm, about 4 nm-about 5 nm, 5 nm-about 20 nm, about 5 nm-about 19 nm, about 5 nm-about 18 nm, about 5 nm-about 17 nm, about 5 nm-about 16 nm, about 5 nm-about 15 nm, about 5 nm-about 14 nm, about 5 nm-about 13 nm, about 5 nm-about 12 nm, about 5 nm-about 11 nm, about 5 nm-about 10 nm, about 5 nm-about 9 nm, about 5 nm-about 8 nm, about 5 nm-about 7 nm, about 5 nm-about 6 nm, 6 nm-about 20 nm, about 6 nm-about 19 nm, about 6 nm-about 18 nm, about 6 nm-about 17 nm, about 6 nm-about 16 nm, about 6 nm-about 15 nm, about 6 nm-about 14 nm, about 6 nm-about 13 nm, about 6 nm-about 12 nm, about 6 nm-about 11 nm, about 6 nm-about 10 nm, about 6 nm-about 9 nm, about 6 nm-about 8 nm, about 6 nm-about 7 nm, 7 nm-about 20 nm, about 7 nm-about 19 nm, about 7 nm-about 18 nm, about 7 nm-about 17 nm, about 7 nm-about 16 nm, about 7 nm-about 15 nm, about 7 nm-about 14 nm, about 7 nm-about 13 nm, about 7 nm-about 12 nm, about 7 nm-about 11 nm, about 7 nm-about 10 nm, about 7 nm-about 9 nm, about 7 nm-about 8 nm, 8 nm-about 20 nm, about 8 nm-about 19 nm, about 8 nm-about 18 nm, about 8 nm-about 17 nm, about 8 nm-about 16 nm, about 8 nm-about 15 nm, about 8 nm-about 14 nm, about 8 nm-about 13 nm, about 8 nm-about 12 nm, about 8 nm-about 11 nm, about 8 nm-about 10 nm, about 8 nm-about 9 nm, 9 nm-about 20 nm, about 9 nm-about 19 nm, about 9 nm-about 18 nm, about 9 nm-about 17 nm, about 9 nm-about 16 nm, about 9 nm-about 15 nm, about 9 nm-about 14 nm, about 9 nm-about 13 nm, about 9 nm-about 12 nm, about 9 nm-about 11 nm, about 9 nm-about 10 nm, or about 20 nm-about 10 nm are typically used. The skilled person will readily understand that the diameter of nanopore used will be suitable for detecting structural unit(s) along the target nucleic acid.

A biological nanopore may be a transmembrane protein nanopore. Examples of transmembrane protein pores include β-barrel pores and α-helix bundle pores. β-barrel pores comprise a barrel or channel that is formed from β-strands. β-barrel pores include, but are not limited to, β-toxins, such as α-hemolysin(α-HL), anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NaIP) and other pores, such as lysenin. α-helix bundle pores comprise a barrel or channel that is formed from α-helices. α-helix bundle pores include, but are not limited to, inner membrane proteins and a outer membrane proteins, such as WZA and ClyA toxin. A biological nanopore may be a transmembrane pore derived from or based on MspA, α-HL, lysenin, CsgG, ClyA, or haemolytic protein fragaceatoxin C (FraC).

Examples of transmembrane pores derived from or based on MspA are described in WO 2012/107778. Examples of transmembrane pores derived from or based on α-hemolysin are described in WO 2010/109197. Examples of transmembrane pores derived from or based on lysenin are described in WO 2013/153359. Examples of transmembrane pores derived from or based on CsgG are described in WO 2016/034591 and WO 2019/002893. Examples of transmembrane pores derived from or based on ClyA are described in WO 2017/098322. Examples of transmembrane pores derived from or based on FraC are described in WO 2020/055246. The nanopore may be a DNA origami pore. Examples of DNA origami pores are described in WO 2013/083983, WO 2018/011603, and WO 2020/025974. The nanopore may be a solid state nanopore. Examples of solid state nanopores are described in WO 2016/127007.

Nanopores used for detection of structural colours that are produced by an integer number of adjacent linearising-structural units are chosen to ensure that a single signal is detected for each structural colour, e.g. structural colour ‘10’ (corresponding to 10 sequentially positioned linearising-structural units) will produce a single current signal on the nanopore current signature rather than 10 discrete signals. To ensure a single signal is detected for each structural colour, the region of target nucleic acid to which the structural colour binds is below the resolution limit of the nanopore. As used herein, the resolution limit of a nanopore is the minimum distance required between two structures to ensure two distinct signals are produced on the nanopore current signature when the structures are translocated through the nanopore.

In some embodiments, structural unit(s) comprise a biotin, avidin (e.g. avidin, streptavidin, traptavidin or neutravidin) or biotin/avidin label and structural unit(s) and/or structural colour(s) along the target nucleic acid are detected by detecting the presence or absence of biotin, avidin or biotin/avidin using nanopore-based detection methods. In some embodiments, the structural unit(s) comprise a biotin label and the target nucleic acid is contacted with avidin (e.g. avidin, streptavidin, traptavidin or neutravidin). In this embodiment, structural unit(s) and/or structural colour(s) along the target nucleic acid are detected by detecting the presence or absence of biotin/avidin complexes using nanopore-based detection methods.

In some embodiments, structural unit(s) comprise a DNA nanostructure label (e.g. a DNA cuboid label or double hairpin structure) and structural unit(s) and/or structural colour(s) along the target nucleic acid are detected by detecting the presence or absence of the DNA nanostructure using nanopore-based detection methods.

In some embodiments, the method further comprises characterising the length of target nucleic acids. For example, RNA transcripts having long and short (or truncated) isoforms can be differentiated using nanopore-based detection methods, wherein long isoforms comprise a native structural unit corresponding to the single-stranded region of the long isoform that is not present in the short isoform. The length of target nucleic acids may also be determined by measuring the time taken to translocate through the nanopore.

The inventors have also demonstrated that nanopore-based detection methods allow target nucleic acids to be differentiated by their conformation. Single stranded and double stranded nucleic acids produce different current signatures when translocated through a nanopore because double stranded nucleic acids have a greater diameter, and therefore produce a greater reduction in current during translocation. Using the same principles, circular nucleic acids can be differentiated from linear nucleic acids because circular nucleic acids have a greater diameter than linear nucleic acids. Thus, two target nucleic acids comprising the same sequence (and therefore the same structural unit/colour ID) can be differentiated by the conformation (circular or linear). This is particularly advantageous for applications where it is useful to determine the structural purity of a sample containing target nucleic acid, e.g. therapeutic circular RNA, exosome RNA (exoRNA), circular RNA, sponge RNAs, antisense RNAs. The structural purity of a sample may be characterised by determining the ratio of linear to circular nucleic acids.

In embodiments wherein structural units comprise fluorescent labels, structural unit(s) and/or structural colour(s) along the target nucleic acid may be detected by fluorescent microscopy. In some embodiments, target nucleic acids are applied to a surface, separated and stretched prior to detecting structural unit(s) and/or structural colour(s) along the target nucleic acid e.g. by fluorescence microscopy.

In some embodiments, structural units comprise a fluorescent label (e.g. a fluorophore) and structural unit(s) and/or structural colour(s) along the target nucleic acid are detected by detecting the presence or absence of the fluorescent label using fluorescent microscopy or fluorescence spectroscopy based detection methods. In some embodiments, the fluorescent label is detected by binding activated localisation microscopy (BALM), total internal reflection fluorescence (TIRF) microscopy, stochastic optical reconstruction microscopy (STORM), or stimulated emission depletion (STED) microscopy.

In some embodiments, structural units comprise a fluorophore label and the method comprises contacting the target nucleic acid with a quencher prior to detecting structural unit(s) and/or colour(s) along the target nucleic acid. In this embodiment, fluorophores that are not bound to the target nucleic acid are quenched, thereby reducing the background fluorescence whereas the fluorescence produced by fluorophores present on structural units along the target nucleic acid is not quenched and can be detected.

The method of the invention may comprise determining the presence or absence of target nucleic acid(s).

The method of the invention may comprise quantifying the abundance of target nucleic acid(s). In some embodiments, the abundance of target nucleic acid(s) may be determined by counting the number of target nucleic acid molecules comprising a particular sequence ID. The method may comprise quantifying the relative abundance of target nucleic acid(s). In some embodiments, the abundance of target nucleic acid(s) is determined relative to an internal control, e.g. 18S rRNA or 28s rRNA. The method may comprise quantifying the abundance of target nucleic acid(s) relative to an external control of a known concentration.

In some embodiments, structural unit(s) and/or structural colour(s) along the target nucleic acid are detected by super-resolution microscopy, e.g. binding-activated localization microscopy (BALM). In some embodiments, nucleic acid staining dyes bind to assembled IDs, but do not bind to structural unit(s) and/or structural colour(s). In this embodiment structural unit(s) and/or structural colour(s) are identified by fluorescent-depleted regions. In some embodiments, these fluorescent-depleted regions are identified using localization super-resolution microscopy, e.g. BALM.

In some embodiments, structural unit(s) and/or structural colour(s) along the target nucleic acid are detected by size-specific readout methods such as mass photometry or size-dependent lateral-flow assays. In this embodiment, RNA may be reshaped to provide a molecule with different shape and/or size, to help distinguish between different RNA IDs.

### Target Nucleic Acids

As used herein, the term “target nucleic acid” encompasses a single target nucleic acid and multiple (i.e. more than one) target nucleic acids. The target nucleic acid may comprise RNA, e.g. single-stranded RNA (ssRNA) or double-stranded RNA (dsRNA), or DNA, e.g. single-stranded DNA (ssDNA) or double-stranded DNA (dsDNA), or combinations thereof. The target nucleic acid may be messenger RNA (mRNA), precursor-mRNA (pre-mRNA), microRNA (miRNA), non-coding RNA, small interfering RNA (siRNA), short hairpin RNA (shRNA) or ribosomal RNA (rRNA). The target nucleic acid may be autosomal DNA, or mitochondrial DNA. The target nucleic acid may be a naturally occurring or synthetic nucleic acid. In some embodiments, the target nucleic acid is complementary DNA (cDNA).

In some embodiments, the target nucleic acid is single-stranded RNA.

The methods of the invention can be used to characterise target nucleic acid in its native form. As used herein, characterising target nucleic acid in its “native form” means that the target nucleic acid is not modified prior to characterisation.

When the target nucleic acid is a double-stranded nucleic acid, the method may comprise denaturing the target nucleic acid to produce single-stranded nucleic acid prior to contacting the target nucleic acid with linearising units.

The method of the invention may be used to characterise more than one target nucleic acid. For example, the method of the invention may be used to characterise 2, 3, 4, 5, 6, 7, 8, 9, 10, 11, 12, 13, 14, 15, 20, 25, 30, 40, 50, 60, 70, 80, 90, 100, 150, 200, 250, 500, or 1000 target nucleic acids. For example, the method of the invention may be used to characterise 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, 25 or more, 30 or more, 40 or more, 50 or more, 60 or more, 70 or more, 80 or more, 90 or more, 100 or more, 150 or more, 200 or more, 250 or more, 500 or more, or 1000 or more target nucleic acids.

In some embodiments, the target nucleic acid is present in a sample. In some embodiments, the sample comprises non-target nucleic acid(s). The sample may be obtained from a cell culture. The sample may be obtained from a subject. The subject may be selected from a human or a non-human animal, such as a murine, bovine, equine, ovine, canine, or feline animal. The sample may be selected from the group consisting of, but not limited to, blood, serum, plasma, saliva, sputum, urine, faeces, cerebrospinal fluid, a lung tissue sample, a bronchoalveolar lavage sample, a nose and/or throat swab sample, or a biopsy sample.

The sample may be treated prior to use in the method of the invention. For example, the sample may be treated to lyse cells and/or to remove and/or denature proteins. Nucleic acid extraction may be performed on the sample prior to use in the method of the invention. Suitable nucleic acid extraction methods are known in the art and include methods that extract total DNA and/or RNA from samples.

In one embodiment, the step of contacting the target nucleic acid with one or more linearising unit(s) comprises: (A) contacting a sample comprising, or suspected of comprising, a cell and/or a virus having the target nucleic acid with one or more linearising unit(s); and (B) lysing the cell and/or the virus. In embodiments wherein the sample comprises a cell and/or a virus having the target nucleic acid, lysis immediately contacts the linearising unit(s) with the target nucleic acid to provide one or more structural unit(s) interspaced by one or more regions of double-stranded nucleic acid. The structural unit(s) along the target nucleic acid may then be detected as described herein. In embodiments wherein the sample does not comprise a cell and/or a virus having the target nucleic acid, the linearising unit(s) remain substantially unhybridized.

In one embodiment, the sample comprises, or is suspected of comprising, a virus, optionally wherein the virus is selected from a coronavirus, Influenza virus, Zika virus, Ebola virus, Dengue virus, Hantavirus, Nairovirus, Orthobunyavirus, Phlebovirus, Flavivirus, and Alphavirus. In one embodiment, the sample comprises, or is suspected of comprising, a coronavirus, optionally SARS-CoV-2. In one embodiment, the cell is a microorganism cell, optionally a bacterial cell or a fungal cell. In one embodiment, the microorganism is a pathogen, optionally wherein the pathogen is a bacterial pathogen, fungal pathogen, protozoan pathogen or pathogenic worm. In one embodiment, the cell is a eukaryotic cell, such as a mammalian cell, e.g. a human cell. In one embodiment, the sample is selected from blood, serum, plasma, saliva, sputum, urine, faeces, cerebrospinal fluid, a lung tissue sample, a bronchoalveolar lavage sample, a nose and/or throat swab sample, or a biopsy sample.

In some embodiments, lysing the cell and/or virus comprises mechanical and/or enzymatic lysis processes. In some embodiments, lysing the cell and/or virus comprises heating the sample to at least 50° C., at least 60° C., at least 70° C., at least 80° C., at least 90° C., at least 100° C., or at least 110° C.

Thermal lysis is rapid and efficient, but is typically avoided in methods known in the art because it is associated with unwanted nucleic acid degradation, particularly RNA degradation. Advantageously, the inventors have discovered that thermal lysis may be used in the methods of the invention to allow rapid and efficient cell lysis, without risking degradation of target nucleic acid. In more detail, the inventors have discovered that the hybridisation of linearising units to target nucleic acid, e.g. RNA, at high temperatures reduced degradation of target nucleic acid as compared to control nucleic acid in the absence of linearising units. Advantageously, the inventors also found that combining the target nucleic acid with linearising units at high temperatures reduced degradation of the target nucleic acid, but not of non-target nucleic acid, thereby enriching target nucleic acid within the sample.

The invention provides a method wherein the target nucleic acid can be extracted from cells and/or viruses and hybridised to linearising units in a single reaction step. This enables target nucleic acids to be characterised directly from a sample containing a cell and/or virus of interest without the need for a separate nucleic acid extraction process. This likewise enables the identification of an absence of target nucleic acids in a sample suspected of comprising (but not comprising) a cell and/or virus of interest without the need for a separate nucleic acid extraction process. While various lysis methods may be used in the methods of the invention, the invention is advantageously compatible with thermal lysis because hybridisation to linearising units reduces thermal degradation of the target nucleic acid as compared to thermal lysis in the absence of linearising units. The methods of the invention therefore offer a number of real-world advantages, including rapid and efficient characterisation of target nucleic acids in a small number of processing steps.

RNA is a fragile molecule that easily degrades due to enzymatic cutting by RNases, and the autocatalytic hydrolysis of phosphodiester bonds. Advantageously, through the assembly of RNA:DNA identifiers (RNA ID) which include target RNA fully complemented with short DNA linearising units, RNA becomes stable for extensive periods of time, even when stored at 4° C. This increased stability is due, in part, to the inability of RNases to recognise RNA:DNA duplexes. Additionally, the RNA:DNA duplex (which may have a persistence length of about 62 nm) has more than a 50 times higher persistence length than RNA (which may have a persistence length of about 1 nm) which physically prevents close contact between the active hydroxyl group (OH) and the phosphodiester bond. Furthermore, due to the duplex structure, the OH group may be hidden within the A-form RNA:DNA hybrid groove, further enhancing stability.

Given the fragility of RNA, it is generally desirable to select buffers which are well-suited to RNA based methods. Suitable buffers are well-known in the art. For example, citrate buffers and buffers having an acidic pH are known to promote RNA stability. To promote interaction between negatively charged DNA and RNA, the buffer may contain a salt, e.g. a monovalent salt or a divalent salt. Wherein the method of the invention is performed in the presence of nucleases (e.g. RNase) and/or at or above temperatures typically associated with thermal degradation of RNA (e.g. over 70° C.), monovalent salts should be used. In such embodiments, the presence of magnesium ions is generally undesirable because magnesium ions are cofactors for various nucleases and also promote RNA fragmentation at high temperatures. In embodiments comprising the use of monovalent salts, the buffer may comprise a divalent ion chelator, particularly a magnesium chelator such as EDTA. Wherein the method of the invention is performed in the absence of nucleases (e.g. when the target RNA has been isolated) and/or at temperatures which are not typically associated with thermal degradation of RNA (e.g. up to 70° C.), buffers containing divalent and/or monovalent salts may be used. Buffers containing monovalent salts, e.g. lithium chloride, potassium chloride and/or sodium chloride, typically comprise 1×TE buffer (10 mM Tris, pH 8.0; 1 mM EDTA) to control pH and chelate divalent (e.g. magnesium) ions. Buffers containing divalent salts, e.g. magnesium chloride, typically comprise T buffer (10 mM Tris, pH 8.0). Tris-HCl may be replaced with another buffer, particularly a neutral or acidic buffer.

In some embodiments, the method further comprises contacting the sample with a RNase to degrade single-stranded and/or double-stranded RNA after formation of an RNA ID. Advantageously, the RNA ID comprises fully complementary RNA:DNA hybrid which is not recognised by RNase. Thus, addition of RNases enables enrichment and isolation of RNA ID(s) from a mixture of RNA molecules such as total RNA samples.

### Characterisation of RNA Transcript Isoforms

In some embodiments, the target nucleic acid(s) is an RNA transcript or RNA transcript isoform(s). In some embodiments, a sample comprising transcript isoform(s) is contacted with linearising units to provide one or more structural unit(s) at distinct regions of the transcript, e.g. exons, interspaced by one or more regions of double-stranded nucleic acid.

Typically, transcript isoforms are contacted with linearising units to provide distinct structural units and/or colours at distinct exons. Detecting the order of structural units and/or colours along the transcript allows the order of exons to be determined.

The method may comprise quantifying the relative abundance of transcript(s). In some embodiments, 18S rRNA or 28S rRNA is used as an internal control and the abundance of transcript(s) is determined relative to the abundance of 18S rRNA and/or 28S rRNA.

The target transcript may be contacted with linearising units to provide structural unit(s) and/or structural colour(s) that are specific to each distinct exon present in a pre-mRNA sequence. The linearising units may form isoform-specific IDs represented by the sequence of structural units and/or colours along the target transcript. For example, transcript isoforms derived from a pre-mRNA sequence comprising three exons may be contacted with linearising units to provide three distinct structural colours (e.g. ‘1’, ‘2’, and ‘3’) which correspond to each of the three exons. An RNA transcript isoform comprising the first and second exons sequentially would exhibit sequence ID ‘12’, whereas an RNA transcript isoform comprising the third and first exons would exhibit sequence ID ‘31’. The methods described herein can be used to characterise any transcript structural arrangement including but not limited to alternative splicing, alternative transcription start sites, and alternative polyadenylation signals.

The method of the invention advantageously omits amplification and enzyme-based processing steps and allows detection of multiple native RNA transcripts and alternative splicing variants in-parallel. The development of structural colours significantly increases the multiplexing potential of the invention and provides a method for affordable, simple, targeted isoform profiling of the whole transcriptome.

### Pathogen Detection

Methods of the invention may be used to characterise target nucleic acid(s) derived from pathogen(s). In some embodiments, several target nucleic acids derived from different pathogens are characterised. In this embodiment, target nucleic acids are contacted with linearising units to provide structural unit(s) and/or colour(s), or a sequence (ID) thereof, that is unique to a particular pathogen. In some embodiments, the method of the invention is used to characterise pathogen variants.

Methods of the invention may be used to characterise target nucleic acid(s) derived from a viral pathogen, a bacterial pathogen, fungal pathogen, protozoan pathogen or pathogenic worm. The target nucleic acid may be viral nucleic acid, e.g. a viral genome, such as a ssRNA viral genome. The ssRNA viral genome may be derived from a virus selected from e.g. an Influenza virus, Zika virus, Ebola virus, coronavirus, Dengue virus, Hantavirus, Nairovirus, Orthobunyavirus, Phlebovirus, Flavivirus, and Alphavirus. In some embodiments, the target nucleic acid is derived from a coronavirus, such as SARS-CoV-2.

In some embodiments, methods of the invention are used to quantify the relative abundance of multiple pathogens in the sample. Advantageously, the methods of the invention may be used to identify the predominant pathogen, or pathogen variant, in a sample.

### Nucleic Acid Binding Molecule Characterisation

The method of the invention can be used to characterise interactions between a target nucleic acid and a nucleic acid binding molecule. In some embodiments, the target nucleic acid is contacted with nucleic acid binding molecules prior to being contacted with linearising units. The nucleic acid binding molecule may be selected from a protein, nucleic acid, ligand, or small molecule. The nucleic acid binding molecule may be a drug. Nucleic acid binding molecule(s) bind to the target nucleic acid and block the interaction between the target nucleic acid and linearising units, thereby preventing the formation of double-stranded regions. In some embodiments, when nucleic acid binding molecule(s) are removed from the target nucleic acid, the region that has not interacted with linearising units provides a native structural unit which can be detected using the methods described herein, e.g. nanopore-based detection methods. In some embodiments, the nucleic acid binding molecule(s) stabilise a native secondary structure and prevent binding of linearising units to the native secondary structure. In this embodiment, the native secondary structure provides a native structural unit which can be detected using the methods described herein. The native structural unit(s) which correspond to nucleic acid binding molecule binding sites may be localised and/or quantified.

In some embodiments, the nucleic acid binding molecule stabilises secondary structures within the target nucleic acid and blocks the interaction between regions of the target nucleic acid forming said secondary structures and linearising units. In some embodiments, the nucleic acid binding molecule interacts with specific regions of the target nucleic acid and blocks the interaction between these regions of the target nucleic acid and linearising units.

In some embodiments, the current trace/signature produced by the target nucleic acid that has been treated with the nucleic acid binding molecule is compared to a negative control, e.g. the current trace/signature produced by the target nucleic acid that has not been treated with the nucleic acid binding molecule.

In some embodiments, the target nucleic acid is single-stranded RNA and the nucleic acid binding molecule is an RNA binding molecule, e.g. an RNA binding protein (RBP).

In some embodiments, the target nucleic acid is contacted with linearising units comprising docking strands that are complementary to the full length of the target nucleic acid. In some embodiments, the linearising units provide linearising-structural units.

### RNA Nanotechnology

In some embodiments, the target nucleic acid is an RNA molecule and contacting the RNA molecule with linearising units results in reshaping the target RNA molecule into a linear RNA ID comprising structural units interspaced by double stranded regions of nucleic acid. As used herein, a linear RNA means that the 3D secondary structure of the target RNA molecule is reduced as compared to the structure of the RNA prior to contacting with the linearising units.

Due to low yields and high production costs, RNA has not been widely and commercially used as a scaffold molecule for RNA nanotechnology and origami. The inventors have demonstrated that native RNA can be used as an RNA scaffold for RNA nanotechnology and RNA origami. In particular, MS2 bacteriophage (single-stranded) RNA (3.6 kb in length; SEQ ID NO: 1031) can be used as a scaffold for linearising units (short oligonucleotides), e.g. linearising units comprising DNA docking strands can be used for RNA:DNA nanotechnology applications.

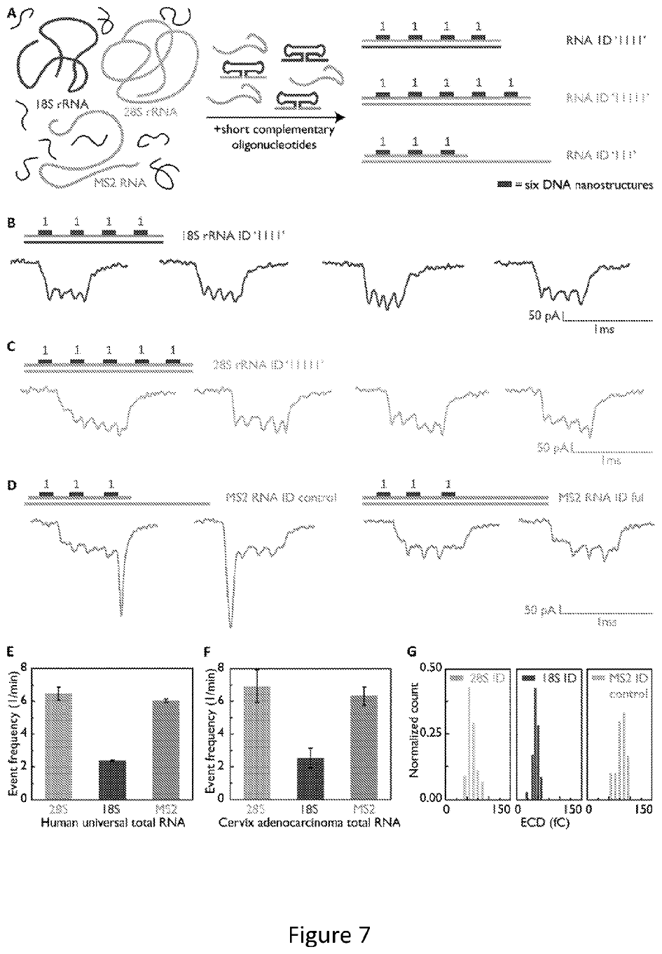

Furthermore, as demonstrated herein, ribosomal RNAs from native total RNA extract can be used for the same purpose. The inventors made identifiers (IDs) with linearising units that provide multiple structural units to create a unique sequence of protrusions ‘1111’, ‘111’, and ‘11111’ for 18S rRNA (1.9 kb) MS2 (3.6 kb), and 28S rRNA (5 kb), respectively (see FIG. 7). These findings clearly demonstrate that native RNA can be used as cheaper, shorter, and linear alternatives for nucleic acid scaffolds than those currently used (at present, there is only one commercially available circular single-stranded M13 DNA (7.2, 7.3, 7.6, and 8 kb scaffolds)).

Advantageously, RNA scaffolds are already linear in comparison to the ssM13 DNA which needs to be linearized prior to use as a scaffold molecule e.g. a DNA carrier. DNA origami and nanostructure designs are typically based on generic single-stranded M13 scaffolds and are therefore severely limited in terms of the range of applications they can be used to solve. Many properties of the target nanostructure are determined by details of the generic scaffold sequences, and so limited availability of scaffold sequences limits the application of nucleic acid origami. The inventors have overcome these problems by demonstrating that native RNAs can be used as scaffolds for linearising units.

### 3D RNA Structure Screening

Target RNA molecules can be linearized using the approach presented here (e.g. by contacting with linearising units) and characterised by detecting structural units using nanopore and/or fluorescence based detection methods. Advantageously, the occurrence and localization of secondary structures formed by parts of the target RNA molecule which are not bound to linearising units (single-stranded regions) can be detected and quantified at the single-molecule level. In some embodiments, native structural units are provided by regions of the target nucleic acid that are prevented from interacting with linearising units due to stable intramolecular interactions, e.g. secondary structures.

### Repeat Region Characterisation

The methods of the invention may be used to determine the number of repeated sequences in a target nucleic acid. For example, the target nucleic acid may be contacted with one or more linearising unit(s) to provide one or more structural unit(s) at each repeated sequence interspaced by one or more regions of double-stranded nucleic acid. The number of repeated sequences can be determined by counting the number of structural unit(s) along the target nucleic acid. In some embodiments, the methods of the invention are used to characterise tandem repeats in RNA, or large-scale repeat-associated arrangements.

The method of the invention can be used to determine the length of a poly(adenine (A)) tail. In some embodiments, the target nucleic acid is an mRNA and the mRNA is contacted with linearising units to provide a number of adjacent structural units along the poly(A) tail of the mRNA. The number of adjacent structural units along the poly(A) tail is determined by the length of the poly(A) tail. In some embodiments, the adjacent structural units provide a structural colour wherein the strength of the signal produced by the structural colour is determined by the number of linearising-structural units, which in turn is determined by the length of the poly(A) sequence. For example, a longer poly(A) tail will interact with more linearising-structural units resulting in the production of a larger structural colour and therefore a stronger signal than a shorter poly(A) tail.

### EXAMPLES

### Example 1

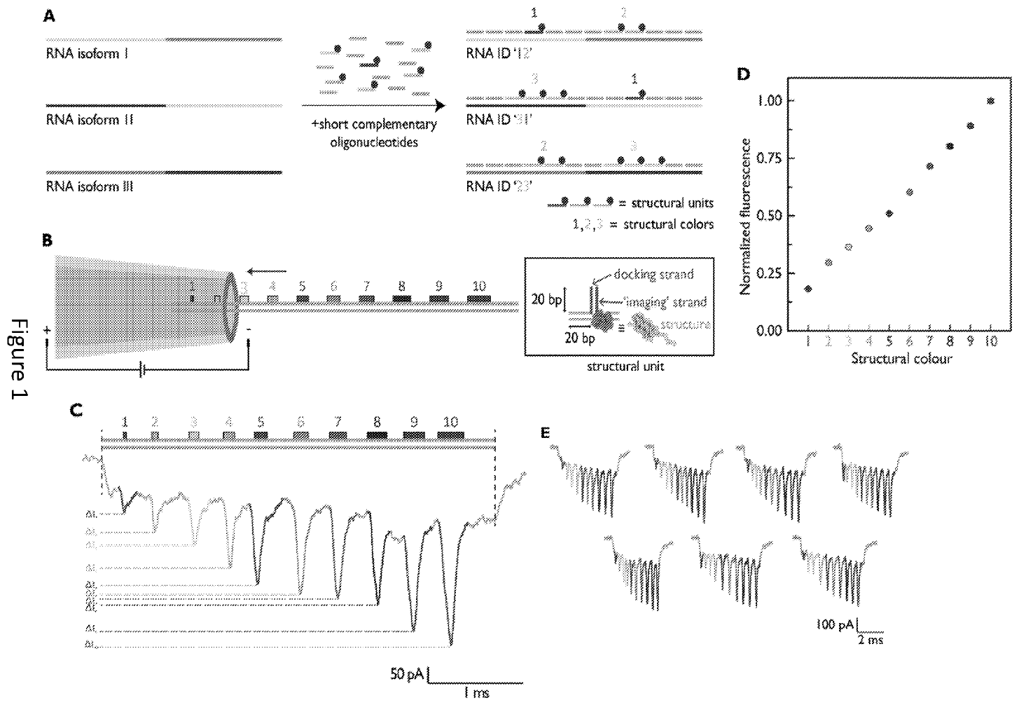

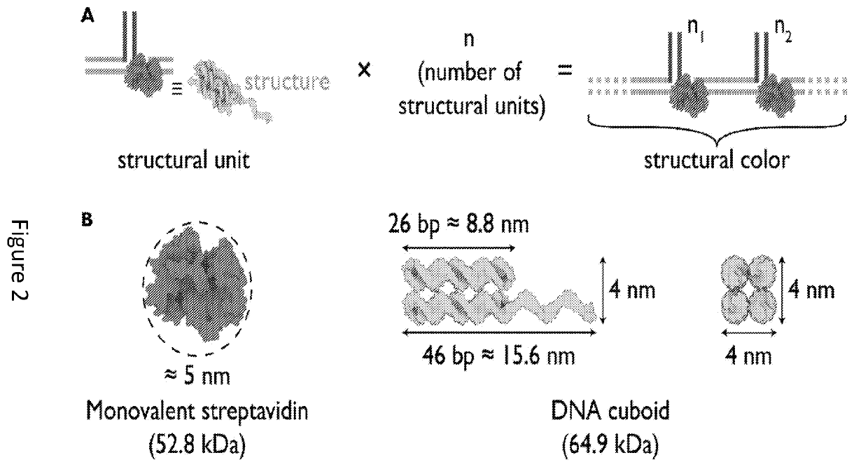

A representative experimental design is provided in FIG. 1. RNA isoforms are contacted with complementary linearising units (FIG. 1A). Exemplary linearising-structural units comprising protein (streptavidin) and DNA (DNA cuboid) labels are provided in FIG. 2. The inventors employed monovalent streptavidin-biotin or DNA cuboid nanostructures as labels to produce linearising-structural units (DNA cuboid oligonucleotides are listed in Table 4). In this example, a structural colour is composed of adjacent linearising-structural units, wherein the number of linearising-structural units corresponds to the structural colour. For example, the structural colour ‘2’ is equivalent to two adjacent linearising-structural units (FIG. 1A).

The linearising units anneal to complementary regions of the target RNA isoforms to produce an isoform-specific RNA ID which corresponds to the sequence of linearising-structural units and/or colours bound to the target RNA isoform (FIG. 1A). For example, structural colour ‘1’ bound downstream of structural colour ‘2’ corresponds to the RNA ID ‘12’. The sequence of linearising-structural units and/or colours in an RNA ID can be conveniently read using nanopore-based detection methods, also referred to herein as nanopore microscopy.

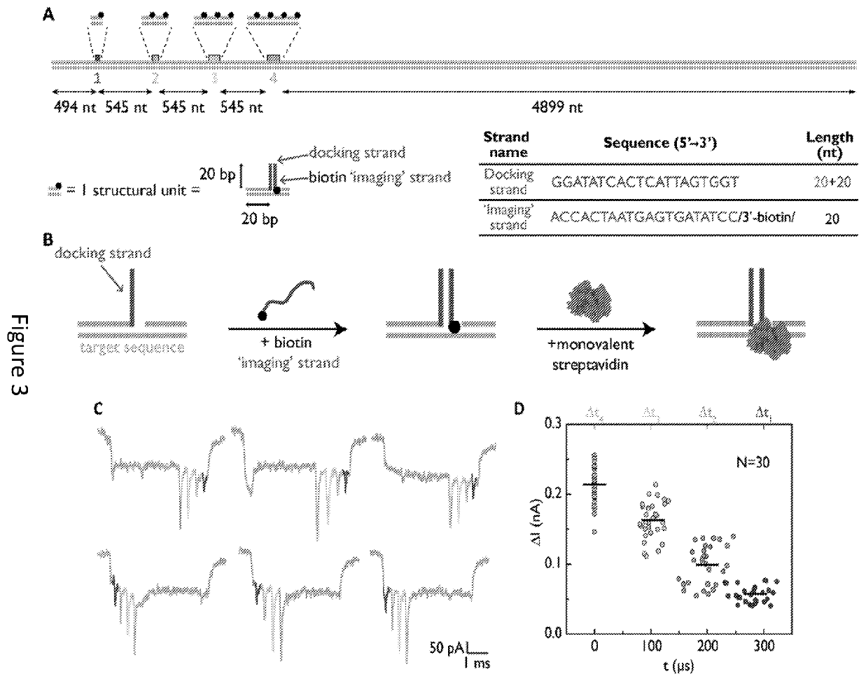

The inventors have demonstrated that multiple structural colours can be differentiated by their molecular weight using nanopore microscopy (FIG. 1B-E). The inventors first tested the ability of nanopore microscopy to differentiate between four structural colours. Single-stranded M13 (ssM13) was contacted with linearising units to provide four structural colours (linearising-structural units providing the structural colours are provided in Table 2) interspaced by regions of double-stranded nucleic acid (linearising units providing the double-stranded nucleic acid regions are provided in Table 1). The ssM13 was then translocated through a nanopore microscope to detect the structural colours. Each structural colour was identifiable by a distinct current signal, with structural colours of increasing molecular weight producing greater reductions in ionic current (FIGS. 3C and 3D).

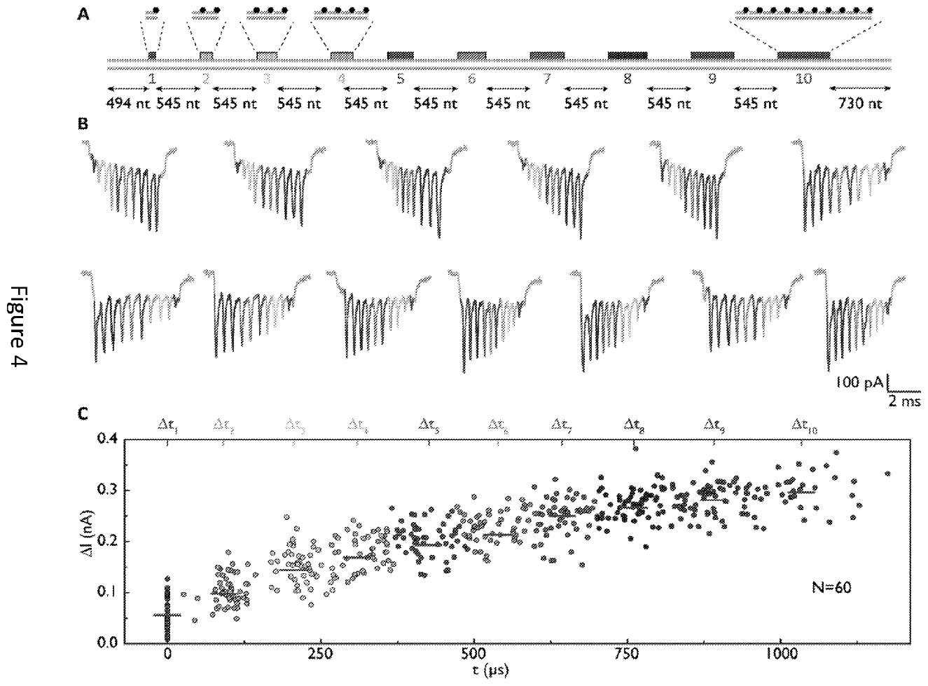

To further enhance the multiplexing capabilities of the method and the feasibility for large-scale transcriptome profiling, ssM13 was contacted with linearising units to provide 10 distinct structural colours interspaced with double-stranded regions of nucleic acid (linearising-structural units providing the 10 structural colours and double-stranded regions along ssM13 DNA are provided in Table 1 and Table 3). The nanopore microscope successfully detected and differentiated each of the 10 structural colours (FIGS. 1B-E and FIG. 4). Representative current traces are shown in FIGS. 1C and E.

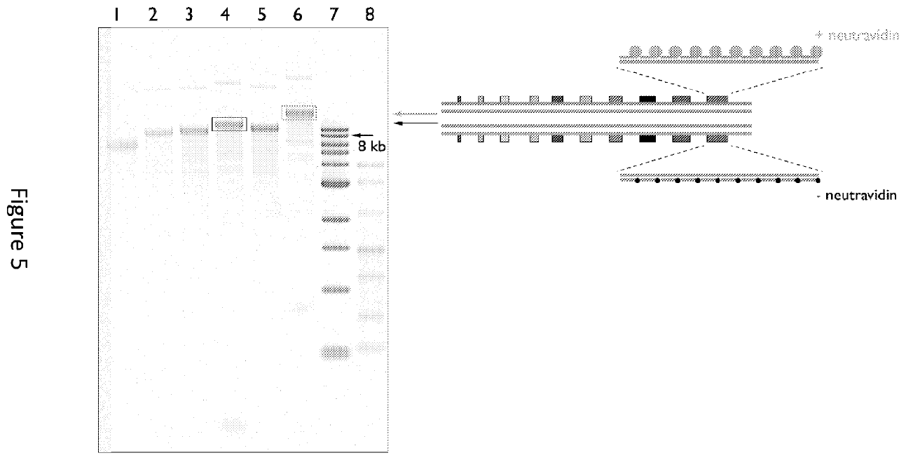

The inventors validated the fabrication of both 4-colour and 10-colour rulers (ssM13 comprising four and ten structural colours, respectively) using linearising-structural units comprising biotinylated labelling strand and polyacrylamide gel electrophoresis (PAGE) with and without the addition of neutravidin (FIG. 5).

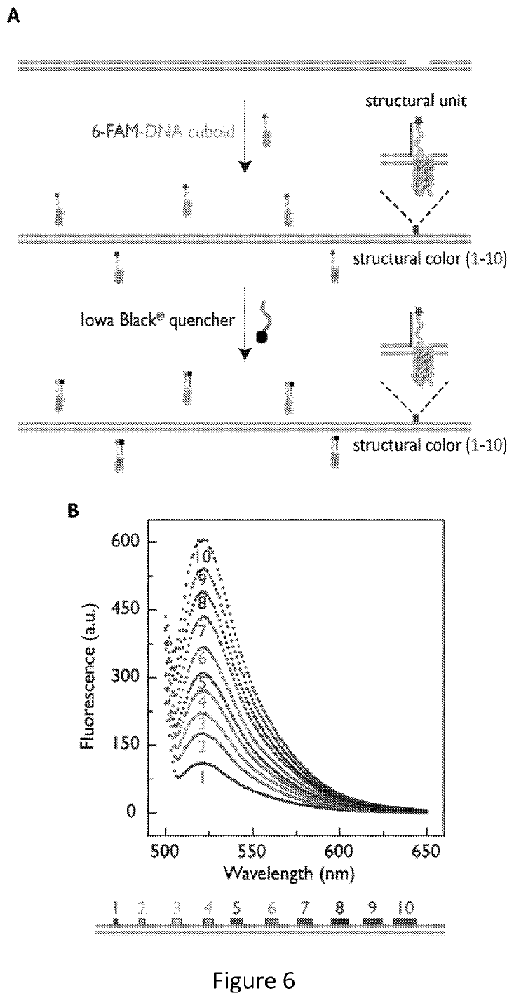

Correct assembly of the ten structural colours was also confirmed using a fluorescence quenching assay using fluorescein (6-FAM) labelled linearising-structural units (FIG. 1D and FIG. 6). IDs were produced comprising a single structural colour at equimolar concentrations. Excess 6-FAM DNA cuboid strands are quenched by binding of Iowa Black quencher. After quenching, only 6-FAM DNA cuboids in the linearising-structural unit(s) along the target nucleic acid emit a fluorescence signal. The fluorescence output produced by each ID (corresponding to structural colours 1-10) confirms accurate fabrication of structural colours (FIG. 1D and FIG. 6B).

Using multiple structural colours and nanopore microscopy, the methods developed herein offer excellent potential for multiplexing. This is an essential feature to allow the characterisation of a vast number of target nucleic acids, e.g. structural isoforms, including their order, length, and conformation.

### Example 2

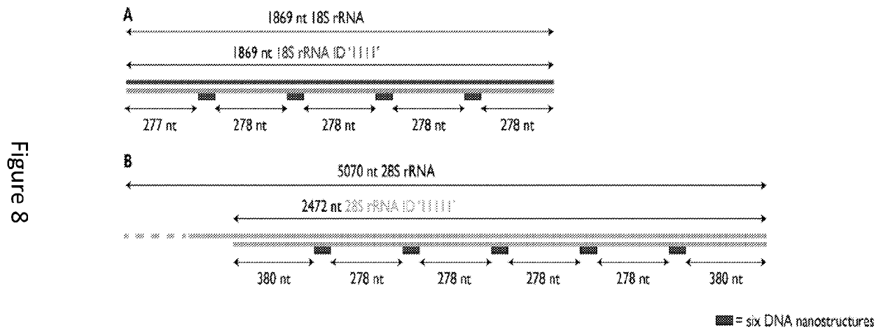

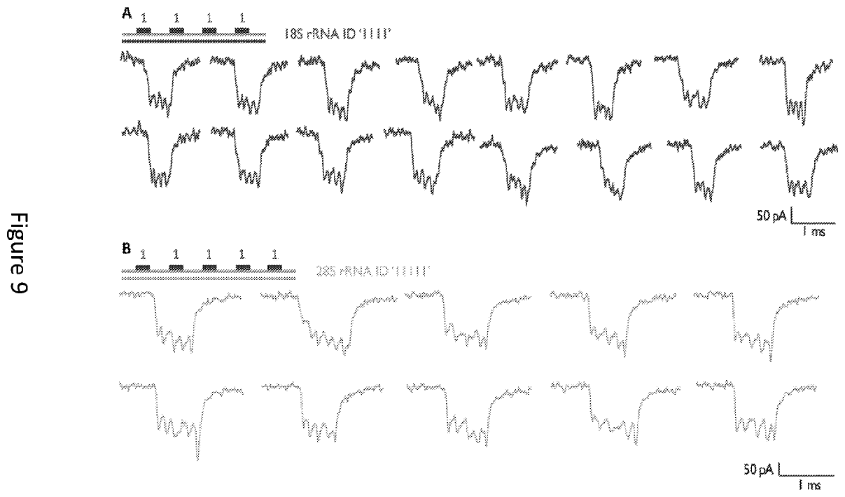

The method of the invention can be used to identify and quantify various target nucleic acids in a single reaction mixture as schematized in FIG. 7A. As a proof-of-concept, the inventors created distinct IDs (FIG. 7A and FIG. 8) for human 18S and 28S rRNA (linearising units used for fabrication of 18S rRNA ID and 28S rRNA ID are listed in Table 6 and Table 7, respectively) and external MS2 RNA ID control with known concentration (linearising units used for fabrication of MS2 RNA ID are listed in Table 5) in a complex nucleic acid mixture. Each RNA ID was identified with the nanopore microscope and respective events for 18S rRNA ID with four linearising-structural units (‘1111’), 28S rRNA with five linearising-structural units (‘11111’), and external RNA ID control with three linearising-structural units (‘111’) are depicted in FIGS. 7B, C, and D, respectively (additional events for 18S rRNA and 28S rRNA are shown in FIG. 9). In this example, each linearising-structural unit comprises a labelling region having DNA nanostructure labels (see FIG. 7A).

The inventors demonstrated the quantification of multiple RNAs in a background of human total universal RNA (composition listed in Table 9) and adenocarcinoma total RNA (FIGS. 7E and 7F, respectively). The concentration of each RNA is calculated from the nanopore event frequency of RNA IDs using a previously introduced model for these particular experimental conditions (Bell et al. Phys. Rev. E. 2016; 93(2):022401). An internal reference ID can further improve transcript isoform-level quantification. 18S rRNA can be used as an intrasample reference for relative gene expression quantification.

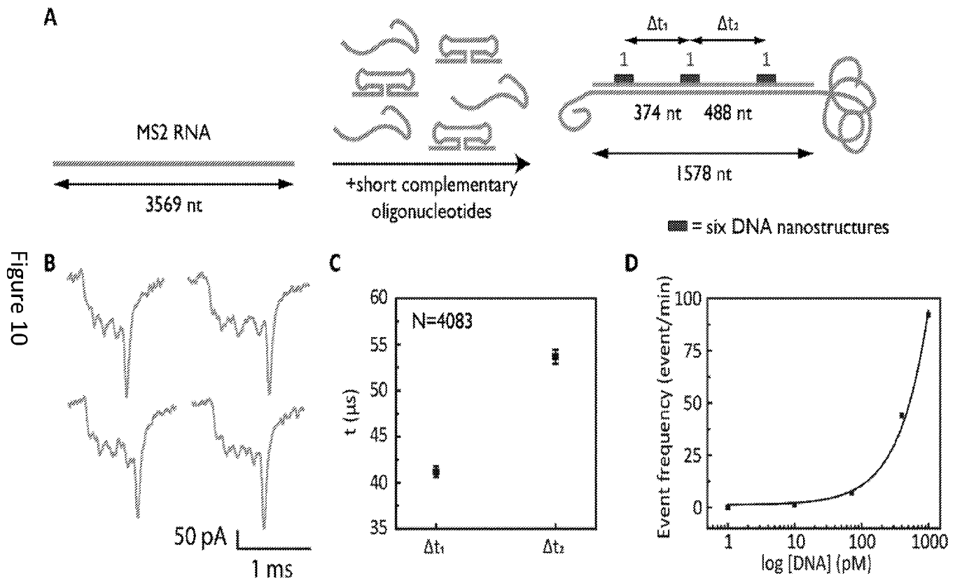

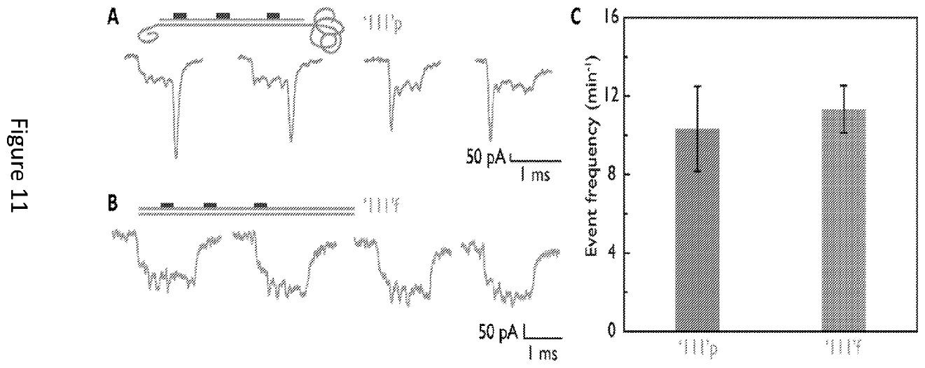

RNA ID ‘111’ was fabricated for 3.6 kb long MS2 RNA (FIG. 7D and FIG. 10; linearising units and linearising-structural units are provided in Table 5). The inventors compared detection of MS2 RNA ID in the presence of partially or fully complementary linearising units. Nanopore data indicated that RNA ID can be fabricated with linearising units that anneal to only part of the target RNA or to the whole target RNA (FIG. 7D, left and right respectively; FIGS. 10 and 11B). By measuring distances between structural units/colours, the inventors demonstrated that velocity fluctuations do not affect correct readout and position sequencing of structural units along target RNA (FIG. 10C). The data show that it is possible to use only a part of the target RNA to fabricate an ID (FIG. 7D). The MS2 ID provides an example where part of the sequence is left unpaired and is detectable as a native structural unit, represented by a deeper signal at the beginning/end of nanopore signal ID event in nanopore measurements (fully complementary linearising units for MS2 RNA ID are listed in Table 8).

Quantification is based on nanopore capture rate and so the inventors confirmed that the capture rate is independent of the level of complementarity between target RNA and linearising units (FIG. 11). The normalized histograms of event charge deficit (ECD) of identified RNA IDs indicate the shift in a length-dependent manner (FIG. 7G).

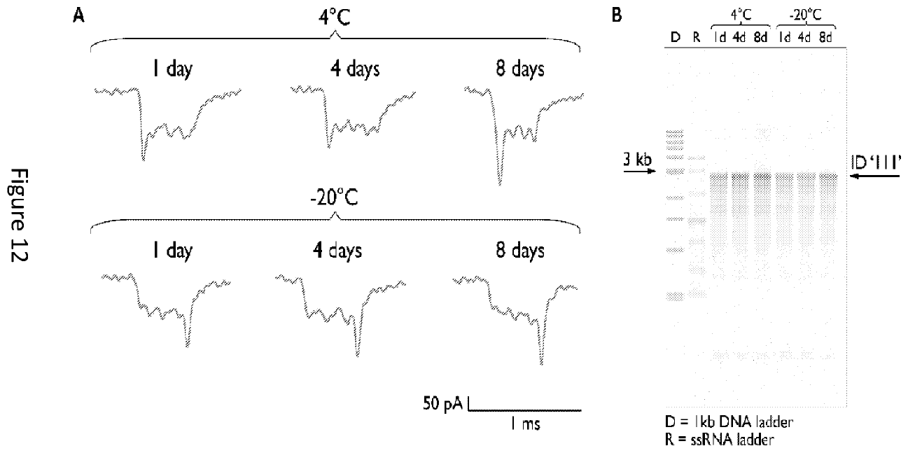

RNA IDs formed from RNA:DNA hybrids were tested for adequate storage conditions and temperature stability. The inventors tested the stability of fabricated RNA IDs over time using nanopores and gel electrophoresis (FIG. 12) and demonstrated that RNA ID RNA:DNA hybrids exhibited only minimal degradation with standard storage conditions, e.g. stored at 4° C. and −20° C. for 1, 4, and 8 days.

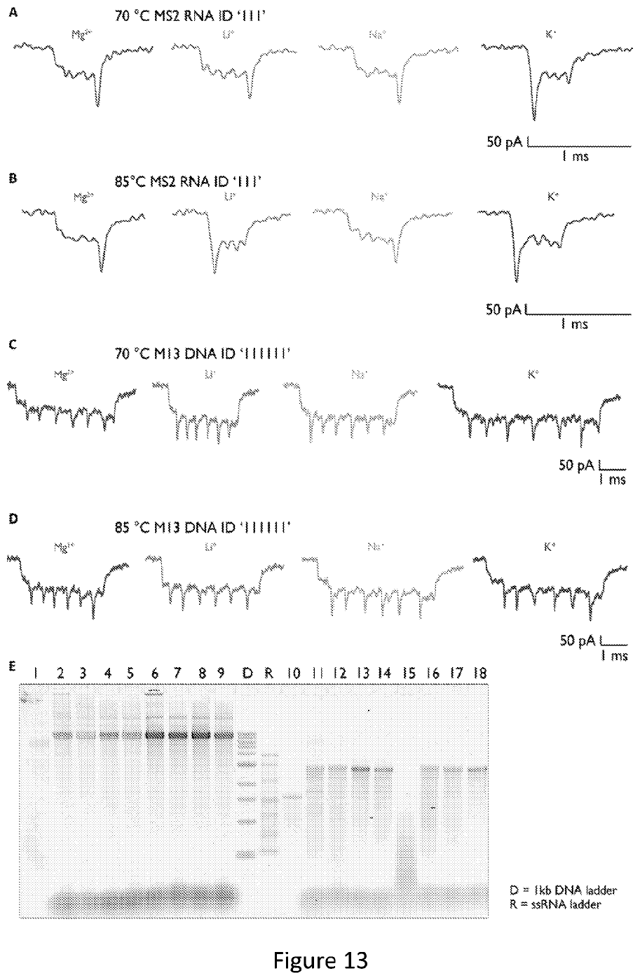

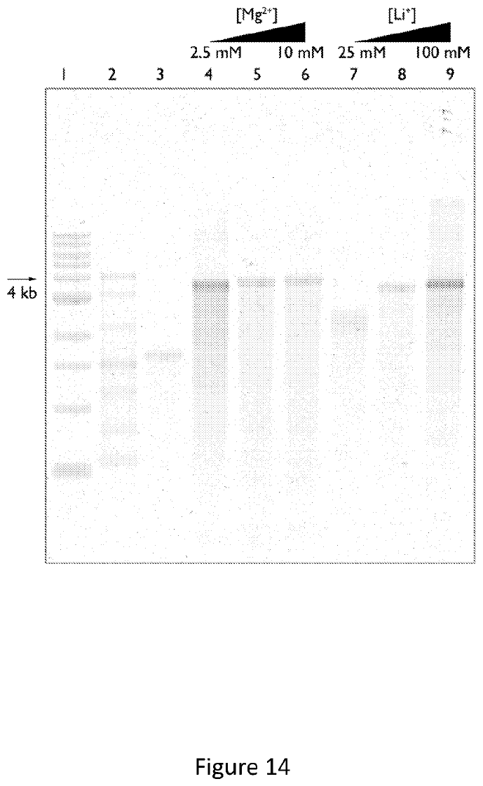

The inventors demonstrated that divalent ions can be replaced by various alkali monovalent ions, therefore, limiting magnesium RNA structure stabilization and fragmentation for RNA ID fabrication (FIG. 13). Furthermore, the inventors examined the concentration effects on RNA ID fabrication and identified the minimal salt concentration for the ID fabrication in the experimental conditions (FIG. 14).

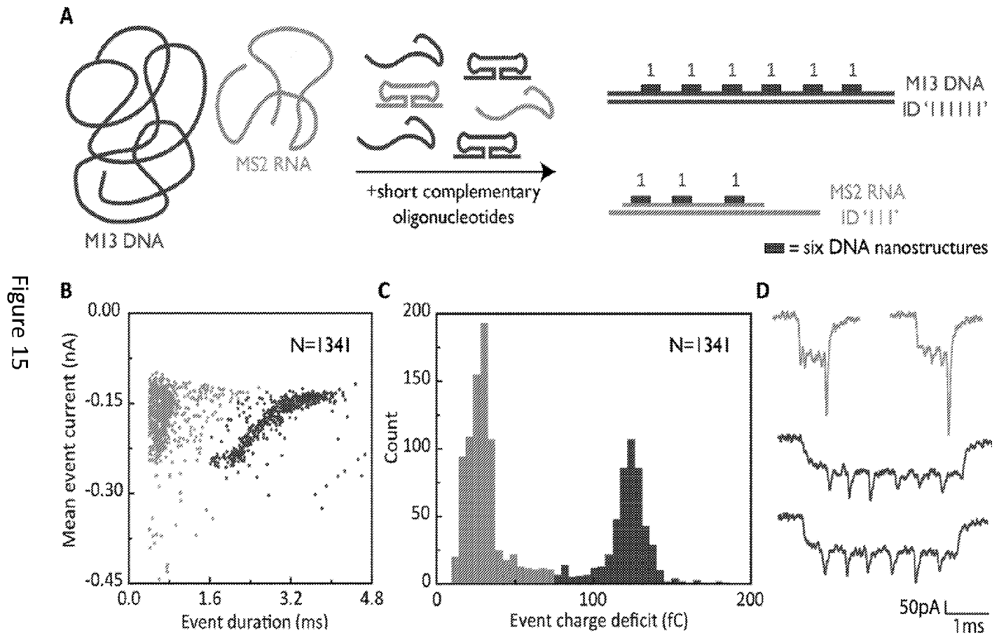

The inventors employed the method of the invention to detect two Escherichia viruses: MS2 RNA virus and M13 DNA virus in parallel (FIG. 15). The inventors fabricated MS2 RNA ID ‘111’ and M13 DNA ID ‘111111’ (FIG. 15A; linearising units for MS2 and M13 IDs are listed in Table 5 and Table 10, respectively) in-parallel and successfully identified expected readouts (FIG. 15B-D). This approach demonstrates that the methods of the invention enable multiplexed, viral nucleic acid identification, and quantification in a one-step reaction.

### Example 3

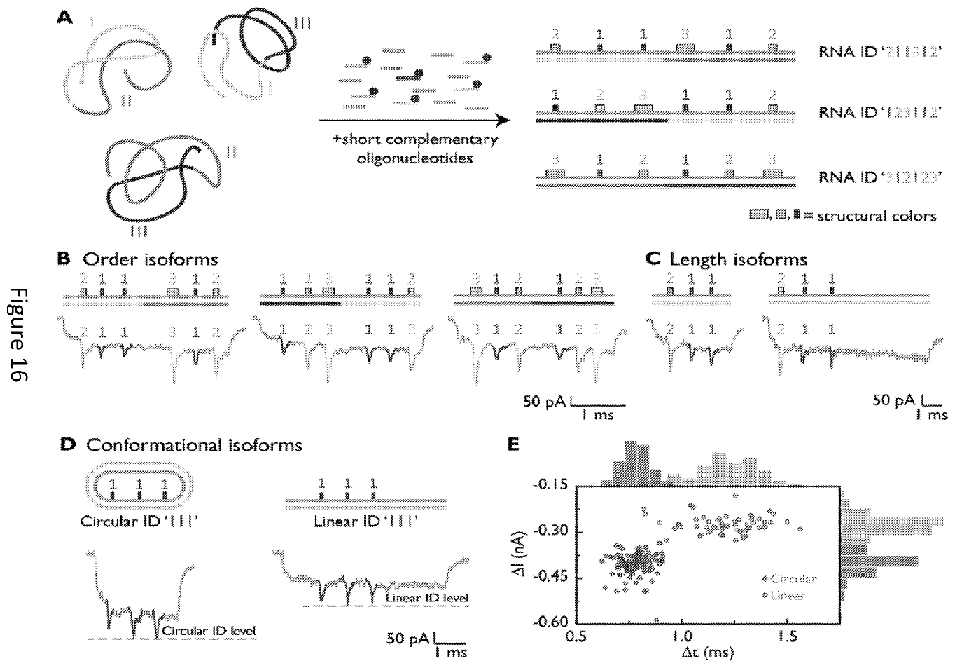

By employing multiplexed experimental designs, RNA ID fabrication can be used to detect, and optionally quantify, transcript variants that are formed as a result of alternative transcript processing and structural arrangements in a premature transcript (pre-mRNA) (FIG. 16).

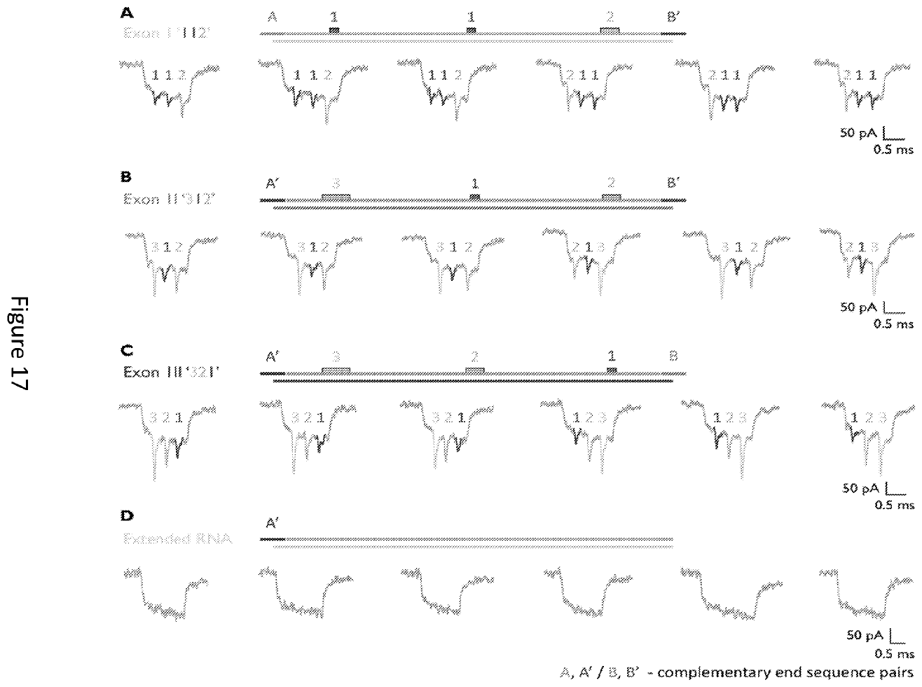

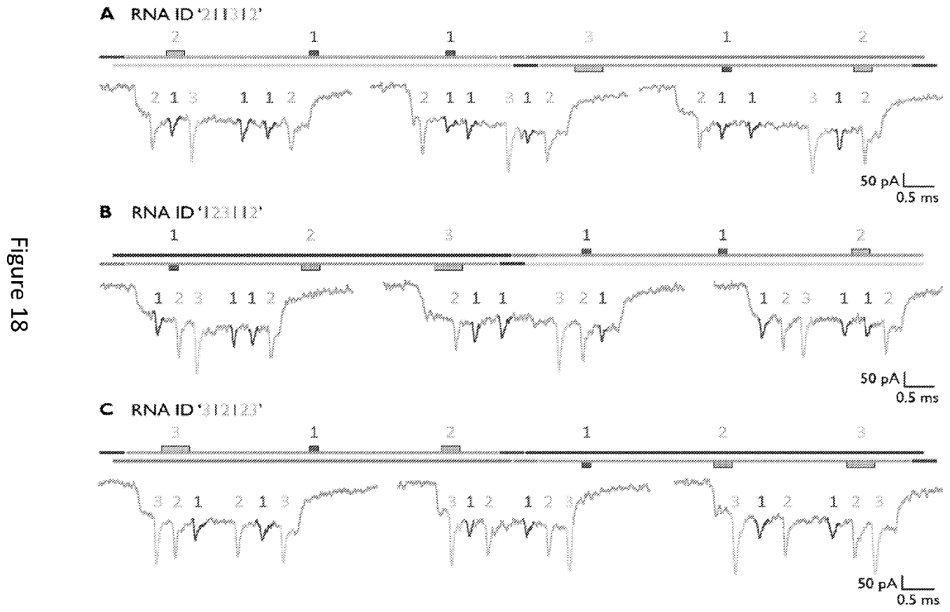

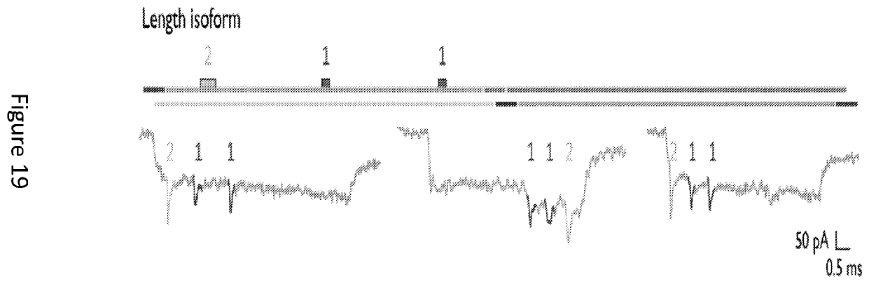

The method developed herein is capable of identifying order, length, and conformational isoforms (FIGS. 16B, C, and D, respectively). The inventors designed asymmetric, exon-specific IDs (ID designs with example events are presented in FIG. 17; and linearising units used to produce IDs are listed in Tables 11 and 12) to enable the identification of distinct transcript isoforms. The combination of exons results in multiple transcript isoforms with the same length but different sequences (FIG. 18). In FIG. 16B, three correctly identified isoforms with the same length but a different order of exons that contain either exons I and II (RNA ID ‘211312’), exons I and III (RNA ID ‘123112’), or exons II and III (RNA ID ‘312123’) are shown. Isoforms of different lengths can also be differentiated by the length of time taken to translocate through the nanopore (FIG. 16C, FIG. 19).

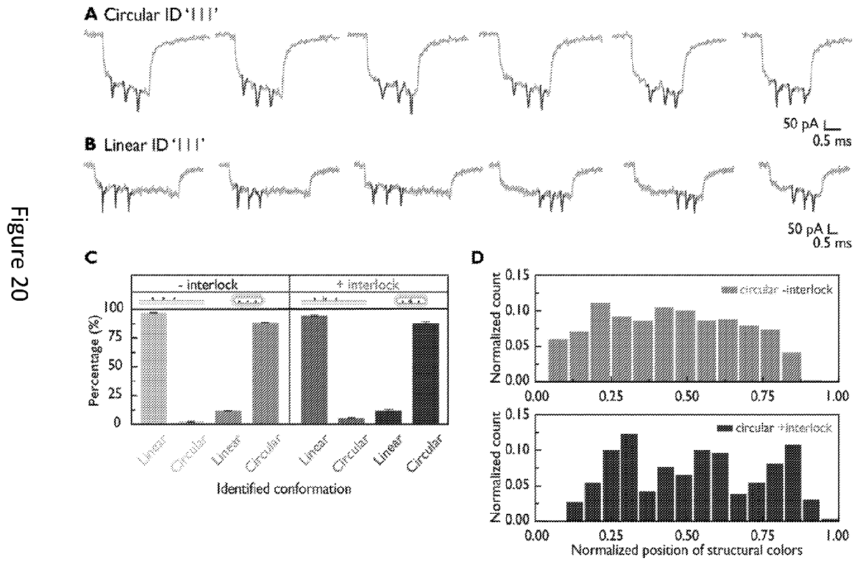

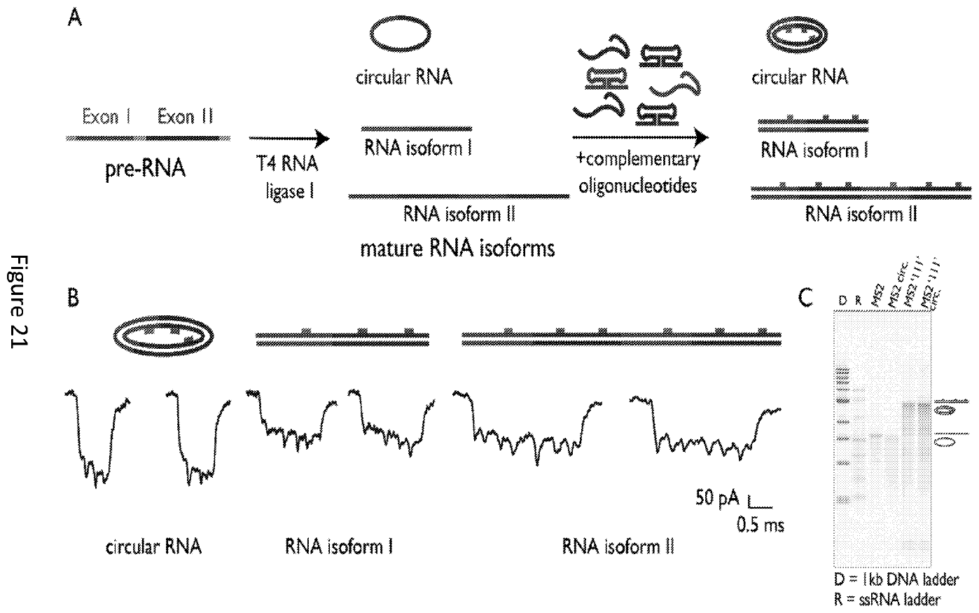

Another critical feature that is not achievable with RNA-seq includes discrimination of transcript conformations, e.g. circular and linear RNA conformations (FIG. 16D-E). The inventors performed in vitro RNA circularization (FIG. 21) of linear MS2 RNA ID ‘111’ using T4 RNA ligase I. Synthetic circular and linear IDs were generated with the sequence of linearising-structural units ‘111’ using the same linearising units mixture (FIG. 16D; linearising units used to generate the linear and circular IDs are listed in Table 13). Representative nanopore events are shown in FIG. 20. As demonstrated by the scatter plot in FIG. 16E, the two non-overlapping populations of circular and linear IDs can be readily detected and differentiated based on the translocation time (Δt) which is ^{˜}2 times shorter for the circular isoform and the event current blockage (ΔI) which is ^{˜}2 times higher for the circular isoform than for the linear isoform. Interlock oligonucleotides were used to fix the position of colours in the circular ID conformation and to increase readout quality (FIG. 20C-D).

These data confirm that circular RNA and linear RNA can be differentiated by methods of the invention. It is important to note that RNA ID design allows simultaneous quantification of RNA structural arrangements and conformation without requiring any design modification.

### Example 4

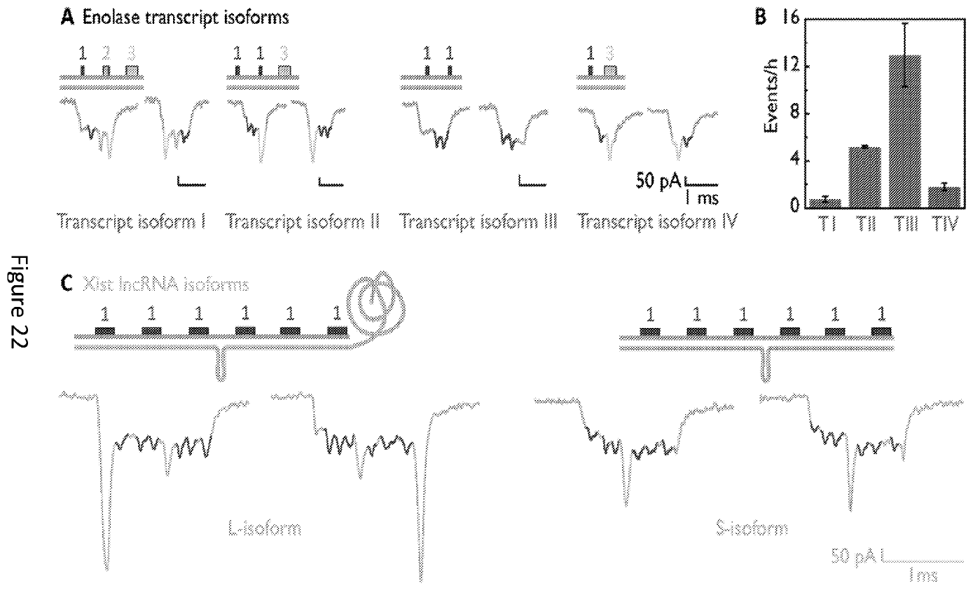

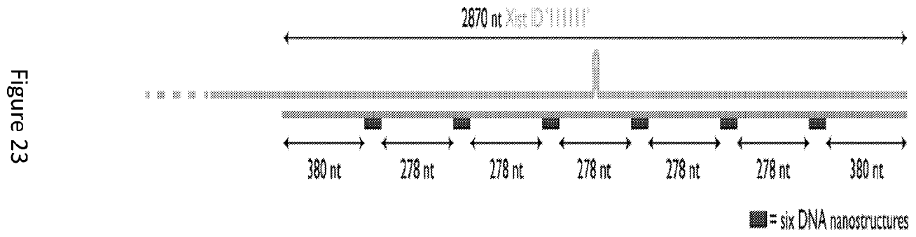

The inventors employed the method of the invention for targeted identification of enolase 1 (ENO1) isoforms in the human transcriptome (FIG. 22A-B). ENO1 is known to have multiple transcript isoforms that differ in length or sequence as a result of alternative splicing of pre-mRNA. The inventors employed three structural colours to identify four transcript isoforms (FIG. 22A; linearising units to provide isoform IDs are provided in Table 14). RNA isoform ID designs and example nanopore detection events are illustrated in FIG. 22A.

Methods of the invention successfully discriminated between four ENO1 splicing isoforms in a complex human transcriptome mixture (human cervix adenocarcinoma total RNA). These results demonstrate that three structural colours are sufficient to easily identify desired targets at the whole-transcriptome level without relying on enrichment of target nucleic acid and/or rRNA depletion. Each ENO1 transcript variant was quantified based on three individual nanopore measurements (FIG. 22B). 18S rRNA ‘1111’ was used as an internal control with 107±12 events/h. Total events detected were 39521 for three nanopores.

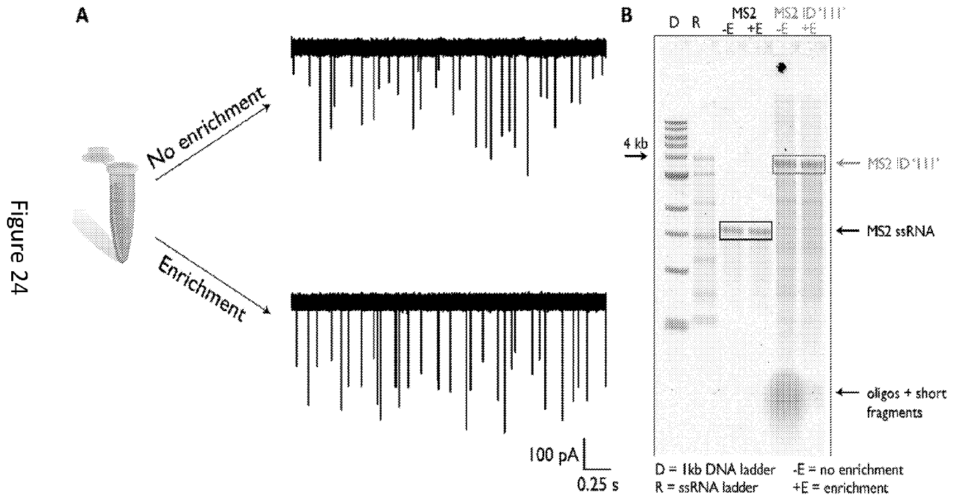

Using X-chromosome inactivation transcript long-non-coding RNA (Xist lncRNA) as an example, the inventors demonstrated length isoform discrimination in the native transcriptome (FIG. 22C). The inventors targeted part of Xist RNA to fabricate ID ‘111111’ (design of Xist lncRNA ID is schematized in FIG. 24; linearising units used for fabrication of the RNA ID are provided in Table 15). The part of the sequence that differs between long (L-isoform) and short (S-isoform) isoforms is left unpaired.

The expected ID nanopore events should depict the sequence of six linearising-structural colours, the terminal unpaired RNA coil (native structural unit), and a potential internal secondary structure (native structural unit) as predicted from the sequence (FIG. 22C). Representative examples of Xist lncRNA isoform IDs that match the predicted design and previously identified Xist lncRNA isoforms are shown in FIG. 22C. L- and S-isoforms differ in the presence or absence of the terminal native structural unit produced by the terminal unpaired RNA coil that is observable as the deep downward signal at one end of L-isoform. The inventors demonstrated that without requiring any design adaptation, the method of the invention discriminates structural isoforms of Xist by their length, large (L-isoform) and short (S-isoform) (FIG. 22C), and also detects internal secondary structures.

### Example 5

### Self-Assembled RNA Origami Native Structural Colours

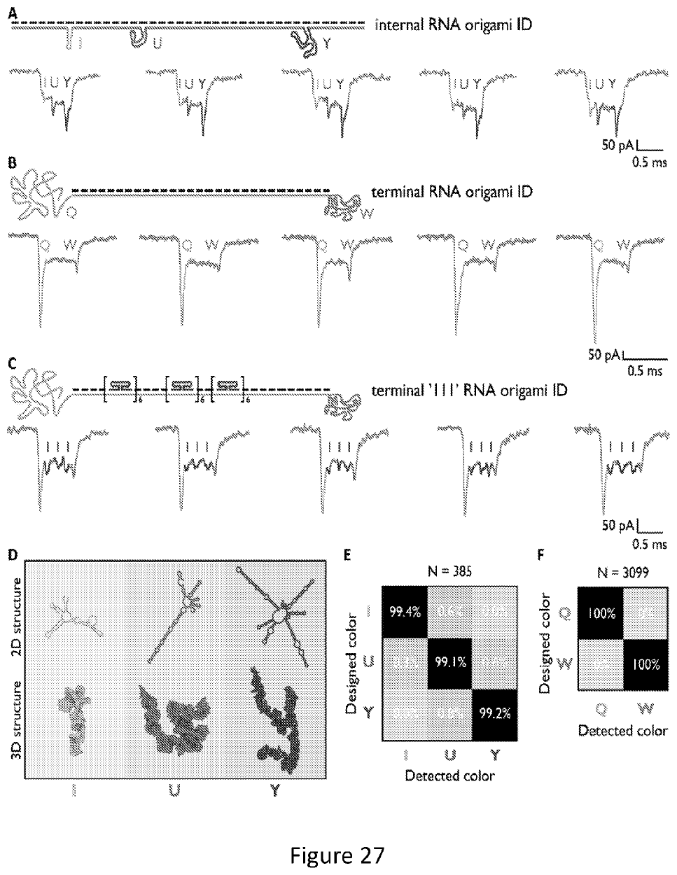

Some transcripts are (ultra)long or contain strong RNA secondary structures that are challenging to complement. For ultralong transcripts complementing the whole RNA may be undesirable because it would require a large number of linearising units. The inventors have demonstrated that RNA ID can be assembled by contacting the target with linearising units that are complementary to only a region of interest/part of the RNA target (FIG. 27).

The inventors assembled native structural unit (RNA origami) IDs by employing secondary structure formation in pre-designed locations (FIG. 27; linearising units are provided in Table 16). Three structural colours have been assembled by nanoscale folding of 114 nt, 190 nt, and 342 nt single-stranded RNA to provide native structural colours ‘I’, ‘U’, and ‘Y’, respectively (2D and 3D structures are shown in FIG. 27D). Each of these self-assembled native structural units has a specific downward current signature, that can be observed from nanopore events. The internal RNA IDs from nanopore recordings show three structural colours I, U, and Y as expected from predesigned local assembly (FIG. 27A). The accuracy of identification of each structural colour is over 99% as displayed in FIG. 27E. These results clearly demonstrate that single-stranded regions of the target nucleic acid, interspaced by double-stranded regions of nucleic acid, can be used to produce native structural units/colours.

To demonstrate that linearising units that are complementary to only a part of the target RNA can be used to produce accurate readout of IDs, the inventors linearised only a middle region of RNA as shown in FIG. 27B (linearising units are provided in Table 17). The resulting RNA ID comprised terminal native structural unit (RNA origamis) that are 401 nt and 1230 nt in length (represented by Q and W, respectively). Terminal native structural units (RNA origami) translocation through a nanopore induced two terminal downward signals that correspond to these two terminal native structural units. The accuracy of detection of terminal RNA origami structural units Q and W is 100% (FIG. 27F).

Finally, the inventors designed terminal ID ‘111’ (FIG. 27C; linearising units are provided in Tables 5 and 8) comprising: native structural units (Q and W) at both ends of the target; double-stranded nucleic acid regions (RNA-DNA hybrid nanostructure); and linearising-structural units comprising a labelling region having self-assembled DNA double-hairpins (FIG. 27C, square brackets). These IDs are efficiently read out as can be seen from nanopore events. These data indicate that both native structural units and linearising-structural units can be used to produce RNA IDs.

### Example 6

Thermal cell lysis is not typically used for nucleic acid extraction because it can lead to undesirable nucleic acid degradation, particularly of RNA. The inventors have made the surprising discovery that coupling thermal cell lysis with RNA identifier (ID) assembly reduces unwanted degradation of target RNA.

Advantageously, RNA ID assembly is successfully achieved even at elevated temperatures. Linearising units bind to complementary sequences of the target RNA to create a double-stranded RNA:DNA hybrid that is specific to the target of interest. The inventors have shown that RNA:DNA hybrids formed by this method demonstrate increased RNA stability, even at elevated temperatures. Without wishing to be bound by theory, the inventors believe that this stability is due to the prevention of RNase degradation (i.e. lack of single-stranded RNA target) and increased persistence length by inhibition of self-cleavage mediated by the 2′ hydroxyl group (OH).

Escherichia coli identifier was assembled by mixing 5 μL of E. coli total RNA, 4 μL of 1M LiCl (pH 7.4), 4 μL of 10×TE (100 mM Tris-HCl pH 8.0, 10 mM EDTA), 2.4 μL of linearising unit mixture designed to complement 16S ribosomal RNA fully (1 μM of each linearising unit), 2 μL of biotin labelling strand (25 μM) and 22.6 μL of nuclease-free water.

The mixes were heated for 5 min at 70° C., 80° C., 90° C., or 100° C. using a thermomixer. The mixes were purified of excess linearising units using Amicon 0.5 mL filters with 100 kDa cut off by adding 460 μL of washing buffer (10 mM Tris-HCl pH 8.0, 0.5 mM MgCl2) and centrifuged at 9200×g for 10 min. This step was repeated twice. The filter was turned around, placed in the fresh tube, and centrifuged at 1000×g for 2 min.

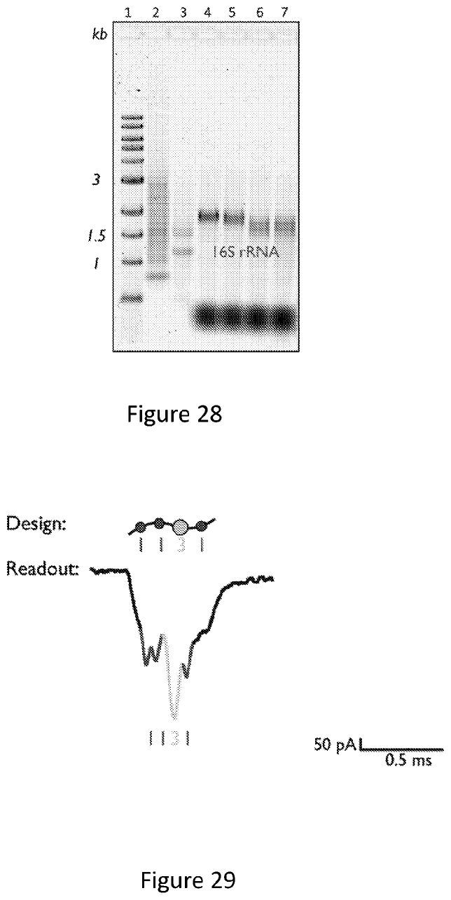

RNA IDs were run on an agarose gel as shown in FIG. 28. Surprisingly, regardless of incubation temperature (lanes 4-7), 16S rRNA IDs were successfully assembled. Advantageously, 23S rRNA which forms a clear band in lane 3 is not evident in lanes 4-7 indicating that unwanted RNAs are degraded at higher temperatures, but target RNA in the form of RNA IDs remains.

FIG. 29 provides an exemplary nanopore event for an RNA ID generated at 100° C., indicating that the E. coli 16S rRNA ID design ‘1131’ is identified from nanopore readout successfully.

### Materials and Methods

### Materials

The commercial buffers used in the examples were Tris-EDTA buffer solution 100×concentrate (Sigma-Aldrich, catalog number T9285), 0.2 μm filtered 1M MgCl₂ (Invitrogen by Thermo Fisher Scientific, catalog number AM9530G), 0.2 μm filtered and autoclaved nuclease-free water (Ambion, catalog number AM9937). Lithium chloride for molecular biology 99% purity (Sigma-Aldrich, catalog number L9650), sodium chloride for molecular biology 99% purity (Sigma-Aldrich, catalog number S3014), Tris-HCl BioPerformance certified, >99% purity (Sigma-Aldrich, catalog number T5941). All buffers used in this study were filtered with 0.22 μm Millipore syringe filter units (Merck).

Glass quartz capillaries with filament (inner diameter 0.2 mm, outer diameter 0.5 mm) were purchased from Sutter Instrument Company. PDMS was purchased from Sylgard 184, Dow Corning (catalog number 101697), microscope slides clear ground 1.0-1.2 mm (Thermo Fisher Scientific, catalog number 1238-3118), silver wire with 1.0 mm diameter (Advent Research Materials Ltd, catalog number AG548711). Amicon 0.5 mL filter units (100 kDa cut-off) were purchased from Merck (catalog number UFC5100BK). Membrane Filter, 0.22 μm pore size membrane filters (MF-Merck Millipore™, catalog number GSWP04700).

DNA LoBind® Tubes (Eppendorf) were purchased from Thermo Fisher Scientific, and thin-walled, frosted lid, RNase-free PCR tubes (0.2 mL) were purchased from Thermo Fisher Scientific (catalog number AM12225).

RNA from bacteriophage MS2 3569 nt in length was purchased from Roche (catalog number 10165948001), total RNA from human cervical adenocarcinoma was purchased from Thermo Fisher Scientific, Invitrogen (catalog number AM7852) and human universal reference total RNA was purchased from Thermo Fisher Scientific, Invitrogen (catalog number QS0639). Single-stranded circular m13mp18 7249 nt in length was purchased from Guild Biosciences (foundation m13).

### DNA Cuboid

DNA cuboid was assembled by using six oligonucleotides provided in Table 4. 1 μL of each oligonucleotide (100 μM, IDTE buffer (10 mM Tris-HCl, 0.1 mM EDTA), pH=8.0), 10 μL of filtered 10×TE buffer (100 mM Tris-CI, 10 mM EDTA, pH=8.0), 20 μL of filtered 100 mM MgCl₂, and 64 μL of filtered Milli-Q ultrapure water were mixed. Buffers were filtered with the MF-Millipore™ Membrane Filter, 0.22 μm pore size. The mix is vortexed and spun down before the structure assembly. All oligonucleotides were purified by desalting and ordered in IDTE buffer in 100 μM concentration. The mix was heated to 95° C. for 5 minutes and slowly cooled down to 25° C. for 18 h. The mix was stored at 4° C. without additional purification until further use. Further details of DNA cuboid assembly can be found as CP3 short DNA origami nanopore (Heid, C. A. et al. Genome Research. 6, 986-994 (1996) and Stark, R. et al. Nature Reviews Genetics. 20, 631-656 (2019)) without additional structural changes required for the structural unit.

The DNA cuboid for fluorescence/quenching assay was assembled using the same protocol. Oligonucleotide 1M1 is replaced with the 5′ labelled end of oligo 1M1 with 6-FAM (100 μM, IDTE buffer (10 mM Tris-HCl, 0.1 mM EDTA), pH=8.0). The 6-FAM 1M1 oligonucleotide was purified with high-performance liquid chromatography (HPLC).

### Design of Linearising Units, Linearising-Structural Units and Structural Colours

Linearising units comprise a docking strand having a region that is complementary to the target nucleic acid. Linearising-structural units used in the examples comprise a docking strand and a labelling strand or labelling region. In embodiments comprising labelling strands, the docking strand has two parts: a first part having a 20 nt sequence that is complementary to the specific position in a target RNA; and a second overhang part having a 20 nt sequence that is complementary to the labelling strand. The labelling strand harbours at the 3′ end a structure (FIG. 2A, left). This structure can be a protein such as monovalent streptavidin bound to biotin or DNA cuboid (FIG. 2B). In examples using linearising-structural units comprising a docking strand having a labelling region, the labelling region comprises DNA double-hairpin structures.

Structural colours used in the examples were made by designing an integer number of linearising-structural units that anneal to the target nucleic acid sequentially. For example, structural colour two corresponds to two adjacent linearising-structural units (FIG. 2A, right). The inventors have demonstrated the fabrication of ten structural colours (eleven including structural colour 0). The inventors used streptavidin-based structural colours for data shown in FIG. 1, and DNA cuboids for data shown in FIGS. 16 and 22. For the one-colour system (as shown in FIGS. 7 and 15), the number of structural colours was varied rather than the structural colour itself.

### Fabrication of 4-Colour and 10-Colour Rulers

To fabricate multicolour rulers the inventors used linearising unit mixes containing linearising units and linearising-structural units; (linearising units used to complement the whole target are listed in Table 1 and linearising units replaced with linearising-structural units to provide 4-colour and 10-colour rulers are provided in Table 2 and Table 3, respectively). A 40 μL reaction was prepared by mixing linearized ssDNA (to 20 nM or 800 fmoles) and linearising units (to 60 nM each or 2400 fmoles), in 10 mM MgCl₂, 1×TE (10 mM Tris-HCl, 1 mM EDTA, pH 8.0) buffer, and nuclease-free water was added to the final reaction volume. Buffers were filtered with the MF-Millipore™ Membrane Filter, 0.22 μm pore size. The mix was mixed by pipetting and spinning down; then heated to 70° C. for 30 s and gradually cooled down (−0.5° C./cycle, 90 cycles each 30 s) over 45 minutes to room temperature, and held at 4° C. Terminal oligonucleotides contain four dT nucleotides that should prevent IDs base stacking. 4-colour and 10-colour designs are illustrated in FIGS. 3 and 4, respectively. Biotin ‘labelling’ strand in oligo mix was in 1.5×excess to docking sites. The fabrication was performed as described above.

### Native Agarose Gel Electrophoresis Analysis

Samples were run on a 1% (w/v) agarose gel prepared in fresh 1×TBE buffer in autoclaved Milli-Q water for 90 minutes, at 70 V on ice. 150 ng or otherwise indicated for each RNA sample was loaded and fresh 1×purple loading dye without SDS (NEB) was used. The gel was poststained in 3×GelRed buffer (Biotium) and imaged with a GelDoc-It™ (UVP). Gel images were processed using ImageJ (Fiji) by inverting grayscale and subsequent homogenous background subtracted with 100-150 pixels rolling ball.

### Native Agarose Gel Electrophoresis Analysis of the Molecular 4-Colour and 10-Colour Rulers

4-colour and 10-colour molecular rulers were filtered using 0.5 mL 100 kDa cutoff Amicon filter units. The washing buffer used for filtration is composed of filtered 10 mM Tris-HCl (pH 8.0), 0.5 mM MgCl₂. All samples were pre-mixed with 6×purple loading dye without sodium dodecyl sulfate (SDS) purchased from NEB. 1×loading dye components are 2.5% Ficoll®-400, 10 mM EDTA, 3.3 mM Tris-HCl, 0.02% Dye 1, 0.001% Dye 2, pH 8 at 25° C. Additionally, the samples were mixed with filtered 10×buffer to 1×TBE buffer (Tris-borate-EDTA). The amount of loaded nucleic acids per well was aimed to be from 80-150 ng. All comparable samples were added in the same volume to prevent a salt difference-driven shift.

As shown in FIG. 5, two molecular rulers with 4 colours and 10 colours (lanes 2 and 3 respectively) with biotin-labelling strand without added streptavidin have expected shift from the single-stranded form. Moreover, 10-colour molecular ruler runs slightly slower than a 4-colour ruler as expected from design, since a 10-colour ruler has 45 more structural units (forming structural colours 5, 6, 7, 8, 9, 10) with each having 23 bp and 3 nt dT linker. The 4-colour and 10-colour ruler samples incubated with 10 times excess of neutravidin (ThermoFisher Scientific, catalog number 31050) prior to the PAGE are shown in lanes 5 and 6, respectively. Both rulers were significantly shifted after the addition of neutravidin (lanes 5 and 6) in comparison to the rulers without neutravidin added. 1 kb DNA ladder (NEB, 10 mM Tris-HCl, 1 mM EDTA, pH=8.0 at 25° C.) clearly indicates the expected running speed of the molecular rulers.

### Fluorescence-Quenching Assay for Validation of Structural Colour Assembly

The inventors assembled 10 different molecular rulers where each had only one structural colour from 1 to 10 (1-10 adjacent linearising-structural units). In this case, as a structure 5′ 6-FAM labelled DNA cuboid was used. Firstly, 20 μL of a molecular ruler mix (20 nM) after assembly was mixed with 15 μL of 6-FAM labelled DNA cuboid (1 μM), filtered 4 μL of 1M NaCl, and 2 μL 100 mM MgCl₂ for 2 h at room temperature. After the incubation of the DNA cuboid with a molecular ruler, the inventors added 1 μL the complementary strand with a 3′ Iowa Black fluorescent quencher (100 μM) and incubated it for 1-2 h (FIG. 6A). Iowa Black® quencher is known to quench 6-FAM well since it has broad absorbance spectra ranging from 420 to 620 nm with a peak absorbance at 531 nm (according to Integrated DNATechnologies Inc).

The mixtures were vortexed and spun down after final incubation with quencher strand and diluted with 38 μL of the filtered washing buffer (10 mM Tris-HCl (pH 8.0), 0.5 mM MgCl₂). The spectra were recorded with the Cary Eclipse fluorescence spectrophotometer with Peltier thermostat multicell holder and temperature controller (Agilent) using a glass quartz cuvette. Fluorescent intensity was recorded at the excitation wavelength of 495 nm (absorbance max) and emission spectra are obtained in a range from 500 to 650 nm with the emission max at 520 nm (FIG. 6B). All measurements were recorded at room temperature (20° C.). The measurements for each sample were repeated three times and error bars are presented as a standard error (FIG. 1D).

### MS2 RNA ID Fabrication Using a Part and the Whole RNA Sequence

The inventors assembled MS2 RNA ID using MS2 RNA (Roche through Sigma-Aldrich, catalog number 10165948001). Linearising units (32-48 nt in length) were annealed to the part of MS2 RNA (Table 5) to fabricate MS2 RNA ID ‘111’ partially complementary ID (MS2 RNA ID ‘111’p) as illustrated in FIG. 10A, while for the fully complementary ID (MS2 RNA ID ‘111’f) additional linearising units were added (Table 8). The six interspaced DNA double-hairpin protrusions (labelled as ‘1’) were used to induce a current signal detectable with a nanopore microscope. The distance between colour positions were designed to be Δt1=374 nt (^{˜}127 nm) and Δt2=488 nt (^{˜}166 nm). These two distances were successfully discriminated as shown in nanopore events and by the positional analysis (FIG. 10B-C). The inventors also demonstrated the dependence of event frequency on the concentration of 8 kb DNA (FIG. 10D).

### RNA ID Fabrication in a Complex Mixture of Human Total RNA

The inventors prepared 40 μL reaction by mixing human total RNA (to 12.5 ng/μL) and linearising units specific for all RNA targets (to 60 nM each), in 10 mM MgCl₂ (or 100 mM LiCl), 1×TE (10 mM Tris-HCl, 1 mM EDTA, pH 8.0) buffer, and nuclease-free water was added to the final reaction volume. Buffers were filtered with the MF-Millipore™ Membrane Filter, 0.22 μm pore size. The reaction was mixed by pipetting and spun down. The mixture was heated up to 70° C. for 30 s and gradually cooled down (−0.5° C./cycle, 90 cycles each 30 s) over 45 minutes to room temperature, and hold at 4° C.

Two samples were used for studying RNA identification in a complex mixture e.g. background of total RNA. The first was human universal reference RNA (Invitrogen, catalog number QS0639) that represents a pool of total RNAs from ten different human cell lines/tissues (as listed in Table 9) that were DNase-treated. The second was total RNA originating from cervical adenocarcinoma (HeLa-S3; Invitrogen, catalog number AM7852). Both total RNAs were diluted in nuclease-free water (ThermoFisher) to the final concentration of 100 ng/μL, aliquoted, and stored at −20° C. for short-term use or −80° C. for long-term storage.

For data shown in FIG. 7, the inventors verified that linearising unit mixes for 18S rRNA, 28S rRNA, and Xist lncRNA with M13 and MS2 controls can be assembled in a single-pot reaction. For FIG. 22, the inventors verified the assembly of IDs using linearising unit mixes for ENO1 (linearising units listed in Table 13) and Xist lncRNA (linearising units listed in Table 14).

### RNA ID Temperature Storage Conditions

The inventors assembled MS2 RNA ID ‘111’p and stored it at 4° C. or −20° C. for 1, 4, and 8 days (FIG. 12). IDs were run on 1% (w/v) agarose gel (SigmaAldrich, BioReagent for molecular biology, low EEO; catalog number A9539) prepared in 1×TBE buffer, and cooked in the microwave oven for three minutes and after boiling were stirred and returned. The gel was cooled down under running water, poured, and cast for 1 h at room temperature (20° C.). The samples were run on the gel in 1×TBE buffer for 3 h, 70 V, in the ice water bath. The gel was post-stained in 3×GelRed® in water (Biotium, catalog number 41001) for 10 minutes. The gel was imaged with a UVP GelDoc-It™ imaging system. Gel images were post-processed with an image processing package Fiji (ImageJ). All gel display colours were inverted, and the contrast and brightness were adjusted. The subtract background function was equally applied to the whole gel image using a rolling ball radius of 100-150 pixels (light background). All samples have shown a similar band running on the gel without visible difference also in the nanopore events (FIG. 12B).

### Temperature and Salt Type Effects on the RNA ID Fabrication

The inventors assembled M13 ID ‘11111’ and MS2 ID ‘111’p using either 10 mM MgCl₂ or 100 mM of monovalent salts (LiCl, NaCl, or KCl) with two temperature regimes (starting at 70° C. or 85° C. and gradually cooling to room temperature) as shown in FIG. 13. Nanopore events for both M13 and MS2 and both temperature regimes look as designed. The agarose gel prepared as previously described confirms the correct ID fabrication. However, in the condition with MgCl₂ at 85° C. the gel indicates that almost all RNAs are fragmented and in nanopore events, only a few events were detectable for a 2 h measurement time due to magnesium fragmentation. M13 IDs assembled with magnesium show significant aggregation that is even more prominent at 85° C. (FIG. 13E, lanes 2 and 6). This indicates that magnesium can be omitted from the ID fabrication step. Hence, eliminating magnesium fragmentation and nuclease-degradation of RNA that relies on magnesium ions.

### Salt Concentration Effects on the RNA ID Fabrication

The inventors assembled MS2 ID ‘111’f using either MgCl₂ or LiCl at various concentrations (at 70° C. temperature regime). For magnesium, the inventors used 2.5 mM, 5 mM, or 10 mM MgCl₂, and for lithium the inventors used 25 mM, 50 mM, or 100 mM LiCl (FIG. 14). It can be observed that RNA IDs are assembled under all magnesium concentrations while at 25 mM LiCl RNA ID was not fabricated. The difference in band intensity might be due to the variable amount of recovered RNA IDs after the Amicon filtration.

### Fabrication of IDs for Multiplex Viral Nucleic Acids Identification

The inventors assembled together MS2 RNA ID ‘111’ (grey) and M13 DNA ID ‘111111’. Linearising units (32-48 nt in length) were annealed to the part of MS2 RNA and the whole M13 DNA (linearising units are listed in Table 5 and Table 10. respectively) as illustrated in FIG. 15. The six interspaced DNA double-hairpin protrusions are used to induce a current signal detectable with a nanopore microscope (labelled as ‘1’ in FIG. 15A). These six DNA double-hairpin protrusions are read as one downward signal i.e. current drop. The distance between colours in M13 DNA ID is 1032 bp. The scatter plot of mean event current (nA) versus event duration (in ms) is shown in FIG. 15B. The two populations are assigned to an ID-specific colour based on their ID. In FIG. 15C it can be seen that two populations have distinct event charge deficit (ECD) i.e. surface area of the event. The negligible overlap originates from the fragmented IDs according to the nanopore measurements. FIG. 15D provides example events for MS2 ID ‘111’ (partially complemented with oligos) and M13 ID ‘111111’. These results indicate that the method of the invention is suitable for screening of viruses and pathogens in-parallel.

The inventors prepared 40 μL reaction by mixing linearized M13 ssDNA and MS2 RNA (20 nM or 800 fmoles) and linearising units (60 nM or 2400 fmoles), in 10 mM MgCl₂, 10 mM Tris-HCl, pH 8.0 buffer, and nuclease-free water (Invitrogen™) was added to the final reaction volume. M13 linearization, its purification, and excess oligos removal were done as previously described (J. S. Gootenberg et al., Science. 360, 439-444 (2018)).

Enrichment of RNA IDs from a Complex Sample

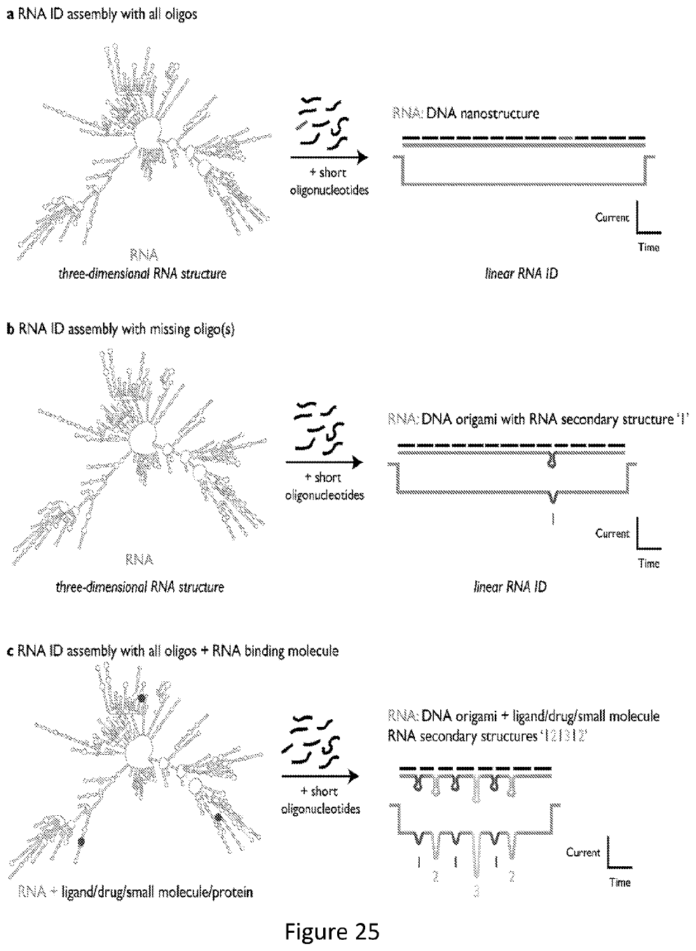

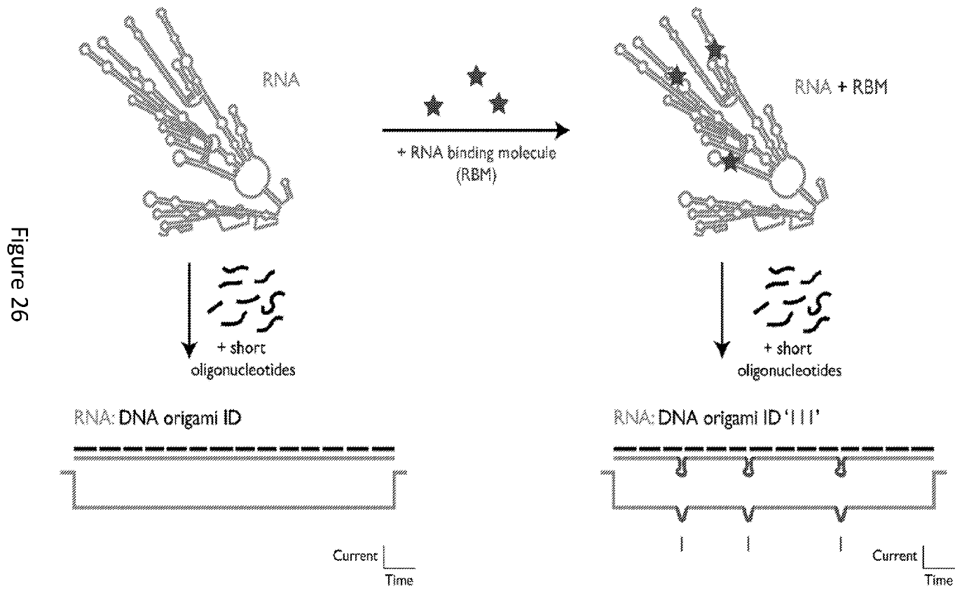

The inventors established an RNA ID enrichment protocol that depletes background <100 kDa single-stranded nucleic acids (FIG. 25) to further decrease background in nanopore measurements. The enrichment of RNA IDs after fabrication was performed by employing Amicon 0.5 mL filters with 100 kDa cut-off using filtered washing buffer (0.5 mM MgCl₂, 10 mM Tris-HCl pH 8.0). RNA ID (40 μL reaction) was filtered with washing buffer (460 μL) two times for 10 minutes, 9,200×g at 3° C. The sample is collected from the tube after enrichment removal and kept on ice until further experimental steps.

### Synthetic Exons Fabrication

Synthetic exons that mimic exons as units that undergo alternative splicing are designed as follows. Each synthetic exon is characterized by a unique three-colour site ID with 20 nt terminal overhangs (FIG. 17). The inventors employed 3.6 kb RNA as a unit length measure and fabricated four different exons. The exon I has ID ‘112’ (FIG. 17A) with terminal ends A and B′ (each 20 nt in length). The exon II has ID ‘312’ (FIG. 17B) with terminal ends A′ and B′ (A and A′ i.e., B and B′ are complementary end sequence pairs). The exon III has ID ‘321’ (FIG. 17C) with terminal ends A′ and B (each 20 nt in length). The exon IV (extended RNA; FIG. 17D) is designed to not carry structural colours and it only has the A′ terminal end sequence. These synthetic exons are characterized by asymmetric ID designs that demonstrate not only the identification of targeted exons but also their directionality. Both are important features for accessing results of alternative processing of transcript.

The linearising units used for the fabrication of synthetic exons are listed in Table 11. Linearising units replaced with linearising-structural units for fabrication of exon I, exon II, exon III, and exon IV are listed in Table 12.

The inventors prepared 40 μL reaction for RNA ID fabrication by mixing RNA sample (20 nM for known target MS2 RNA concentration or 800 fmoles) and linearising units (60 nM each or 2400 fmoles) where some of them contain the linearising-structural units, in 10 mM MgCl₂, 1×TE (10 mM Tris-HCl, 1 mM EDTA, pH 8.0) buffer, and nuclease-free water was added to the final reaction volume. Buffers are filtered with the MF-Millipore™ Membrane Filter, 0.22 μm pore size. The reaction was mixed by pipetting and spun down. The mixture was heated up to 70° C. for 30 s and after gradually cooled down (−0.5° C./cycle, 90 cycles each 30 s) over 45 minutes to room temperature, and hold at 4° C.

The removal of short oligonucleotides after RNA ID fabrication was performed with Amicon 0.5 mL filters with 100 kDa cut-off using filtered washing buffer (0.5 mM MgCl₂, 10 mM Tris-HCl pH 8.0). Synthetic exon mix (40 μL reaction) was filtered with 460 μL washing buffer (460 μL) two times for 10 minutes, 9,200×g at 3° C. The sample was collected by reversing the filter after transfer in a new tube and spun down for 2 minutes, 1,000×g at 3° C. The concentrations of the synthetic exons are estimated from a NanoDrop spectrophotometer.

### Synthetic Isoforms Fabrication

Synthetic isoforms were assembled by linking synthetic exons. The inventors fabricated four isoforms of which three are order isoforms (same length but different synthetic exon IDs) and one length isoform that has one synthetic exon and extended RNA. The three order isoforms were fabricated using exon I and exon II (RNA isoform ID ‘211312’; FIG. 18A), exon I and exon III (RNA isoform ID ‘123112’; FIG. 18B), and exon II and exon II (RNA isoform ID ‘312123’; FIG. 18C). The length isoform was fabricated with exon I and extended RNA (RNA isoform ID ‘211’ extended; FIG. 19).

The inventors mixed 10 μL of each exon (=20 μL), 2 μL 100 mM MgCl₂, 4 μL 1 M NaCl, 14 μL of DNA cuboid (1 μM). The mixtures were incubated at room temperature (20° C.) overnight. After incubation excess DNA cuboid was filtered using afore introduced Amicon 0.5 mL filters (100 kDa cutoff). Buffers are filtered with the MF-Millipore™ Membrane Filter, 0.22 μm pore size.

### Circular and Linear IDs Fabrication

To verify that the method of the invention can discriminate circular and linear conformations, the inventors used circular single-stranded m13mp18 (Guild BioSciences). The linear version was made by annealing a 39 nt oligonucleotide (5′-TCTAGAGGATCCCCGGGTACCGAGCTCGAATTCGTAATC-3′, IDT, IDTE buffer, pH 8.0) to circular form and then subsequent restriction digestion.

Firstly, 40 μL of m13mp18 DNA (100 nM) was mixed with 2 μL oligonucleotide (100 μM), 8 μL 10×Cutsmart buffer (New England Biolabs), and 28 μL of filtered Milli-Q water. This mixture was heated to 65° C. for 30 seconds and gradually cooled down to 25° C. over 40 minutes.

After oligonucleotide annealing 1 μL of BamHI-HF (100.000 units/mL, NEB, catalog number R3136T) and 1 μL of EcoRI-HF (100.000 units/mL, NEB, catalog number R3101T) were added, mixed by pipetting, and incubated at 37° C. for 1 hour. The linear form is purified with Macherey-Nagel™ NucleoSpin™ Gel and PCR Clean-up Kit (Macherey-Nagel™, catalog number 740609.50). The inventors mixed by pipetting 400 μL (5×40 μL mix) of cut ss m13mp18 with 800 μL of binding buffer and separated to three columns. The inventors followed the manufacturer's manual regarding the washing step and centrifugation conditions. Elution buffer was preheated to 70° C. to improve elution from the column. The elution step was repeated twice with 30 μL of elution buffer, after 5 minutes incubation. The concentration of linear m13mp18 is estimated from a NanoDrop spectrophotometer.

To fabricate circular and linear IDs the same linearising unit mixture was used (linearising units listed in Table 1 and Table 13). The inventors prepared 40 μL reaction by mixing linear or circular form (20 nM or 800 fmoles) and linearising units (60 nM each or 2400 fmoles), in 10 mM MgCl₂, 1×TE (10 mM Tris-HCl, 1 mM EDTA, pH 8.0) buffer, and nuclease-free water was added to the final reaction volume. Buffers are filtered with the MF-Millipore™ Membrane Filter, 0.22 μm pore size. The mix is mixed by pipetting and spin down. The mixture was heated up to 70° C. for 30 s and after gradually cooled down (−0.5° C./cycle, 90 cycles each 30 s) over 45 minutes to room temperature, and hold at 4° C.

### In Vitro RNA Circularization

To create circular RNA, the inventors ligated MS2 RNA using T4 RNA ligase 1 and PEG8000 (New England Biolabs (NEB), M0204) that should lead to single-stranded RNA circularization. A 20 μL reaction contained 1×Reaction Buffer (50 mM Tris-HCl, pH 7.5, 10 mM MgCl₂, 1 mM DTT), MS2 RNA (150 nM), 1 μL (10 units) T4 RNA Ligase, 10% PEG8000 and 30 μM ATP. The reaction was incubated overnight at 16° C. To create exclusively circular MS2 RNA ID ‘111’/MS2 a complementary oligonucleotide (1.25 μm) that should join MS2 ends was added to the RNA ID fabrication step.

### Nanopore Fabrication

The inventors fabricated 10-15 nm nanopores using a laser-assisted capillary puller (P2000F, Sutter Instruments). Glass capillaries with an outer diameter of 0.5 mm and an inner diameter of 0.2 mm with filament were purchased from Sutter Instruments, USA. The nanopore diameter was determined with scanning electron microscopy (SEM) and calculated from the conductance of nanopores as previously described (J. S. Gootenberg et al., Science. 360, 439-444 (2018)).

### Nanopore Measurement and Data Analysis

All measurements were performed in 4 M LiCl, 1×TE, pH 9.4 using Axopatch 200B, and data were collected under a constant voltage of 600 mV. Single events in ionic current recordings were firstly isolated according to threshold parameters such as duration, current drop, and event charge deficit (ECD). From isolated events, the conformation of nucleic acids can be determined and for analysis of linear barcodes, unfolded events were used. For analysis of circular RNA, all data were included, and since fully folded events were present at a negligible level in control measurements their effect on data interpretation was minimal.

Amplification-Free RNA Quantification from ID Frequency

The model based on Bell et al. offers an accurate equation for the calculation of DNA concentration based on translocation frequency obtained using glass nanopores. A few considerations have been taken into account for this model. Firstly, the effects of electro-osmotic flows can be neglect, since high salts conditions are used. Secondly, it is of great importance to account for DNA length since the diffusion coefficient is length-dependent. Diffusion coefficients of RNA IDs are calculated from DLS recordings. No significant deviations from data obtained for DNA in 100 mM NaCl, 10 Tris-HCL (pH 8.0), 1 mM EDTA at 20° C. were found. Lastly, it has been demonstrated that the electrophoretic mobility of double-stranded DNA larger than 100 bp (and it is scalable also to RNA) is independent of its length.

The flux i.e. translocation frequency is expressed by a 1D convection-diffusion equation:

$$\\ J ( L ) = \frac{{Dc}_{0}}{L} {( \frac{1}{( \frac{2 {\tilde{u}}_{0}}{η} - \vert \tilde{𝓏} {V}_{m} \vert )} [ {e}^{{}_{} ( \frac{2 {\tilde{u}}_{{0}_{}}}{η} - \vert \tilde{𝓏} {V}_{m} \vert ) \frac{η}{2}} - 1 ] - \frac{{e}^{{}_{} 2 {\tilde{u}}_{0}}}{( \frac{2 {\tilde{u}}_{0}}{η} - \vert \tilde{𝓏} {V}_{m} \vert )} [ {e}^{{}_{} - ( \frac{2 {\tilde{u}}_{{0}_{}}}{η} - \vert \tilde{𝓏} {V}_{m} \vert ) η} - {e}^{{}_{} - ( \frac{2 {\tilde{u}}_{{0}_{}}}{η} - \vert \tilde{𝓏} {V}_{m} \vert ) \frac{η}{2}} ] - \frac{1}{\vert \tilde{𝓏} {V}_{m} \vert} [ {e}^{{}_{} - \vert \tilde{𝓏} {V}_{m} \vert} - {e}^{{}_{} - \vert \tilde{𝓏} {V}_{m} \vert η} ] )}^{- 1}$$

where D is the diffusion coefficient, c₀ is concentration, L is the effective length, ũ₀ is entropic barrier height, η is the distance the entropic barrier extends, \tilde{Z} is an effective charge ( [symbol] ) divided by k_{B}T (k_{B}−Boltzmann constant; T—temperature) and Vₘ is the applied voltage.

The diffusion coefficient is length-dependent (N-DNA length) in 4M LiCl and the following equation can be employed:

$$\\ D = {D}_{0} {N}^{{}_{} - 0.6}$$

For unimolecular RNA ID samples as described before we determined D₀ using DLS. The total charge on 2e⁻ is estimated to be 3.2×10⁻¹⁹ C per base pair and at 20° C. k_{B}T has a value of 4.11×10⁻²¹ J. In all experiments Vₘ was 600 mV. L for the glass nanocapillary system was estimated to be 200 nm.

### TABLES

TABLE 1
Linearising units complementary to linearized ssM13 (7228 nt).

| SEQ | | SEQ | |
|---|---|---|---|
| ID | | ID | |
| NO | Sequence (5′→′3′) | NO | Sequence (5′→′3′) |
| 1 | TTTTCGTAATCATGGTCATAGCTGTTTCCTGTG | 96 | CTTGAGCCATTTGGGAATTAGAGCCAGCAAAATCA |
| | TGAAATTGTTATC | | CCA |
| 2 | CGCTCACAATTCCACACAACATACGAGCCGGAA | 97 | GTAGCACCATTACCATTAGCAAGGCCGGAAACGTC |
| | GCATA | | ACC |
| 3 | AAGTGTAAAGCCTGGGGTGCCTAATGAGTGAGC | 98 | AATGAAACCATCGATAGCAGCACCGTAATCAGTAG |
| | TAACT | | CGA |
| 4 | CACATTAATTGCGTTGCGCTCACTGCCCGCTTT | 99 | CAGAATCAAGTTTGCCTTTAGCGTCAGACTGTAGC |
| | CCAGT | | GCG |
| 5 | CGGGAAACCTGTCGTGCCAGCTGCATTAATGAA | 100 | TTTTCATCGGCATTTTCGGTCATAGCCCCCTTATT |
| | TCGGC | | AGC |
| 6 | CAACGCGCGGGGAGAGGCGGTTTGCGTATTGGG | 101 | GTTTGCCATCTTTTCATAATCAAAATCACCGGAAC |
| | CGCCA | | CAG |
| 7 | GGGTGGTTTTTCTTTTCACCAGTGAGACGGGCA | 102 | AGCCACCACCGGAACCGCCTCCCTCAGAGCCGCCA |
| | ACAGC | | CCC |
| 8 | TGATTGCCCTTCACCGCCTGGCCCTGAGAGAGT | 103 | TCAGAACCGCCACCCTCAGAGCCACCACCCTCAGA |
| | TGCAG | | GCC |
| 9 | CAAGCGGTCCACGCTGGTTTGCCCCAGCAGGCG | 104 | GCCACCAGAACCACCACCAGAGCCGCCGCCAGCAT |
| | AAAAT | | TGA |
| 10 | CCTGTTTGATGGTGGTTCCGAAATCGGCAAAAT | 105 | CAGGAGGTTGAGGCAGGTCAGACGATTGGCCTTGA |
| | CCCTT | | TAT |
| 11 | ATAAATCAAAAGAATAGCCCGAGATAGGGTTGA | 106 | TCACAAACAAATAAATCCTCATTAAAGCCAGAATG |
| | GTGTT | | GAA |
| 12 | GTTCCAGTTTGGAACAAGAGTCCACTATTAAAG | 107 | AGCGCAGTCTCTGAATTTACCGTTCCAGTAAGCGT |
| | AACGT | | CAT |
| 13 | GGACTCCAACGTCAAAGGGCGAAAAACCGTCTA | 108 | ACATGGCTTTTGATGATACAGGAGTGTACTGGTAA |
| | TCAGG | | TAA |
| 14 | GCGATGGCCCACTACGTGAACCATCACCCAAAT | 109 | GTTTTAACGGGGTCAGTGCCTTGAGTAACAGTGCC |
| | CAAGT | | CGT |
| 15 | TTTTTGGGGTCGAGGTGCCGTAAAGCACTAAAT | 110 | ATAAACAGTTAATGCCCCCTGCCTATTTCGGAACC |
| | CGGAA | | TAT |
| 16 | CCCTAAAGGGAGCCCCCGATTTAGAGCTTGACG | 111 | TATTCTGAAACATGAAAGTATTAAGAGGCTGAGAC |
| | GGGAA | | TCC |
| 17 | AGCCGGCGAACGTGGCGAGAAAGGAAGGGAAGA | 112 | TCAAGAGAAGGATTAGGATTAGCGGGGTTTTGCTC |
| | AAGCG | | AGT |
| 18 | AAAGGAGCGGGCGCTAGGGCGCTGGCAAGTGTA | 113 | ACCAGGCGGATAAGTGCCGTCGAGAGGGTTGATAT |
| | GCGGT | | AAG |
| 19 | CACGCTGCGCGTAACCACCACACCCGCCGCGCT | 114 | TATAGCCCGGAATAGGTGTATCACCGTACTCAGGA |
| | TAATG | | GGT |
| 20 | CGCCGCTACAGGGCGCGTACTATGGTTGCTTTG | 115 | TTAGTACCGCCACCCTCAGAACCGCCACCCTCAGA |
| | ACGAG | | ACC |
| 21 | CACGTATAACGTGCTTTCCTCGTTAGAATCAGA | 116 | GCCACCCTCAGAGCCACCACCCTCATTTTCAGGGA |
| | GCGGG | | TAG |
| 22 | AGCTAAACAGGAGGCCGATTAAAGGGATTTTAG | 117 | CAAGCCCAATAGGAACCCATGTACCGTAACACTGA |
| | ACAGG | | GTT |
| 23 | AACGGTACGCCAGAATCCTGAGAAGTGTTTTTA | 118 | TCGTCACCAGTACAAACTACAACGCCTGTAGCATT |
| | TAATC | | CCA |
| 24 | AGTGAGGCCACCGAGTAAAAGAGTCTGTCCATC | 119 | CAGACAGCCCTCATAGTTAGCGTAACGATCTAAAG |
| | ACGCA | | TTT |
| 25 | AATTAACCGTTGTAGCAATACTTCTTTGATTAG | 120 | TGTCGTCTTTCCAGACGTTAGTAAATGAATTTTCT |
| | TAATA | | GTA |
| 26 | ACATCACTTGCCTGAGTAGAAGAACTCAAACTA | 121 | TGGGATTTTGCTAAACAACTTTCAACAGTTTCAGC |
| | TCGGC | | GGA |
| 27 | CTTGCTGGTAATATCCAGAACAATATTACCGCC | 122 | GTGAGAATAGAAAGGAACAACTAAAGGAATTGCGA |
| | AGCCA | | ATA |
| 28 | TTGCAACAGGAAAAACGCTCATGGAAATACCTA | 123 | ATAATTTTTTCACGTTGAAAATCTCCAAAAAAAAG |
| | CATTT | | GCT |
| 29 | TGACGCTCAATCGTCTGAAATGGATTATTTACA | 124 | CCAAAAGGAGCCTTTAATTGTATCGGTTTATCAGC |
| | TTGGC | | TTG |
| 30 | AGATTCACCAGTCACACGACCAGTAATAAAAGG | 125 | CTTTCGAGGTGAATTTCTTAAACAGCTTGATACCG |
| | GACAT | | ATA |
| 31 | TCTGGCCAACAGAGATAGAACCCTTCTGACCTG | 126 | GTTGCGCCGACAATGACAACAACCATCGCCCACGC |
| | AAAGC | | ATA |
| 32 | GTAAGAATACGTGGCACAGACAATATTTTTGAA | 127 | ACCGATATATTCGGTCGCTGAGGCTTGCAGGGAGT |
| | TGGCT | | TAA |
| 33 | ATTAGTCTTTAATGCGCGAACTGATAGCCCTAA | 128 | AGGCCGCTTTTGCGGGATCGTCACCCTCAGCAGCG |
| | AACAT | | AAA |
| 34 | CGCCATTAAAAATACCGAACGAACCACCAGCAG | 129 | GACAGCATCGGAACGAGGGTAGCAACGGCTACAGA |
| | AAGAT | | GGC |
| 35 | AAAACAGAGGTGAGGCGGTCAGTATTAACACCG | 130 | TTTGAGGACTAAAGACTTTTTCATGAGGAAGTTTC |
| | CCTGC | | CAT |
| 36 | AACAGTGCCACGCTGAGAGCCAGCAGCAAATGA | 131 | TAAACGGGTAAAATACGTAATGCCACTACGAAGGC |
| | AAAAT | | ACC |
| 37 | CTAAAGCATCACCTTGCTGAACCTCAAATATCA | 132 | AACCTAAAACGAAAGAGGCAAAAGAATACACTAAA |
| | AACCC | | ACA |
| 38 | TCAATCAATATCTGGTCAGTTGGCAAATCAACA | 133 | CTCATCTTTGACCCCCAGCGATTATACCAAGCGCG |
| | GTTGA | | AAA |
| 39 | AAGGAATTGAGGAAGGTTATCTAAAATATCTTT | 134 | CAAAGTACAACGGAGATTTGTATCATCGCCTGATA |
| | AGGAG | | AAT |
| 40 | CACTAACAACTAATAGATTAGAGCCGTCAATAG | 135 | TGTGTCGAAATCCGCGACCTGCTCCATGTTACTTA |
| | ATAAT | | GCC |
| 41 | ACATTTGAGGATTTAGAAGTATTAGACTTTACA | 136 | GGAACGAGGCGCAGACGGTCAATCATAAGGGAACC |
| | AACAA | | GAA |
| 42 | TTCGACAACTCGTATTAAATCCTTTGCCCGAAC | 137 | CTGACCAACTTTGAAAGAGGACAGATGAACGGTGT |
| | GTTAT | | ACA |
| 43 | TAATTTTAAAAGTTTGAGTAACATTATCATTTT | 138 | GACCAGGCGCATAGGCTGGCTGACCTTCATCAAGA |
| | GCGGA | | GTA |
| 44 | ACAAAGAAACCACCAGAAGGAGCGGAATTATCA | 139 | ATCTTGACAAGAACCGGATATTCATTACCCAAATC |
| | TCATA | | AAC |
| 45 | TTCCTGATTATCAGATGATGGCAATTCATCAAT | 140 | GTAACAAAGCTGCTCATTCAGTGAATAAGGCTTGC |
| | ATAAT | | CCT |
| 46 | CCTGATTGTTTGGATTATACTTCTGAATAATGG | 141 | GACGAGAAACACCAGAACGAGTAGTAAATTGGGCT |
| | AAGGG | | TGA |
| 47 | TTAGAACCTACCATATCAAAATTATTTGCACGT | 142 | GATGGTTTAATTTCAACTTTAATCATTGTGAATTA |
| | AAAAC | | CCT |
| 48 | AGAAATAAAGAAATTGCGTAGATTTTCAGGTTT | 143 | TATGCGATTTTAAGAACTGGCTCATTATACCAGTC |
| | AACGT | | AGG |
| 49 | CAGATGAATATACAGTAACAGTACCTTTTACAT | 144 | ACGTTGGGAAGAAAAATCTACGTTAATAAAACGAA |
| | CGGGA | | CTA |
| 50 | GAAACAATAACGGATTCGCCTGATTGCTTTGAA | 145 | ACGGAACAACATTATTACAGGTAGAAAGATTCATC |
| | TACCA | | AGT |
| 51 | AGTTACAAAATCGCGCAGAGGCGAATTATTCAT | 146 | TGAGATTTAGGAATACCACATTCAACTAATGCAGA |
| | TTCAA | | TAC |
| 52 | TTACCTGAGCAAAAGAAGATGATGAAACAAACA | 147 | ATAACGCCAAAAGGAATTACGAGGCATAGTAAGAG |
| | TCAAG | | CAA |
| 53 | AAAACAAAATTAATTACATTTAACAATTTCATT | 148 | CACTATCATAACCCTCGTTTACCAGACGACGATAA |
| | TGAAT | | AAA |
| 54 | TACCTTTTTTAATGGAAACAGTACATAAATCAA | 149 | CCAAAATAGCGAGAGGCTTTTGCAAAAGAAGTTTT |
| | TATAT | | GCC |
| 55 | GTGAGTGAATAACCTTGCTTCTGTAAATCGTCG | 150 | AGAGGGGGTAATAGTAAAATGTTTAGACTGGATAG |
| | CTATT | | CGT |
| 56 | AATTAATTTTCCCTTAGAATCCTTGAAAACATA | 151 | CCAATACTGCGGAATCGTCATAAATATTCATTGAA |
| | GCGAT | | TCC |
| 57 | AGCTTAGATTAAGACGCTGAGAAGAGTCAATAG | 152 | CCCTCAAATGCTTTAAACAGTTCAGAAAACGAGAA |
| | TGAAT | | TGA |
| 58 | TTATCAAAATCATAGGTCTGAGAGACTACCTTT | 153 | CCATAAATCAAAAATCAGGTCTTTACCCTGACTAT |
| | TTAAC | | TAT |
| 59 | CTCCGGCTTAGGTTGGGTTATATAACTATATGT | 154 | AGTCAGAAGCAAAGCGGATTGCATCAAAAAGATTA |
| | AAATG | | AGA |
| 60 | CTGATGCAAATCCAATCGCAAGACAAAGAACGC | 155 | GGAAGCCCGAAAGACTTCAAATATCGCGTTTTAAT |
| | GAGAA | | TCG |
| 61 | AACTTTTTCAAATATATTTTAGTTAATTTCATC | 156 | AGCTTCAAAGCGAACCAGACCGGAAGCAAACTCCA |
| | TTCTG | | ACA |
| 62 | ACCTAAATTTAATGGTTTGAAATACCGACCGTG | 157 | GGTCAGGATTAGAGAGTACCTTTAATTGCTCCTTT |
| | TGATA | | TGA |
| 63 | AATAAGGCGTTAAATAAGAATAAACACCGGAAT | 158 | TAAGAGGTCATTTTTGCGGATGGCTTAGAGCTTAA |
| | CATAA | | TTG |
| 64 | TTACTAGAAAAAGCCTGTTTAGTATCATATGCG | 159 | CTGAATATAATGCTGTAGCTCAACATGTTTTAAAT |
| | TTATA | | ATG |
| 65 | CAAATTCTTACCAGTATAAAGCCAACGCTCAAC | 160 | CAACTAAAGTACGGTGTCTGGAAGTTTCATTCCAT |
| | AGTAG | | ATA |
| 66 | GGCTTAATTGAGAATCGCCATATTTAACAACGC | 161 | ACAGTTGATTCCCAATTCTGCGAACGAGTAGATTT |
| | CAACA | | AGT |
| 67 | TGTAATTTAGGCAGAGGCATTTTCGAGCCAGTA | 162 | TTGACCATTAGATACATTTCGCAAATGGTCAATAA |
| | ATAAG | | CCT |
| 68 | AGAATATAAAGTACCGACAAAAGGTAAAGTAAT | 163 | GTTTAGCTATATTTTCATTTGGGGCGCGAGCTGAA |
| | TCTGT | | AAG |
| 69 | CCAGACGACGACAATAAACAACATGTTCAGCTA | 164 | GTGGCATCAATTCTACTAATAGTAGTAGCATTAAC |
| | ATGCA | | ATC |
| 70 | GAACGCGCCTGTTTATCAACAATAGATAAGTCC | 165 | CAATAAATCATACAGGCAAGGCAAAGAATTAGCAA |
| | TGAAC | | AAT |
| 71 | AAGAAAAATAATATCCCATCCTAATTTACGAGC | 166 | TAAGCAATAAAGCCTCAGAGCATAAAGCTAAATCG |
| | ATGTA | | GTT |
| 72 | GAAACCAATCAATAATCGGCTGTCTTTCCTTAT | 167 | GTACCAAAAACATTATGACCCTGTAATACTTTTGC |
| | CATTC | | GGG |
| 73 | CAAGAACGGGTATTAAACCAAGTACCGCACTCA | 168 | AGAAGCCTTTATTTCAACGCAAGGATAAAAATTTT |
| | TCGAG | | TAG |
| 74 | AACAAGCAAGCCGTTTTTATTTTCATCGTAGGA | 169 | AACCCTCATATATTTTAAATGCAATGCCTGAGTAA |
| | ATCAT | | TGT |
| 75 | TACCGCGCCCAATAGCAAGCAAATCAGATATAG | 170 | GTAGGTAAAGATTCAAAAGGGTGAGAAAGGCCGGA |
| | AAGGC | | GAC |
| 76 | TTATCCGGTATTCTAAGAACGCGAGGCGTTTTA | 171 | AGTCAAATCACCATCAATATGATATTCAACCGTTC |
| | GCGAA | | TAG |
| 77 | CCTCCCGACTTGCGGGAGGTTTTGAAGCCTTAA | 172 | CTGATAAATTAATGCCGGAGAGGGTAGCTATTTTT |
| | ATCAA | | GAG |
| 78 | GATTAGTTGCTATTTTGCACCCAGCTACAATTT | 173 | AGATCTACAAAGGCTATCAGGTCATTGCCTGAGAG |
| | TATCC | | TCT |
| 79 | TGAATCTTACCAACGCTAACGAGCGTCTTTCCA | 174 | GGAGCAAACAAGAGAATCGATGAACGGTAATCGTA |
| | GAGCC | | AAA |
| 80 | TAATTTGCCAGTTACAAAATAAACAGCCATATT | 175 | CTAGCATGTCAATCATATGTACCCCGGTTGATAAT |
| | ATTTA | | CAG |
| 81 | TCCCAATCCAAATAAGAAACGATTTTTTGTTTA | 176 | AAAAGCCCCAAAAACAGGAAGATTGTATAAGCAAA |
| | ACGTC | | TAT |
| 82 | AAAAATGAAAATAGCAGCCTTTACAGAGAGAAT | 177 | TTAAATTGTAAACGTTAATATTTTGTTAAAATTCG |
| | AACAT | | CAT |
| 83 | AAAAACAGGGAAGCGCATTAGACGGGAGAATTA | 178 | TAAATTTTTGTTAAATCAGCTCATTTTTTAACCAA |
| | ACTGA | | TAG |
| 84 | ACACCCTGAACAAAGTCAGAGGGTAATTGAGCG | 179 | GAACGCCATCAAAAATAATTCGCGTCTGGCCTTCC |
| | CTAAT | | TGT |
| 85 | ATCAGAGAGATAACCCACAAGAATTGAGTTAAG | 180 | AGCCAGCTTTCATCAACATTAAATGTGAGCGAGTA |
| | CCCAA | | ACA |
| 86 | TAATAAGAGCAAGAAACAATGAAATAGCAATAG | 181 | ACCCGTCGGATTCTCCGTGGGAACAAACGGCGGAT |
| | CTATC | | TGA |
| 87 | TTACCGAAGCCCTTTTTAAGAAAAGTAAGCAGA | 182 | CCGTAATGGGATAGGTCACGTTGGTGTAGATGGGC |
| | TAGCC | | GCA |
| 88 | GAACAAAGTTACCAGAAGGAAACCGAGGAAACG | 183 | TCGTAACCGTGCATCTGCCAGTTTGAGGGGACGAC |
| | CAATA | | GAC |
| 89 | ATAACGGAATACCCAAAAGAACTGGCATGATTA | 184 | AGTATCGGCCTCAGGAAGATCGCACTCCAGCCAGC |
| | AGACT | | TTT |
| 90 | CCTTATTACGCAGTATGTTAGCAAACGTAGAAA | 185 | CCGGCACCGCTTCTGGTGCCGGAAACCAGGCAAAG |
| | ATACA | | CGC |
| 91 | TACATAAAGGTGGCAACATATAAAAGAAACGCA | 186 | CATTCGCCATTCAGGCTGCGCAACTGTTGGGAAGG |
| | AAGAC | | GCG |
| 92 | ACCACGGAATAAGTTTATTTTGTCACAATCAAT | 187 | ATCGGTGCGGGCCTCTTCGCTATTACGCCAGCTGG |
| | AGAAA | | CGA |
| 93 | ATTCATATGGTTTACCAGCGCCAAAGACAAAAG | 188 | AAGGGGGATGTGCTGCAAGGCGATTAAGTTGGGTA |
| | GGCGA | | ACG |
| 94 | CATTCAACCGATTGAGGGAGGGAAGGTAAATAT | 189 | CCAGGGTTTTCCCAGTCACGACGTTGTAAAACGAC |
| | TGACG | | GGC |
| 95 | GAAATTATTCATTAAAGGTGAATTATCACCGTC | 190 | CAGTGCCAAGCTTGCATGCCTGCAGGTCGACTCTA |
| | ACCGA | | GAGGATCTTTT |

TABLE 2
Linearising-structural units providing four structural colours along ssM13 (4-colour
ruler fabrication). Replaced SEQ ID NO correspond to the linearising unit SEQ IDs
listed in Table 1 that are replaced to produce structural colours.

| Structural | Replaced | SEQ ID | | |
|---|---|---|---|---|
| colour | SEQ ID NO | NO | Name | Sequence (5′→′3′) |
| 1 | 14, 15 | 191 | B-1 | GCGATGGCCCACTACGTGAACCATC TTT |
| | | | | GGATATCACTCATTAGTGGT |
| | | 192 | B-2 | ACCCAAATCAAGTTTTTTGGGGTCG |
| | | 193 | B-3 | AGGTGCCGTAAAGCACTAAATCGGAA |
| 2 | 28, 29 | 194 | B-4 | TTGCAACAGGAAAAACGCTCATGGA TTT |
| | | | | GGATATCACTCATTAGTGGT |
| | | 195 | B-5 | AATACCTACATTTTGACGCTCAATC TTT |
| | | | | GGATATCACTCATTAGTGGT |
| | | 196 | B-6 | GTCTGAAATGGATTATTTACATTGGC |
| 3 | 43, 44 | 197 | B-7 | TAATTTTAAAAGTTTGAGTAACATT TTT |
| | | | | GGATATCACTCATTAGTGGT |
| | | 198 | B-8 | ATCATTTTGCGGAACAAAGAAACCA TTT |
| | | | | GGATATCACTCATTAGTGGT |
| | | 199 | B-9 | CCAGAAGGAGCGGAATTATCATCATA TTT |
| | | | | GGATATCACTCATTAGTGGT |
| 4 | 58, 59, 60, | 200 | B-10 | TTATCAAAATCATAGGTCTGAGAGA TTT |
| | 61 | | | GGATATCACTCATTAGTGGT |
| | | 201 | B-11 | CTACCTTTTTAACCTCCGGCTTAGG TTT |
| | | | | GGATATCACTCATTAGTGGT |
| | | 202 | B-12 | TTGGGTTATATAACTATATGTAAAT TTT |
| | | | | GGATATCACTCATTAGTGGT |
| | | 203 | B-13 | GCTGATGCAAATCCAATCGCAAGAC TTT |
| | | | | GGATATCACTCATTAGTGGT |
| | | 204 | B-14 | AAAGAACGCGAGAAAACTTTTTCAAA |
| | | 205 | B-15 | TATATTTTAGTTAATTTCATCTTCTG |
| | ‘Labelling | 206 | Bio-strand | ACCACTAATGAGTGATATCC/3′-biotin/ |
| | strand’ | | HPLC | |

TABLE 3
Linearising-structural units providing ten structural colours along ssM13 (10-colour
ruler fabrication). Replaced SEQ ID NO correspond to the linearising unit SEQ IDs
listed in Table 1 that are replaced to produce structural colours.

| Structural | Replaced | SEQ ID | | |
|---|---|---|---|---|
| colour | SEQ ID NO | NO | Name | Sequence (5′→′3′) |
| 1 | 14, 15 | 191 | B-1 | GCGATGGCCCACTACGTGAACCATC TTT |
| | | | | GGATATCACTCATTAGTGGT |
| | | 192 | B-2 | ACCCAAATCAAGTTTTTTGGGGTCG |
| | | 193 | B-3 | AGGTGCCGTAAAGCACTAAATCGGAA |
| 2 | 28, 29 | 194 | B-4 | TTGCAACAGGAAAAACGCTCATGGA TTT |
| | | | | GGATATCACTCATTAGTGGT |
| | | 195 | B-5 | AATACCTACATTTTGACGCTCAATC TTT |
| | | | | GGATATCACTCATTAGTGGT |
| | | 196 | B-6 | GTCTGAAATGGATTATTTACATTGGC |
| 3 | 43, 44 | 197 | B-7 | TAATTTTAAAAGTTTGAGTAACATT TTT |
| | | | | GGATATCACTCATTAGTGGT |
| | | 198 | B-8 | ATCATTTTGCGGAACAAAGAAACCA TTT |
| | | | | GGATATCACTCATTAGTGGT |
| | | 199 | B-9 | CCAGAAGGAGCGGAATTATCATCATA TTT |
| | | | | GGATATCACTCATTAGTGGT |
| 4 | 58, 59, 60, | 200 | B-10 | TTATCAAAATCATAGGTCTGAGAGA TTT |
| | 61 | | | GGATATCACTCATTAGTGGT |
| | | 20 | B-11 | CTACCTTTTTAACCTCCGGCTTAGG TTT |
| | | | | GGATATCACTCATTAGTGGT |
| | | 202 | B-12 | TTGGGTTATATAACTATATGTAAAT TTT |
| | | | | GGATATCACTCATTAGTGGT |
| | | 203 | B-13 | GCTGATGCAAATCCAATCGCAAGAC TTT |
| | | | | GGATATCACTCATTAGTGGT |
| | | 204 | B-14 | AAAGAACGCGAGAAAACTTTTTCAAA |
| | | 205 | B-15 | TATATTTTAGTTAATTTCATCTTCTG |
| 5 | 74, 75, 76, | 207 | B-16 | AACAAGCAAGCCGTTTTTATTTTCA TTT |
| | 77 | | | GGATATCACTCATTAGTGGT |
| | | 208 | B-17 | TCGTAGGAATCATTACCGCGCCCAA TTT |
| | | | | GGATATCACTCATTAGTGGT |
| | | 209 | B-18 | TAGCAAGCAAATCAGATATAGAAGG TTT |
| | | | | GGATATCACTCATTAGTGGT |
| | | 210 | B-19 | CTTATCCGGTATTCTAAGAACGCGA TTT |
| | | | | GGATATCACTCATTAGTGGT |
| | | 211 | B-20 | GGCGTTTTAGCGAACCTCCCGACTT TTT |
| | | | | GGATATCACTCATTAGTGGT |
| | | 212 | B-21 | GCGGGAGGTTTTGAAGCCTTAAATCAA |
| 6 | 90, 91, 92, | 213 | B-22 | CCTTATTACGCAGTATGTTAGCAAA TTT |
| | 93 | | | GGATATCACTCATTAGTGGT |
| | | 214 | B-23 | CGTAGAAAATACATACATAAAGGTG TTT |
| | | | | GGATATCACTCATTAGTGGT |
| | | 215 | B-24 | GCAACATATAAAAGAAACGCAAAGA TTT |
| | | | | GGATATCACTCATTAGTGGT |
| | | 216 | B-25 | CACCACGGAATAAGTTTATTTTGTC TTT |
| | | | | GGATATCACTCATTAGTGGT |
| | | 217 | B-26 | ACAATCAATAGAAAATTCATATGGT TTT |
| | | | | GGATATCACTCATTAGTGGT |
| | | 218 | B-27 | TTACCAGCGCCAAAGACAAAAGGGCGA TTT |
| | | | | GGATATCACTCATTAGTGGT |
| 7 | 107, 108, | 219 | B-28 | AGCGCAGTCTCTGAATTTACCGTTC TTT |
| | 109, 110, | | | GGATATCACTCATTAGTGGT |
| | 111 | 220 | B-29 | CAGTAAGCGTCATACATGGCTTTTG TTT |
| | | | | GGATATCACTCATTAGTGGT |
| | | 221 | B-30 | ATGATACAGGAGTGTACTGGTAATA TTT |
| | | | | GGATATCACTCATTAGTGGT |
| | | 222 | B-31 | AGTTTTAACGGGGTCAGTGCCTTGA TTT |
| | | | | GGATATCACTCATTAGTGGT |
| | | 223 | B-32 | GTAACAGTGCCCGTATAAACAGTTA TTT |
| | | | | GGATATCACTCATTAGTGGT |
| | | 224 | B-33 | ATGCCCCCTGCCTATTTCGGAACCT TTT |
| | | | | GGATATCACTCATTAGTGGT |
| | | 225 | B-34 | ATTATTCTGAAACATGAAAGTATTA TTT |
| | | | | GGATATCACTCATTAGTGGT |
| | | 226 | B-35 | AGAGGCTGAGACTCC |
| 8 | 125, 126, | 227 | B-36 | CTTTCGAGGTGAATTTCTTAAACAG TTT |
| | 127, 128, | | | GGATATCACTCATTAGTGGT |
| | 129, 130 | 228 | B-37 | CTTGATACCGATAGTTGCGCCGACA TTT |
| | | | | GGATATCACTCATTAGTGGT |
| | | 229 | B-38 | ATGACAACAACCATCGCCCACGCAT TTT |
| | | | | GGATATCACTCATTAGTGGT |
| | | 230 | B-39 | AACCGATATATTCGGTCGCTGAGGC TTT |
| | | | | GGATATCACTCATTAGTGGT |
| | | 231 | B-40 | TTGCAGGGAGTTAAAGGCCGCTTTT TTT |
| | | | | GGATATCACTCATTAGTGGT |
| | | 232 | B-41 | GCGGGATCGTCACCCTCAGCAGCGA TTT |
| | | | | GGATATCACTCATTAGTGGT |
| | | 233 | B-42 | AAGACAGCATCGGAACGAGGGTAGC TTT |
| | | | | GGATATCACTCATTAGTGGT |
| | | 234 | B-43 | AACGGCTACAGAGGCTTTGAGGACT TTT |
| | | | | GGATATCACTCATTAGTGGT |
| | | 235 | B-44 | AAAGACTTTTTCATGAGGAAGTTTCCAT |
| 9 | 143, 144, | 236 | B-45 | TATGCGATTTTAAGAACTGGCTCAT TTT |
| | 145, 146, | | | GGATATCACTCATTAGTGGT |
| | 147, 148 | 237 | B-46 | TATACCAGTCAGGACGTTGGGAAGA TTT |
| | | | | GGATATCACTCATTAGTGGT |
| | | 238 | B-47 | AAAATCTACGTTAATAAAACGAACT TTT |
| | | | | GGATATCACTCATTAGTGGT |
| | | 239 | B-48 | AACGGAACAACATTATTACAGGTAG TTT |
| | | | | GGATATCACTCATTAGTGGT |
| | | 240 | B-49 | AAAGATTCATCAGTTGAGATTTAGG TTT |
| | | | | GGATATCACTCATTAGTGGT |
| | | 241 | B-50 | AATACCACATTCAACTAATGCAGAT TTT |
| | | | | GGATATCACTCATTAGTGGT |
| | | 242 | B-51 | ACATAACGCCAAAAGGAATTACGAG TTT |
| | | | | GGATATCACTCATTAGTGGT |
| | | 243 | B-52 | GCATAGTAAGAGCAACACTATCATA TTT |
| | | | | GGATATCACTCATTAGTGGT |
| | | 244 | B-53 | ACCCTCGTTTACCAGACGACGATAA AAA TTT |
| | | | | GGATATCACTCATTAGTGGT |
| 10 | 162, 163, | 245 | B-54 | TTGACCATTAGATACATTTCGCAAA TTT |
| | 164, 165, | | | GGATATCACTCATTAGTGGT |
| | 166, 167, | 246 | B-55 | TGGTCAATAACCTGTTTAGCTATAT TTT |
| | 168, 169 | | | GGATATCACTCATTAGTGGT |
| | | 247 | B-56 | TTTCATTTGGGGCGCGAGCTGAAAA TTT |
| | | | | GGATATCACTCATTAGTGGT |
| | | 248 | B-57 | GGTGGCATCAATTCTACTAATAGTA TTT |
| | | | | GGATATCACTCATTAGTGGT |
| | | 249 | B-58 | GTAGCATTAACATCCAATAAATCAT TTT |
| | | | | GGATATCACTCATTAGTGGT |
| | | 250 | B-59 | ACAGGCAAGGCAAAGAATTAGCAAA TTT |
| | | | | GGATATCACTCATTAGTGGT |
| | | 251 | B-60 | ATTAAGCAATAAAGCCTCAGAGCAT TTT |
| | | | | GGATATCACTCATTAGTGGT |
| | | 252 | B-61 | AAAGCTAAATCGGTTGTACCAAAAA TTT |
| | | | | GGATATCACTCATTAGTGGT |
| | | 253 | B-62 | CATTATGACCCTGTAATACTTTTGC TTT |
| | | | | GGATATCACTCATTAGTGGT |
| | | 254 | B-63 | GGGAGAAGCCTTTATTTCAACGCAA TTT |
| | | | | GGATATCACTCATTAGTGGT |
| | | 255 | B-64 | GGATAAAAATTTTTAGAACCCTCATAT |
| | | 256 | B-65 | ATTTTAAATGCAATGCCTGAGTAATGT |
| | ‘Labelling | 206 | Bio- | ACCACTAATGAGTGATATCC/3′-biotin/ |
| | strand’ | | strand | |
| | | | HPLC | |

TABLE 4
DNA oligonucleotides for DNA cuboid. In bold underline is highlighted ′labelling
strand′ region complementary to the linearising-structural unit ′docking strand′.
SEQ ID NO 263 is labelled oligo with 6-fluorescein (6-FAM) used for the
fluorescence-quenching assay instead of 1M1.

| SEQ ID | | | Length |
|---|---|---|---|
| NO | Name | Sequence (5′→3′) | (nt) |
| 257 | 2S1 | GCCGACGTGTACGGATCTGGCA | 22 |
| 258 | 2S2 | GCGACATTGCGGCCGATTCGGA | 22 |
| 259 | 1M1 | ACCACTAATGAGTGATATCCTTTTCGCATCAGGGCACATTGGCTTT | 46 |
| 260 | 1M2 | TTTTGCGGCTAAGACTTGCCAGGTTT | 26 |
| 261 | 3L1 | TTTTACACGTCGGCCCTGATGCGACCTGGCAAGTTCCGAATCGGCTTT | 48 |
| 262 | 3L2 | TTTCGCAATGTCGCCTTAGCCGCAGCCAATGTGCTGCCAGATCCGTTT | 48 |
| 263 | 1M1_FAM | /56-FAM-/ | 46 |
| | | ACCACTAATGAGTGATATCCTTTTCGCATCAGGGCACATTGGCTTT | |
| 264 | 1M1c_Black | GGATATCACTCATTAGTGGT/3IABKFQ/ | 20 |
| | Iowa | | |
| | Quencher | | |

TABLE 5
Linearising units for fabrication of partially complementary MS2 RNA ID ′111′.
Linearising units that form structural colours are SEQ ID NOs 274-279; 293-298;
and 309-314 (highlighted in bold).

| SEQ ID | | | Length |
|---|---|---|---|
| NO | Name | Sequence (5′→′3′)) | (nt) |
| 265 | M_1 | CACTCCGTTCCCTACAACGAGCCTAAATTCATATGACT | 38 |
| 266 | M_2 | CGTTATAGCGGACCGCGTGTCTGATCCACGGCGCACAT | 38 |
| 267 | M_3 | TGGTCTCGGACCAATAGAGCCGCTCTCAGAGCGCGGGG | 38 |
| 268 | M_4 | GGTAACGGTTGCTTGTTCAGCGAACTTCTTGTAAGGCG | 38 |
| 269 | M_5 | CTGCATCCTGCAACTTGTGCCCCATAGGAGCACCGTTG | 38 |
| 270 | M_6 | GAGAACGTGCATTGCCCAAACAACGACGATCGGTAGCC | 38 |
| 271 | M_7 | AGAGAGGAGGTTGCCAATAAGGCTACGGATGCTGGTTT | 38 |
| 272 | M_8 | GTAAAACATCCGGATCCCATGACAAGGATTTGTCATGT | 38 |
| 273 | M_9 | AAGAAACCTTCTCTATTTATCTGACCGCGATCACCATT | 38 |
| 274 | M_10 | CGCCTCCCGTTCCTCTTTTGAGGAACAAGTTTTCTTGTAGCTTAGCGA | 48 |
| 275 | M_11 | TAGCTAAGGTTCCTCTTTTGAGGAACAAGTTTTCTTGTACGACGGGTC | 48 |
| 276 | M_12 | GCCTCGTCATTCCTCTTTTGAGGAACAAGTTTTCTTGTTACCAGAACC | 48 |
| 277 | M_13 | TAAGGTCGGATCCTCTTTTGAGGAACAAGTTTTCTTGTTGCTTTGTGA | 48 |
| 278 | M_14 | GCAATTCGTCTCCTCTTTTGAGGAACAAGTTTTCTTGTCCTTAAGTAA | 48 |
| 279 | M_15 | GCAATTGCTGTCCTCTTTTGAGGAACAAGTTTTCTTGTTAAAGTCGTC | 48 |
| 280 | M_16 | ACTGTGCGGATCACCGCTTCCAGTAGCGACAG | 32 |
| 281 | M_17 | AAGCAATTGATTGGTAAATTTCGAGAGAAAGATCGCGA | 38 |
| 282 | M_18 | GGAAGATCAATACATAAAGAGTTGAACTTCTTTGTTGT | 38 |
| 283 | M_19 | CTTCGACATGGGTAATCCTCATGTTTGAATGGCCGGCG | 38 |
| 284 | M_20 | TCTATTAGTAGATGCCGGAGTTTGCTGCGATTGCTGAG | 38 |
| 285 | M_21 | GGAATCGGGTTTCCATCTTTTAGGAGACCTTGCATTGC | 38 |
| 286 | M_22 | CTTAACAATAAGCTCGCAGTCGGAATTCGTAGCGAAAA | 38 |
| 287 | M_23 | TTGGAATGGTTAGTTCCATATTTAAGTACGAACGCCAT | 38 |
| 288 | M_24 | GCGGCTACAGGAAGCTCTACACCACCAACAGTCTGGGT | 38 |
| 289 | M_25 | TGCCACTTTAGGCACCTCGACTTTGATGGTGTATTTGC | 38 |
| 290 | M_26 | GATTCTGCGCAGAGCTCTGACGAACGCTACAGGTTACT | 38 |
| 291 | M_27 | TTGTAAGCCTGTGAACGCGAGTTAGAGCTGATCCATTC | 38 |
| 292 | M_28 | AGCGACCCCGTTAGCGAAGTTGCTTGGGGCGACAGTCA | 38 |
| 293 | M_29 | CGTCGCCAGTTCCTCTTTTGAGGAACAAGTTTTCTTGTTCCGCCATTG | 48 |
| 294 | M_30 | TCGACGAGAATCCTCTTTTGAGGAACAAGTTTTCTTGTCGAACTGAGT | 48 |
| 295 | M_31 | AAAGTTAGAATCCTCTTTTGAGGAACAAGTTTTCTTGTGCCATGCTTC | 48 |
| 296 | M_32 | AAACTCCGGTTCCTCTTTTGAGGAACAAGTTTTCTTGTTGAGGGCTCT | 48 |
| 297 | M_33 | ATCTAGAGAGTCCTCTTTTGAGGAACAAGTTTTCTTGTCCGTTGCCTG | 48 |
| 298 | M_34 | ATTAATGCTATCCTCTTTTGAGGAACAAGTTTTCTTGTACGCATCTAA | 48 |
| 299 | M_35 | GGTATGGACCATCGAGAAAGGAGACTTTACGT | 32 |
| 300 | M_36 | ACGCGCCAGTTGTTGGCCATACGGATTGTACCCCTCGA | 38 |
| 301 | M_37 | TGCATGGCTGAGATTTGGGCCTTAGCAGTGCCCTGTCT | 38 |
| 302 | M_38 | CTCCACAGTCCACCCGTAGGGAGCGTCAACGCTTATGA | 38 |
| 303 | M_39 | TGGACTCACCCGTTATTACGTCAGTAACTGTTCCTGAC | 38 |
| 304 | M_40 | ATGTAGGAGCATCCCACGGGGGCCGTAAGGCCCTCGAG | 38 |
| 305 | M_41 | CATGTTACCTACAGGTAGGAGCCAGTCGACAACGAATG | 38 |
| 306 | M_42 | AGAAAGGCACCTTTTCCCACACTATACCTAGTGGGTTC | 38 |
| 307 | M_43 | AAGATACCTAGAGACGACAACCATGCCAAACGTGCATC | 38 |
| 308 | M_44 | GTTTATGTAAAACCATATCACGATACGTCGCGATATGT | 38 |
| 309 | M_45 | TGCACGTTGTTCCTCTTTTGAGGAACAAGTTTTCTTGTCTGGAAGTTT | 48 |
| 310 | M_46 | GCAGCTGGATTCCTCTTTTGAGGAACAAGTTTTCTTGTACGACAGACG | 48 |
| 311 | M_47 | GCCATCTAACTCCTCTTTTGAGGAACAAGTTTTCTTGTTTGATGTTAG | 48 |
| 312 | M_48 | TACCGACCTGTCCTCTTTTGAGGAACAAGTTTTCTTGTACGTACGGCT | 48 |
| 313 | M_49 | CTCATAGGAATCCTCTTTTGAGGAACAAGTTTTCTTGTGAAACTCTTG | 48 |
| 314 | M_50 | AAGGTGAACCTCCTCTTTTGAGGAACAAGTTTTCTTGTTTCGTAAGCA | 48 |
| 315 | M_51 | TCTCATATGCACCCTGGATATCACTCATTAGT | 32 |
| 316 | M_52 | GGTAACCAACCGAACTGCAACTCCAACCACCTGCCGGC | 38 |
| 317 | M_53 | CACGTGTTTTGATCGAAACTTTCGATCTTCGTTTAGGG | 38 |
| 318 | M_54 | CAAGGTAGCGGAGCGCCTGGCGCCAATTACCGCGACGA | 38 |
| 319 | M_55 | GCGGCAGTGTACGCCTTCACGAGCGCAATGGTTTGCGT | 38 |
| 320 | M_56 | CGCGAGTTGTGAGGCTGTCGACCTGGCCTCTGCTAAAG | 38 |
| 321 | M_57 | CAACACCAAGGTTAAAATTACCCTGGGTGACCTTTTGC | 38 |
| 322 | M_58 | AGGACTTCGGTCGACGCCCGGTTCGCAACGTTCTGCGG | 38 |
| 323 | M_59 | CACTTCGATGTAAGTCAAGTTTTGGCTTACAGGGAAGA | 38 |
| 324 | M_60 | GGCTGTAGCAGGAGCGTGCGTCGAGGGAGAAGCCGAAA | 38 |

TABLE 6
Linearising units for 18S rRNA ID ′1111′. Linearising units that form structural
colours are SEQ ID NOs 333-338; 347-352; 361-366; and 375-380 (highlighted in bold).

| SEQ | | | Length |
|---|---|---|---|
| ID NO | Name | Sequence (5′→′3′) | (nt) |
| 325 | 18S_rRNA_1 | TAATGATCCTTCCGCAGGTTCACCTACGGAAACCTTGT | 38 |
| 326 | 18S_rRNA_2 | TACGACTTTTACTTCCTCTAGATAGTCAAGTTCGACCG | 38 |
| 327 | 18S_rRNA_3 | TCTTCTCAGCGCTCCGCCAGGGCCGTGGGCCGACCCCG | 38 |
| 328 | 18S_rRNA_4 | GCGGGGCCGATCCGAGGGCCTCACTAAACCATCCAATC | 38 |
| 329 | 18S_rRNA_5 | GGTAGTAGCGACGGGCGGTGTGTACAAAGGGCAGGGAC | 38 |
| 330 | 18S_rRNA_6 | TTAATCAACGCAAGCTTATGACCCGCACTTACTGGGAA | 38 |
| 331 | 18S_rRNA_7 | TTCCTCGTTCATGGGGAATAATTGC | 25 |
| 332 | 18S_rRNA_8 | AATCCCCGATCCCCATCACGAATGG | 25 |
| 333 | 18S_rRNA_9 | GGTTCAACGGTCCTCTTTTGAGGAACAAGTTTTCTTGTGTTACCCGCG | 48 |
| 334 | 18S_rRNA_10 | CCTGCCGGCGTCCTCTTTTGAGGAACAAGTTTTCTTGTTAGGGTAGGC | 48 |
| 335 | 18S_rRNA_11 | ACACGCTGAGTCCTCTTTTGAGGAACAAGTTTTCTTGTCCAGTCAGTG | 48 |
| 336 | 18S_rRNA_12 | TAGCGCGCGTTCCTCTTTTGAGGAACAAGTTTTCTTGTGCAGCCCCGG | 48 |
| 337 | 18S_rRNA_13 | ACATCTAAGGTCCTCTTTTGAGGAACAAGTTTTCTTGTGCATCACAGA | 48 |
| 338 | 18S_rRNA_14 | CCTGTTATTGTCCTCTTTTGAGGAACAAGTTTTCTTGTCTCAATCTCG | 48 |
| 339 | 18S_rRNA_15 | GGTGGCTGAACGCCACTTGTCCCTCTAAGAAGTTGGGG | 38 |
| 340 | 18S_rRNA_16 | GACGCCGACCGCTCGGGGGTCGCGTAACTAGTTAGCAT | 38 |
| 341 | 18S_rRNA_17 | GCCAGAGTCTCGTTCGTTATCGGAATTAACCAGACAAA | 38 |
| 342 | 18S_rRNA_18 | TCGCTCCACCAACTAAGAACGGCCATGCACCACCACCC | 38 |
| 343 | 18S_rRNA_19 | ACGGAATCGAGAAAGAGCTATCAATCTGTCAATCCTGT | 38 |
| 344 | 18S_rRNA_20 | CCGTGTCCGGGCCGGGTGAGGTTTCCCGTGTTGAGTCA | 38 |
| 345 | 18S_rRNA_21 | AATTAAGCCGCAGGCTCCACTCCTG | 25 |
| 346 | 18S_rRNA_22 | GTGGTGCCCTTCCGTCAATTCCTTT | 25 |
| 347 | 18S_rRNA_23 | AAGTTTCAGCTCCTCTTTTGAGGAACAAGTTTTCTTGTTTTGCAACCA | 48 |
| 348 | 18S_rRNA_24 | TACTCCCCCCTCCTCTTTTGAGGAACAAGTTTTCTTGTGGAACCCAAA | 48 |
| 349 | 18S_rRNA_25 | GACTTTGGTTTCCTCTTTTGAGGAACAAGTTTTCTTGTTCCCGGAAGC | 48 |
| 350 | 18S_rRNA_26 | TGCCCGGCGGTCCTCTTTTGAGGAACAAGTTTTCTTGTGTCATGGGAA | 48 |
| 351 | 18S_rRNA_27 | TAACGCCGCCTCCTCTTTTGAGGAACAAGTTTTCTTGTGCATCGCCGG | 48 |
| 352 | 18S_rRNA_28 | TCGGCATCGTTCCTCTTTTGAGGAACAAGTTTTCTTGTTTATGGTCGG | 48 |
| 353 | 18S_rRNA_29 | AACTACGACGGTATCTGATCGTCTTCGAACCTCCGACT | 38 |
| 354 | 18S_rRNA_30 | TTCGTTCTTGATTAATGAAAACATTCTTGGCAAATGCT | 38 |
| 355 | 18S_rRNA_31 | TTCGCTCTGGTCCGTCTTGCGCCGGTCCAAGAATTTCA | 38 |
| 356 | 18S_rRNA_32 | CCTCTAGCGGCGCAATACGAATGCCCCCGGCCGTCCCT | 38 |
| 357 | 18S_rRNA_33 | CTTAATCATGGCCTCAGTTCCGAAAACCAACAAAATAG | 38 |
| 358 | 18S_rRNA_34 | AACCGCGGTCCTATTCCATTATTCCTAGCTGCGGTATC | 38 |
| 359 | 18S_rRNA_35 | CAGGCGGCTCGGGCCTGCTTTGAAC | 25 |
| 360 | 18S_rRNA_36 | ACTCTAATTTTTTCAAAGTAAACGC | 25 |
| 361 | 18S_rRNA_37 | TTCGGGCCCCTCCTCTTTTGAGGAACAAGTTTTCTTGTGCGGGACACT | 48 |
| 362 | 18S_rRNA_38 | CAGCTAAGAGTCCTCTTTTGAGGAACAAGTTTTCTTGTCATCGAGGGG | 48 |
| 363 | 18S_rRNA_39 | GCGCCGAGAGTCCTCTTTTGAGGAACAAGTTTTCTTGTGCAAGGGGCG | 48 |
| 364 | 18S_rRNA_40 | GGGACGGGCGTCCTCTTTTGAGGAACAAGTTTTCTTGTGTGGCTCGCC | 48 |
| 365 | 18S_rRNA_41 | TCGCGGCGGATCCTCTTTTGAGGAACAAGTTTTCTTGTCCGCCCGCCC | 48 |
| 366 | 18S_rRNA_42 | GCTCCCAAGATCCTCTTTTGAGGAACAAGTTTTCTTGTTCCAACTACG | 48 |
| 367 | 18S_rRNA_43 | AGCTTTTTAACTGCAGCAACTTTAATATACGCTATTGG | 38 |
| 368 | 18S_rRNA_44 | AGCTGGAATTACCGCGGCTGCTGGCACCAGACTTGCCC | 38 |
| 369 | 18S_rRNA_45 | TCCAATGGATCCTCGTTAAAGGATTTAAAGTGGACTCA | 38 |
| 370 | 18S_rRNA_46 | TTCCAATTACAGGGCCTCGAAAGAGTCCTGTATTGTTA | 38 |
| 371 | 18S_rRNA_47 | TTTTTCGTCACTACCTCCCCGGGTCGGGAGTGGGTAAT | 38 |
| 372 | 18S_rRNA_48 | TTGCGCGCCTGCTGCCTTCCTTGGATGTGGTAGCCGTT | 38 |
| 373 | 18S_rRNA_49 | TCTCAGGCTCCCTCTCCGGAATCGA | 25 |
| 374 | 18S_rRNA_50 | ACCCTGATTCCCCGTCACCCGTGGT | 25 |
| 375 | 18S_rRNA_51 | CACCATGGTATCCTCTTTTGAGGAACAAGTTTTCTTGTGGCACGGCGA | 48 |
| 376 | 18S_rRNA_52 | CTACCATCGATCCTCTTTTGAGGAACAAGTTTTCTTGTAAGTTGATAG | 48 |
| 377 | 18S_rRNA_53 | GGCAGACGTTTCCTCTTTTGAGGAACAAGTTTTCTTGTCGAATGGGTC | 48 |
| 378 | 18S_rRNA_54 | GTCGCCGCCATCCTCTTTTGAGGAACAAGTTTTCTTGTCGGGGGGCGT | 48 |
| 379 | 18S_rRNA_55 | GCGATCGGCCTCCTCTTTTGAGGAACAAGTTTTCTTGTCGAGGTTATC | 48 |
| 380 | 18S_rRNA_56 | TAGAGTCACCTCCTCTTTTGAGGAACAAGTTTTCTTGTAAAGCCGCCG | 48 |
| 381 | 18S_rRNA_57 | GCGCCCGCCCCCCGGCCGGGGCCGGAGAGGGGCTGACC | 38 |
| 382 | 18S_rRNA_58 | GGGTTGGTTTTGATCTGATAAATGCACGCATCCCCCCC | 38 |
| 383 | 18S_rRNA_59 | GCGAAGGGGGTCAGCGCCCGTCGGCATGTATTAGCTCT | 38 |
| 384 | 18S_rRNA_60 | AGAATTACCACAGTTATCCAAGTGGGAGAGGAGCGAGC | 38 |
| 385 | 18S_rRNA_61 | GACCAAAGGAACCATAACTGATTTAATGAGCCATTCGC | 38 |
| 386 | 18S_rRNA_62 | AGTTTCACTGTACCGGCCGTGCGTACTTAGACATGCAT | 38 |
| 387 | 18S_rRNA_63 | GGCTTAATCTTTGAGACAAGCATAT | 25 |
| 388 | 18S_rRNA_64 | GCTACTGGCAGGATCAACCAGGTA | 25 |

TABLE 7
Linearising units for 28S rRNA ID '11111'. Linearising units that form structural colours
are SEQ ID NOs 399-404; 413-418; 427-432; 441-446; and 455-460 (highlighted in bold).

| SEQ | | | Length |
|---|---|---|---|
| ID NO | Name | Sequence (5′→3′) | (nt) |
| 389 | 28S_rRNA_1 | TCGGAACGGCGCTCGCCCATCTCTCAGGACCGACTGAC | 38 |
| 390 | 28S_rRNA_2 | CCATGTTCAACTGCTGTTCACATGGAACCCTTCTCCAC | 38 |
| 391 | 28S_rRNA_3 | TTCGGCCTTCAAAGTTCTCGTTTGAATATTTGCTACTA | 38 |
| 392 | 28S_rRNA_4 | CCACCAAGATCTGCACCTGCGGCGGCTCCACCCGGGCC | 38 |
| 393 | 28S_rRNA_5 | CGCGCCCTAGGCTTCAAGGCTCACCGCAGCGGCCCTCC | 38 |
| 394 | 28S_rRNA_6 | TACTCGTCGCGGCGTAGCGTCCGCGGGGCTCCGGGGGC | 38 |
| 395 | 28S_rRNA_7 | GGGGAGCGGGGCGTGGGCGGGAGGAGGGGAGGAGGCGT | 38 |
| 396 | 28S_rRNA_8 | GGGGGGGGGGGGGGGGGAAGGACCCCACACCCCCGCCG | 38 |
| 397 | 28S_rRNA_9 | CCGCCGCCGCCGCCGCCCTCCGACGCACACCACACGCG | 38 |
| 398 | 28S_rRNA_10 | CGCGCGCGCGCGCCGCCCCCGCCGCTCCCGTCCACTCT | 38 |
| 399 | 28S_rRNA_11 | CGACTGCCGGTCCTCTTTTGAGGAACAAGTTTTCTTGTCGACGGCCGG | 48 |
| 400 | 28S_rRNA_12 | GTATGGGCCCTCCTCTTTTGAGGAACAAGTTTTCTTGTGACGCTCCAG | 48 |
| 401 | 28S_rRNA_13 | CGCCATCCATTCCTCTTTTGAGGAACAAGTTTTCTTGTTTTCAGGGCT | 48 |
| 402 | 28S_rRNA_14 | AGTTGATTCGTCCTCTTTTGAGGAACAAGTTTTCTTGTGCAGGTGAGT | 48 |
| 403 | 28S_rRNA_15 | TGTTACACACTCCTCTTTTGAGGAACAAGTTTTCTTGTTCCTTAGCGG | 48 |
| 404 | 28S_rRNA_16 | ATTCCGACTTTCCTCTTTTGAGGAACAAGTTTTCTTGTCCATGGCCAC | 48 |
| 405 | 28S_rRNA_17 | CGTCCTGCTGTCTATATCAACCAACACCTTTTCTGGGG | 38 |
| 406 | 28S_rRNA_18 | TCTGATGAGCGTCGGCATCGGGCGCCTTAACCCGGCGT | 38 |
| 407 | 28S_rRNA_19 | TCGGTTCATCCCGCAGCGCCAGTTCTGCTTACCAAAAG | 38 |
| 408 | 28S_rRNA_20 | TGGCCCACTAGGCACTCGCATTCCACGCCCGGCTCCAC | 38 |
| 409 | 28S_rRNA_21 | GCCAGCGAGCCGGGCTTCTTACCCATTTAAAGTTTGAG | 38 |
| 410 | 28S_rRNA_22 | AATAGGTTGAGATCGTTTCGGCCCCAAGACCTCTAATC | 38 |
| 411 | 28S_rRNA_23 | ATTCGCTTTACCGGATAAAACTGCG | 25 |
| 412 | 28S_rRNA_24 | TGGCGGGGGTGCGTCGGGTCTGCGA | 25 |
| 413 | 28S_rRNA_25 | GAGCGCCAGCTCCTCTTTTGAGGAACAAGTTTTCTTGTTATCCTGAGG | 48 |
| 414 | 28S_rRNA_26 | GAAACTTCGGTCCTCTTTTGAGGAACAAGTTTTCTTGTAGGGAACCAG | 48 |
| 415 | 28S_rRNA_27 | CTACTAGATGTCCTCTTTTGAGGAACAAGTTTTCTTGTGTTCGATTAG | 48 |
| 416 | 28S_rRNA_28 | TCTTTCGCCCTCCTCTTTTGAGGAACAAGTTTTCTTGTCTATACCCAG | 48 |
| 417 | 28S_rRNA_29 | GTCGGACGACTCCTCTTTTGAGGAACAAGTTTTCTTGTCGATTTGCAC | 48 |
| 418 | 28S_rRNA_30 | GTCAGGACCGTCCTCTTTTGAGGAACAAGTTTTCTTGTCTACGGACCT | 48 |
| 419 | 28S_rRNA_31 | CCACCAGAGTTTCCTCTGGCTTCGCCCTGCCCAGGCAT | 38 |
| 420 | 28S_rRNA_32 | AGTTCACCATCTTTCGGGTCCTAACACGTGCGCTCGTG | 38 |
| 421 | 28S_rRNA_33 | CTCCACCTCCCCGGCGCGGGGGCGAGACGGGCCGGTG | 38 |
| 422 | 28S_rRNA_34 | GTGCGCCCTCGGCGGACTGGAGAGGCCTCGGGATCCCA | 38 |
| 423 | 28S_rRNA_35 | CCTCGGCCGGCGAGCGCGCCGGCCTTCACCTTCATTGC | 38 |
| 424 | 28S_rRNA_36 | GCCACGGCGGCTTTCGTGCGAGCCCCCGACTCGCGCAC | 38 |
| 425 | 28S_rRNA_37 | GTGTTAGACTCCTTGGTCCGTGTTT | 25 |
| 426 | 28S_rRNA_38 | CAAGACGGGTCGGGTGGGTAGCCGA | 25 |
| 427 | 28S_rRNA_39 | CGTCGCCGCCTCCTCTTTTGAGGAACAAGTTTTCTTGTGACCCCGTGC | 48 |
| 428 | 28S_rRNA_40 | GCTCGCTCCGTCCTCTTTTGAGGAACAAGTTTTCTTGTCCGTCCCCCT | 48 |
| 429 | 28S_rRNA_41 | CTTCGGGGGATCCTCTTTTGAGGAACAAGTTTTCTTGTCGCGCGCGTG | 48 |
| 430 | 28S_rRNA_42 | GCCCCGAGAGTCCTCTTTTGAGGAACAAGTTTTCTTGTAACCTCCCCC | 48 |
| 431 | 28S_rRNA_43 | GGGCCCGACGTCCTCTTTTGAGGAACAAGTTTTCTTGTGCGCGACCCG | 48 |
| 432 | 28S_rRNA_44 | CCCGGGGCGCTCCTCTTTTGAGGAACAAGTTTTCTTGTACTGGGGACA | 48 |
| 433 | 28S_rRNA_45 | GTCCGCCCCGCCCCCCGACCCGCGCGCGGCACCCCCCC | 38 |
| 434 | 28S_rRNA_46 | CGTCGCCGGGGCGGGGGCGCGGGGAGGAGGGGTGGGAG | 38 |
| 435 | 28S_rRNA_47 | AGCGGTCGCGCCGTGGGAGGGGTGGCCCGGCCCCCCCA | 38 |
| 436 | 28S_rRNA_48 | CGAGGAGACGCCGGCGCGCCCCCGCGGGGGAGACCCCC | 38 |
| 437 | 28S_rRNA_49 | CTCGCGGGGGATTCCCCGCGGGGGTGGGCGCCGGGAGG | 38 |
| 438 | 28S_rRNA_50 | GGGGAGAGCGCGGCGACGGGTCTCGCTCCCTCGGCCCC | 38 |
| 439 | 28S_rRNA_51 | GGGATTCGGCGAGTGCTGCTGCCGG | 25 |
| 440 | 28S_rRNA_52 | GGGGGCTGTAACACTCGGGGGGGGT | 25 |
| 441 | 28S_rRNA_53 | TTCGGTCCCGTCCTCTTTTGAGGAACAAGTTTTCTTGTCCGCCGCCGC | 48 |
| 442 | 28S_rRNA_54 | CGCCGCCGCCTCCTCTTTTGAGGAACAAGTTTTCTTGTACCGCCGCCG | 48 |
| 443 | 28S_rRNA_55 | CCGCCGCCGCTCCTCTTTTGAGGAACAAGTTTTCTTGTCCCGACCCGC | 48 |
| 444 | 28S_rRNA_56 | GCGCCCTCCCTCCTCTTTTGAGGAACAAGTTTTCTTGTGAGGGAGGAC | 48 |
| 445 | 28S_rRNA_57 | GCGGGGCCGGTCCTCTTTTGAGGAACAAGTTTTCTTGTGGGGCGGAGA | 48 |
| 446 | 28S_rRNA_58 | CGGGGGAGGATCCTCTTTTGAGGAACAAGTTTTCTTGTGGAGGACGGA | 48 |
| 447 | 28S_rRNA_59 | CGGACGGACGGACGGGGCCCCCCGAGCCACCTTCCCCG | 38 |
| 448 | 28S_rRNA_60 | CCGGGCCTTCCCAGCCGTCCCGGAGCCGGTCGCGGCGC | 38 |
| 449 | 28S_rRNA_61 | ACCGCCGCGGTGGAAATGCGCCCGGCGGCGGCCGGTCG | 38 |
| 450 | 28S_rRNA_62 | CCGGTCGGGGGACGGTCCCCCGCCGACCCCACCCCCGG | 38 |
| 451 | 28S_rRNA_63 | CCCCGCCCGCCCACCCCCGCACCCGCCGGAGCCCGCCC | 38 |
| 452 | 28S_rRNA_64 | CCTCCGGGGAGGAGGAGGAGGGGCGGCGGGGGAAGGGA | 38 |
| 453 | 28S_rRNA_65 | GGGCGGGTGGAGGGGTCGGGAGGAA | 25 |
| 454 | 28S_rRNA_66 | CGGGGGGCGGGAAAGATCCGCCGGG | 25 |
| 455 | 28S_rRNA_67 | CCGCCGACACTCCTCTTTTGAGGAACAAGTTTTCTTGTGGCCGGACCC | 48 |
| 456 | 28S_rRNA_68 | GCCGCCGGGTTCCTCTTTTGAGGAACAAGTTTTCTTGTTGAATCCTCC | 48 |
| 457 | 28S_rRNA_69 | GGGCGGACTGTCCTCTTTTGAGGAACAAGTTTTCTTGTCGCGGACCCC | 48 |
| 458 | 28S_rRNA_70 | ACCCGTTTACTCCTCTTTTGAGGAACAAGTTTTCTTGTCTCTTAACGG | 48 |
| 459 | 28S_rRNA_71 | TTTCACGCCCTCCTCTTTTGAGGAACAAGTTTTCTTGTTCTTGAACTC | 48 |
| 460 | 28S_rRNA_72 | TCTCTTCAAATCCTCTTTTGAGGAACAAGTTTTCTTGTGTTCTTTTCA | 48 |
| 461 | 28S_rRNA_73 | ACTTTCCCTTACGGTACTTGTTGACTATCGGTCTCGTG | 38 |
| 462 | 28S_rRNA_74 | CCGGTATTTAGCCTTAGATGGAGTTTACCACCCGCTTT | 38 |
| 463 | 28S_rRNA_75 | GGGCTGCATTCCCAAGCAACCCGACTCCGGGAAGACCC | 38 |
| 464 | 28S_rRNA_76 | GGGCCCGGCGCGCCGGGGGCCGCTACCGGCCTCACACC | 38 |
| 465 | 28S_rRNA_77 | GTCCACGGGCTGGGCCTCGATCAGAAGGACTTGGGCCC | 38 |
| 466 | 28S_rRNA_78 | CCCACGAGCGGCGCCGGGGAGCGGGTCTTCCGTACGCC | 38 |
| 467 | 28S_rRNA_79 | ACATGTCCCGCGCCCCGCCGCGGGGCGGGGATTCGGCG | 38 |
| 468 | 28S_rRNA_80 | CTGGGCTCTTCCCTGTTCACTCGCCGTTACTGAGGGAA | 38 |
| 469 | 28S_rRNA_81 | TCCTGGTTAGTTTCTTCTCCTCCGCTGACTAATATGCT | 38 |
| 470 | 28S_rRNA_82 | TAAATTCAGCGGGTCGCCACGTCTGATCTGAGGTCGCG | 38 |

TABLE 8
Linearising units for the MS2 RNA fully complementary ID '111'.

| SEQ ID | | | Length |
|---|---|---|---|
| NO | Name | Sequence (5′→3′) | (nt) |
| 471 | M-1 | CCGGCTTTCTCCTCGTACGGGCGACCCCACGATGACCCACTTCGCTTGTAG | 51 |
| 472 | M-2 | GCACCTTGATCTATCGATGTGACACTTAACGCCCCCCGTGAATACGGAGA | 50 |
| 473 | M-3 | GGGGTAGTGCCACTGTTTCGTTTTGGCCCCAGTCGAGTTAAAACGACCGG | 50 |
| 474 | M-4 | GAGTCCAGTTCGAACGATATTTTAAAGAGAATGAGTTATCTTCAGTCTCA | 50 |
| 475 | M-5 | CCGTCCGCGTAAACGCGAACGGAGGGGACGAAGGTCTCGTTCTCCCTATC | 50 |
| 476 | M-6 | AAGGGTACTAAAAGCTCGCACAGGTCAAACCTCCTAGGAATGGAATTCCG | 50 |
| 477 | M-7 | GCTACCTACAGCGATAGCCATGGTAGCGTCTCGCTAAAGACATTAAAAAT | 50 |
| 478 | M-8 | GGCATTAGCTCGACAGGAAGTTGAGCAGGACCCCGAAAGGGGTCCCACCC | 50 |
| 479 | M61 | TGGGTGGTAACTAGCCAAGCAGCTAGTTACCAAATCGGGAGAATCCCGGG | 50 |
| 480 | M62 | TCCTCTCTTTAGGGGGAGGTCCCTGGGCCGAAGCCCGCCCACCTTTCGGT | 50 |
| 481 | M63 | GGAGCCGGACCGCTTTCGCACCCGTGCTCTTTCGAGCACACCCACCCCGT | 50 |
| 482 | M64 | TTACGGGGGTCCCTCGGTCAGCTACCGAGGAGAGCTCGCTGGCCCACACT | 50 |
| 483 | M65 | CCTGAGGGAATGTGGGAACCGGCGTTAGCCACTCCGAAGTGCGTATAACG | 50 |
| 484 | M66 | CGCACGCCGGCGGACTTCATGCTGTCGGTGATTTCACCTCCAGTATGGAA | 50 |
| 485 | M67 | CCACGCTATGTAGCGACCACTGTCGTGCTTTTCGCTGAAGAACTTGCGTT | 50 |
| 486 | M68 | CTCGAGCGATACGAGCAAGACGGAAACCCGAGGTACGGGTATCCGCGAGC | 50 |
| 487 | M69 | AGCCGCCCGTACGGAGTCTTGGTGTATACCGAGACTGCCGTAGGCGGGCT | 50 |
| 488 | M70 | GACTACGTAGTAGTCGGCAGCGAGGTCCGTCCCACCGAAGAACATCGAAG | 50 |
| 489 | M71 | GCACCTGGGAGGAGAGCCGTACCCACACCTTATAGAGGCGTGGATCTGAC | 50 |
| 490 | M72 | ATACCTCCGACAACTCCCCAACCCCGTAGCCGATTTAATATCAGCATCAG | 50 |
| 491 | M73 | GGCGAAGAGATTGTCAACAGGTTTCTTGATGTAAAACGGTTTGACATCGA | 50 |
| 492 | M74 | CACCACGGTAAAAGTGCGCGCCGCAGCTCTCGCGAAAGAGCCCGGACACG | 50 |
| 493 | M75 | AACGTTTTACGAAGATTCGGTTTAAAACCGTAGTAGGCAAGTGCCTCTAG | 50 |
| 494 | M76 | CACACGGGGTGCAATCTCACTGGGACATATAATATCGTCCCCGTAGATGC | 50 |
| 495 | M77 | CTATGGTTCCGGCGTTACCAAAATGGATTTGGGTCGCTTTGACTATTGCC | 50 |
| 496 | M78 | CAGAATATCATGGACTCTAGCTCAAATGTGAACCCATTTCCCATTGTGGA | 50 |
| 497 | M79 | AAATAGTTCCCATCGTATCGTCTCGCCATCTACGATTCCGTAGTGTGAGC | 50 |
| 498 | M80 | GGATACGATCGAGATATGAATATAGCTCTGGTGGGAGAAAACTCCACACC | 50 |
| 499 | M81 | AGGCGATCGGAGATGGAATCGGATGCAGACGATAAGTCTATCGTCGCAAG | 50 |
| 500 | M82 | CGAACCATCTACGCTGCCCTGCTGAGCCAGACGCTGGTTGATCGATTGAT | 50 |
| 501 | M83 | CATTCAGGTCTATACCAACGGATTTGAGCCGGCGTCTGATGAAAGCACCG | 50 |
| 502 | M84 | ACCCCTTTCTGGAGGTACATATTCATATCAGGCTCCTTAC | 40 |
| 503 | M85 | AGGCAGCCCGATCTATTTTATTATTCTTCGGAACTGTAAA | 40 |

TABLE 9
Human universal total RNA contains equal
quantities of DNase-treated total RNA from ten
different human tissues/cell lines.

| Sample type | Origin |
|---|---|
| Adenocarcinoma | Mammary gland |
| Melanoma | Skin |
| Hepatoblastoma | Liver |
| Liposarcoma | Fat cells |
| Adenocarcinoma | Cervix |
| Histiocytic lymphoma | Macrophage and histiocyte |
| Embryonal carcinoma | Testis |
| Lymphoblastic leukemia | T lymphoblast |
| Glioblastoma | Brain |
| Plasmacytoma; myeloma | B lymphocyte |

TABLE 10
Linearising units for the M13 ID '111111'. Replaced SEQ ID NO corresponds to the linearising
unit SEQ IDs listed in Table 1 that are replaced to produce sequence ID.

| | | | | Replaced |
|---|---|---|---|---|
| | SEQ ID | | Length | SEQ ID |
| Site | NO | Sequence (5′→3′) | (nt) | NO |
| 1 | 504 | ACATCACTTGTCCTCTTTTGAGGAACAAGTTTTCTTGTCCTGAGTAGA | 48 | 26-30 |
| | 505 | AGAACTCAAATCCTCTTTTGAGGAACAAGTTTTCTTGTCTATCGGCCT | 48 | |
| | 506 | TGCTGGTAATTCCTCTTTTGAGGAACAAGTTTTCTTGTATCCAGAACA | 48 | |
| | 507 | ATATTACCGCTCCTCTTTTGAGGAACAAGTTTTCTTGTCAGCCATTGC | 48 | |
| | 508 | AACAGGAAAATCCTCTTTTGAGGAACAAGTTTTCTTGTACGCTCATGG | 48 | |
| | 509 | AAATACCTACTCCTCTTTTGAGGAACAAGTTTTCTTGTATTTTGACGC | 48 | |
| | 510 | TCAATCGTCTTCCTCTTTTGAGGAACAAGTTTTCTTGTGAAATGGATT | 48 | |
| | 511 | ATTTACATTGTCCTCTTTTGAGGAACAAGTTTTCTTGTGCAGATTCAC | 48 | |
| | 512 | CAGTCACACGACCAGTAATAAAAGGGACAT | 30 | |
| 2 | 513 | TTACCTGAGCAAAAGAAGATGATGAAACAAACATCAAGAAAACA | 44 | 52-57 |
| | 514 | AAATTAATTATCCTCTTTTGAGGAACAAGTTTTCTTGTCATTTAACAA | 48 | |
| | 515 | TTTCATTTGATCCTCTTTTGAGGAACAAGTTTTCTTGTATTACCTTTT | 48 | |
| | 516 | TTAATGGAAATCCTCTTTTGAGGAACAAGTTTTCTTGTCAGTACATAA | 48 | |
| | 517 | ATCAATATATTCCTCTTTTGAGGAACAAGTTTTCTTGTGTGAGTGAAT | 48 | |
| | 518 | AACCTTGCTTTCCTCTTTTGAGGAACAAGTTTTCTTGTCTGTAAATCG | 48 | |
| | 519 | TCGCTATTAATCCTCTTTTGAGGAACAAGTTTTCTTGTTTAATTTTCC | 48 | |
| | 520 | CTTAGAATCCTCCTCTTTTGAGGAACAAGTTTTCTTGTTTGAAAACAT | 48 | |
| | 521 | AGCGATAGCTTCCTCTTTTGAGGAACAAGTTTTCTTGTTAGATTAAGA | 48 | |
| | 522 | CGCTGAGAAGAGTCAATAGTGAAT | 24 | |
| 3 | 523 | TGAATCTTACCAACGCTAACGAGCGTCTTTCCAGAGCCTAATTTGCCAGT | 50 | 79-85 |
| | 524 | TACAAAATAATCCTCTTTTGAGGAACAAGTTTTCTTGTACAGCCATAT | 48 | |
| | 525 | TATTTATCCCTCCTCTTTTGAGGAACAAGTTTTCTTGTAATCCAAATA | 48 | |
| | 526 | AGAAACGATTTCCTCTTTTGAGGAACAAGTTTTCTTGTTTTTGTTTAA | 48 | |
| | 527 | CGTCAAAAATTCCTCTTTTGAGGAACAAGTTTTCTTGTGAAAATAGCA | 48 | |
| | 528 | GCCTTTACAGTCCTCTTTTGAGGAACAAGTTTTCTTGTAGAGAATAAC | 48 | |
| | 529 | ATAAAAACAGTCCTCTTTTGAGGAACAAGTTTTCTTGTGGAAGCGCAT | 48 | |
| | 530 | TAGACGGGAGTCCTCTTTTGAGGAACAAGTTTTCTTGTAATTAACTGA | 48 | |
| | 531 | ACACCCTGAATCCTCTTTTGAGGAACAAGTTTTCTTGTCAAAGTCAGA | 48 | |
| | 532 | GGGTAATTGAGCGCTAATATCAGAGAGATAACCCACAAGAATTGAGTTAAGCCCAA | 56 | |
| 4 | 533 | TCACAAACAAATAAATCCTCATTAAAGCCAGAATGGAAAGCGCAGTCTCTGAATTT | 56 | 106-112 |
| | 534 | ACCGTTCCAGTCCTCTTTTGAGGAACAAGTTTTCTTGTTAAGCGTCAT | 48 | |
| | 535 | ACATGGCTTTTCCTCTTTTGAGGAACAAGTTTTCTTGTTGATGATACA | 48 | |
| | 536 | GGAGTGTACTTCCTCTTTTGAGGAACAAGTTTTCTTGTGGTAATAAGT | 48 | |
| | 537 | TTTAACGGGGTCCTCTTTTGAGGAACAAGTTTTCTTGTTCAGTGCCTT | 48 | |
| | 538 | GAGTAACAGTTCCTCTTTTGAGGAACAAGTTTTCTTGTGCCCGTATAA | 48 | |
| | 539 | ACAGTTAATGTCCTCTTTTGAGGAACAAGTTTTCTTGTCCCCCTGCCT | 48 | |
| | 540 | ATTTCGGAACTCCTCTTTTGAGGAACAAGTTTTCTTGTCTATTATTCT | 48 | |
| | 541 | GAAACATGAATCCTCTTTTGAGGAACAAGTTTTCTTGTAGTATTAAGA | 48 | |
| | 542 | GGCTGAGACTCCTCAAGAGAAGGATTAGGATTAGCGGGGTTTTGCTCAGT | 50 | |
| 5 | 543 | CAAAGTACAACGGAGATTTGTATC | 24 | 134-139 |
| | 544 | ATCGCCTGATTCCTCTTTTGAGGAACAAGTTTTCTTGTAAATTGTGTC | 48 | |
| | 545 | GAAATCCGCGTCCTCTTTTGAGGAACAAGTTTTCTTGTACCTGCTCCA | 48 | |
| | 546 | TGTTACTTAGTCCTCTTTTGAGGAACAAGTTTTCTTGTCCGGAACGAG | 48 | |
| | 547 | GCGCAGACGGTCCTCTTTTGAGGAACAAGTTTTCTTGTTCAATCATAA | 48 | |
| | 548 | GGGAACCGAATCCTCTTTTGAGGAACAAGTTTTCTTGTCTGACCAACT | 48 | |
| | 549 | TTGAAAGAGGTCCTCTTTTGAGGAACAAGTTTTCTTGTACAGATGAAC | 48 | |
| | 550 | GGTGTACAGATCCTCTTTTGAGGAACAAGTTTTCTTGTCCAGGCGCAT | 48 | |
| | 551 | AGGCTGGCTGTCCTCTTTTGAGGAACAAGTTTTCTTGTACCTTCATCA | 48 | |
| | 552 | AGAGTAATCTTGACAAGAACCGGATATTCATTACCCAAATCAAC | 44 | |
| 6 | 553 | ACAGTTGATTCCCAATTCTGCGAACGAGTA | 30 | 161-165 |
| | 554 | GATTTAGTTTTCCTCTTTTGAGGAACAAGTTTTCTTGTGACCATTAGA | 48 | |
| | 555 | TACATTTCGCTCCTCTTTTGAGGAACAAGTTTTCTTGTAAATGGTCAA | 48 | |
| | 556 | TAACCTGTTTTCCTCTTTTGAGGAACAAGTTTTCTTGTAGCTATATTT | 48 | |
| | 557 | TCATTTGGGGTCCTCTTTTGAGGAACAAGTTTTCTTGTCGCGAGCTGA | 48 | |
| | 558 | AAAGGTGGCATCCTCTTTTGAGGAACAAGTTTTCTTGTTCAATTCTAC | 48 | |
| | 559 | TAATAGTAGTTCCTCTTTTGAGGAACAAGTTTTCTTGTAGCATTAACA | 48 | |
| | 560 | TCCAATAAATTCCTCTTTTGAGGAACAAGTTTTCTTGTCATACAGGCA | 48 | |
| | 561 | AGGCAAAGAATCCTCTTTTGAGGAACAAGTTTTCTTGTTTAGCAAAAT | 48 | |

TABLE 11
Linearising units for MS2 RNA exons' ID fabrication.

| SEQ ID | | | Length |
|---|---|---|---|
| NO | Name | Sequence (5′→3′) | (nt) |
| 562 | M_AS_1 | TGGGTGGTAACTAGCCAAGCAGCTA | 25 |
| 563 | M_AS_2 | GTTACCAAATCGGGAGAATCCCGGGTCCTCTC | 30 |
| 564 | M_AS_3 | TTTAGGGGGAGGTCCCTGGGCCGAAGCCCGCCCACCTTTC | 40 |
| 565 | M_AS_4 | GGTGGAGCCGGACCGCTTTCGCACCCGTGCTCTTTCGAGC | 40 |
| 566 | M_AS_5 | ACACCCACCCCGTTTACGGGGGTCCCTCGGTCAGCTACCG | 40 |
| 567 | M_AS_6 | AGGAGAGCTCGCTGGCCCACACTCCTGAGGGAATGTGGGA | 40 |
| 568 | M_AS_7 | ACCGGCGTTAGCCACTCCGAAGTGCGTATAACGCGCACGC | 40 |
| 569 | M_AS_8 | CGGCGGACTTCATGCTGTCGGTGATTTCACCTCCAGTATG | 40 |
| 570 | M_AS_9 | GAACCACGCTATGTAGCGACCACTGTCGTGCTTTTCGCTG | 40 |
| 571 | M_AS_10 | AAGAACTTGCGTTCTCGAGCGATACGAGCAAGACGGAAAC | 40 |
| 572 | M_AS_11 | CCGAGGTACGGGTATCCGCGAGCAGCCGCCCGTACGGAGT | 40 |
| 573 | M_AS_12 | CTTGGTGTATACCGAGACTGCCGTAGGCGGGCTGACTACG | 40 |
| 574 | M_AS_13 | TAGTAGTCGGCAGCGAGGTCCGTCCCACCGAAGAACATCG | 40 |
| 575 | M_AS_14 | AAGGCACCTGGGAGGAGAGCCGTACCCACACCTTATAGAG | 40 |
| 576 | M_AS_15 | GCGTGGATCTGACATACCTCCGACAACTCCCCAACCCCGT | 40 |
| 577 | M_AS_16 | AGCCGATTTAATATCAGCATCAGGGCGAAGAGATTGTCAA | 40 |
| 578 | M_AS_17 | CAGGTTTCTTGATGTAAAACGGTTTGACATCGACACCACG | 40 |
| 579 | M_AS_18 | GTAAAAGTGCGCGCCGCAGCTCTCGCGAAAGAGCCCGGAC | 40 |
| 580 | M_AS_19 | ACGAACGTTTTACGAAGATTCGGTTTAAAACCGTAGTAGG | 40 |
| 581 | M_AS_20 | CAAGTGCCTCTAGCACACGGGGTGCAATCTCACTGGGACA | 40 |
| 582 | M_AS_21 | TATAATATCGTCCCCGTAGATGCCTATGGTTCCGGCGTTA | 40 |
| 583 | M_AS_22 | CCAAAATGGATTTGGGTCGCTTTGACTATTGCCCAGAATA | 40 |
| 584 | M_AS_23 | TCATGGACTCTAGCTCAAATGTGAA | 25 |
| 585 | M_AS_24 | CCCATTTCCCATTGTGGAAAATAGT | 25 |
| 586 | M_AS_25 | TCCCATCGTATCGTCTCGCCATCTA | 25 |
| 587 | M_AS_26 | CGATTCCGTAGTGTGAGCGGATACGATCGAGATATGAATA | 40 |
| 588 | M_AS_27 | TAGCTCTGGTGGGAGAAAACTCCACACCAGGCGATCGGAG | 40 |
| 589 | M_AS_28 | ATGGAATCGGATGCAGACGATAAGTCTATCGTCGCAAGCG | 40 |
| 590 | M_AS_29 | AACCATCTACGCTGCCCTGCTGAGCCAGACGCTGGTTGAT | 40 |
| 591 | M_AS_30 | CGATTGATCATTCAGGTCTATACCAACGGATTTGAGCCGG | 40 |
| 592 | M_AS_31 | CGTCTGATGAAAGCACCGACCCCTTTCTGGAGGTACATAT | 40 |
| 593 | M_AS_32 | TCATATCAGGCTCCTTACAGGCAGCCCGATCTATTTTATT | 40 |
| 594 | M_AS_33 | ATTCTTCGGAACTGTAAACACTCCGTTCCCTACAACGAGC | 40 |
| 595 | M_AS_34 | CTAAATTCATATGACTCGTTATAGCGGACCGCGTGTCTGA | 40 |
| 596 | M_AS_35 | TCCACGGCGCACATTGGTCTCGGACCAATAGAGCCGCTCT | 40 |
| 597 | M_AS_36 | CAGAGCGCGGGGGGTAACGGTTGCTTGTTCAGCGAACTTC | 40 |
| 598 | M_AS_37 | TTGTAAGGCGCTGCATCCTGCAACTTGTGCCCCATAGGAG | 40 |
| 599 | M_AS_38 | CACCGTTGGAGAACGTGCATTGCCCAAACAACGACGATCG | 40 |
| 600 | M_AS_39 | GTAGCCAGAGAGGAGGTTGCCAATAAGGCTACGGATGCTG | 40 |
| 601 | M_AS_40 | GTTTGTAAAACATCCGGATCCCATGACAAGGATTTGTCAT | 40 |
| 602 | M_AS_41 | GTAAGAAACCTTCTCTATTTATCTGACCGCGATCACCATT | 40 |
| 603 | M_AS_42 | CGCCTCCCGTAGCTTAGCGATAGCTAAGGTACGACGGGTC | 40 |
| 604 | M_AS_43 | GCCTCGTCATTACCAGAACCTAAGGTCGGATGCTTTGTGA | 40 |
| 605 | M_AS_44 | GCAATTCGTCCCTTAAGTAAGCAATTGCTGTAAAGTCGTC | 40 |
| 606 | M_AS_45 | ACTGTGCGGATCACCGCTTCCAGTAGCGACAGAAGCAATT | 40 |
| 607 | M_AS_46 | GATTGGTAAATTTCGAGAGAAAGATCGCGAGGAAGATCAA | 40 |
| 608 | M_AS_47 | TACATAAAGAGTTGAACTTCTTTGT | 25 |
| 609 | M_AS_48 | TGTCTTCGACATGGGTAATCCTCAT | 25 |
| 610 | M_AS_49 | GTTTGAATGGCCGGCGTCTATTAGTAGATGCCGGAGTTTG | 40 |
| 611 | M_AS_50 | CTGCGATTGCTGAGGGAATCGGGTTTCCATCTTTTAGGAG | 40 |
| 612 | M_AS_51 | ACCTTGCATTGCCTTAACAATAAGCTCGCAGTCGGAATTC | 40 |
| 613 | M_AS_52 | GTAGCGAAAATTGGAATGGTTAGTTCCATATTTAAGTACG | 40 |
| 614 | M_AS_53 | AACGCCATGCGGCTACAGGAAGCTCTACACCACCAACAGT | 40 |
| 615 | M_AS_54 | CTGGGTTGCCACTTTAGGCACCTCGACTTTGATGGTGTAT | 40 |
| 616 | M_AS_55 | TTGCGATTCTGCGCAGAGCTCTGACGAACGCTACAGGTTA | 40 |
| 617 | M_AS_56 | CTTTGTAAGCCTGTGAACGCGAGTTAGAGCTGATCCATTC | 40 |
| 618 | M_AS_57 | AGCGACCCCGTTAGCGAAGTTGCTTGGGGCGACAGTCACG | 40 |
| 619 | M_AS_58 | TCGCCAGTTCCGCCATTGTCGACGAGAACGAACTGAGTAA | 40 |
| 620 | M_AS_59 | AGTTAGAAGCCATGCTTCAAACTCCGGTTGAGGGCTCTAT | 40 |
| 621 | M_AS_60 | CTAGAGAGCCGTTGCCTGATTAATGCTAACGCATCTAAGG | 40 |
| 622 | M_AS_61 | TATGGACCATCGAGAAAGGAGACTTTACGTACGCGCCAGT | 40 |
| 623 | M_AS_62 | TGTTGGCCATACGGATTGTACCCCTCGATGCATGGCTGAG | 40 |
| 624 | M_AS_63 | ATTTGGGCCTTAGCAGTGCCCTGTCTCTCCACAGTCCACC | 40 |
| 625 | M_AS_64 | CGTAGGGAGCGTCAACGCTTATGATGGACTCACCCGTTAT | 40 |
| 626 | M_AS_65 | TACGTCAGTAACTGTTCCTGACATGTAGGAGCATCCCACG | 40 |
| 627 | M_AS_66 | GGGGCCGTAAGGCCCTCGAGCATGTTACCTACAGGTAGGA | 40 |
| 628 | M_AS_67 | GCCAGTCGACAACGAATGAGAAAGGCACCTTTTCCCACAC | 40 |
| 629 | M_AS_68 | TATACCTAGTGGGTTCAAGATACCTAGAGACGACAACCAT | 40 |
| 630 | M_AS_69 | GCCAAACGTGCATCGTTTATGTAAAACCATATCACGATAC | 40 |
| 631 | M_AS_70 | GTCGCGATATGTTGCACGTTGTCTG | 25 |
| 632 | M_AS_71 | GAAGTTTGCAGCTGGATACGACAGA | 25 |
| 633 | M_AS_72 | CGGCCATCTAACTTGATGTTAGTACCGACCTGACGTACGG | 40 |
| 634 | M_AS_73 | CTCTCATAGGAAGAAACTCTTGAAGGTGAACCTTCGTAAG | 40 |
| 635 | M_AS_74 | CATCTCATATGCACCCTGGATATCACTCATTAGTGGTAAC | 40 |
| 636 | M_AS_75 | CAACCGAACTGCAACTCCAACCACCTGCCGGCCACGTGTT | 40 |
| 637 | M_AS_76 | TTGATCGAAACTTTCGATCTTCGTTTAGGGCAAGGTAGCG | 40 |
| 638 | M_AS_77 | GAGCGCCTGGCGCCAATTACCGCGACGAGCGGCAGTGTAC | 40 |
| 639 | M_AS_78 | GCCTTCACGAGCGCAATGGTTTGCGTCGCGAGTTGTGAGG | 40 |
| 640 | M_AS_79 | CTGTCGACCTGGCCTCTGCTAAAGCAACACCAAGGTTAAA | 40 |
| 641 | M_AS_80 | ATTACCCTGGGTGACCTTTTGCAGGACTTCGGTCGACGCC | 40 |
| 642 | M_AS_81 | CGGTTCGCAACGTTCTGCGGCACTTCGATGTAAGTCAAGT | 40 |
| 643 | M_AS_82 | TTTGGCTTACAGGGAAGAGGCTGTAGCAGGAGCGTGCGTC | 40 |
| 644 | M_AS_83 | GAGGGAGAAGCCGAAACCGGCTTTCTCCTCGTACGGGCGA | 40 |
| 645 | M_AS_84 | CCCCACGATGACCCACTTCGCTTGTAGGCACCTTGATCTA | 40 |
| 646 | M_AS_85 | TCGATGTGACACTTAACGCCCCCCGTGAATACGGAGAGGG | 40 |
| 647 | M_AS_86 | GTAGTGCCACTGTTTCGTTTTGGCCCCAGTCGAGTTAAAA | 40 |
| 648 | M_AS_87 | CGACCGGGAGTCCAGTTCGAACGATATTTTAAAGAGAATG | 40 |
| 649 | M_AS_88 | AGTTATCTTCAGTCTCACCGTCCGCGTAAACGCGAACGGA | 40 |
| 650 | M_AS_89 | GGGGACGAAGGTCTCGTTCTCCCTATCAAGGGTACTAAAA | 40 |
| 651 | M_AS_90 | GCTCGCACAGGTCAAACCTCCTAGGAATGGAATTCCGGCT | 40 |
| 652 | M_AS_91 | ACCTACAGCGATAGCCATGGTAGCGTCTCGCTAAAGACAT | 40 |
| 653 | M_AS_92 | TAAAAATGGCATTAGCTCGACAGGAAGTTGAG | 32 |
| 654 | M_AS_93 | CAGGACCCCGAAAGGGGTCCCACCC | 25 |

TABLE 12
Linearising units used for each exon type. Replaced SEQ ID NO correspond to the linearising
unit SEQ IDs listed in Table 11 that are replaced to produce the sequence ID.

| | SEQ | | | Replaced |
|---|---|---|---|---|
| | ID | | Length | SEQ |
| Exon | NO | Sequence (5′→3′) | (nt) | ID NO |
| Exon I | 655 | GAGATGGAATCGGATGCAGATGGGTGGTAACTAGCCAAGCAGCTA | 45 | 562 |
| ‘112’ | 656 | TCATGGACTCTAGCTCAAATGTGAA TTT GGATATCACTCATTAGTGGT | 48 | 584 |
| | 657 | TACATAAAGAGTTGAACTTCTTTGT TTT GGATATCACTCATTAGTGGT | 48 | 608 |
| | 658 | GTCGCGATATGTTGCACGTTGTCTG TTT GGATATCACTCATTAGTGGT | 48 | 631 |
| | 659 | GAAGTTTGCAGCTGGATACGACAGA TTT GGATATCACTCATTAGTGGT | 48 | 632 |
| | 660 | CAGGACCCCGAAAGGGGTCCCACCC CCTGATATGAATATGTACCT | 45 | 654 |
| Exon II | 661 | TCTGCATCCGATTCCATCTCTGGGTGGTAACTAGCCAAGCAGCTA | 45 | 562 |
| ‘312’ | 662 | TCATGGACTCTAGCTCAAATGTGAA TTT GGATATCACTCATTAGTGGT | 48 | 584 |
| | 663 | CCCATTTCCCATTGTGGAAAATAGT TTT GGATATCACTCATTAGTGGT | 48 | 585 |
| | 664 | TCCCATCGTATCGTCTCGCCATCTA TTT GGATATCACTCATTAGTGGT | 48 | 586 |
| | 665 | TACATAAAGAGTTGAACTTCTTTGT TTT GGATATCACTCATTAGTGGT | 48 | 608 |
| | 666 | GTCGCGATATGTTGCACGTTGTCTG TTT GGATATCACTCATTAGTGGT | 48 | 631 |
| | 667 | GAAGTTTGCAGCTGGATACGACAGA TTT GGATATCACTCATTAGTGGT | 48 | 632 |
| | 668 | CAGGACCCCGAAAGGGGTCCCACCC CCTGATATGAATATGTACCT | 45 | 654 |
| Exon III | 669 | TCTGCATCCGATTCCATCTCTGGGTGGTAACTAGCCAAGCAGCTA | 45 | 562 |
| ‘321’ | 670 | TCATGGACTCTAGCTCAAATGTGAA TTT GGATATCACTCATTAGTGGT | 48 | 584 |
| | 671 | CCCATTTCCCATTGTGGAAAATAGT TTT GGATATCACTCATTAGTGGT | 48 | 585 |
| | 672 | TCCCATCGTATCGTCTCGCCATCTA TTT GGATATCACTCATTAGTGGT | 48 | 586 |
| | 673 | TACATAAAGAGTTGAACTTCTTTGT TTT GGATATCACTCATTAGTGGT | 48 | 608 |
| | 674 | TGTCTTCGACATGGGTAATCCTCAT TTT GGATATCACTCATTAGTGGT | 48 | 609 |
| | 675 | GTCGCGATATGTTGCACGTTGTCTG TTT GGATATCACTCATTAGTGGT | 48 | 631 |
| | 676 | CAGGACCCCGAAAGGGGTCCCACCC AGGTACATATTCATATCAGG | 45 | 654 |
| Extended | 677 | TCTGCATCCGATTCCATCTCTGGGTGGTAACTAGCCAAGCAGCTA | 45 | 562 |
| RNA | | | | |

TABLE 13
Linearising units for the linear and circular ID. Replaced SEQ ID NO correspond to the
linearising unit SEQ IDs listed in Table 1 that are replaced to produce the sequence ID.

| | SEQ | | | Replaced |
|---|---|---|---|---|
| | ID | | Length | SEQ |
| Site | NO | Sequence (5′→3′) | (nt) | ID NO |
| 1 | 678 | ACATCACTTGTCCTCTTTTGAGGAACAAGTTTTCTTGTCCTGAGTAGA | 48 | 26-30 |
| | 679 | AGAACTCAAATCCTCTTTTGAGGAACAAGTTTTCTTGTCTATCGGCCT | 48 | |
| | 680 | TGCTGGTAATTCCTCTTTTGAGGAACAAGTTTTCTTGTATCCAGAACA | 48 | |
| | 681 | ATATTACCGCTCCTCTTTTGAGGAACAAGTTTTCTTGTCAGCCATTGC | 48 | |
| | 682 | AACAGGAAAATCCTCTTTTGAGGAACAAGTTTTCTTGTACGCTCATGG | 48 | |
| | 683 | AAATACCTACTCCTCTTTTGAGGAACAAGTTTTCTTGTATTTTGACGC | 48 | |
| | 684 | TCAATCGTCTTCCTCTTTTGAGGAACAAGTTTTCTTGTGAAATGGATT | 48 | |
| | 685 | ATTTACATTGTCCTCTTTTGAGGAACAAGTTTTCTTGTGCAGATTCAC | 48 | |
| | 686 | CAGTCACACGACCAGTAATAAAAGGGACAT | 30 | |
| 2 | 687 | TTACCTGAGCAAAAGAAGATGATGAAACAAACATCAAGAAAACA | 44 | 52-57 |
| | 688 | AAATTAATTATCCTCTTTTGAGGAACAAGTTTTCTTGTCATTTAACAA | 48 | |
| | 689 | TTTCATTTGATCCTCTTTTGAGGAACAAGTTTTCTTGTATTACCTTTT | 48 | |
| | 690 | TTAATGGAAATCCTCTTTTGAGGAACAAGTTTTCTTGTCAGTACATAA | 48 | |
| | 691 | ATCAATATATTCCTCTTTTGAGGAACAAGTTTTCTTGTGTGAGTGAAT | 48 | |
| | 692 | AACCTTGCTTTCCTCTTTTGAGGAACAAGTTTTCTTGTCTGTAAATCG | 48 | |
| | 693 | TCGCTATTAATCCTCTTTTGAGGAACAAGTTTTCTTGTTTAATTTTCC | 48 | |
| | 694 | CTTAGAATCCTCCTCTTTTGAGGAACAAGTTTTCTTGTTTGAAAACAT | 48 | |
| | 695 | AGCGATAGCTTCCTCTTTTGAGGAACAAGTTTTCTTGTTAGATTAAGA | 48 | |
| | 696 | CGCTGAGAAGAGTCAATAGTGAAT | 24 | |
| 3 | 697 | TGAATCTTACCAACGCTAACGAGCGTCTTTCCAGAGCCTAATTTGCCAGT | 50 | 79-85 |
| | 698 | TACAAAATAATCCTCTTTTGAGGAACAAGTTTTCTTGTACAGCCATAT | 48 | |
| | 699 | TATTTATCCCTCCTCTTTTGAGGAACAAGTTTTCTTGTAATCCAAATA | 48 | |
| | 700 | AGAAACGATTTCCTCTTTTGAGGAACAAGTTTTCTTGTTTTTGTTTAA | 48 | |
| | 701 | CGTCAAAAATTCCTCTTTTGAGGAACAAGTTTTCTTGTGAAAATAGCA | 48 | |
| | 702 | GCCTTTACAGTCCTCTTTTGAGGAACAAGTTTTCTTGTAGAGAATAAC | 48 | |
| | 703 | ATAAAAACAGTCCTCTTTTGAGGAACAAGTTTTCTTGTGGAAGCGCAT | 48 | |
| | 704 | TAGACGGGAGTCCTCTTTTGAGGAACAAGTTTTCTTGTAATTAACTGA | 48 | |
| | 705 | ACACCCTGAATCCTCTTTTGAGGAACAAGTTTTCTTGTCAAAGTCAGA | 48 | |
| | 706 | GGGTAATTGAGCGCTAATATCAGAGAGATAACCCACAAGAATTGAGTTAAGCCCAA | 56 | |

TABLE 14
Linearising units for the ENO1.

| SEQ ID | | | Length |
|---|---|---|---|
| NO | Name | Sequence (5′→3′) | (nt) |
| 707 | ENO1_1 | TATTCTCATGGGTCACTGAGGCTTTTTATTTTGAGCAC | 38 |
| 708 | ENO1_2 | AAAACCACCGGGGATCTAGCCTGTGGCCACCCCGGAGA | 38 |
| 709 | ENO1_3 | TGACACGAGGCTCACATGACTCTAGACACTTGGTGGAA | 38 |
| 710 | ENO1_4 | AGTGAGGCGAGAAAAACAATGACTTGGGCCAATTACAC | 38 |
| 711 | ENO1_5 | GACTGCAAAGCTAGAGCTGCCAACAGGGCTCCAGGGAG | 38 |
| 712 | ENO1_6 | CTTGGCTTCTGTAGAAGTTCTAAGGAAGCGGTACGAAC | 38 |
| 713 | ENO1_7 | TCCACGGCGGTGGGGCGCTAACTAGCAGGGACCCCTGC | 38 |
| 714 | ENO1_8 | AAGTGTTGGTCGGGGGCCTCGAGCTGCCTGAGCTGACA | 38 |
| 715 | ENO1_9 | CGAGGGGAGGGGTCTGTGTAGCCAACAGGTGACCGAAG | 38 |
| 716 | ENO1_10 | GGCTTGCCTGCCCACAGCTTACTTGGCCAAGGGGTTTC | 38 |
| 717 | ENO1_11 | TGAAGTTCCTGCCGGCAAACTTAGCCTTGCTGCCCAGC | 38 |
| 718 | ENO1_12 | TCCTCTTCAATTCTGAGGAGCTGGTTGTACTTGGCCAA | 38 |
| 719 | ENO1_13 | GCGCTCAGATCGGCAAGGGGCACCAGTCTTGATCTGCC | 38 |
| 720 | ENO1_14 | CAGTGCACAGCCCCACAACCAGGTCAGCGATGAAGGTA | 38 |
| 721 | ENO1_15 | TCTTCAGTCTCCCCCGAACGATGAGACACCATGA | 34 |
| 722 | ENO1_16 | CGCCCCAACCTCCTCTTTTGAGGAACAAGTTTTCTTGTATTGGCCTGG | 48 |
| 723 | ENO1_17 | GCCAGCTTGCTCCTCTTTTGAGGAACAAGTTTTCTTGTACGCCTGAAG | 48 |
| 724 | ENO1_18 | AGACTCGGTCTCCTCTTTTGAGGAACAAGTTTTCTTGTACGGAGCCAA | 48 |
| 725 | ENO1_19 | TCTGGTTGACTCCTCTTTTGAGGAACAAGTTTTCTTGTTTTGAGCAGG | 48 |
| 726 | ENO1_20 | AGGCAGTTGCTCCTCTTTTGAGGAACAAGTTTTCTTGTAGGACTTCTC | 48 |
| 727 | ENO1_21 | GTTCACGGCCTCCTCTTTTGAGGAACAAGTTTTCTTGTTTGGCGATCC | 48 |
| 728 | ENO1_22 | TCTTTGGGTTGGTCACTGTGAGATCATCCCCCACTACC | 38 |
| 729 | ENO1_23 | TGGATTCCTGCACTGGCTGTGAACTTCTGCCAAGCTCC | 38 |
| 730 | ENO1_24 | CCAGTCATCCTGGTCAAAGGGATCTTCGATAGACACCA | 38 |
| 731 | ENO1_25 | CTGGGTAGTCCTTGATGAAGGACTTGTACAGGTCAGCC | 38 |
| 732 | ENO1_26 | AGCTGGTCAGGCGAGATGTACCTGCTGGGGTCATCGGG | 38 |
| 733 | ENO1_27 | AGACTTGAAGTCCAGGTCATACTTCCCAGACCTGAAGA | 38 |
| 734 | ENO1_28 | ACTCGGAGGCCGCTACGTCCATGCCGATGACCACCTTATCAGTGTAGCCA | 48 |
| 735 | ENO1_29 | GCTTTCCCAATCCTCTTTTGAGGAACAAGTTTTCTTGTTAGCAGTCTT | 48 |
| 736 | ENO1_30 | CAGCAGCTCCTCCTCTTTTGAGGAACAAGTTTTCTTGTAGGCCTTCTT | 48 |
| 737 | ENO1_31 | TATTCTCCAGTCCTCTTTTGAGGAACAAGTTTTCTTGTGATGTTGGGA | 48 |
| 738 | ENO1_32 | GCAAACCCGCTCCTCTTTTGAGGAACAAGTTTTCTTGTCTTCATCCCC | 48 |
| 739 | ENO1_33 | CACATTGGTGTCCTCTTTTGAGGAACAAGTTTTCTTGTGCATCTTTCC | 48 |
| 740 | ENO1_34 | CATATTTCTCTCCTCTTTTGAGGAACAAGTTTTCTTGTCTTGATGACA | 48 |
| 741 | ENO1_35 | TTCTTCAGGTTGTGGTAAACCTCTGCTCCAATGCGCAT | 38 |
| 742 | ENO1_36 | GGCTTCCCTGAAGTTTGCTGCACCGACTGGGAGGATCA | 38 |
| 743 | ENO1_37 | TGAACTCCTGCATGGCCAGCTTGTTGCCAGCATGAGAA | 38 |
| 744 | ENO1_38 | CCGCCATTGATGACATTGAACGCCGGGACTGGCAGGAT | 38 |
| 745 | ENO1_39 | GACTTCAGAGTTGCCAGCCAAGTCAGCGATGTGGCGGT | 38 |
| 746 | ENO1_40 | ACAGGGGGACCCCCTTCTCAACGGCACCAGCTTTGCAG | 38 |
| 747 | ENO1_41 | ACGGCAAGGGACACCCCCAGAATGGCGTTCGCACCAAACTTAGATTTATT | 48 |
| 748 | ENO1_42 | TTCTGTTCCATCCTCTTTTGAGGAACAAGTTTTCTTGTTCCATCTCGA | 48 |
| 749 | ENO1_43 | TCATCAGTTTTCCTCTTTTGAGGAACAAGTTTTCTTGTGTCAATCTTC | 48 |
| 750 | ENO1_44 | TCTTGTTCTGTCCTCTTTTGAGGAACAAGTTTTCTTGTTGACGTTCAG | 48 |
| 751 | ENO1_45 | TTTCTTGCTATCCTCTTTTGAGGAACAAGTTTTCTTGTACCAGGGCAG | 48 |
| 752 | ENO1_46 | GCGCAATAGTTCCTCTTTTGAGGAACAAGTTTTCTTGTTTTATTGATG | 48 |
| 753 | ENO1_47 | TGCTCAACAGTCCTCTTTTGAGGAACAAGTTTTCTTGTCCTTTGAGAC | 48 |
| 754 | ENO1_48 | ACCTGGAAGGAACAATCAAATCAAATTACTGACATTAA | 38 |
| 755 | ENO1_49 | CTTTAAAACAGGCTTGAGCAGCATTGTTAGAGAGAACC | 38 |
| 756 | ENO1_50 | ACTGTTGAGGCCGACGCGGAAGCCCACCAAGGGACCTT | 38 |
| 757 | ENO1_51 | CTGTGGGACCTCTTCCCCCTCTCCCCTTTCCCCCCAGA | 38 |
| 758 | ENO1_52 | ATCGGAGGACTTTCCGGCCCAGTCTGAGTGCTCCTACC | 38 |
| 759 | ENO1_53 | TAGGACCCCAGAGGGTTAGAGCCACACAAAACAGCAGC | 38 |
| 760 | ENO1_54 | TGGCTCAGAACGATCTGACTGGAGGTTAGTTTGCAAGA | 38 |
| 761 | ENO1_55 | CCATGCACTGGAAACGCCAACTGCAGACTAAAGGCTGT | 38 |
| 762 | ENO1_56 | TCAATCAGTTACCTGAGTGCACAGGGCTGGTGCCAGGA | 38 |
| 763 | ENO1_57 | ACTCATTAATATACTTAATGGGTCTGGAGACGCCTGCC | 38 |
| 764 | ENO1_58 | CAGCAGAGGATGAAGAAGGAAAAATGAGAGAGACTCAG | 38 |
| 765 | ENO1_59 | AAGAGAACCGGTGATTAAGGACTGCGAGTGAGAGACAG | 38 |
| 766 | ENO1_60 | TGAGGGGGCAGGAAAGCAGAGGAGAGCATTTCAGGGGC | 38 |
| 767 | ENO1_61 | CATGCGTTCAATCTTCCACAAATGCTTACTTACTGAGC | 38 |
| 768 | ENO1_62 | ACTCATTACCATCAAGGCACAGCATCATCCTGCTCCGG | 38 |
| 769 | ENO1_63 | CTAAGTCCCCACGTACGCCATTAAACAACGGTCAAATG | 38 |
| 770 | ENO1_64 | GTAACATGTTCGTGTGTGTAGAGTGCTTCTTACAGCAT | 38 |
| 771 | ENO1_65 | TAAAGCGGGACTGAACACCCAGCACCACCACTGCGACA | 38 |
| 772 | ENO1_66 | CCAGCCCAGTGTTTCACAACCTGATTTCATCATTGTCC | 38 |
| 773 | ENO1_67 | CCTCCCTGCAGCCTTTTTGGAGTTTTCCGGGTGCCCCA | 38 |
| 774 | ENO1_68 | CTGAGAATGCGTGATCTAGCCCTATGTGCTTTTCTGTA | 38 |
| 775 | ENO1_69 | ATTTGGCCACATGTTCTATCTCTAGAAAGGTCAATGTT | 38 |
| 776 | ENO1_70 | GCACACACACTAAGTCAATTTGAATA | 26 |
| 777 | ENO1_71 | CAGCCAGAGGTCCTCTTTTGAGGAACAAGTTTTCTTGTCTAGCTCTGC | 48 |
| 778 | ENO1_72 | CACAGAGCAGTCCTCTTTTGAGGAACAAGTTTTCTTGTGGCTCCTATG | 48 |
| 779 | ENO1_73 | AGTCAGTCAATCCTCTTTTGAGGAACAAGTTTTCTTGTCAGGAAAATC | 48 |
| 780 | ENO1_74 | ACACTAAATGTCCTCTTTTGAGGAACAAGTTTTCTTGTACACACCTGC | 48 |
| 781 | ENO1_75 | AGCTCACCATTCCTCTTTTGAGGAACAAGTTTTCTTGTTCAGGGCAGG | 48 |
| 782 | ENO1_76 | TCATTGGTTATCCTCTTTTGAGGAACAAGTTTTCTTGTGACCTTCCCT | 48 |
| 783 | ENO1_77 | GGATCCTTTCTAGCCCGTGAAGAGGCTGATGATCTCTGGAGGGCCCA | 47 |
| 784 | ENO1_78 | CAGAACCCTCCTGGGAGATGGGGAAACTTTTCTTTAGTTAACAGGCC | 47 |
| 785 | ENO1_79 | CTGTTCTCATGCTTGGGTTCCTGTCCTAACCGTGAAGGAGTATTTCG | 47 |
| 786 | ENO1_80 | CAGATTGGGAACAAACTGCAATAGAAGCAAGCATTGGAACTCTCGAC | 47 |
| 787 | ENO1_81 | CCAGAGCATGCAGGCTCGGGAACCCCGGAATCCACACACCAACTTTC | 47 |
| 788 | ENO1_82 | CTGTCCACAAGGTGGTGCACTGCTTCCATCAACATCATGGGTC | 43 |
| 789 | ENO1_83 | ACAGCAGGTTTCCTCTTTTGAGGAACAAGTTTTCTTGTTACCTGCCAT | 48 |
| 790 | ENO1_84 | AAACCTGCAATCCTCTTTTGAGGAACAAGTTTTCTTGTGTGCAAGTGC | 48 |
| 791 | ENO1_85 | CACCCAGAGATCCTCTTTTGAGGAACAAGTTTTCTTGTGGACAGGACT | 48 |
| 792 | ENO1_86 | GGGCAAGGCCTCCTCTTTTGAGGAACAAGTTTTCTTGTAGGAATGGGG | 48 |
| 793 | ENO1_87 | CTGGAAGCAGTCCTCTTTTGAGGAACAAGTTTTCTTGTGGAGGCCATG | 48 |
| 794 | ENO1_88 | GGCTGTGGGTTCCTCTTTTGAGGAACAAGTTTTCTTGTTCTAAGGCTT | 48 |
| 795 | ENO1_89 | ACCCTTCCCCATATAGCGAGTCTTATCATTGTCCCGGAGCTCTAGGGCCT | 50 |
| 796 | ENO1_90 | CATAGATACCAGTTGAAGCACCACTGGGCACAGCAGCTCTGAAGAGACCT | 50 |
| 797 | ENO1_91 | TTTGAGGTGAAGAGATCAACCTCAACAGTGGGATTCCCGCGAGAGTCAAA | 50 |
| 798 | ENO1_92 | GATCTCCCTGGCATGGATCTTGAGAATAGACATGGTGAACTTCTAGCCAC | 50 |
| 799 | ENO1_93 | TGGGTCTCGTCGCCTAGGAGAGGAAGCGGAGGGTGCTGCAGACACCGAGG | 50 |
| 800 | ENO1_94 | TGAACGTAAAGCCGGCGAGATCTCCGTGCTCCGGGTACCCACAGATACT | 49 |
| 801 | ENO1_95 | GAGTGCACAGGGCTGGTGCCAGGAACTC | 28 |
| 802 | ENO1_96 | ATTAATATACTTAATGGGTCTGGAGACG | 28 |

TABLE 15
Xist IncRNA ID '111111' linearising units. Linearising units that form structural
colours are SEQ ID NOs 813-818; 827-832; 841-846; 855-860; 869-874; and 883-888.

| SEQ | | | Length |
|---|---|---|---|
| ID NO | Name | Sequence (5′→3′) | (nt) |
| 803 | Xist_1 | TTTTTTTTTTTTTTTTCAAAGTTTTCAAAACAGTATAT | 38 |
| 804 | Xist_2 | TTTATTTTACAATAGCAACCAACTCCCCAGTTTGTTTC | 38 |
| 805 | Xist_3 | AATTGTGACATCTAGATGGCTTAAGATTACTTTCTGGT | 38 |
| 806 | Xist_4 | GGTCACCCATGCTGAACAATATTTTTCAATCTTCCAAA | 38 |
| 807 | Xist_5 | CAGCAAAGACTCAAAAGAGATTCTGCATTTCACATCAG | 38 |
| 808 | Xist_6 | TTCACAAGTTCAAGAGTCTTCCATTTATCTTAGCTTTT | 38 |
| 809 | Xist_7 | GGAATAAATTATCTTTGAGGTAGAAGGACAATGACGAA | 38 |
| 810 | Xist_8 | GCCACTTAATTCCTTGTGTCTGCATAAAAGCAGATTTA | 38 |
| 811 | Xist_9 | TTCATCACAACTTCATTTATGTGAATAAAGCAGATGAT | 38 |
| 812 | Xist_10 | GATAAAATGTTCTCTTATTCTTGTTTAATCAGTAGTGG | 38 |
| 813 | Xist_11 | TAGTGATGCCTCCTCTTTTGAGGAACAAGTTTTCTTGTAGAAACTGTG | 48 |
| 814 | Xist_12 | AAAGGAAGGCTCCTCTTTTGAGGAACAAGTTTTCTTGTTTTTAGTTAC | 48 |
| 815 | Xist_13 | TTTCTTCTTTTCCTCTTTTGAGGAACAAGTTTTCTTGTCCATTTTCCA | 48 |
| 816 | Xist_14 | ATAATCCATTTCCTCTTTTGAGGAACAAGTTTTCTTGTCCCCATCCCC | 48 |
| 817 | Xist_15 | AGCTGAAGAATCCTCTTTTGAGGAACAAGTTTTCTTGTAGGGGTGTTA | 48 |
| 818 | Xist_16 | CTGAGTCCAGTCCTCTTTTGAGGAACAAGTTTTCTTGTCTGATACCAC | 48 |
| 819 | Xist_17 | ACATTGAAAGGTAAACATTAATATTTCAAATCTGATGT | 38 |
| 820 | Xist_18 | CTAACTAAAAATGTACAGAATGAAAACTAGAAAATTTC | 38 |
| 821 | Xist_19 | AACCCCAGATTATCTTCAACCTTGCTCCCTCCACCAAT | 38 |
| 822 | Xist_20 | CATACTTTGACATTTATCTATTTCCTTCTCCACTTATG | 38 |
| 823 | Xist_21 | GATGTAATTGGCTTGCTATAGAAACTACAGTTCAGATG | 38 |
| 824 | Xist_22 | CTTTGAATGTATGAACTACAATGAACAATAAAGTCCTC | 38 |
| 825 | Xist_23 | TTCTTTTGAAGCATATTTTGGCTTC | 25 |
| 826 | Xist_24 | AGCTTTAAGATAATCTTATGACAAG | 25 |
| 827 | Xist_25 | AAGGGTCACATCCTCTTTTGAGGAACAAGTTTTCTTGTCTGATTCACT | 48 |
| 828 | Xist_26 | TAATAAATTCTCCTCTTTTGAGGAACAAGTTTTCTTGTCATTCTTACC | 48 |
| 829 | Xist_27 | TAACACAAGGTCCTCTTTTGAGGAACAAGTTTTCTTGTTTTAGTTGAT | 48 |
| 830 | Xist_28 | AAGCACTTGGTCCTCTTTTGAGGAACAAGTTTTCTTGTACAAAAATAA | 48 |
| 831 | Xist_29 | TACTTTTCAATCCTCTTTTGAGGAACAAGTTTTCTTGTAAATGTAAAG | 48 |
| 832 | Xist_30 | CAAACTAGTGTCCTCTTTTGAGGAACAAGTTTTCTTGTAGGACAAAGG | 48 |
| 833 | Xist_31 | ATTTTGTCCTCATCTCAACAATGATCAGCTATTGGAAC | 38 |
| 834 | Xist_32 | TGCATGAAACTGAACAATTTAAACCTGGAGCTGGTAAT | 38 |
| 835 | Xist_33 | GTTCTTAAGACCAATTCAGAACAAAGGCAGGTTGCCCT | 38 |
| 836 | Xist_34 | TAAAACAGGTTTGACCTTTTCCTTCACTCTTCCTCCTG | 38 |
| 837 | Xist_35 | TCCCACCCTCTGTGAGTGATTTAAAAACGGAAAAGGTC | 38 |
| 838 | Xist_36 | AAAGCCCAGCCAGGCCTACATTTAGAGAAATTTTAAAA | 38 |
| 839 | Xist_37 | AAATTTTTCCTTTCAATTTTGGCCA | 25 |
| 840 | Xist_38 | ATTATTTCCAATTTTTATTTTATTC | 25 |
| 841 | Xist_39 | TTAAAACTTATCCTCTTTTGAGGAACAAGTTTTCTTGTAGCATGGTAA | 48 |
| 842 | Xist_40 | AATGTTAAGCTCCTCTTTTGAGGAACAAGTTTTCTTGTTGTTTTCATC | 48 |
| 843 | Xist_41 | CACTGATATTTCCTCTTTTGAGGAACAAGTTTTCTTGTATTCTACTAT | 48 |
| 844 | Xist_42 | AAAAAGCCCTTCCTCTTTTGAGGAACAAGTTTTCTTGTTCTTGAGCAG | 48 |
| 845 | Xist_43 | ATTTGATGATTCCTCTTTTGAGGAACAAGTTTTCTTGTAAAAAGGAGA | 48 |
| 846 | Xist_44 | ACATTTCTAATCCTCTTTTGAGGAACAAGTTTTCTTGTGGTATAATTA | 48 |
| 847 | Xist_45 | GAGAAGCTGTTGCATGAGAAATCATGTCTCCATCTCCA | 38 |
| 848 | Xist_46 | TTTTGCTATGCGTTATCTGAGGATTGTTTCTGAAAGAG | 38 |
| 849 | Xist_47 | ATCTATTTAGGCAGTGTATGTATGTGTCAGCATGTAAA | 38 |
| 850 | Xist_48 | AAGTAAAGAACTGAAAATAGACAAACCTTGTAAATGCA | 38 |
| 851 | Xist_49 | CTTCAAAACCAATTGTGGCTCAAGTGTAGGTGGTTCCC | 38 |
| 852 | Xist_50 | CAAGGCTGGTACCAATGAGACTGGGGTTTGGGAATTAG | 38 |
| 853 | Xist_51 | TTGGTCATCATCCCTCCTGCTGCCC | 25 |
| 854 | Xist_52 | AGCAGTGGTCAGTCATTTTTCATGA | 25 |
| 855 | Xist_53 | GGGATGGACTTCCTCTTTTGAGGAACAAGTTTTCTTGTAGGAAAATGA | 48 |
| 856 | Xist_54 | AGTGTCTATTTCCTCTTTTGAGGAACAAGTTTTCTTGTATAAGGAGAG | 48 |
| 857 | Xist_55 | TTCCTGTAACTCCTCTTTTGAGGAACAAGTTTTCTTGTTTAAGGAGCA | 48 |
| 858 | Xist_56 | AGCTCACTACTCCTCTTTTGAGGAACAAGTTTTCTTGTAAAACCCAGC | 48 |
| 859 | Xist_57 | TACCTGCTTATCCTCTTTTGAGGAACAAGTTTTCTTGTTCGTAGTGGC | 48 |
| 860 | Xist_58 | CAGAGTGGTATCCTCTTTTGAGGAACAAGTTTTCTTGTGAAGAGATAC | 48 |
| 861 | Xist_59 | GGAGTAGGAATTAAACCACACAATGTTATTTAGGGACT | 38 |
| 862 | Xist_60 | AAGCCATGCCCCTAACAAGAAAACAAGCCAAAAGGAAA | 38 |
| 863 | Xist_61 | GTATTAGGCATTCTCTGGGAAGGCATGCATTTTTTTCC | 38 |
| 864 | Xist_62 | CATGTCTCTGGGGCCAAAAACCTTATACCAAGTACCTA | 38 |
| 865 | Xist_63 | TTGGCACCCGAATATATTTGTAGAATGAATGAATACAT | 38 |
| 866 | Xist_64 | GAAAAAAAATAAACAGTAACCTTTCTCCTATATTCTAC | 38 |
| 867 | Xist_65 | TTTCCAAGCCAATTAATAAGCAAGT | 25 |
| 868 | Xist_66 | GTCTTTTCGTCATGATTTTTTTTGT | 25 |
| 869 | Xist_67 | TTTCTGTTTATCCTCTTTTGAGGAACAAGTTTTCTTGTGGATTTAACA | 48 |
| 870 | Xist_68 | AAATGGTTGATCCTCTTTTGAGGAACAAGTTTTCTTGTGATAACAGTC | 48 |
| 871 | Xist_69 | ACTTCTGTTTTCCTCTTTTGAGGAACAAGTTTTCTTGTGATGAAGAGT | 48 |
| 872 | Xist_70 | ATCACTTCATTCCTCTTTTGAGGAACAAGTTTTCTTGTTCCATTTTTG | 48 |
| 873 | Xist_71 | TGTTTTTGTTTCCTCTTTTGAGGAACAAGTTTTCTTGTGCATCTCCAA | 48 |
| 874 | Xist_72 | GTCAGAATAATCCTCTTTTGAGGAACAAGTTTTCTTGTATGCCTTTTG | 48 |
| 875 | Xist_73 | GAGCAGATATATTTCATTTAGCATTTAGTATCATCTTC | 38 |
| 876 | Xist_74 | ATCAATACTCGTATGAACGAAAAAATAAAAAGCCCTCT | 38 |
| 877 | Xist_75 | CTTATTCCCACTCTACAACGCATGTCAAAGGTGATCTG | 38 |
| 878 | Xist_76 | TTTAGTTTTTCCTTAGTATCGAACATATCACAGCTACT | 38 |
| 879 | Xist_77 | CAATGAAGTTTCTTCTCAACTAAAGAAACACAGTCTCT | 38 |
| 880 | Xist_78 | TAGAGAATTTGTTCCTGTGTTTCCACCATAAGATAAAT | 38 |
| 881 | Xist_79 | GAGATAAAGTGCACTTTGGTTTACG | 25 |
| 882 | Xist_80 | TCTTTCAAGCACTTAAGAACAGCAC | 25 |
| 883 | Xist_81 | TTTATTCAGTTCCTCTTTTGAGGAACAAGTTTTCTTGTTTTAATAAAT | 48 |
| 884 | Xist_82 | TTTTACTAAATCCTCTTTTGAGGAACAAGTTTTCTTGTTTGGTAAATG | 48 |
| 885 | Xist_83 | CAAAAGAATCTCCTCTTTTGAGGAACAAGTTTTCTTGTTTTTTTTTAT | 48 |
| 886 | Xist_84 | TGCTCATATGTCCTCTTTTGAGGAACAAGTTTTCTTGTTCTTCCTGTC | 48 |
| 887 | Xist_85 | TCTAAGCCAGTCCTCTTTTGAGGAACAAGTTTTCTTGTACCAATGAGC | 48 |
| 888 | Xist_86 | AAATACCTTTTCCTCTTTTGAGGAACAAGTTTTCTTGTAAAACTAGTT | 48 |
| 889 | Xist_87 | GTTACATCTTGAACCATTTAACTGTAATAAAAGCAGAA | 38 |
| 890 | Xist_88 | TGTTTAGTTAATGAATTAAAGAACAAACCCTGAGCCCT | 38 |
| 891 | Xist_89 | TTTATCAGTCTCCTGGCTTTAAACTAAGCCAATGAGGA | 38 |
| 892 | Xist_90 | AGTGATTTGGGGGATTCCTGAAACTAGGAAAAATGTCT | 38 |
| 893 | Xist_91 | CTTTATTTTGAAAGAAACTTGCATTTTTCTTTCTTTTT | 38 |
| 894 | Xist_92 | TTTTTTTTTTTTAATCTCAAAGGCAATTGAGTGGGTCT | 38 |
| 895 | Xist_93 | TCTGGGCCAGACCTATTTAATTTACGAAACATAGTACC | 38 |
| 896 | Xist_94 | TTGCAGAGAATAGGCATTGAAATATTATTTAAACAATC | 38 |
| 897 | Xist_95 | AAACCAAAGATGTTCTTCTATCTTCAGCTGTCAGTGAT | 38 |
| 898 | Xist_96 | CTAATGCCCTCATCTCTCTTATCCTCAGGACCCAGAAT | 38 |

TABLE 16
Linearising units for fabrication of MS2 native structural unit ID.

| SEQ | | | |
|---|---|---|---|
| ID | | | Length |
| NO | Name | Sequence (5′→3′) | (nt) |
| 899 | internal M1 | TGGGTGGTAACTAGCCAAGCAGCTAGTTACCAAATCGG | 38 |
| 900 | internal M2 | GAGAATCCCGGGTCCTCTCTTTAGGGGGAGGTCCCTGG | 38 |
| 901 | internal M3 | GCCGAAGCCCGCCCACCTTTCGGTGGAGCCGGACCGCT | 38 |
| 902 | internal M4 | TTCGCACCCGTGCTCTTTCGAGCACACCCACCCCGTTT | 38 |
| 903 | internal M5 | ACGGGGGTCCCTCGGTCAGCTACCGAGGAGAGCTCGCT | 38 |
| 904 | internal M6 | GGCCCACACTCCTGAGGGAATGTGGGAACCGGCGTTAG | 38 |
| 905 | internal M7 | CCACTCCGAAGTGCGTATAACGCGCACGCCGGCGGACT | 38 |
| 906 | internal M8 | TCATGCTGTCGGTGATTTCACCTCCAGTATGGAACCAC | 38 |
| 907 | internal M9 | GCTATGTAGCGACCACTGTCGTGCTTTTCGCTGAAGAA | 38 |
| 908 | internal M10 | CTTGCGTTCTCGAGCGATACGAGCA | 25 |
| 909 | internal M11 | AGACGGAAACCCGAGGTACGGGTATC | 26 |
| 910 | internal M12 | CGCGAGCAGCCGCCCGTACGGAGTCTAGAGGCGTGGATCTGACATACCTC | 50 |
| 911 | internal M13 | CGACAACTCCCCAACCCCGTAGCCGA | 26 |
| 912 | internal M14 | TTTAATATCAGCATCAGGGCGAAGA | 25 |
| 913 | internal M15 | GATTGTCAACAGGTTTCTTGATGTAAAACGGTTTGACA | 38 |
| 914 | internal M16 | TCGACACCACGGTAAAAGTGCGCGCCGCAGCTCTCGCG | 38 |
| 915 | internal M17 | AAAGAGCCCGGACACGAACGTTTTACGAAGATTCGGTT | 38 |
| 916 | internal M18 | TAAAACCGTAGTAGGCAAGTGCCTCTAGCACACGGGGT | 38 |
| 917 | internal M19 | GCAATCTCACTGGGACATATAATATCGTCCCCGTAGAT | 38 |
| 918 | internal M20 | GCCTATGGTTCCGGCGTTACCAAAATGGATTTGGGTCG | 38 |
| 919 | internal M21 | CTTTGACTATTGCCCAGAATATCATGGACTCTAGCTCA | 38 |
| 920 | internal M22 | AATGTGAACCCATTTCCCATTGTGGAAAATAGTTCCCA | 38 |
| 921 | internal M23 | TCGTATCGTCTCGCCATCTACGATTCCGTAGTGTGAGC | 38 |
| 922 | internal M24 | GGATACGATCGAGATATGAATATAGCTCTGGTGGGAGA | 38 |
| 923 | internal M25 | AAACTCCACACCAGGCGATCGGAGATGGAATCGGATGC | 38 |
| 924 | internal M26 | AGACGATAAGTCTATCGTCGCAAGCGAACCATCTACGC | 38 |
| 925 | internal M27 | TGCCCTGCTGAGCCAGACGCTGGTTGATCGATTGATCA | 38 |
| 926 | internal M28 | TTCAGGTCTATACCAACGGATTTGAGCCGGCGTCTGAT | 38 |
| 927 | internal M29 | GAAAGCACCGACCCCTTTCTGGAGGTACATATTCATAT | 38 |
| 928 | internal M30 | CAGGCTCCTTACAGGCAGCCCGATC | 25 |
| 929 | internal M31 | TATTTTATTATTCTTCGGAACTGTAA | 26 |
| 930 | internal M32 | ACACTCCGTTCCCTACAACGAGCCTAAATTCATATGACTCGTTATAGCGG | 50 |
| 931 | internal M33 | ACCGCGTGTCTGAACGGATGCTGGT | 25 |
| 932 | internal M34 | TTGTAAAACATCCGGATCCCATGACA | 26 |
| 933 | internal M35 | AGGATTTGTCATGTAAGAAACCTTCTCTATTTATCTGA | 38 |
| 934 | internal M36 | CCGCGATCACCATTCGCCTCCCGTAGCTTAGCGATAGC | 38 |
| 935 | internal M37 | TAAGGTACGACGGGTCGCCTCGTCATTACCAGAACCTA | 38 |
| 936 | internal M38 | AGGTCGGATGCTTTGTGAGCAATTCGTCCCTTAAGTAA | 38 |
| 937 | internal M39 | GCAATTGCTGTAAAGTCGTCACTGTGCGGATCACCGCT | 38 |
| 938 | internal M40 | TCCAGTAGCGACAGAAGCAATTGATTGGTAAATTTCGA | 38 |
| 939 | internal M41 | GAGAAAGATCGCGAGGAAGATCAATACATAAAGAGTTG | 38 |
| 940 | internal M42 | AACTTCTTTGTTGTCTTCGACATGGGTAATCCTCATGT | 38 |
| 941 | internal M43 | TTGAATGGCCGGCGTCTATTAGTAGATGCCGGAGTTTG | 38 |
| 942 | internal M44 | CTGCGATTGCTGAGGGAATCGGGTTTCCATCTTTTAGG | 38 |
| 943 | internal M45 | AGACCTTGCATTGCCTTAACAATAAGCTCGCAGTCGGA | 38 |
| 944 | internal M46 | ATTCGTAGCGAAAATTGGAATGGTTAGTTCCATATTTA | 38 |
| 945 | internal M47 | AGTACGAACGCCATGCGGCTACAGGAAGCTCTACACCA | 38 |
| 946 | internal M48 | CCAACAGTCTGGGTTGCCACTTTAGGCACCTCGACTTT | 38 |
| 947 | internal M49 | GATGGTGTATTTGCGATTCTGCGCAGAGCTCTGACGAA | 38 |
| 948 | internal M50 | CGCTACAGGTTACTTTGTAAGCCTGTGAACGCGAGTTA | 38 |
| 949 | internal M51 | GAGCTGATCCATTCAGCGACCCCGTTAGCGAAGTTGCT | 38 |
| 950 | internal M52 | TGGGGCGACAGTCACGTCGCCAGTTCCGCCATTGTCGA | 38 |
| 951 | internal M53 | CGAGAACGAACTGAGTAAAGTTAGAAGCCATGCTTCAA | 38 |
| 952 | internal M54 | ACTCCGGTTGAGGGCTCTATCTAGAGAGCCGTTGCCTG | 38 |
| 953 | internal M55 | ATTAATGCTAACGCATCTAAGGTATGGACCATCGAGAA | 38 |
| 954 | internal M56 | AGGAGACTTTACGTACGCGCCAGTTGTTGGCCATACGG | 38 |
| 955 | internal M57 | ATTGTACCCCTCGATGCATGGCTGAGATTTGGGCCTTA | 38 |
| 956 | internal M58 | GCAGTGCCCTGTCTCTCCACAGTCCACCCGTAGGGAGC | 38 |
| 957 | internal M59 | GTCAACGCTTATGATGGACTCACCCGTTATTACGTCAG | 38 |
| 958 | internal M60 | TAACTGTTCCTGACATGTAGGAGCATCCCACGGGGGCC | 38 |
| 959 | internal M61 | GTAAGGCCCTCGAGCATGTTACCTACAGGTAGGAGCCA | 38 |
| 960 | internal M62 | GTCGACAACGAATGAGAAAGGCACCTTTTCCCACACTA | 38 |
| 961 | internal M63 | TACCTAGTGGGTTCAAGATACCTAGAGACGACAACCAT | 38 |
| 962 | internal M64 | GCCAAACGTGCATCGTTTATGTAAAACCATATCACGAT | 38 |
| 963 | internal M65 | ACGTCGCGATATGTTGCACGTTGTC | 25 |
| 964 | internal M66 | TGGAAGTTTGCAGCTGGATACGACAG | 26 |
| 965 | internal M67 | ACGGCCATCTAACTTGATGTTAGTAGCAACGTTCTGCGGCACTTCGATGT | 50 |
| 966 | internal M68 | AAGTCAAGTTTTGGCTTACAGGGAA | 25 |
| 967 | internal M69 | GAGGCTGTAGCAGGAGCGTGCGTCGA | 26 |
| 968 | internal M70 | GGGAGAAGCCGAAACCGGCTTTCTCCTCGTACGGGCGA | 38 |
| 969 | internal M71 | CCCCACGATGACCCACTTCGCTTGTAGGCACCTTGATC | 38 |
| 970 | internal M72 | TATCGATGTGACACTTAACGCCCCCCGTGAATACGGAG | 38 |
| 971 | internal M73 | AGGGGTAGTGCCACTGTTTCGTTTTGGCCCCAGTCGAG | 38 |
| 972 | internal M74 | TTAAAACGACCGGGAGTCCAGTTCGAACGATATTTTAA | 38 |
| 973 | internal M75 | AGAGAATGAGTTATCTTCAGTCTCACCGTCCGCGTAAA | 38 |
| 974 | internal M76 | CGCGAACGGAGGGGACGAAGGTCTCGTTCTCCCTATCA | 38 |
| 975 | internal M77 | AGGGTACTAAAAGCTCGCACAGGTCAAACCTCCTAGGA | 38 |
| 976 | internal M78 | ATGGAATTCCGGCTACCTACAGCGATAGCCATGGTAGC | 38 |
| 977 | internal M79 | GTCTCGCTAAAGACATTAAAAATGGCATTAGCTCGACA | 38 |
| 978 | internal M80 | GGAAGTTGAGCAGGACCCCGAAAGGGGTCCCACCC | 35 |

TABLE 17
Linearising units for fabrication of terminal native structural unit ID.

| SEQ ID | | | Length |
|---|---|---|---|
| NO | Name | Sequence (5′→3′) | (nt) |
| 979 | terminal M1 | CACTCCGTTCCCTACAACGAGCCTAAATTCATATGACT | 38 |
| 980 | terminal M2 | CGTTATAGCGGACCGCGTGTCTGATCCACGGCGCACAT | 38 |
| 981 | terminal M3 | TGGTCTCGGACCAATAGAGCCGCTCTCAGAGCGCGGGG | 38 |
| 982 | terminal M4 | GGTAACGGTTGCTTGTTCAGCGAACTTCTTGTAAGGCG | 38 |
| 983 | terminal M5 | CTGCATCCTGCAACTTGTGCCCCATAGGAGCACCGTTG | 38 |
| 984 | terminal M6 | GAGAACGTGCATTGCCCAAACAACGACGATCGGTAGCC | 38 |
| 985 | terminal M7 | AGAGAGGAGGTTGCCAATAAGGCTACGGATGCTGGTTT | 38 |
| 986 | terminal M8 | GTAAAACATCCGGATCCCATGACAAGGATTTGTCATGT | 38 |
| 987 | terminal M9 | AAGAAACCTTCTCTATTTATCTGACCGCGATCACCATT | 38 |
| 988 | terminal M10 | CGCCTCCCGTAGCTTAGCGATAGCTAAGGTACGACGGGTC | 40 |
| 989 | terminal M11 | GCCTCGTCATTACCAGAACCTAAGGTCGGATGCTTTGTGA | 40 |
| 990 | terminal M12 | GCAATTCGTCCCTTAAGTAAGCAATTGCTGTAAAGTCGTC | 40 |
| 991 | terminal M13 | ACTGTGCGGATCACCGCTTCCAGTAGCGACAG | 32 |
| 992 | terminal M14 | AAGCAATTGATTGGTAAATTTCGAGAGAAAGATCGCGA | 38 |
| 993 | terminal M15 | GGAAGATCAATACATAAAGAGTTGAACTTCTTTGTTGT | 38 |
| 994 | terminal M16 | CTTCGACATGGGTAATCCTCATGTTTGAATGGCCGGCG | 38 |
| 995 | terminal M17 | TCTATTAGTAGATGCCGGAGTTTGCTGCGATTGCTGAG | 38 |
| 996 | terminal M18 | GGAATCGGGTTTCCATCTTTTAGGAGACCTTGCATTGC | 38 |
| 997 | terminal M19 | CTTAACAATAAGCTCGCAGTCGGAATTCGTAGCGAAAA | 38 |
| 998 | terminal M20 | TTGGAATGGTTAGTTCCATATTTAAGTACGAACGCCAT | 38 |
| 999 | terminal M21 | GCGGCTACAGGAAGCTCTACACCACCAACAGTCTGGGT | 38 |
| 1000 | terminal M22 | TGCCACTTTAGGCACCTCGACTTTGATGGTGTATTTGC | 38 |
| 1001 | terminal M23 | GATTCTGCGCAGAGCTCTGACGAACGCTACAGGTTACT | 38 |
| 1002 | terminal M24 | TTGTAAGCCTGTGAACGCGAGTTAGAGCTGATCCATTC | 38 |
| 1003 | terminal M25 | AGCGACCCCGTTAGCGAAGTTGCTTGGGGCGACAGTCA | 38 |
| 1004 | terminal M26 | CGTCGCCAGTTCCGCCATTGTCGACGAGAACGAACTGAGT | 40 |
| 1005 | terminal M27 | AAAGTTAGAAGCCATGCTTCAAACTCCGGTTGAGGGCTCT | 40 |
| 1006 | terminal M28 | ATCTAGAGAGCCGTTGCCTGATTAATGCTAACGCATCTAA | 40 |
| 1007 | terminal M29 | GGTATGGACCATCGAGAAAGGAGACTTTACGT | 32 |
| 1008 | terminal M30 | ACGCGCCAGTTGTTGGCCATACGGATTGTACCCCTCGA | 38 |
| 1009 | terminal M31 | TGCATGGCTGAGATTTGGGCCTTAGCAGTGCCCTGTCT | 38 |
| 1010 | terminal M32 | CTCCACAGTCCACCCGTAGGGAGCGTCAACGCTTATGA | 38 |
| 1011 | terminal M33 | TGGACTCACCCGTTATTACGTCAGTAACTGTTCCTGAC | 38 |
| 1012 | terminal M34 | ATGTAGGAGCATCCCACGGGGGCCGTAAGGCCCTCGAG | 38 |
| 1013 | terminal M35 | CATGTTACCTACAGGTAGGAGCCAGTCGACAACGAATG | 38 |
| 1014 | terminal M36 | AGAAAGGCACCTTTTCCCACACTATACCTAGTGGGTTC | 38 |
| 1015 | terminal M37 | AAGATACCTAGAGACGACAACCATGCCAAACGTGCATC | 38 |
| 1016 | terminal M38 | GTTTATGTAAAACCATATCACGATACGTCGCGATATGT | 38 |
| 1017 | terminal M39 | TGCACGTTGTCTGGAAGTTTGCAGCTGGATACGACAGACG | 40 |
| 1018 | terminal M40 | GCCATCTAACTTGATGTTAGTACCGACCTGACGTACGGCT | 40 |
| 1019 | terminal M41 | CTCATAGGAAGAAACTCTTGAAGGTGAACCTTCGTAAGCA | 40 |
| 1020 | terminal M42 | TCTCATATGCACCCTGGATATCACTCATTAGT | 32 |
| 1021 | terminal M43 | GGTAACCAACCGAACTGCAACTCCAACCACCTGCCGGC | 38 |
| 1022 | terminal M44 | CACGTGTTTTGATCGAAACTTTCGATCTTCGTTTAGGG | 38 |
| 1023 | terminal M45 | CAAGGTAGCGGAGCGCCTGGCGCCAATTACCGCGACGA | 38 |
| 1024 | terminal M46 | GCGGCAGTGTACGCCTTCACGAGCGCAATGGTTTGCGT | 38 |
| 1025 | terminal M47 | CGCGAGTTGTGAGGCTGTCGACCTGGCCTCTGCTAAAG | 38 |
| 1026 | terminal M48 | CAACACCAAGGTTAAAATTACCCTGGGTGACCTTTTGC | 38 |
| 1027 | terminal M49 | AGGACTTCGGTCGACGCCCGGTTCGCAACGTTCTGCGG | 38 |
| 1028 | terminal M50 | CACTTCGATGTAAGTCAAGTTTTGGCTTACAGGGAAGA | 38 |
| 1029 | terminal M51 | GGCTGTAGCAGGAGCGTGCGTCGAGGGAGAAGCCGAAA | 38 |

Phage MS2 nucleotide sequence,3569 nt, NC_001417.2

DNA form
(SEQ ID NO: 1030)
GGGTGGGACCCCTTTCGGGGTCCTGCTCAACTTCCTGTCGAGCTAATGCCATTTTTAATGTCTTTAGCGA
GACGCTACCATGGCTATCGCTGTAGGTAGCCGGAATTCCATTCCTAGGAGGTTTGACCTGTGCGAGCTTT
TAGTACCCTTGATAGGGAGAACGAGACCTTCGTCCCCTCCGTTCGCGTTTACGCGGACGGTGAGACTGAA
GATAACTCATTCTCTTTAAAATATCGTTCGAACTGGACTCCCGGTCGTTTTAACTCGACTGGGGCCAAAA
CGAAACAGTGGCACTACCCCTCTCCGTATTCACGGGGGGCGTTAAGTGTCACATCGATAGATCAAGGTGC
CTACAAGCGAAGTGGGTCATCGTGGGGTCGCCCGTACGAGGAGAAAGCCGGTTTCGGCTTCTCCCTCGAC
GCACGCTCCTGCTACAGCCTCTTCCCTGTAAGCCAAAACTTGACTTACATCGAAGTGCCGCAGAACGTTG
CGAACCGGGCGTCGACCGAAGTCCTGCAAAAGGTCACCCAGGGTAATTTTAACCTTGGTGTTGCTTTAGC
AGAGGCCAGGTCGACAGCCTCACAACTCGCGACGCAAACCATTGCGCTCGTGAAGGCGTACACTGCCGCT
CGTCGCGGTAATTGGCGCCAGGCGCTCCGCTACCTTGCCCTAAACGAAGATCGAAAGTTTCGATCAAAAC
ACGTGGCCGGCAGGTGGTTGGAGTTGCAGTTCGGTTGGTTACCACTAATGAGTGATATCCAGGGTGCATA
TGAGATGCTTACGAAGGTTCACCTTCAAGAGTTTCTTCCTATGAGAGCCGTACGTCAGGTCGGTACTAAC
ATCAAGTTAGATGGCCGTCTGTCGTATCCAGCTGCAAACTTCCAGACAACGTGCAACATATCGCGACGTA
TCGTGATATGGTTTTACATAAACGATGCACGTTTGGCATGGTTGTCGTCTCTAGGTATCTTGAACCCACT
AGGTATAGTGTGGGAAAAGGTGCCTTTCTCATTCGTTGTCGACTGGCTCCTACCTGTAGGTAACATGCTC
GAGGGCCTTACGGCCCCCGTGGGATGCTCCTACATGTCAGGAACAGTTACTGACGTAATAACGGGTGAGT
CCATCATAAGCGTTGACGCTCCCTACGGGTGGACTGTGGAGAGACAGGGCACTGCTAAGGCCCAAATCTC
AGCCATGCATCGAGGGGTACAATCCGTATGGCCAACAACTGGCGCGTACGTAAAGTCTCCTTTCTCGATG
GTCCATACCTTAGATGCGTTAGCATTAATCAGGCAACGGCTCTCTAGATAGAGCCCTCAACCGGAGTTTG
AAGCATGGCTTCTAACTTTACTCAGTTCGTTCTCGTCGACAATGGCGGAACTGGCGACGTGACTGTCGCC
CCAAGCAACTTCGCTAACGGGGTCGCTGAATGGATCAGCTCTAACTCGCGTTCACAGGCTTACAAAGTAA
CCTGTAGCGTTCGTCAGAGCTCTGCGCAGAATCGCAAATACACCATCAAAGTCGAGGTGCCTAAAGTGGC
AACCCAGACTGTTGGTGGTGTAGAGCTTCCTGTAGCCGCATGGCGTTCGTACTTAAATATGGAACTAACC
ATTCCAATTTTCGCTACGAATTCCGACTGCGAGCTTATTGTTAAGGCAATGCAAGGTCTCCTAAAAGATG
GAAACCCGATTCCCTCAGCAATCGCAGCAAACTCCGGCATCTACTAATAGACGCCGGCCATTCAAACATG
AGGATTACCCATGTCGAAGACAACAAAGAAGTTCAACTCTTTATGTATTGATCTTCCTCGCGATCTTTCT
CTCGAAATTTACCAATCAATTGCTTCTGTCGCTACTGGAAGCGGTGATCCGCACAGTGACGACTTTACAG
CAATTGCTTACTTAAGGGACGAATTGCTCACAAAGCATCCGACCTTAGGTTCTGGTAATGACGAGGCGAC
CCGTCGTACCTTAGCTATCGCTAAGCTACGGGAGGCGAATGGTGATCGCGGTCAGATAAATAGAGAAGGT
TTCTTACATGACAAATCCTTGTCATGGGATCCGGATGTTTTACAAACCAGCATCCGTAGCCTTATTGGCA
ACCTCCTCTCTGGCTACCGATCGTCGTTGTTTGGGCAATGCACGTTCTCCAACGGTGCTCCTATGGGGCA
CAAGTTGCAGGATGCAGCGCCTTACAAGAAGTTCGCTGAACAAGCAACCGTTACCCCCCGCGCTCTGAGA
GCGGCTCTATTGGTCCGAGACCAATGTGCGCCGTGGATCAGACACGCGGTCCGCTATAACGAGTCATATG
AATTTAGGCTCGTTGTAGGGAACGGAGTGTTTACAGTTCCGAAGAATAATAAAATAGATCGGGCTGCCTG
TAAGGAGCCTGATATGAATATGTACCTCCAGAAAGGGGTCGGTGCTTTCATCAGACGCCGGCTCAAATCC
GTTGGTATAGACCTGAATGATCAATCGATCAACCAGCGTCTGGCTCAGCAGGGCAGCGTAGATGGTTCGC
TTGCGACGATAGACTTATCGTCTGCATCCGATTCCATCTCCGATCGCCTGGTGTGGAGTTTTCTCCCACC
AGAGCTATATTCATATCTCGATCGTATCCGCTCACACTACGGAATCGTAGATGGCGAGACGATACGATGG
GAACTATTTTCCACAATGGGAAATGGGTTCACATTTGAGCTAGAGTCCATGATATTCTGGGCAATAGTCA
AAGCGACCCAAATCCATTTTGGTAACGCCGGAACCATAGGCATCTACGGGGACGATATTATATGTCCCAG
TGAGATTGCACCCCGTGTGCTAGAGGCACTTGCCTACTACGGTTTTAAACCGAATCTTCGTAAAACGTTC
GTGTCCGGGCTCTTTCGCGAGAGCTGCGGCGCGCACTTTTACCGTGGTGTCGATGTCAAACCGTTTTACA
TCAAGAAACCTGTTGACAATCTCTTCGCCCTGATGCTGATATTAAATCGGCTACGGGGTTGGGGAGTIGT
CGGAGGTATGTCAGATCCACGCCTCTATAAGGTGTGGGTACGGCTCTCCTCCCAGGTGCCTTCGATGTTC
TTCGGTGGGACGGACCTCGCTGCCGACTACTACGTAGTCAGCCCGCCTACGGCAGTCTCGGTATACACCA
AGACTCCGTACGGGCGGCTGCTCGCGGATACCCGTACCTCGGGTTTCCGTCTTGCTCGTATCGCTCGAGA
ACGCAAGTTCTTCAGCGAAAAGCACGACAGTGGTCGCTACATAGCGTGGTTCCATACTGGAGGTGAAATC
ACCGACAGCATGAAGTCCGCCGGCGTGCGCGTTATACGCACTTCGGAGTGGCTAACGCCGGTTCCCACAT
TCCCTCAGGAGTGTGGGCCAGCGAGCTCTCCTCGGTAGCTGACCGAGGGACCCCCGTAAACGGGGTGGGT
GTGCTCGAAAGAGCACGGGTGCGAAAGCGGTCCGGCTCCACCGAAAGGTGGGCGGGCTTCGGCCCAGGGA
CCTCCCCCTAAAGAGAGGACCCGGGATTCTCCCGATTTGGTAACTAGCTGCTTGGCTAGTTACCACCCA
RNA form
(SEQ ID NO: 1031)
GGGUGGGACCCCUUUCGGGGUCCUGCUCAACUUCCUGUCGAGCUAAUGCCAUUUUUAAUGUCUUUAGCGAGACGCUA
CCAUGGCUAUCGCUGUAGGUAGCCGGAAUUCCAUUCCUAGGAGGUUUGACCUGUGCGAGCUUUUAGUACCCUUGAUA
GGGAGAACGAGACCUUCGUCCCCUCCGUUCGCGUUUACGCGGACGGUGAGACUGAAGAUAACUCAUUCUCUUUAAAA
UAUCGUUCGAACUGGACUCCCGGUCGUUUUAACUCGACUGGGGCCAAAACGAAACAGUGGCACUACCCCUCUCCGUA
UUCACGGGGGGCGUUAAGUGUCACAUCGAUAGAUCAAGGUGCCUACAAGCGAAGUGGGUCAUCGUGGGGUCGCCCGU
ACGAGGAGAAAGCCGGUUUCGGCUUCUCCCUCGACGCACGCUCCUGCUACAGCCUCUUCCCUGUAAGCCAAAACUUG
ACUUACAUCGAAGUGCCGCAGAACGUUGCGAACCGGGCGUCGACCGAAGUCCUGCAAAAGGUCACCCAGGGUAAUUU
UAACCUUGGUGUUGCUUUAGCAGAGGCCAGGUCGACAGCCUCACAACUCGCGACGCAAACCAUUGCGCUCGUGAAGG
CGUACACUGCCGCUCGUCGCGGUAAUUGGCGCCAGGCGCUCCGCUACCUUGCCCUAAACGAAGAUCGAAAGUUUCGA
UCAAAACACGUGGCCGGCAGGUGGUUGGAGUUGCAGUUCGGUUGGUUACCACUAAUGAGUGAUAUCCAGGGUGCAUA
UGAGAUGCUUACGAAGGUUCACCUUCAAGAGUUUCUUCCUAUGAGAGCCGUACGUCAGGUCGGUACUAACAUCAAGU
UAGAUGGCCGUCUGUCGUAUCCAGCUGCAAACUUCCAGACAACGUGCAACAUAUCGCGACGUAUCGUGAUAUGGUUU
UACAUAAACGAUGCACGUUUGGCAUGGUUGUCGUCUCUAGGUAUCUUGAACCCACUAGGUAUAGUGUGGGAAAAGGU
GCCUUUCUCAUUCGUUGUCGACUGGCUCCUACCUGUAGGUAACAUGCUCGAGGGCCUUACGGCCCCCGUGGGAUGCU
CCUACAUGUCAGGAACAGUUACUGACGUAAUAACGGGUGAGUCCAUCAUAAGCGUUGACGCUCCCUACGGGUGGACU
GUGGAGAGACAGGGCACUGCUAAGGCCCAAAUCUCAGCCAUGCAUCGAGGGGUACAAUCCGUAUGGCCAACAACUGG
CGCGUACGUAAAGUCUCCUUUCUCGAUGGUCCAUACCUUAGAUGCGUUAGCAUUAAUCAGGCAACGGCUCUCUAGAU
AGAGCCCUCAACCGGAGUUUGAAGCAUGGCUUCUAACUUUACUCAGUUCGUUCUCGUCGACAAUGGCGGAACUGGCG
ACGUGACUGUCGCCCCAAGCAACUUCGCUAACGGGGUCGCUGAAUGGAUCAGCUCUAACUCGCGUUCACAGGCUUAC
AAAGUAACCUGUAGCGUUCGUCAGAGCUCUGCGCAGAAUCGCAAAUACACCAUCAAAGUCGAGGUGCCUAAAGUGGC
AACCCAGACUGUUGGUGGUGUAGAGCUUCCUGUAGCCGCAUGGCGUUCGUACUUAAAUAUGGAACUAACCAUUCCAA
UUUUCGCUACGAAUUCCGACUGCGAGCUUAUUGUUAAGGCAAUGCAAGGUCUCCUAAAAGAUGGAAACCCGAUUCCC
UCAGCAAUCGCAGCAAACUCCGGCAUCUACUAAUAGACGCCGGCCAUUCAAACAUGAGGAUUACCCAUGUCGAAGAC
AACAAAGAAGUUCAACUCUUUAUGUAUUGAUCUUCCUCGCGAUCUUUCUCUCGAAAUUUACCAAUCAAUUGCUUCUG
UCGCUACUGGAAGCGGUGAUCCGCACAGUGACGACUUUACAGCAAUUGCUUACUUAAGGGACGAAUUGCUCACAAAG
CAUCCGACCUUAGGUUCUGGUAAUGACGAGGCGACCCGUCGUACCUUAGCUAUCGCUAAGCUACGGGAGGCGAAUGG
UGAUCGCGGUCAGAUAAAUAGAGAAGGUUUCUUACAUGACAAAUCCUUGUCAUGGGAUCCGGAUGUUUUACAAACCA
GCAUCCGUAGCCUUAUUGGCAACCUCCUCUCUGGCUACCGAUCGUCGUUGUUUGGGCAAUGCACGUUCUCCAACGGU
GCUCCUAUGGGGCACAAGUUGCAGGAUGCAGCGCCUUACAAGAAGUUCGCUGAACAAGCAACCGUUACCCCCCGCGC
UCUGAGAGCGGCUCUAUUGGUCCGAGACCAAUGUGCGCCGUGGAUCAGACACGCGGUCCGCUAUAACGAGUCAUAUG
AAUUUAGGCUCGUUGUAGGGAACGGAGUGUUUACAGUUCCGAAGAAUAAUAAAAUAGAUCGGGCUGCCUGUAAGGAG
CCUGAUAUGAAUAUGUACCUCCAGAAAGGGGUCGGUGCUUUCAUCAGACGCCGGCUCAAAUCCGUUGGUAUAGACCU
GAAUGAUCAAUCGAUCAACCAGCGUCUGGCUCAGCAGGGCAGCGUAGAUGGUUCGCUUGCGACGAUAGACUUAUCGU
CUGCAUCCGAUUCCAUCUCCGAUCGCCUGGUGUGGAGUUUUCUCCCACCAGAGCUAUAUUCAUAUCUCGAUCGUAUC
CGCUCACACUACGGAAUCGUAGAUGGCGAGACGAUACGAUGGGAACUAUUUUCCACAAUGGGAAAUGGGUUCACAUU
UGAGCUAGAGUCCAUGAUAUUCUGGGCAAUAGUCAAAGCGACCCAAAUCCAUUUUGGUAACGCCGGAACCAUAGGCA
UCUACGGGGACGAUAUUAUAUGUCCCAGUGAGAUUGCACCCCGUGUGCUAGAGGCACUUGCCUACUACGGUUUUAAA
CCGAAUCUUCGUAAAACGUUCGUGUCCGGGCUCUUUCGCGAGAGCUGCGGCGCGCACUUUUACCGUGGUGUCGAUGU
CAAACCGUUUUACAUCAAGAAACCUGUUGACAAUCUCUUCGCCCUGAUGCUGAUAUUAAAUCGGCUACGGGGUUGGG
GAGUUGUCGGAGGUAUGUCAGAUCCACGCCUCUAUAAGGUGUGGGUACGGCUCUCCUCCCAGGUGCCUUCGAUGUUC
UUCGGUGGGACGGACCUCGCUGCCGACUACUACGUAGUCAGCCCGCCUACGGCAGUCUCGGUAUACACCAAGACUCC
GUACGGGCGGCUGCUCGCGGAUACCCGUACCUCGGGUUUCCGUCUUGCUCGUAUCGCUCGAGAACGCAAGUUCUUCA
GCGAAAAGCACGACAGUGGUCGCUACAUAGCGUGGUUCCAUACUGGAGGUGAAAUCACCGACAGCAUGAAGUCCGCC
GGCGUGCGCGUUAUACGCACUUCGGAGUGGCUAACGCCGGUUCCCACAUUCCCUCAGGAGUGUGGGCCAGCGAGCUC
UCCUCGGUAGCUGACCGAGGGACCCCCGUAAACGGGGUGGGUGUGCUCGAAAGAGCACGGGUGCGAAAGCGGUCCGG
CUCCACCGAAAGGUGGGCGGGCUUCGGCCCAGGGACCUCCCCCUAAAGAGAGGACCCGGGAUUCUCCCGAUUUGGUA
ACUAGCUGCUUGGCUAGUUACCACCCA

1. A method for characterizing a target nucleic acid, the method including the steps of:
(a) contacting the target nucleic acid with one or more linearizing unit(s) to provide one or more structural unit(s) interspaced by one or more regions of double-stranded nucleic acid; and (b) detecting structural unit(s) along the target nucleic acid;
where:
(i) each linearising unit comprised a docking strand having a region that is complementary to distinct region(s) of the target nucleic acid;
(ii) one or more regions of said double-stranded nucleic acid comprised a docking strand of said linearizing unit hybridized to said distinct region(s) of the target nucleic acid; and
(iii) binding of the docking strand(s) to the target nucleic acid reduces secondary structure in the distinct region(s) of the target nucleic acid.

2. The method of claim 1, wherein one or more of the structural unit(s) is provided by the linearizing unit(s).

3. The method of claim 2, wherein one or more of the linearizing unit(s) includes: (i) a docking strand having a region that is complementary to distinct region(s) of the target nucleic acid and an overhang region; and (ii) a labeling strand that is complementary to the overhang region of the docking strand and includes a label.

4. The method of claim 2, wherein one or more of the linearizing unit(s) comprises a docking strand having a region that is complementary to distinct region(s) of the target nucleic acid and a labeling region.

5. The method of claim 1, in which one or more of the linearizing unit(s) are separated by single-stranded region(s) of the target nucleic acid, and in which one or more of the structural unit(s) is provided by secondary structures formed by said single-stranded region(s) of the target nucleic acid.

6. The method of claim 1, in which the linearising units provide one or more structural color(s) in which each structural color comprises: (a) an integer number of adjacent structural units detectable as a single signal; and/or (b) structural unit(s) which provide a signal that is distinct from other structural unit(s) and/or structural color(s).

7. The method of claim 1, wherein the method includes detecting the sequence of structural unit(s) and/or structural color(s) along the target nucleic acid.

8. The method of claim 1, wherein the target nucleic acid is RNA, optionally wherein the target nucleic acid is selected from single-stranded RNA (ssRNA), pre-mRNA, mRNA, miRNA, and non-coding RNA.

9. The method of claim 8, wherein the target nucleic acid is an RNA transcript.

10. The method of claim 1, wherein the method comprises characterizing more than one target nucleic acid.

11. The method of claim 3, wherein the labeling strand(s) comprises a structural, chemical and/or fluorescent label.

12. The method of claim 11, wherein the labeling strand comprises a ligand label.

13. The method of claim 12, wherein the method further comprised contacting the target nucleic acid with a receptor for the ligand, and wherein detecting structural unit(s) and/or structural color(s) comprised detecting ligand/receptor complexes.

14. The method of claim 12, wherein the ligand is biotin and the receptor is selected from streptavidin, neutravidin, traptavidin and avidin.

15. The method of claim 13, in which the ligand is an antigen and the receptor is an antibody.

16. The method of claim 11, wherein the labeling strand comprises a fluorescent label.

17. The method of claim 11, wherein the labeling strand comprises a DNA nanostructure; optionally where the DNA nanostructure is a DNA cuboid.

18. The method of claim 4, wherein the labeling region comprises a structural label, optionally wherein the structural label is a nucleic acid nanostructure such as a DNA double hairpin structure.

19. The method of claim 1, wherein structural unit(s) along the target nucleic acid are detected using a nanopore-based detection method.

20. The method of claim 16, wherein structural unit(s) and/or structural color(s) along the target nucleic acid are detected using a fluorescence-based detection method, optionally wherein the fluorescence-based detection method includes fluorescence microscopy.

21. The method of claim 1, wherein structural unit(s) and/or structural color(s) along the target nucleic acid are detected by a size-specific readout method, optionally wherein the size-specific readout method is mass photometry or a size-dependent lateral-flow assay.

22. The method of claim 1, wherein the method further comprised quantifying the amount of target nucleic acid in a sample, optionally wherein the target nucleic acid is quantified relative to an internal or external control.

23. The method of claim 1, wherein the target nucleic acid is derived from a virus, optionally wherein the virus is selected from a coronavirus, Influenza virus, Zika virus, Ebola virus, Dengue virus, Hantavirus, Nairovirus, Orthobunyavirus, Phlebovirus, Flavivirus, and Alphavirus.

24. The method of claim 23, wherein the target nucleic acid is a coronavirus genome, optionally the SARS-CoV-2 genome.

25. The method of claim 1, wherein the target nucleic acid is derived from a microorganism, optionally wherein the target nucleic acid is derived from a bacteria or a fungi.

26. The method of claim 1, wherein the target nucleic acid is derived from a pathogen, optionally wherein the pathogen is a viral pathogen, bacterial pathogen, fungal pathogen, protozoan pathogen or pathogenic worm.

27. The method of claim 1, wherein the method comprised characterizing one or more RNA transcript isoforms, optionally wherein the method further comprised quantifying each of the one or more transcript isoforms.

28. The method of claim 5, wherein the single-stranded region(s) of the target nucleic acid that provide the structural unit(s) and/or structural color(s) do not hybridize with linearizing units.

29. The method of claim 28, wherein the single-stranded region(s) comprises a secondary structure that prevents or reduces hybridization of the single-stranded region(s) with linearizing units.

30. The method of claim 28, wherein the presence of a nucleic acid binding molecule prevents or reduces hybridization of the single-stranded region(s) with linearizing units, optionally wherein the nucleic acid binding molecule binds to the single-stranded region or stabilizes a secondary structure thereof.

31. The method of claim 30, wherein the nucleic acid binding molecule is a drug, a protein, nucleic acid, ligand, small molecule, or an RNA binding protein (RBP).

32. The method of claim 30, wherein the method further understood characterizing the presence and/or location of binding between the target nucleic acid and nucleic acid binding molecule.

33. The method of claim 1, wherein the target nucleic acid is an RNA molecule and contacting the RNA molecule with linearizing units reshapes the target RNA molecule into a linear RNA comprising structural units and/or structural color(s) interspaced by double stranded regions of nucleic acid.

34. The method of claim 1, wherein the method further understood characterizing the length of a repeated sequence or the number of repeated sequences present in the target nucleic acid.

35. The method of claim 34, wherein the method includes characterizing the length of a poly(adenine) tail.

36. The method of claim 1, wherein the target nucleic acid is present in a sample obtained from a subject, optionally wherein the subject is a human.

37. The method of claim 36, in which the sample is selected from blood, serum, plasma, saliva, sputum, urine, faeces, cerebrospinal fluid, a lung tissue sample, a bronchoalveolar lavage sample, a nose and/or throat swab sample, or a biopsy sample.

38. The method of claim 1, wherein the step of contacting the target nucleic acid with one or more linearising unit(s) comprising:
(A) contacting a sample comprising a cell and/or a virus having the target nucleic acid with one or more linearizing unit(s); and
(B) lysing the cell and/or the virus.

39. The method of claim 38, wherein lysing the cell and/or the virus includes heating the cell and/or the virus.

40. The method of claim 38, wherein:
(a) the virus is selected from a coronavirus, Influenza virus, Zika virus, Ebola virus, Dengue virus, Hantavirus, Nairovirus, Orthobunyavirus, Phlebovirus, Flavivirus, and Alphavirus;
(b) the cell is a microorganism cell, optionally a bacterial cell or a fungal cell; and/or
(c) the cell is a eukaryotic cell, optionally a mammalian cell, optionally a human cell.


SEQUENCE LISTING (1031 sequences)

SEQ ID NO: 1 - DNA, 46 bp, synthetic construct
  source 1..46: mol_type = other DNA
ttttcgtaatcatggtcatagctgtttcctgtgtgaaattgttatc

SEQ ID NO: 2 - DNA, 38 bp, synthetic construct
  source 1..38: mol_type = other DNA
cgctcacaattccacacaacatacgagccggaagcata

SEQ ID NO: 3 - DNA, 38 bp, synthetic construct
  source 1..38: mol_type = other DNA
aagtgtaaagcctggggtgcctaatgagtgagctaact

SEQ ID NO: 4 - DNA, 38 bp, synthetic construct
  source 1..38: mol_type = other DNA
cacattaattgcgttgcgctcactgcccgctttccagt

SEQ ID NO: 5 - DNA, 38 bp, synthetic construct
  source 1..38: mol_type = other DNA
cgggaaacctgtcgtgccagctgcattaatgaatcggc

SEQ ID NO: 6 - DNA, 38 bp, synthetic construct
  source 1..38: mol_type = other DNA
caacgcgcggggagaggcggtttgcgtattgggcgcca

SEQ ID NO: 7 - DNA, 38 bp, synthetic construct
  source 1..38: mol_type = other DNA
gggtggtttttcttttcaccagtgagacgggcaacagc

SEQ ID NO: 8 - DNA, 38 bp, synthetic construct
  source 1..38: mol_type = other DNA
tgattgcccttcaccgcctggccctgagagagttgcag

SEQ ID NO: 9 - DNA, 38 bp, synthetic construct
  source 1..38: mol_type = other DNA
caagcggtccacgctggtttgccccagcaggcgaaaat

SEQ ID NO: 10 - DNA, 38 bp, synthetic construct
  source 1..38: mol_type = other DNA
cctgtttgatggtggttccgaaatcggcaaaatccctt

SEQ ID NO: 11 - DNA, 38 bp, synthetic construct
  source 1..38: mol_type = other DNA
ataaatcaaaagaatagcccgagatagggttgagtgtt

SEQ ID NO: 12 - DNA, 38 bp, synthetic construct
  source 1..38: mol_type = other DNA
gttccagtttggaacaagagtccactattaaagaacgt

SEQ ID NO: 13 - DNA, 38 bp, synthetic construct
  source 1..38: mol_type = other DNA
ggactccaacgtcaaagggcgaaaaaccgtctatcagg

SEQ ID NO: 14 - DNA, 38 bp, synthetic construct
  source 1..38: mol_type = other DNA
gcgatggcccactacgtgaaccatcacccaaatcaagt

SEQ ID NO: 15 - DNA, 38 bp, synthetic construct
  source 1..38: mol_type = other DNA
tttttggggtcgaggtgccgtaaagcactaaatcggaa

SEQ ID NO: 16 - DNA, 38 bp, synthetic construct
  source 1..38: mol_type = other DNA
ccctaaagggagcccccgatttagagcttgacggggaa

SEQ ID NO: 17 - DNA, 38 bp, synthetic construct
  source 1..38: mol_type = other DNA
agccggcgaacgtggcgagaaaggaagggaagaaagcg

SEQ ID NO: 18 - DNA, 38 bp, synthetic construct
  source 1..38: mol_type = other DNA
aaaggagcgggcgctagggcgctggcaagtgtagcggt

SEQ ID NO: 19 - DNA, 38 bp, synthetic construct
  source 1..38: mol_type = other DNA
cacgctgcgcgtaaccaccacacccgccgcgcttaatg

SEQ ID NO: 20 - DNA, 38 bp, synthetic construct
  source 1..38: mol_type = other DNA
cgccgctacagggcgcgtactatggttgctttgacgag

SEQ ID NO: 21 - DNA, 38 bp, synthetic construct
  source 1..38: mol_type = other DNA
cacgtataacgtgctttcctcgttagaatcagagcggg

SEQ ID NO: 22 - DNA, 38 bp, synthetic construct
  source 1..38: mol_type = other DNA
agctaaacaggaggccgattaaagggattttagacagg

SEQ ID NO: 23 - DNA, 38 bp, synthetic construct
  source 1..38: mol_type = other DNA
aacggtacgccagaatcctgagaagtgtttttataatc

SEQ ID NO: 24 - DNA, 38 bp, synthetic construct
  source 1..38: mol_type = other DNA
agtgaggccaccgagtaaaagagtctgtccatcacgca

SEQ ID NO: 25 - DNA, 38 bp, synthetic construct
  source 1..38: mol_type = other DNA
aattaaccgttgtagcaatacttctttgattagtaata

SEQ ID NO: 26 - DNA, 38 bp, synthetic construct
  source 1..38: mol_type = other DNA
acatcacttgcctgagtagaagaactcaaactatcggc

SEQ ID NO: 27 - DNA, 38 bp, synthetic construct
  source 1..38: mol_type = other DNA
cttgctggtaatatccagaacaatattaccgccagcca

SEQ ID NO: 28 - DNA, 38 bp, synthetic construct
  source 1..38: mol_type = other DNA
ttgcaacaggaaaaacgctcatggaaatacctacattt

SEQ ID NO: 29 - DNA, 38 bp, synthetic construct
  source 1..38: mol_type = other DNA
tgacgctcaatcgtctgaaatggattatttacattggc

SEQ ID NO: 30 - DNA, 38 bp, synthetic construct
  source 1..38: mol_type = other DNA
agattcaccagtcacacgaccagtaataaaagggacat

SEQ ID NO: 31 - DNA, 38 bp, synthetic construct
  source 1..38: mol_type = other DNA
tctggccaacagagatagaacccttctgacctgaaagc

SEQ ID NO: 32 - DNA, 38 bp, synthetic construct
  source 1..38: mol_type = other DNA
gtaagaatacgtggcacagacaatatttttgaatggct

SEQ ID NO: 33 - DNA, 38 bp, synthetic construct
  source 1..38: mol_type = other DNA
attagtctttaatgcgcgaactgatagccctaaaacat

SEQ ID NO: 34 - DNA, 38 bp, synthetic construct
  source 1..38: mol_type = other DNA
cgccattaaaaataccgaacgaaccaccagcagaagat

SEQ ID NO: 35 - DNA, 38 bp, synthetic construct
  source 1..38: mol_type = other DNA
aaaacagaggtgaggcggtcagtattaacaccgcctgc

SEQ ID NO: 36 - DNA, 38 bp, synthetic construct
  source 1..38: mol_type = other DNA
aacagtgccacgctgagagccagcagcaaatgaaaaat

SEQ ID NO: 37 - DNA, 38 bp, synthetic construct
  source 1..38: mol_type = other DNA
ctaaagcatcaccttgctgaacctcaaatatcaaaccc

SEQ ID NO: 38 - DNA, 38 bp, synthetic construct
  source 1..38: mol_type = other DNA
tcaatcaatatctggtcagttggcaaatcaacagttga

SEQ ID NO: 39 - DNA, 38 bp, synthetic construct
  source 1..38: mol_type = other DNA
aaggaattgaggaaggttatctaaaatatctttaggag

SEQ ID NO: 40 - DNA, 38 bp, synthetic construct
  source 1..38: mol_type = other DNA
cactaacaactaatagattagagccgtcaatagataat

SEQ ID NO: 41 - DNA, 38 bp, synthetic construct
  source 1..38: mol_type = other DNA
acatttgaggatttagaagtattagactttacaaacaa

SEQ ID NO: 42 - DNA, 38 bp, synthetic construct
  source 1..38: mol_type = other DNA
ttcgacaactcgtattaaatcctttgcccgaacgttat

SEQ ID NO: 43 - DNA, 38 bp, synthetic construct
  source 1..38: mol_type = other DNA
taattttaaaagtttgagtaacattatcattttgcgga

SEQ ID NO: 44 - DNA, 38 bp, synthetic construct
  source 1..38: mol_type = other DNA
acaaagaaaccaccagaaggagcggaattatcatcata

SEQ ID NO: 45 - DNA, 38 bp, synthetic construct
  source 1..38: mol_type = other DNA
ttcctgattatcagatgatggcaattcatcaatataat

SEQ ID NO: 46 - DNA, 38 bp, synthetic construct
  source 1..38: mol_type = other DNA
cctgattgtttggattatacttctgaataatggaaggg

SEQ ID NO: 47 - DNA, 38 bp, synthetic construct
  source 1..38: mol_type = other DNA
ttagaacctaccatatcaaaattatttgcacgtaaaac

SEQ ID NO: 48 - DNA, 38 bp, synthetic construct
  source 1..38: mol_type = other DNA
agaaataaagaaattgcgtagattttcaggtttaacgt

SEQ ID NO: 49 - DNA, 38 bp, synthetic construct
  source 1..38: mol_type = other DNA
cagatgaatatacagtaacagtaccttttacatcggga

SEQ ID NO: 50 - DNA, 38 bp, synthetic construct
  source 1..38: mol_type = other DNA
gaaacaataacggattcgcctgattgctttgaatacca

SEQ ID NO: 51 - DNA, 38 bp, synthetic construct
  source 1..38: mol_type = other DNA
agttacaaaatcgcgcagaggcgaattattcatttcaa

SEQ ID NO: 52 - DNA, 38 bp, synthetic construct
  source 1..38: mol_type = other DNA
ttacctgagcaaaagaagatgatgaaacaaacatcaag

SEQ ID NO: 53 - DNA, 38 bp, synthetic construct
  source 1..38: mol_type = other DNA
aaaacaaaattaattacatttaacaatttcatttgaat

SEQ ID NO: 54 - DNA, 38 bp, synthetic construct
  source 1..38: mol_type = other DNA
taccttttttaatggaaacagtacataaatcaatatat

SEQ ID NO: 55 - DNA, 38 bp, synthetic construct
  source 1..38: mol_type = other DNA
gtgagtgaataaccttgcttctgtaaatcgtcgctatt

SEQ ID NO: 56 - DNA, 38 bp, synthetic construct
  source 1..38: mol_type = other DNA
aattaattttcccttagaatccttgaaaacatagcgat

SEQ ID NO: 57 - DNA, 38 bp, synthetic construct
  source 1..38: mol_type = other DNA
agcttagattaagacgctgagaagagtcaatagtgaat

SEQ ID NO: 58 - DNA, 38 bp, synthetic construct
  source 1..38: mol_type = other DNA
ttatcaaaatcataggtctgagagactacctttttaac

SEQ ID NO: 59 - DNA, 38 bp, synthetic construct
  source 1..38: mol_type = other DNA
ctccggcttaggttgggttatataactatatgtaaatg

SEQ ID NO: 60 - DNA, 38 bp, synthetic construct
  source 1..38: mol_type = other DNA
ctgatgcaaatccaatcgcaagacaaagaacgcgagaa

SEQ ID NO: 61 - DNA, 38 bp, synthetic construct
  source 1..38: mol_type = other DNA
aactttttcaaatatattttagttaatttcatcttctg

SEQ ID NO: 62 - DNA, 38 bp, synthetic construct
  source 1..38: mol_type = other DNA
acctaaatttaatggtttgaaataccgaccgtgtgata

SEQ ID NO: 63 - DNA, 38 bp, synthetic construct
  source 1..38: mol_type = other DNA
aataaggcgttaaataagaataaacaccggaatcataa

SEQ ID NO: 64 - DNA, 38 bp, synthetic construct
  source 1..38: mol_type = other DNA
ttactagaaaaagcctgtttagtatcatatgcgttata

SEQ ID NO: 65 - DNA, 38 bp, synthetic construct
  source 1..38: mol_type = other DNA
caaattcttaccagtataaagccaacgctcaacagtag

SEQ ID NO: 66 - DNA, 38 bp, synthetic construct
  source 1..38: mol_type = other DNA
ggcttaattgagaatcgccatatttaacaacgccaaca

SEQ ID NO: 67 - DNA, 38 bp, synthetic construct
  source 1..38: mol_type = other DNA
tgtaatttaggcagaggcattttcgagccagtaataag

SEQ ID NO: 68 - DNA, 38 bp, synthetic construct
  source 1..38: mol_type = other DNA
agaatataaagtaccgacaaaaggtaaagtaattctgt

SEQ ID NO: 69 - DNA, 38 bp, synthetic construct
  source 1..38: mol_type = other DNA
ccagacgacgacaataaacaacatgttcagctaatgca

SEQ ID NO: 70 - DNA, 38 bp, synthetic construct
  source 1..38: mol_type = other DNA
gaacgcgcctgtttatcaacaatagataagtcctgaac

SEQ ID NO: 71 - DNA, 38 bp, synthetic construct
  source 1..38: mol_type = other DNA
aagaaaaataatatcccatcctaatttacgagcatgta

SEQ ID NO: 72 - DNA, 38 bp, synthetic construct
  source 1..38: mol_type = other DNA
gaaaccaatcaataatcggctgtctttccttatcattc

SEQ ID NO: 73 - DNA, 38 bp, synthetic construct
  source 1..38: mol_type = other DNA
caagaacgggtattaaaccaagtaccgcactcatcgag

SEQ ID NO: 74 - DNA, 38 bp, synthetic construct
  source 1..38: mol_type = other DNA
aacaagcaagccgtttttattttcatcgtaggaatcat

SEQ ID NO: 75 - DNA, 38 bp, synthetic construct
  source 1..38: mol_type = other DNA
taccgcgcccaatagcaagcaaatcagatatagaaggc

SEQ ID NO: 76 - DNA, 38 bp, synthetic construct
  source 1..38: mol_type = other DNA
ttatccggtattctaagaacgcgaggcgttttagcgaa

SEQ ID NO: 77 - DNA, 38 bp, synthetic construct
  source 1..38: mol_type = other DNA
cctcccgacttgcgggaggttttgaagccttaaatcaa

SEQ ID NO: 78 - DNA, 38 bp, synthetic construct
  source 1..38: mol_type = other DNA
gattagttgctattttgcacccagctacaattttatcc

SEQ ID NO: 79 - DNA, 38 bp, synthetic construct
  source 1..38: mol_type = other DNA
tgaatcttaccaacgctaacgagcgtctttccagagcc

SEQ ID NO: 80 - DNA, 38 bp, synthetic construct
  source 1..38: mol_type = other DNA
taatttgccagttacaaaataaacagccatattattta

SEQ ID NO: 81 - DNA, 38 bp, synthetic construct
  source 1..38: mol_type = other DNA
tcccaatccaaataagaaacgattttttgtttaacgtc

SEQ ID NO: 82 - DNA, 38 bp, synthetic construct
  source 1..38: mol_type = other DNA
aaaaatgaaaatagcagcctttacagagagaataacat

SEQ ID NO: 83 - DNA, 38 bp, synthetic construct
  source 1..38: mol_type = other DNA
aaaaacagggaagcgcattagacgggagaattaactga

SEQ ID NO: 84 - DNA, 38 bp, synthetic construct
  source 1..38: mol_type = other DNA
acaccctgaacaaagtcagagggtaattgagcgctaat

SEQ ID NO: 85 - DNA, 38 bp, synthetic construct
  source 1..38: mol_type = other DNA
atcagagagataacccacaagaattgagttaagcccaa

SEQ ID NO: 86 - DNA, 38 bp, synthetic construct
  source 1..38: mol_type = other DNA
taataagagcaagaaacaatgaaatagcaatagctatc

SEQ ID NO: 87 - DNA, 38 bp, synthetic construct
  source 1..38: mol_type = other DNA
ttaccgaagccctttttaagaaaagtaagcagatagcc

SEQ ID NO: 88 - DNA, 38 bp, synthetic construct
  source 1..38: mol_type = other DNA
gaacaaagttaccagaaggaaaccgaggaaacgcaata

SEQ ID NO: 89 - DNA, 38 bp, synthetic construct
  source 1..38: mol_type = other DNA
ataacggaatacccaaaagaactggcatgattaagact

SEQ ID NO: 90 - DNA, 38 bp, synthetic construct
  source 1..38: mol_type = other DNA
ccttattacgcagtatgttagcaaacgtagaaaataca

SEQ ID NO: 91 - DNA, 38 bp, synthetic construct
  source 1..38: mol_type = other DNA
tacataaaggtggcaacatataaaagaaacgcaaagac

SEQ ID NO: 92 - DNA, 38 bp, synthetic construct
  source 1..38: mol_type = other DNA
accacggaataagtttattttgtcacaatcaatagaaa

SEQ ID NO: 93 - DNA, 38 bp, synthetic construct
  source 1..38: mol_type = other DNA
attcatatggtttaccagcgccaaagacaaaagggcga

SEQ ID NO: 94 - DNA, 38 bp, synthetic construct
  source 1..38: mol_type = other DNA
cattcaaccgattgagggagggaaggtaaatattgacg

SEQ ID NO: 95 - DNA, 38 bp, synthetic construct
  source 1..38: mol_type = other DNA
gaaattattcattaaaggtgaattatcaccgtcaccga

SEQ ID NO: 96 - DNA, 38 bp, synthetic construct
  source 1..38: mol_type = other DNA
cttgagccatttgggaattagagccagcaaaatcacca

SEQ ID NO: 97 - DNA, 38 bp, synthetic construct
  source 1..38: mol_type = other DNA
gtagcaccattaccattagcaaggccggaaacgtcacc

SEQ ID NO: 98 - DNA, 38 bp, synthetic construct
  source 1..38: mol_type = other DNA
aatgaaaccatcgatagcagcaccgtaatcagtagcga

SEQ ID NO: 99 - DNA, 38 bp, synthetic construct
  source 1..38: mol_type = other DNA
cagaatcaagtttgcctttagcgtcagactgtagcgcg

SEQ ID NO: 100 - DNA, 38 bp, synthetic construct
  source 1..38: mol_type = other DNA
ttttcatcggcattttcggtcatagcccccttattagc

SEQ ID NO: 101 - DNA, 38 bp, synthetic construct
  source 1..38: mol_type = other DNA
gtttgccatcttttcataatcaaaatcaccggaaccag

SEQ ID NO: 102 - DNA, 38 bp, synthetic construct
  source 1..38: mol_type = other DNA
agccaccaccggaaccgcctccctcagagccgccaccc

SEQ ID NO: 103 - DNA, 38 bp, synthetic construct
  source 1..38: mol_type = other DNA
tcagaaccgccaccctcagagccaccaccctcagagcc

SEQ ID NO: 104 - DNA, 38 bp, synthetic construct
  source 1..38: mol_type = other DNA
gccaccagaaccaccaccagagccgccgccagcattga

SEQ ID NO: 105 - DNA, 38 bp, synthetic construct
  source 1..38: mol_type = other DNA
caggaggttgaggcaggtcagacgattggccttgatat

SEQ ID NO: 106 - DNA, 38 bp, synthetic construct
  source 1..38: mol_type = other DNA
tcacaaacaaataaatcctcattaaagccagaatggaa

SEQ ID NO: 107 - DNA, 38 bp, synthetic construct
  source 1..38: mol_type = other DNA
agcgcagtctctgaatttaccgttccagtaagcgtcat

SEQ ID NO: 108 - DNA, 38 bp, synthetic construct
  source 1..38: mol_type = other DNA
acatggcttttgatgatacaggagtgtactggtaataa

SEQ ID NO: 109 - DNA, 38 bp, synthetic construct
  source 1..38: mol_type = other DNA
gttttaacggggtcagtgccttgagtaacagtgcccgt

SEQ ID NO: 110 - DNA, 38 bp, synthetic construct
  source 1..38: mol_type = other DNA
ataaacagttaatgccccctgcctatttcggaacctat

SEQ ID NO: 111 - DNA, 38 bp, synthetic construct
  source 1..38: mol_type = other DNA
tattctgaaacatgaaagtattaagaggctgagactcc

SEQ ID NO: 112 - DNA, 38 bp, synthetic construct
  source 1..38: mol_type = other DNA
tcaagagaaggattaggattagcggggttttgctcagt

SEQ ID NO: 113 - DNA, 38 bp, synthetic construct
  source 1..38: mol_type = other DNA
accaggcggataagtgccgtcgagagggttgatataag

SEQ ID NO: 114 - DNA, 38 bp, synthetic construct
  source 1..38: mol_type = other DNA
tatagcccggaataggtgtatcaccgtactcaggaggt

SEQ ID NO: 115 - DNA, 38 bp, synthetic construct
  source 1..38: mol_type = other DNA
ttagtaccgccaccctcagaaccgccaccctcagaacc

SEQ ID NO: 116 - DNA, 38 bp, synthetic construct
  source 1..38: mol_type = other DNA
gccaccctcagagccaccaccctcattttcagggatag

SEQ ID NO: 117 - DNA, 38 bp, synthetic construct
  source 1..38: mol_type = other DNA
caagcccaataggaacccatgtaccgtaacactgagtt

SEQ ID NO: 118 - DNA, 38 bp, synthetic construct
  source 1..38: mol_type = other DNA
tcgtcaccagtacaaactacaacgcctgtagcattcca

SEQ ID NO: 119 - DNA, 38 bp, synthetic construct
  source 1..38: mol_type = other DNA
cagacagccctcatagttagcgtaacgatctaaagttt

SEQ ID NO: 120 - DNA, 38 bp, synthetic construct
  source 1..38: mol_type = other DNA
tgtcgtctttccagacgttagtaaatgaattttctgta

SEQ ID NO: 121 - DNA, 38 bp, synthetic construct
  source 1..38: mol_type = other DNA
tgggattttgctaaacaactttcaacagtttcagcgga

SEQ ID NO: 122 - DNA, 38 bp, synthetic construct
  source 1..38: mol_type = other DNA
gtgagaatagaaaggaacaactaaaggaattgcgaata

SEQ ID NO: 123 - DNA, 38 bp, synthetic construct
  source 1..38: mol_type = other DNA
ataattttttcacgttgaaaatctccaaaaaaaaggct

SEQ ID NO: 124 - DNA, 38 bp, synthetic construct
  source 1..38: mol_type = other DNA
ccaaaaggagcctttaattgtatcggtttatcagcttg

SEQ ID NO: 125 - DNA, 38 bp, synthetic construct
  source 1..38: mol_type = other DNA
ctttcgaggtgaatttcttaaacagcttgataccgata

SEQ ID NO: 126 - DNA, 38 bp, synthetic construct
  source 1..38: mol_type = other DNA
gttgcgccgacaatgacaacaaccatcgcccacgcata

SEQ ID NO: 127 - DNA, 38 bp, synthetic construct
  source 1..38: mol_type = other DNA
accgatatattcggtcgctgaggcttgcagggagttaa

SEQ ID NO: 128 - DNA, 38 bp, synthetic construct
  source 1..38: mol_type = other DNA
aggccgcttttgcgggatcgtcaccctcagcagcgaaa

SEQ ID NO: 129 - DNA, 38 bp, synthetic construct
  source 1..38: mol_type = other DNA
gacagcatcggaacgagggtagcaacggctacagaggc

SEQ ID NO: 130 - DNA, 38 bp, synthetic construct
  source 1..38: mol_type = other DNA
tttgaggactaaagactttttcatgaggaagtttccat

SEQ ID NO: 131 - DNA, 38 bp, synthetic construct
  source 1..38: mol_type = other DNA
taaacgggtaaaatacgtaatgccactacgaaggcacc

SEQ ID NO: 132 - DNA, 38 bp, synthetic construct
  source 1..38: mol_type = other DNA
aacctaaaacgaaagaggcaaaagaatacactaaaaca

SEQ ID NO: 133 - DNA, 38 bp, synthetic construct
  source 1..38: mol_type = other DNA
ctcatctttgacccccagcgattataccaagcgcgaaa

SEQ ID NO: 134 - DNA, 38 bp, synthetic construct
  source 1..38: mol_type = other DNA
caaagtacaacggagatttgtatcatcgcctgataaat

SEQ ID NO: 135 - DNA, 38 bp, synthetic construct
  source 1..38: mol_type = other DNA
tgtgtcgaaatccgcgacctgctccatgttacttagcc

SEQ ID NO: 136 - DNA, 38 bp, synthetic construct
  source 1..38: mol_type = other DNA
ggaacgaggcgcagacggtcaatcataagggaaccgaa

SEQ ID NO: 137 - DNA, 38 bp, synthetic construct
  source 1..38: mol_type = other DNA
ctgaccaactttgaaagaggacagatgaacggtgtaca

SEQ ID NO: 138 - DNA, 38 bp, synthetic construct
  source 1..38: mol_type = other DNA
gaccaggcgcataggctggctgaccttcatcaagagta

SEQ ID NO: 139 - DNA, 38 bp, synthetic construct
  source 1..38: mol_type = other DNA
atcttgacaagaaccggatattcattacccaaatcaac

SEQ ID NO: 140 - DNA, 38 bp, synthetic construct
  source 1..38: mol_type = other DNA
gtaacaaagctgctcattcagtgaataaggcttgccct

SEQ ID NO: 141 - DNA, 38 bp, synthetic construct
  source 1..38: mol_type = other DNA
gacgagaaacaccagaacgagtagtaaattgggcttga

SEQ ID NO: 142 - DNA, 38 bp, synthetic construct
  source 1..38: mol_type = other DNA
gatggtttaatttcaactttaatcattgtgaattacct

SEQ ID NO: 143 - DNA, 38 bp, synthetic construct
  source 1..38: mol_type = other DNA
tatgcgattttaagaactggctcattataccagtcagg

SEQ ID NO: 144 - DNA, 38 bp, synthetic construct
  source 1..38: mol_type = other DNA
acgttgggaagaaaaatctacgttaataaaacgaacta

SEQ ID NO: 145 - DNA, 38 bp, synthetic construct
  source 1..38: mol_type = other DNA
acggaacaacattattacaggtagaaagattcatcagt

SEQ ID NO: 146 - DNA, 38 bp, synthetic construct
  source 1..38: mol_type = other DNA
tgagatttaggaataccacattcaactaatgcagatac

SEQ ID NO: 147 - DNA, 38 bp, synthetic construct
  source 1..38: mol_type = other DNA
ataacgccaaaaggaattacgaggcatagtaagagcaa

SEQ ID NO: 148 - DNA, 38 bp, synthetic construct
  source 1..38: mol_type = other DNA
cactatcataaccctcgtttaccagacgacgataaaaa

SEQ ID NO: 149 - DNA, 38 bp, synthetic construct
  source 1..38: mol_type = other DNA
ccaaaatagcgagaggcttttgcaaaagaagttttgcc

SEQ ID NO: 150 - DNA, 38 bp, synthetic construct
  source 1..38: mol_type = other DNA
agagggggtaatagtaaaatgtttagactggatagcgt

SEQ ID NO: 151 - DNA, 38 bp, synthetic construct
  source 1..38: mol_type = other DNA
ccaatactgcggaatcgtcataaatattcattgaatcc

SEQ ID NO: 152 - DNA, 38 bp, synthetic construct
  source 1..38: mol_type = other DNA
ccctcaaatgctttaaacagttcagaaaacgagaatga

SEQ ID NO: 153 - DNA, 38 bp, synthetic construct
  source 1..38: mol_type = other DNA
ccataaatcaaaaatcaggtctttaccctgactattat

SEQ ID NO: 154 - DNA, 38 bp, synthetic construct
  source 1..38: mol_type = other DNA
agtcagaagcaaagcggattgcatcaaaaagattaaga

SEQ ID NO: 155 - DNA, 38 bp, synthetic construct
  source 1..38: mol_type = other DNA
ggaagcccgaaagacttcaaatatcgcgttttaattcg

SEQ ID NO: 156 - DNA, 38 bp, synthetic construct
  source 1..38: mol_type = other DNA
agcttcaaagcgaaccagaccggaagcaaactccaaca

SEQ ID NO: 157 - DNA, 38 bp, synthetic construct
  source 1..38: mol_type = other DNA
ggtcaggattagagagtacctttaattgctccttttga

SEQ ID NO: 158 - DNA, 38 bp, synthetic construct
  source 1..38: mol_type = other DNA
taagaggtcatttttgcggatggcttagagcttaattg

SEQ ID NO: 159 - DNA, 38 bp, synthetic construct
  source 1..38: mol_type = other DNA
ctgaatataatgctgtagctcaacatgttttaaatatg

SEQ ID NO: 160 - DNA, 38 bp, synthetic construct
  source 1..38: mol_type = other DNA
caactaaagtacggtgtctggaagtttcattccatata

SEQ ID NO: 161 - DNA, 38 bp, synthetic construct
  source 1..38: mol_type = other DNA
acagttgattcccaattctgcgaacgagtagatttagt

SEQ ID NO: 162 - DNA, 38 bp, synthetic construct
  source 1..38: mol_type = other DNA
ttgaccattagatacatttcgcaaatggtcaataacct

SEQ ID NO: 163 - DNA, 38 bp, synthetic construct
  source 1..38: mol_type = other DNA
gtttagctatattttcatttggggcgcgagctgaaaag

SEQ ID NO: 164 - DNA, 38 bp, synthetic construct
  source 1..38: mol_type = other DNA
gtggcatcaattctactaatagtagtagcattaacatc

SEQ ID NO: 165 - DNA, 38 bp, synthetic construct
  source 1..38: mol_type = other DNA
caataaatcatacaggcaaggcaaagaattagcaaaat

SEQ ID NO: 166 - DNA, 38 bp, synthetic construct
  source 1..38: mol_type = other DNA
taagcaataaagcctcagagcataaagctaaatcggtt

SEQ ID NO: 167 - DNA, 38 bp, synthetic construct
  source 1..38: mol_type = other DNA
gtaccaaaaacattatgaccctgtaatacttttgcggg

SEQ ID NO: 168 - DNA, 38 bp, synthetic construct
  source 1..38: mol_type = other DNA
agaagcctttatttcaacgcaaggataaaaatttttag

SEQ ID NO: 169 - DNA, 38 bp, synthetic construct
  source 1..38: mol_type = other DNA
aaccctcatatattttaaatgcaatgcctgagtaatgt

SEQ ID NO: 170 - DNA, 38 bp, synthetic construct
  source 1..38: mol_type = other DNA
gtaggtaaagattcaaaagggtgagaaaggccggagac

SEQ ID NO: 171 - DNA, 38 bp, synthetic construct
  source 1..38: mol_type = other DNA
agtcaaatcaccatcaatatgatattcaaccgttctag

SEQ ID NO: 172 - DNA, 38 bp, synthetic construct
  source 1..38: mol_type = other DNA
ctgataaattaatgccggagagggtagctatttttgag

SEQ ID NO: 173 - DNA, 38 bp, synthetic construct
  source 1..38: mol_type = other DNA
agatctacaaaggctatcaggtcattgcctgagagtct

SEQ ID NO: 174 - DNA, 38 bp, synthetic construct
  source 1..38: mol_type = other DNA
ggagcaaacaagagaatcgatgaacggtaatcgtaaaa

SEQ ID NO: 175 - DNA, 38 bp, synthetic construct
  source 1..38: mol_type = other DNA
ctagcatgtcaatcatatgtaccccggttgataatcag

SEQ ID NO: 176 - DNA, 38 bp, synthetic construct
  source 1..38: mol_type = other DNA
aaaagccccaaaaacaggaagattgtataagcaaatat

SEQ ID NO: 177 - DNA, 38 bp, synthetic construct
  source 1..38: mol_type = other DNA
ttaaattgtaaacgttaatattttgttaaaattcgcat

SEQ ID NO: 178 - DNA, 38 bp, synthetic construct
  source 1..38: mol_type = other DNA
taaatttttgttaaatcagctcattttttaaccaatag

SEQ ID NO: 179 - DNA, 38 bp, synthetic construct
  source 1..38: mol_type = other DNA
gaacgccatcaaaaataattcgcgtctggccttcctgt

SEQ ID NO: 180 - DNA, 38 bp, synthetic construct
  source 1..38: mol_type = other DNA
agccagctttcatcaacattaaatgtgagcgagtaaca

SEQ ID NO: 181 - DNA, 38 bp, synthetic construct
  source 1..38: mol_type = other DNA
acccgtcggattctccgtgggaacaaacggcggattga

SEQ ID NO: 182 - DNA, 38 bp, synthetic construct
  source 1..38: mol_type = other DNA
ccgtaatgggataggtcacgttggtgtagatgggcgca

SEQ ID NO: 183 - DNA, 38 bp, synthetic construct
  source 1..38: mol_type = other DNA
tcgtaaccgtgcatctgccagtttgaggggacgacgac

SEQ ID NO: 184 - DNA, 38 bp, synthetic construct
  source 1..38: mol_type = other DNA
agtatcggcctcaggaagatcgcactccagccagcttt

SEQ ID NO: 185 - DNA, 38 bp, synthetic construct
  source 1..38: mol_type = other DNA
ccggcaccgcttctggtgccggaaaccaggcaaagcgc

SEQ ID NO: 186 - DNA, 38 bp, synthetic construct
  source 1..38: mol_type = other DNA
cattcgccattcaggctgcgcaactgttgggaagggcg

SEQ ID NO: 187 - DNA, 38 bp, synthetic construct
  source 1..38: mol_type = other DNA
atcggtgcgggcctcttcgctattacgccagctggcga

SEQ ID NO: 188 - DNA, 38 bp, synthetic construct
  source 1..38: mol_type = other DNA
aagggggatgtgctgcaaggcgattaagttgggtaacg

SEQ ID NO: 189 - DNA, 38 bp, synthetic construct
  source 1..38: mol_type = other DNA
ccagggttttcccagtcacgacgttgtaaaacgacggc

SEQ ID NO: 190 - DNA, 46 bp, synthetic construct
  source 1..46: mol_type = other DNA
cagtgccaagcttgcatgcctgcaggtcgactctagaggatctttt

SEQ ID NO: 191 - DNA, 48 bp, synthetic construct
  source 1..48: mol_type = other DNA
gcgatggcccactacgtgaaccatctttggatatcactcattagtggt

SEQ ID NO: 192 - DNA, 25 bp, synthetic construct
  source 1..25: mol_type = other DNA
acccaaatcaagttttttggggtcg

SEQ ID NO: 193 - DNA, 26 bp, synthetic construct
  source 1..26: mol_type = other DNA
aggtgccgtaaagcactaaatcggaa

SEQ ID NO: 194 - DNA, 48 bp, synthetic construct
  source 1..48: mol_type = other DNA
ttgcaacaggaaaaacgctcatggatttggatatcactcattagtggt

SEQ ID NO: 195 - DNA, 48 bp, synthetic construct
  source 1..48: mol_type = other DNA
aatacctacattttgacgctcaatctttggatatcactcattagtggt

SEQ ID NO: 196 - DNA, 26 bp, synthetic construct
  source 1..26: mol_type = other DNA
gtctgaaatggattatttacattggc

SEQ ID NO: 197 - DNA, 48 bp, synthetic construct
  source 1..48: mol_type = other DNA
taattttaaaagtttgagtaacatttttggatatcactcattagtggt

SEQ ID NO: 198 - DNA, 48 bp, synthetic construct
  source 1..48: mol_type = other DNA
atcattttgcggaacaaagaaaccatttggatatcactcattagtggt

SEQ ID NO: 199 - DNA, 49 bp, synthetic construct
  source 1..49: mol_type = other DNA
ccagaaggagcggaattatcatcatatttggatatcactcattagtggt

SEQ ID NO: 200 - DNA, 48 bp, synthetic construct
  source 1..48: mol_type = other DNA
ttatcaaaatcataggtctgagagatttggatatcactcattagtggt

SEQ ID NO: 201 - DNA, 48 bp, synthetic construct
  source 1..48: mol_type = other DNA
ctacctttttaacctccggcttaggtttggatatcactcattagtggt

SEQ ID NO: 202 - DNA, 48 bp, synthetic construct
  source 1..48: mol_type = other DNA
ttgggttatataactatatgtaaattttggatatcactcattagtggt

SEQ ID NO: 203 - DNA, 48 bp, synthetic construct
  source 1..48: mol_type = other DNA
gctgatgcaaatccaatcgcaagactttggatatcactcattagtggt

SEQ ID NO: 204 - DNA, 26 bp, synthetic construct
  source 1..26: mol_type = other DNA
aaagaacgcgagaaaactttttcaaa

SEQ ID NO: 205 - DNA, 26 bp, synthetic construct
  source 1..26: mol_type = other DNA
tatattttagttaatttcatcttctg

SEQ ID NO: 206 - DNA, 20 bp, synthetic construct
  source 1..20: mol_type = other DNA
accactaatgagtgatatcc

SEQ ID NO: 207 - DNA, 48 bp, synthetic construct
  source 1..48: mol_type = other DNA
aacaagcaagccgtttttattttcatttggatatcactcattagtggt

SEQ ID NO: 208 - DNA, 48 bp, synthetic construct
  source 1..48: mol_type = other DNA
tcgtaggaatcattaccgcgcccaatttggatatcactcattagtggt

SEQ ID NO: 209 - DNA, 48 bp, synthetic construct
  source 1..48: mol_type = other DNA
tagcaagcaaatcagatatagaaggtttggatatcactcattagtggt

SEQ ID NO: 210 - DNA, 48 bp, synthetic construct
  source 1..48: mol_type = other DNA
cttatccggtattctaagaacgcgatttggatatcactcattagtggt

SEQ ID NO: 211 - DNA, 48 bp, synthetic construct
  source 1..48: mol_type = other DNA
ggcgttttagcgaacctcccgactttttggatatcactcattagtggt

SEQ ID NO: 212 - DNA, 27 bp, synthetic construct
  source 1..27: mol_type = other DNA
gcgggaggttttgaagccttaaatcaa

SEQ ID NO: 213 - DNA, 48 bp, synthetic construct
  source 1..48: mol_type = other DNA
ccttattacgcagtatgttagcaaatttggatatcactcattagtggt

SEQ ID NO: 214 - DNA, 48 bp, synthetic construct
  source 1..48: mol_type = other DNA
cgtagaaaatacatacataaaggtgtttggatatcactcattagtggt

SEQ ID NO: 215 - DNA, 48 bp, synthetic construct
  source 1..48: mol_type = other DNA
gcaacatataaaagaaacgcaaagatttggatatcactcattagtggt

SEQ ID NO: 216 - DNA, 48 bp, synthetic construct
  source 1..48: mol_type = other DNA
caccacggaataagtttattttgtctttggatatcactcattagtggt

SEQ ID NO: 217 - DNA, 48 bp, synthetic construct
  source 1..48: mol_type = other DNA
acaatcaatagaaaattcatatggttttggatatcactcattagtggt

SEQ ID NO: 218 - DNA, 50 bp, synthetic construct
  source 1..50: mol_type = other DNA
ttaccagcgccaaagacaaaagggcgatttggatatcactcattagtggt

SEQ ID NO: 219 - DNA, 48 bp, synthetic construct
  source 1..48: mol_type = other DNA
agcgcagtctctgaatttaccgttctttggatatcactcattagtggt

SEQ ID NO: 220 - DNA, 48 bp, synthetic construct
  source 1..48: mol_type = other DNA
cagtaagcgtcatacatggcttttgtttggatatcactcattagtggt

SEQ ID NO: 221 - DNA, 48 bp, synthetic construct
  source 1..48: mol_type = other DNA
atgatacaggagtgtactggtaatatttggatatcactcattagtggt

SEQ ID NO: 222 - DNA, 48 bp, synthetic construct
  source 1..48: mol_type = other DNA
agttttaacggggtcagtgccttgatttggatatcactcattagtggt

SEQ ID NO: 223 - DNA, 48 bp, synthetic construct
  source 1..48: mol_type = other DNA
gtaacagtgcccgtataaacagttatttggatatcactcattagtggt

SEQ ID NO: 224 - DNA, 48 bp, synthetic construct
  source 1..48: mol_type = other DNA
atgccccctgcctatttcggaaccttttggatatcactcattagtggt

SEQ ID NO: 225 - DNA, 48 bp, synthetic construct
  source 1..48: mol_type = other DNA
attattctgaaacatgaaagtattatttggatatcactcattagtggt

SEQ ID NO: 226 - DNA, 15 bp, synthetic construct
  source 1..15: mol_type = other DNA
agaggctgagactcc

SEQ ID NO: 227 - DNA, 48 bp, synthetic construct
  source 1..48: mol_type = other DNA
ctttcgaggtgaatttcttaaacagtttggatatcactcattagtggt

SEQ ID NO: 228 - DNA, 48 bp, synthetic construct
  source 1..48: mol_type = other DNA
cttgataccgatagttgcgccgacatttggatatcactcattagtggt

SEQ ID NO: 229 - DNA, 48 bp, synthetic construct
  source 1..48: mol_type = other DNA
atgacaacaaccatcgcccacgcattttggatatcactcattagtggt

SEQ ID NO: 230 - DNA, 48 bp, synthetic construct
  source 1..48: mol_type = other DNA
aaccgatatattcggtcgctgaggctttggatatcactcattagtggt

SEQ ID NO: 231 - DNA, 48 bp, synthetic construct
  source 1..48: mol_type = other DNA
ttgcagggagttaaaggccgctttttttggatatcactcattagtggt

SEQ ID NO: 232 - DNA, 48 bp, synthetic construct
  source 1..48: mol_type = other DNA
gcgggatcgtcaccctcagcagcgatttggatatcactcattagtggt

SEQ ID NO: 233 - DNA, 48 bp, synthetic construct
  source 1..48: mol_type = other DNA
aagacagcatcggaacgagggtagctttggatatcactcattagtggt

SEQ ID NO: 234 - DNA, 48 bp, synthetic construct
  source 1..48: mol_type = other DNA
aacggctacagaggctttgaggacttttggatatcactcattagtggt

SEQ ID NO: 235 - DNA, 28 bp, synthetic construct
  source 1..28: mol_type = other DNA
aaagactttttcatgaggaagtttccat

SEQ ID NO: 236 - DNA, 48 bp, synthetic construct
  source 1..48: mol_type = other DNA
tatgcgattttaagaactggctcattttggatatcactcattagtggt

SEQ ID NO: 237 - DNA, 48 bp, synthetic construct
  source 1..48: mol_type = other DNA
tataccagtcaggacgttgggaagatttggatatcactcattagtggt

SEQ ID NO: 238 - DNA, 48 bp, synthetic construct
  source 1..48: mol_type = other DNA
aaaatctacgttaataaaacgaacttttggatatcactcattagtggt

SEQ ID NO: 239 - DNA, 48 bp, synthetic construct
  source 1..48: mol_type = other DNA
aacggaacaacattattacaggtagtttggatatcactcattagtggt

SEQ ID NO: 240 - DNA, 48 bp, synthetic construct
  source 1..48: mol_type = other DNA
aaagattcatcagttgagatttaggtttggatatcactcattagtggt

SEQ ID NO: 241 - DNA, 48 bp, synthetic construct
  source 1..48: mol_type = other DNA
aataccacattcaactaatgcagattttggatatcactcattagtggt

SEQ ID NO: 242 - DNA, 48 bp, synthetic construct
  source 1..48: mol_type = other DNA
acataacgccaaaaggaattacgagtttggatatcactcattagtggt

SEQ ID NO: 243 - DNA, 48 bp, synthetic construct
  source 1..48: mol_type = other DNA
gcatagtaagagcaacactatcatatttggatatcactcattagtggt

SEQ ID NO: 244 - DNA, 51 bp, synthetic construct
  source 1..51: mol_type = other DNA
accctcgtttaccagacgacgataaaaatttggatatcactcattagtggt

SEQ ID NO: 245 - DNA, 48 bp, synthetic construct
  source 1..48: mol_type = other DNA
ttgaccattagatacatttcgcaaatttggatatcactcattagtggt

SEQ ID NO: 246 - DNA, 48 bp, synthetic construct
  source 1..48: mol_type = other DNA
tggtcaataacctgtttagctatattttggatatcactcattagtggt

SEQ ID NO: 247 - DNA, 48 bp, synthetic construct
  source 1..48: mol_type = other DNA
tttcatttggggcgcgagctgaaaatttggatatcactcattagtggt

SEQ ID NO: 248 - DNA, 48 bp, synthetic construct
  source 1..48: mol_type = other DNA
ggtggcatcaattctactaatagtatttggatatcactcattagtggt

SEQ ID NO: 249 - DNA, 48 bp, synthetic construct
  source 1..48: mol_type = other DNA
gtagcattaacatccaataaatcattttggatatcactcattagtggt

SEQ ID NO: 250 - DNA, 48 bp, synthetic construct
  source 1..48: mol_type = other DNA
acaggcaaggcaaagaattagcaaatttggatatcactcattagtggt

SEQ ID NO: 251 - DNA, 48 bp, synthetic construct
  source 1..48: mol_type = other DNA
attaagcaataaagcctcagagcattttggatatcactcattagtggt

SEQ ID NO: 252 - DNA, 48 bp, synthetic construct
  source 1..48: mol_type = other DNA
aaagctaaatcggttgtaccaaaaatttggatatcactcattagtggt

SEQ ID NO: 253 - DNA, 48 bp, synthetic construct
  source 1..48: mol_type = other DNA
cattatgaccctgtaatacttttgctttggatatcactcattagtggt

SEQ ID NO: 254 - DNA, 48 bp, synthetic construct
  source 1..48: mol_type = other DNA
gggagaagcctttatttcaacgcaatttggatatcactcattagtggt

SEQ ID NO: 255 - DNA, 27 bp, synthetic construct
  source 1..27: mol_type = other DNA
ggataaaaatttttagaaccctcatat

SEQ ID NO: 256 - DNA, 27 bp, synthetic construct
  source 1..27: mol_type = other DNA
attttaaatgcaatgcctgagtaatgt

SEQ ID NO: 257 - DNA, 22 bp, synthetic construct
  source 1..22: mol_type = other DNA
gccgacgtgtacggatctggca

SEQ ID NO: 258 - DNA, 22 bp, synthetic construct
  source 1..22: mol_type = other DNA
gcgacattgcggccgattcgga

SEQ ID NO: 259 - DNA, 46 bp, synthetic construct
  source 1..46: mol_type = other DNA
accactaatgagtgatatccttttcgcatcagggcacattggcttt

SEQ ID NO: 260 - DNA, 26 bp, synthetic construct
  source 1..26: mol_type = other DNA
ttttgcggctaagacttgccaggttt

SEQ ID NO: 261 - DNA, 48 bp, synthetic construct
  source 1..48: mol_type = other DNA
ttttacacgtcggccctgatgcgacctggcaagttccgaatcggcttt

SEQ ID NO: 262 - DNA, 48 bp, synthetic construct
  source 1..48: mol_type = other DNA
tttcgcaatgtcgccttagccgcagccaatgtgctgccagatccgttt

SEQ ID NO: 263 - DNA, 46 bp, synthetic construct; identical to SEQ ID NO: 259
  source 1..46: mol_type = other DNA

SEQ ID NO: 264 - DNA, 20 bp, synthetic construct
  source 1..20: mol_type = other DNA
ggatatcactcattagtggt

SEQ ID NO: 265 - DNA, 38 bp, synthetic construct
  source 1..38: mol_type = other DNA
cactccgttccctacaacgagcctaaattcatatgact

SEQ ID NO: 266 - DNA, 38 bp, synthetic construct
  source 1..38: mol_type = other DNA
cgttatagcggaccgcgtgtctgatccacggcgcacat

SEQ ID NO: 267 - DNA, 38 bp, synthetic construct
  source 1..38: mol_type = other DNA
tggtctcggaccaatagagccgctctcagagcgcgggg

SEQ ID NO: 268 - DNA, 38 bp, synthetic construct
  source 1..38: mol_type = other DNA
ggtaacggttgcttgttcagcgaacttcttgtaaggcg

SEQ ID NO: 269 - DNA, 38 bp, synthetic construct
  source 1..38: mol_type = other DNA
ctgcatcctgcaacttgtgccccataggagcaccgttg

SEQ ID NO: 270 - DNA, 38 bp, synthetic construct
  source 1..38: mol_type = other DNA
gagaacgtgcattgcccaaacaacgacgatcggtagcc

SEQ ID NO: 271 - DNA, 38 bp, synthetic construct
  source 1..38: mol_type = other DNA
agagaggaggttgccaataaggctacggatgctggttt

SEQ ID NO: 272 - DNA, 38 bp, synthetic construct
  source 1..38: mol_type = other DNA
gtaaaacatccggatcccatgacaaggatttgtcatgt

SEQ ID NO: 273 - DNA, 38 bp, synthetic construct
  source 1..38: mol_type = other DNA
aagaaaccttctctatttatctgaccgcgatcaccatt

SEQ ID NO: 274 - DNA, 48 bp, synthetic construct
  source 1..48: mol_type = other DNA
cgcctcccgttcctcttttgaggaacaagttttcttgtagcttagcga

SEQ ID NO: 275 - DNA, 48 bp, synthetic construct
  source 1..48: mol_type = other DNA
tagctaaggttcctcttttgaggaacaagttttcttgtacgacgggtc

SEQ ID NO: 276 - DNA, 48 bp, synthetic construct
  source 1..48: mol_type = other DNA
gcctcgtcattcctcttttgaggaacaagttttcttgttaccagaacc

SEQ ID NO: 277 - DNA, 48 bp, synthetic construct
  source 1..48: mol_type = other DNA
taaggtcggatcctcttttgaggaacaagttttcttgttgctttgtga

SEQ ID NO: 278 - DNA, 48 bp, synthetic construct
  source 1..48: mol_type = other DNA
gcaattcgtctcctcttttgaggaacaagttttcttgtccttaagtaa

SEQ ID NO: 279 - DNA, 48 bp, synthetic construct
  source 1..48: mol_type = other DNA
gcaattgctgtcctcttttgaggaacaagttttcttgttaaagtcgtc

SEQ ID NO: 280 - DNA, 32 bp, synthetic construct
  source 1..32: mol_type = other DNA
actgtgcggatcaccgcttccagtagcgacag

SEQ ID NO: 281 - DNA, 38 bp, synthetic construct
  source 1..38: mol_type = other DNA
aagcaattgattggtaaatttcgagagaaagatcgcga

SEQ ID NO: 282 - DNA, 38 bp, synthetic construct
  source 1..38: mol_type = other DNA
ggaagatcaatacataaagagttgaacttctttgttgt

SEQ ID NO: 283 - DNA, 38 bp, synthetic construct
  source 1..38: mol_type = other DNA
cttcgacatgggtaatcctcatgtttgaatggccggcg

SEQ ID NO: 284 - DNA, 38 bp, synthetic construct
  source 1..38: mol_type = other DNA
tctattagtagatgccggagtttgctgcgattgctgag

SEQ ID NO: 285 - DNA, 38 bp, synthetic construct
  source 1..38: mol_type = other DNA
ggaatcgggtttccatcttttaggagaccttgcattgc

SEQ ID NO: 286 - DNA, 38 bp, synthetic construct
  source 1..38: mol_type = other DNA
cttaacaataagctcgcagtcggaattcgtagcgaaaa

SEQ ID NO: 287 - DNA, 38 bp, synthetic construct
  source 1..38: mol_type = other DNA
ttggaatggttagttccatatttaagtacgaacgccat

SEQ ID NO: 288 - DNA, 38 bp, synthetic construct
  source 1..38: mol_type = other DNA
gcggctacaggaagctctacaccaccaacagtctgggt

SEQ ID NO: 289 - DNA, 38 bp, synthetic construct
  source 1..38: mol_type = other DNA
tgccactttaggcacctcgactttgatggtgtatttgc

SEQ ID NO: 290 - DNA, 38 bp, synthetic construct
  source 1..38: mol_type = other DNA
gattctgcgcagagctctgacgaacgctacaggttact

SEQ ID NO: 291 - DNA, 38 bp, synthetic construct
  source 1..38: mol_type = other DNA
ttgtaagcctgtgaacgcgagttagagctgatccattc

SEQ ID NO: 292 - DNA, 38 bp, synthetic construct
  source 1..38: mol_type = other DNA
agcgaccccgttagcgaagttgcttggggcgacagtca

SEQ ID NO: 293 - DNA, 48 bp, synthetic construct
  source 1..48: mol_type = other DNA
cgtcgccagttcctcttttgaggaacaagttttcttgttccgccattg

SEQ ID NO: 294 - DNA, 48 bp, synthetic construct
  source 1..48: mol_type = other DNA
tcgacgagaatcctcttttgaggaacaagttttcttgtcgaactgagt

SEQ ID NO: 295 - DNA, 48 bp, synthetic construct
  source 1..48: mol_type = other DNA
aaagttagaatcctcttttgaggaacaagttttcttgtgccatgcttc

SEQ ID NO: 296 - DNA, 48 bp, synthetic construct
  source 1..48: mol_type = other DNA
aaactccggttcctcttttgaggaacaagttttcttgttgagggctct

SEQ ID NO: 297 - DNA, 48 bp, synthetic construct
  source 1..48: mol_type = other DNA
atctagagagtcctcttttgaggaacaagttttcttgtccgttgcctg

SEQ ID NO: 298 - DNA, 48 bp, synthetic construct
  source 1..48: mol_type = other DNA
attaatgctatcctcttttgaggaacaagttttcttgtacgcatctaa

SEQ ID NO: 299 - DNA, 32 bp, synthetic construct
  source 1..32: mol_type = other DNA
ggtatggaccatcgagaaaggagactttacgt

SEQ ID NO: 300 - DNA, 38 bp, synthetic construct
  source 1..38: mol_type = other DNA
acgcgccagttgttggccatacggattgtacccctcga

SEQ ID NO: 301 - DNA, 38 bp, synthetic construct
  source 1..38: mol_type = other DNA
tgcatggctgagatttgggccttagcagtgccctgtct

SEQ ID NO: 302 - DNA, 38 bp, synthetic construct
  source 1..38: mol_type = other DNA
ctccacagtccacccgtagggagcgtcaacgcttatga

SEQ ID NO: 303 - DNA, 38 bp, synthetic construct
  source 1..38: mol_type = other DNA
tggactcacccgttattacgtcagtaactgttcctgac

SEQ ID NO: 304 - DNA, 38 bp, synthetic construct
  source 1..38: mol_type = other DNA
atgtaggagcatcccacgggggccgtaaggccctcgag

SEQ ID NO: 305 - DNA, 38 bp, synthetic construct
  source 1..38: mol_type = other DNA
catgttacctacaggtaggagccagtcgacaacgaatg

SEQ ID NO: 306 - DNA, 38 bp, synthetic construct
  source 1..38: mol_type = other DNA
agaaaggcaccttttcccacactatacctagtgggttc

SEQ ID NO: 307 - DNA, 38 bp, synthetic construct
  source 1..38: mol_type = other DNA
aagatacctagagacgacaaccatgccaaacgtgcatc

SEQ ID NO: 308 - DNA, 38 bp, synthetic construct
  source 1..38: mol_type = other DNA
gtttatgtaaaaccatatcacgatacgtcgcgatatgt

SEQ ID NO: 309 - DNA, 48 bp, synthetic construct
  source 1..48: mol_type = other DNA
tgcacgttgttcctcttttgaggaacaagttttcttgtctggaagttt

SEQ ID NO: 310 - DNA, 48 bp, synthetic construct
  source 1..48: mol_type = other DNA
gcagctggattcctcttttgaggaacaagttttcttgtacgacagacg

SEQ ID NO: 311 - DNA, 48 bp, synthetic construct
  source 1..48: mol_type = other DNA
gccatctaactcctcttttgaggaacaagttttcttgtttgatgttag

SEQ ID NO: 312 - DNA, 48 bp, synthetic construct
  source 1..48: mol_type = other DNA
taccgacctgtcctcttttgaggaacaagttttcttgtacgtacggct

SEQ ID NO: 313 - DNA, 48 bp, synthetic construct
  source 1..48: mol_type = other DNA
ctcataggaatcctcttttgaggaacaagttttcttgtgaaactcttg

SEQ ID NO: 314 - DNA, 48 bp, synthetic construct
  source 1..48: mol_type = other DNA
aaggtgaacctcctcttttgaggaacaagttttcttgtttcgtaagca

SEQ ID NO: 315 - DNA, 32 bp, synthetic construct
  source 1..32: mol_type = other DNA
tctcatatgcaccctggatatcactcattagt

SEQ ID NO: 316 - DNA, 38 bp, synthetic construct
  source 1..38: mol_type = other DNA
ggtaaccaaccgaactgcaactccaaccacctgccggc

SEQ ID NO: 317 - DNA, 38 bp, synthetic construct
  source 1..38: mol_type = other DNA
cacgtgttttgatcgaaactttcgatcttcgtttaggg

SEQ ID NO: 318 - DNA, 38 bp, synthetic construct
  source 1..38: mol_type = other DNA
caaggtagcggagcgcctggcgccaattaccgcgacga

SEQ ID NO: 319 - DNA, 38 bp, synthetic construct
  source 1..38: mol_type = other DNA
gcggcagtgtacgccttcacgagcgcaatggtttgcgt

SEQ ID NO: 320 - DNA, 38 bp, synthetic construct
  source 1..38: mol_type = other DNA
cgcgagttgtgaggctgtcgacctggcctctgctaaag

SEQ ID NO: 321 - DNA, 38 bp, synthetic construct
  source 1..38: mol_type = other DNA
caacaccaaggttaaaattaccctgggtgaccttttgc

SEQ ID NO: 322 - DNA, 38 bp, synthetic construct
  source 1..38: mol_type = other DNA
aggacttcggtcgacgcccggttcgcaacgttctgcgg

SEQ ID NO: 323 - DNA, 38 bp, synthetic construct
  source 1..38: mol_type = other DNA
cacttcgatgtaagtcaagttttggcttacagggaaga

SEQ ID NO: 324 - DNA, 38 bp, synthetic construct
  source 1..38: mol_type = other DNA
ggctgtagcaggagcgtgcgtcgagggagaagccgaaa

SEQ ID NO: 325 - DNA, 38 bp, synthetic construct
  source 1..38: mol_type = other DNA
taatgatccttccgcaggttcacctacggaaaccttgt

SEQ ID NO: 326 - DNA, 38 bp, synthetic construct
  source 1..38: mol_type = other DNA
tacgacttttacttcctctagatagtcaagttcgaccg

SEQ ID NO: 327 - DNA, 38 bp, synthetic construct
  source 1..38: mol_type = other DNA
tcttctcagcgctccgccagggccgtgggccgaccccg

SEQ ID NO: 328 - DNA, 38 bp, synthetic construct
  source 1..38: mol_type = other DNA
gcggggccgatccgagggcctcactaaaccatccaatc

SEQ ID NO: 329 - DNA, 38 bp, synthetic construct
  source 1..38: mol_type = other DNA
ggtagtagcgacgggcggtgtgtacaaagggcagggac

SEQ ID NO: 330 - DNA, 38 bp, synthetic construct
  source 1..38: mol_type = other DNA
ttaatcaacgcaagcttatgacccgcacttactgggaa

SEQ ID NO: 331 - DNA, 25 bp, synthetic construct
  source 1..25: mol_type = other DNA
ttcctcgttcatggggaataattgc

SEQ ID NO: 332 - DNA, 25 bp, synthetic construct
  source 1..25: mol_type = other DNA
aatccccgatccccatcacgaatgg

SEQ ID NO: 333 - DNA, 48 bp, synthetic construct
  source 1..48: mol_type = other DNA
ggttcaacggtcctcttttgaggaacaagttttcttgtgttacccgcg

SEQ ID NO: 334 - DNA, 48 bp, synthetic construct
  source 1..48: mol_type = other DNA
cctgccggcgtcctcttttgaggaacaagttttcttgttagggtaggc

SEQ ID NO: 335 - DNA, 48 bp, synthetic construct
  source 1..48: mol_type = other DNA
acacgctgagtcctcttttgaggaacaagttttcttgtccagtcagtg

SEQ ID NO: 336 - DNA, 48 bp, synthetic construct
  source 1..48: mol_type = other DNA
tagcgcgcgttcctcttttgaggaacaagttttcttgtgcagccccgg

SEQ ID NO: 337 - DNA, 48 bp, synthetic construct
  source 1..48: mol_type = other DNA
acatctaaggtcctcttttgaggaacaagttttcttgtgcatcacaga

SEQ ID NO: 338 - DNA, 48 bp, synthetic construct
  source 1..48: mol_type = other DNA
cctgttattgtcctcttttgaggaacaagttttcttgtctcaatctcg

SEQ ID NO: 339 - DNA, 38 bp, synthetic construct
  source 1..38: mol_type = other DNA
ggtggctgaacgccacttgtccctctaagaagttgggg

SEQ ID NO: 340 - DNA, 38 bp, synthetic construct
  source 1..38: mol_type = other DNA
gacgccgaccgctcgggggtcgcgtaactagttagcat

SEQ ID NO: 341 - DNA, 38 bp, synthetic construct
  source 1..38: mol_type = other DNA
gccagagtctcgttcgttatcggaattaaccagacaaa

SEQ ID NO: 342 - DNA, 38 bp, synthetic construct
  source 1..38: mol_type = other DNA
tcgctccaccaactaagaacggccatgcaccaccaccc

SEQ ID NO: 343 - DNA, 38 bp, synthetic construct
  source 1..38: mol_type = other DNA
acggaatcgagaaagagctatcaatctgtcaatcctgt

SEQ ID NO: 344 - DNA, 38 bp, synthetic construct
  source 1..38: mol_type = other DNA
ccgtgtccgggccgggtgaggtttcccgtgttgagtca

SEQ ID NO: 345 - DNA, 25 bp, synthetic construct
  source 1..25: mol_type = other DNA
aattaagccgcaggctccactcctg

SEQ ID NO: 346 - DNA, 25 bp, synthetic construct
  source 1..25: mol_type = other DNA
gtggtgcccttccgtcaattccttt

SEQ ID NO: 347 - DNA, 48 bp, synthetic construct
  source 1..48: mol_type = other DNA
aagtttcagctcctcttttgaggaacaagttttcttgttttgcaacca

SEQ ID NO: 348 - DNA, 48 bp, synthetic construct
  source 1..48: mol_type = other DNA
tactcccccctcctcttttgaggaacaagttttcttgtggaacccaaa

SEQ ID NO: 349 - DNA, 48 bp, synthetic construct
  source 1..48: mol_type = other DNA
gactttggtttcctcttttgaggaacaagttttcttgttcccggaagc

SEQ ID NO: 350 - DNA, 48 bp, synthetic construct
  source 1..48: mol_type = other DNA
tgcccggcggtcctcttttgaggaacaagttttcttgtgtcatgggaa

SEQ ID NO: 351 - DNA, 48 bp, synthetic construct
  source 1..48: mol_type = other DNA
taacgccgcctcctcttttgaggaacaagttttcttgtgcatcgccgg

SEQ ID NO: 352 - DNA, 48 bp, synthetic construct
  source 1..48: mol_type = other DNA
tcggcatcgttcctcttttgaggaacaagttttcttgtttatggtcgg

SEQ ID NO: 353 - DNA, 38 bp, synthetic construct
  source 1..38: mol_type = other DNA
aactacgacggtatctgatcgtcttcgaacctccgact

SEQ ID NO: 354 - DNA, 38 bp, synthetic construct
  source 1..38: mol_type = other DNA
ttcgttcttgattaatgaaaacattcttggcaaatgct

SEQ ID NO: 355 - DNA, 38 bp, synthetic construct
  source 1..38: mol_type = other DNA
ttcgctctggtccgtcttgcgccggtccaagaatttca

SEQ ID NO: 356 - DNA, 38 bp, synthetic construct
  source 1..38: mol_type = other DNA
cctctagcggcgcaatacgaatgcccccggccgtccct

SEQ ID NO: 357 - DNA, 38 bp, synthetic construct
  source 1..38: mol_type = other DNA
cttaatcatggcctcagttccgaaaaccaacaaaatag

SEQ ID NO: 358 - DNA, 38 bp, synthetic construct
  source 1..38: mol_type = other DNA
aaccgcggtcctattccattattcctagctgcggtatc

SEQ ID NO: 359 - DNA, 25 bp, synthetic construct
  source 1..25: mol_type = other DNA
caggcggctcgggcctgctttgaac

SEQ ID NO: 360 - DNA, 25 bp, synthetic construct
  source 1..25: mol_type = other DNA
actctaattttttcaaagtaaacgc

SEQ ID NO: 361 - DNA, 48 bp, synthetic construct
  source 1..48: mol_type = other DNA
ttcgggcccctcctcttttgaggaacaagttttcttgtgcgggacact

SEQ ID NO: 362 - DNA, 48 bp, synthetic construct
  source 1..48: mol_type = other DNA
cagctaagagtcctcttttgaggaacaagttttcttgtcatcgagggg

SEQ ID NO: 363 - DNA, 48 bp, synthetic construct
  source 1..48: mol_type = other DNA
gcgccgagagtcctcttttgaggaacaagttttcttgtgcaaggggcg

SEQ ID NO: 364 - DNA, 48 bp, synthetic construct
  source 1..48: mol_type = other DNA
gggacgggcgtcctcttttgaggaacaagttttcttgtgtggctcgcc

SEQ ID NO: 365 - DNA, 48 bp, synthetic construct
  source 1..48: mol_type = other DNA
tcgcggcggatcctcttttgaggaacaagttttcttgtccgcccgccc

SEQ ID NO: 366 - DNA, 48 bp, synthetic construct
  source 1..48: mol_type = other DNA
gctcccaagatcctcttttgaggaacaagttttcttgttccaactacg

SEQ ID NO: 367 - DNA, 38 bp, synthetic construct
  source 1..38: mol_type = other DNA
agctttttaactgcagcaactttaatatacgctattgg

SEQ ID NO: 368 - DNA, 38 bp, synthetic construct
  source 1..38: mol_type = other DNA
agctggaattaccgcggctgctggcaccagacttgccc

SEQ ID NO: 369 - DNA, 38 bp, synthetic construct
  source 1..38: mol_type = other DNA
tccaatggatcctcgttaaaggatttaaagtggactca

SEQ ID NO: 370 - DNA, 38 bp, synthetic construct
  source 1..38: mol_type = other DNA
ttccaattacagggcctcgaaagagtcctgtattgtta

SEQ ID NO: 371 - DNA, 38 bp, synthetic construct
  source 1..38: mol_type = other DNA
tttttcgtcactacctccccgggtcgggagtgggtaat

SEQ ID NO: 372 - DNA, 38 bp, synthetic construct
  source 1..38: mol_type = other DNA
ttgcgcgcctgctgccttccttggatgtggtagccgtt

SEQ ID NO: 373 - DNA, 25 bp, synthetic construct
  source 1..25: mol_type = other DNA
tctcaggctccctctccggaatcga

SEQ ID NO: 374 - DNA, 25 bp, synthetic construct
  source 1..25: mol_type = other DNA
accctgattccccgtcacccgtggt

SEQ ID NO: 375 - DNA, 48 bp, synthetic construct
  source 1..48: mol_type = other DNA
caccatggtatcctcttttgaggaacaagttttcttgtggcacggcga

SEQ ID NO: 376 - DNA, 48 bp, synthetic construct
  source 1..48: mol_type = other DNA
ctaccatcgatcctcttttgaggaacaagttttcttgtaagttgatag

SEQ ID NO: 377 - DNA, 48 bp, synthetic construct
  source 1..48: mol_type = other DNA
ggcagacgtttcctcttttgaggaacaagttttcttgtcgaatgggtc

SEQ ID NO: 378 - DNA, 48 bp, synthetic construct
  source 1..48: mol_type = other DNA
gtcgccgccatcctcttttgaggaacaagttttcttgtcggggggcgt

SEQ ID NO: 379 - DNA, 48 bp, synthetic construct
  source 1..48: mol_type = other DNA
gcgatcggcctcctcttttgaggaacaagttttcttgtcgaggttatc

SEQ ID NO: 380 - DNA, 48 bp, synthetic construct
  source 1..48: mol_type = other DNA
tagagtcacctcctcttttgaggaacaagttttcttgtaaagccgccg

SEQ ID NO: 381 - DNA, 38 bp, synthetic construct
  source 1..38: mol_type = other DNA
gcgcccgccccccggccggggccggagaggggctgacc

SEQ ID NO: 382 - DNA, 38 bp, synthetic construct
  source 1..38: mol_type = other DNA
gggttggttttgatctgataaatgcacgcatccccccc

SEQ ID NO: 383 - DNA, 38 bp, synthetic construct
  source 1..38: mol_type = other DNA
gcgaagggggtcagcgcccgtcggcatgtattagctct

SEQ ID NO: 384 - DNA, 38 bp, synthetic construct
  source 1..38: mol_type = other DNA
agaattaccacagttatccaagtgggagaggagcgagc

SEQ ID NO: 385 - DNA, 38 bp, synthetic construct
  source 1..38: mol_type = other DNA
gaccaaaggaaccataactgatttaatgagccattcgc

SEQ ID NO: 386 - DNA, 38 bp, synthetic construct
  source 1..38: mol_type = other DNA
agtttcactgtaccggccgtgcgtacttagacatgcat

SEQ ID NO: 387 - DNA, 25 bp, synthetic construct
  source 1..25: mol_type = other DNA
ggcttaatctttgagacaagcatat

SEQ ID NO: 388 - DNA, 24 bp, synthetic construct
  source 1..24: mol_type = other DNA
gctactggcaggatcaaccaggta

SEQ ID NO: 389 - DNA, 38 bp, synthetic construct
  source 1..38: mol_type = other DNA
tcggaacggcgctcgcccatctctcaggaccgactgac

SEQ ID NO: 390 - DNA, 38 bp, synthetic construct
  source 1..38: mol_type = other DNA
ccatgttcaactgctgttcacatggaacccttctccac

SEQ ID NO: 391 - DNA, 38 bp, synthetic construct
  source 1..38: mol_type = other DNA
ttcggccttcaaagttctcgtttgaatatttgctacta

SEQ ID NO: 392 - DNA, 38 bp, synthetic construct
  source 1..38: mol_type = other DNA
ccaccaagatctgcacctgcggcggctccacccgggcc

SEQ ID NO: 393 - DNA, 38 bp, synthetic construct
  source 1..38: mol_type = other DNA
cgcgccctaggcttcaaggctcaccgcagcggccctcc

SEQ ID NO: 394 - DNA, 38 bp, synthetic construct
  source 1..38: mol_type = other DNA
tactcgtcgcggcgtagcgtccgcggggctccgggggc

SEQ ID NO: 395 - DNA, 38 bp, synthetic construct
  source 1..38: mol_type = other DNA
ggggagcggggcgtgggcgggaggaggggaggaggcgt

SEQ ID NO: 396 - DNA, 38 bp, synthetic construct
  source 1..38: mol_type = other DNA
gggggggggggcgggggaaggaccccacacccccgccg

SEQ ID NO: 397 - DNA, 38 bp, synthetic construct
  source 1..38: mol_type = other DNA
ccgccgccgccgccgccctccgacgcacaccacacgcg

SEQ ID NO: 398 - DNA, 38 bp, synthetic construct
  source 1..38: mol_type = other DNA
cgcgcgcgcgcgccgcccccgccgctcccgtccactct

SEQ ID NO: 399 - DNA, 48 bp, synthetic construct
  source 1..48: mol_type = other DNA
cgactgccggtcctcttttgaggaacaagttttcttgtcgacggccgg

SEQ ID NO: 400 - DNA, 48 bp, synthetic construct
  source 1..48: mol_type = other DNA
gtatgggccctcctcttttgaggaacaagttttcttgtgacgctccag

SEQ ID NO: 401 - DNA, 48 bp, synthetic construct
  source 1..48: mol_type = other DNA
cgccatccattcctcttttgaggaacaagttttcttgttttcagggct

SEQ ID NO: 402 - DNA, 48 bp, synthetic construct
  source 1..48: mol_type = other DNA
agttgattcgtcctcttttgaggaacaagttttcttgtgcaggtgagt

SEQ ID NO: 403 - DNA, 48 bp, synthetic construct
  source 1..48: mol_type = other DNA
tgttacacactcctcttttgaggaacaagttttcttgttccttagcgg

SEQ ID NO: 404 - DNA, 48 bp, synthetic construct
  source 1..48: mol_type = other DNA
attccgactttcctcttttgaggaacaagttttcttgtccatggccac

SEQ ID NO: 405 - DNA, 38 bp, synthetic construct
  source 1..38: mol_type = other DNA
cgtcctgctgtctatatcaaccaacaccttttctgggg

SEQ ID NO: 406 - DNA, 38 bp, synthetic construct
  source 1..38: mol_type = other DNA
tctgatgagcgtcggcatcgggcgccttaacccggcgt

SEQ ID NO: 407 - DNA, 38 bp, synthetic construct
  source 1..38: mol_type = other DNA
tcggttcatcccgcagcgccagttctgcttaccaaaag

SEQ ID NO: 408 - DNA, 38 bp, synthetic construct
  source 1..38: mol_type = other DNA
tggcccactaggcactcgcattccacgcccggctccac

SEQ ID NO: 409 - DNA, 38 bp, synthetic construct
  source 1..38: mol_type = other DNA
gccagcgagccgggcttcttacccatttaaagtttgag

SEQ ID NO: 410 - DNA, 38 bp, synthetic construct
  source 1..38: mol_type = other DNA
aataggttgagatcgtttcggccccaagacctctaatc

SEQ ID NO: 411 - DNA, 25 bp, synthetic construct
  source 1..25: mol_type = other DNA
attcgctttaccggataaaactgcg

SEQ ID NO: 412 - DNA, 25 bp, synthetic construct
  source 1..25: mol_type = other DNA
tggcgggggtgcgtcgggtctgcga

SEQ ID NO: 413 - DNA, 48 bp, synthetic construct
  source 1..48: mol_type = other DNA
gagcgccagctcctcttttgaggaacaagttttcttgttatcctgagg

SEQ ID NO: 414 - DNA, 48 bp, synthetic construct
  source 1..48: mol_type = other DNA
gaaacttcggtcctcttttgaggaacaagttttcttgtagggaaccag

SEQ ID NO: 415 - DNA, 48 bp, synthetic construct
  source 1..48: mol_type = other DNA
ctactagatgtcctcttttgaggaacaagttttcttgtgttcgattag

SEQ ID NO: 416 - DNA, 48 bp, synthetic construct
  source 1..48: mol_type = other DNA
tctttcgccctcctcttttgaggaacaagttttcttgtctatacccag

SEQ ID NO: 417 - DNA, 48 bp, synthetic construct
  source 1..48: mol_type = other DNA
gtcggacgactcctcttttgaggaacaagttttcttgtcgatttgcac

SEQ ID NO: 418 - DNA, 48 bp, synthetic construct
  source 1..48: mol_type = other DNA
gtcaggaccgtcctcttttgaggaacaagttttcttgtctacggacct

SEQ ID NO: 419 - DNA, 38 bp, synthetic construct
  source 1..38: mol_type = other DNA
ccaccagagtttcctctggcttcgccctgcccaggcat

SEQ ID NO: 420 - DNA, 38 bp, synthetic construct
  source 1..38: mol_type = other DNA
agttcaccatctttcgggtcctaacacgtgcgctcgtg

SEQ ID NO: 421 - DNA, 38 bp, synthetic construct
  source 1..38: mol_type = other DNA
ctccacctccccggcgcggcgggcgagacgggccggtg

SEQ ID NO: 422 - DNA, 38 bp, synthetic construct
  source 1..38: mol_type = other DNA
gtgcgccctcggcggactggagaggcctcgggatccca

SEQ ID NO: 423 - DNA, 38 bp, synthetic construct
  source 1..38: mol_type = other DNA
cctcggccggcgagcgcgccggccttcaccttcattgc

SEQ ID NO: 424 - DNA, 38 bp, synthetic construct
  source 1..38: mol_type = other DNA
gccacggcggctttcgtgcgagcccccgactcgcgcac

SEQ ID NO: 425 - DNA, 25 bp, synthetic construct
  source 1..25: mol_type = other DNA
gtgttagactccttggtccgtgttt

SEQ ID NO: 426 - DNA, 25 bp, synthetic construct
  source 1..25: mol_type = other DNA
caagacgggtcgggtgggtagccga

SEQ ID NO: 427 - DNA, 48 bp, synthetic construct
  source 1..48: mol_type = other DNA
cgtcgccgcctcctcttttgaggaacaagttttcttgtgaccccgtgc

SEQ ID NO: 428 - DNA, 48 bp, synthetic construct
  source 1..48: mol_type = other DNA
gctcgctccgtcctcttttgaggaacaagttttcttgtccgtccccct

SEQ ID NO: 429 - DNA, 48 bp, synthetic construct
  source 1..48: mol_type = other DNA
cttcgggggatcctcttttgaggaacaagttttcttgtcgcgcgcgtg

SEQ ID NO: 430 - DNA, 48 bp, synthetic construct
  source 1..48: mol_type = other DNA
gccccgagagtcctcttttgaggaacaagttttcttgtaacctccccc

SEQ ID NO: 431 - DNA, 48 bp, synthetic construct
  source 1..48: mol_type = other DNA
gggcccgacgtcctcttttgaggaacaagttttcttgtgcgcgacccg

SEQ ID NO: 432 - DNA, 48 bp, synthetic construct
  source 1..48: mol_type = other DNA
cccggggcgctcctcttttgaggaacaagttttcttgtactggggaca

SEQ ID NO: 433 - DNA, 38 bp, synthetic construct
  source 1..38: mol_type = other DNA
gtccgccccgccccccgacccgcgcgcggcaccccccc

SEQ ID NO: 434 - DNA, 38 bp, synthetic construct
  source 1..38: mol_type = other DNA
cgtcgccggggcgggggcgcggggaggaggggtgggag

SEQ ID NO: 435 - DNA, 38 bp, synthetic construct
  source 1..38: mol_type = other DNA
agcggtcgcgccgtgggaggggtggcccggccccccca

SEQ ID NO: 436 - DNA, 38 bp, synthetic construct
  source 1..38: mol_type = other DNA
cgaggagacgccggcgcgcccccgcgggggagaccccc

SEQ ID NO: 437 - DNA, 38 bp, synthetic construct
  source 1..38: mol_type = other DNA
ctcgcgggggattccccgcgggggtgggcgccgggagg

SEQ ID NO: 438 - DNA, 38 bp, synthetic construct
  source 1..38: mol_type = other DNA
ggggagagcgcggcgacgggtctcgctccctcggcccc

SEQ ID NO: 439 - DNA, 25 bp, synthetic construct
  source 1..25: mol_type = other DNA
gggattcggcgagtgctgctgccgg

SEQ ID NO: 440 - DNA, 25 bp, synthetic construct
  source 1..25: mol_type = other DNA
gggggctgtaacactcggggggggt

SEQ ID NO: 441 - DNA, 48 bp, synthetic construct
  source 1..48: mol_type = other DNA
ttcggtcccgtcctcttttgaggaacaagttttcttgtccgccgccgc

SEQ ID NO: 442 - DNA, 48 bp, synthetic construct
  source 1..48: mol_type = other DNA
cgccgccgcctcctcttttgaggaacaagttttcttgtaccgccgccg

SEQ ID NO: 443 - DNA, 48 bp, synthetic construct
  source 1..48: mol_type = other DNA
ccgccgccgctcctcttttgaggaacaagttttcttgtcccgacccgc

SEQ ID NO: 444 - DNA, 48 bp, synthetic construct
  source 1..48: mol_type = other DNA
gcgccctccctcctcttttgaggaacaagttttcttgtgagggaggac

SEQ ID NO: 445 - DNA, 48 bp, synthetic construct
  source 1..48: mol_type = other DNA
gcggggccggtcctcttttgaggaacaagttttcttgtggggcggaga

SEQ ID NO: 446 - DNA, 48 bp, synthetic construct
  source 1..48: mol_type = other DNA
cgggggaggatcctcttttgaggaacaagttttcttgtggaggacgga

SEQ ID NO: 447 - DNA, 38 bp, synthetic construct
  source 1..38: mol_type = other DNA
cggacggacggacggggccccccgagccaccttccccg

SEQ ID NO: 448 - DNA, 38 bp, synthetic construct
  source 1..38: mol_type = other DNA
ccgggccttcccagccgtcccggagccggtcgcggcgc

SEQ ID NO: 449 - DNA, 38 bp, synthetic construct
  source 1..38: mol_type = other DNA
accgccgcggtggaaatgcgcccggcggcggccggtcg

SEQ ID NO: 450 - DNA, 38 bp, synthetic construct
  source 1..38: mol_type = other DNA
ccggtcgggggacggtcccccgccgaccccacccccgg

SEQ ID NO: 451 - DNA, 38 bp, synthetic construct
  source 1..38: mol_type = other DNA
ccccgcccgcccacccccgcacccgccggagcccgccc

SEQ ID NO: 452 - DNA, 38 bp, synthetic construct
  source 1..38: mol_type = other DNA
cctccggggaggaggaggaggggcggcgggggaaggga

SEQ ID NO: 453 - DNA, 25 bp, synthetic construct
  source 1..25: mol_type = other DNA
gggcgggtggaggggtcgggaggaa

SEQ ID NO: 454 - DNA, 25 bp, synthetic construct
  source 1..25: mol_type = other DNA
cggggggcgggaaagatccgccggg

SEQ ID NO: 455 - DNA, 48 bp, synthetic construct
  source 1..48: mol_type = other DNA
ccgccgacactcctcttttgaggaacaagttttcttgtggccggaccc

SEQ ID NO: 456 - DNA, 48 bp, synthetic construct
  source 1..48: mol_type = other DNA
gccgccgggttcctcttttgaggaacaagttttcttgttgaatcctcc

SEQ ID NO: 457 - DNA, 48 bp, synthetic construct
  source 1..48: mol_type = other DNA
gggcggactgtcctcttttgaggaacaagttttcttgtcgcggacccc

SEQ ID NO: 458 - DNA, 48 bp, synthetic construct
  source 1..48: mol_type = other DNA
acccgtttactcctcttttgaggaacaagttttcttgtctcttaacgg

SEQ ID NO: 459 - DNA, 48 bp, synthetic construct
  source 1..48: mol_type = other DNA
tttcacgccctcctcttttgaggaacaagttttcttgttcttgaactc

SEQ ID NO: 460 - DNA, 48 bp, synthetic construct
  source 1..48: mol_type = other DNA
tctcttcaaatcctcttttgaggaacaagttttcttgtgttcttttca

SEQ ID NO: 461 - DNA, 38 bp, synthetic construct
  source 1..38: mol_type = other DNA
actttcccttacggtacttgttgactatcggtctcgtg

SEQ ID NO: 462 - DNA, 38 bp, synthetic construct
  source 1..38: mol_type = other DNA
ccggtatttagccttagatggagtttaccacccgcttt

SEQ ID NO: 463 - DNA, 38 bp, synthetic construct
  source 1..38: mol_type = other DNA
gggctgcattcccaagcaacccgactccgggaagaccc

SEQ ID NO: 464 - DNA, 38 bp, synthetic construct
  source 1..38: mol_type = other DNA
gggcccggcgcgccgggggccgctaccggcctcacacc

SEQ ID NO: 465 - DNA, 38 bp, synthetic construct
  source 1..38: mol_type = other DNA
gtccacgggctgggcctcgatcagaaggacttgggccc

SEQ ID NO: 466 - DNA, 38 bp, synthetic construct
  source 1..38: mol_type = other DNA
cccacgagcggcgccggggagcgggtcttccgtacgcc

SEQ ID NO: 467 - DNA, 38 bp, synthetic construct
  source 1..38: mol_type = other DNA
acatgtcccgcgccccgccgcggggcggggattcggcg

SEQ ID NO: 468 - DNA, 38 bp, synthetic construct
  source 1..38: mol_type = other DNA
ctgggctcttccctgttcactcgccgttactgagggaa

SEQ ID NO: 469 - DNA, 38 bp, synthetic construct
  source 1..38: mol_type = other DNA
tcctggttagtttcttctcctccgctgactaatatgct

SEQ ID NO: 470 - DNA, 38 bp, synthetic construct
  source 1..38: mol_type = other DNA
taaattcagcgggtcgccacgtctgatctgaggtcgcg

SEQ ID NO: 471 - DNA, 51 bp, synthetic construct
  source 1..51: mol_type = other DNA
ccggctttctcctcgtacgggcgaccccacgatgacccacttcgcttgtag

SEQ ID NO: 472 - DNA, 50 bp, synthetic construct
  source 1..50: mol_type = other DNA
gcaccttgatctatcgatgtgacacttaacgccccccgtgaatacggaga

SEQ ID NO: 473 - DNA, 50 bp, synthetic construct
  source 1..50: mol_type = other DNA
ggggtagtgccactgtttcgttttggccccagtcgagttaaaacgaccgg

SEQ ID NO: 474 - DNA, 50 bp, synthetic construct
  source 1..50: mol_type = other DNA
gagtccagttcgaacgatattttaaagagaatgagttatcttcagtctca

SEQ ID NO: 475 - DNA, 50 bp, synthetic construct
  source 1..50: mol_type = other DNA
ccgtccgcgtaaacgcgaacggaggggacgaaggtctcgttctccctatc

SEQ ID NO: 476 - DNA, 50 bp, synthetic construct
  source 1..50: mol_type = other DNA
aagggtactaaaagctcgcacaggtcaaacctcctaggaatggaattccg

SEQ ID NO: 477 - DNA, 50 bp, synthetic construct
  source 1..50: mol_type = other DNA
gctacctacagcgatagccatggtagcgtctcgctaaagacattaaaaat

SEQ ID NO: 478 - DNA, 50 bp, synthetic construct
  source 1..50: mol_type = other DNA
ggcattagctcgacaggaagttgagcaggaccccgaaaggggtcccaccc

SEQ ID NO: 479 - DNA, 50 bp, synthetic construct
  source 1..50: mol_type = other DNA
tgggtggtaactagccaagcagctagttaccaaatcgggagaatcccggg

SEQ ID NO: 480 - DNA, 50 bp, synthetic construct
  source 1..50: mol_type = other DNA
tcctctctttagggggaggtccctgggccgaagcccgcccacctttcggt

SEQ ID NO: 481 - DNA, 50 bp, synthetic construct
  source 1..50: mol_type = other DNA
ggagccggaccgctttcgcacccgtgctctttcgagcacacccaccccgt

SEQ ID NO: 482 - DNA, 50 bp, synthetic construct
  source 1..50: mol_type = other DNA
ttacgggggtccctcggtcagctaccgaggagagctcgctggcccacact

SEQ ID NO: 483 - DNA, 50 bp, synthetic construct
  source 1..50: mol_type = other DNA
cctgagggaatgtgggaaccggcgttagccactccgaagtgcgtataacg

SEQ ID NO: 484 - DNA, 50 bp, synthetic construct
  source 1..50: mol_type = other DNA
cgcacgccggcggacttcatgctgtcggtgatttcacctccagtatggaa

SEQ ID NO: 485 - DNA, 50 bp, synthetic construct
  source 1..50: mol_type = other DNA
ccacgctatgtagcgaccactgtcgtgcttttcgctgaagaacttgcgtt

SEQ ID NO: 486 - DNA, 50 bp, synthetic construct
  source 1..50: mol_type = other DNA
ctcgagcgatacgagcaagacggaaacccgaggtacgggtatccgcgagc

SEQ ID NO: 487 - DNA, 50 bp, synthetic construct
  source 1..50: mol_type = other DNA
agccgcccgtacggagtcttggtgtataccgagactgccgtaggcgggct

SEQ ID NO: 488 - DNA, 50 bp, synthetic construct
  source 1..50: mol_type = other DNA
gactacgtagtagtcggcagcgaggtccgtcccaccgaagaacatcgaag

SEQ ID NO: 489 - DNA, 50 bp, synthetic construct
  source 1..50: mol_type = other DNA
gcacctgggaggagagccgtacccacaccttatagaggcgtggatctgac

SEQ ID NO: 490 - DNA, 50 bp, synthetic construct
  source 1..50: mol_type = other DNA
atacctccgacaactccccaaccccgtagccgatttaatatcagcatcag

SEQ ID NO: 491 - DNA, 50 bp, synthetic construct
  source 1..50: mol_type = other DNA
ggcgaagagattgtcaacaggtttcttgatgtaaaacggtttgacatcga

SEQ ID NO: 492 - DNA, 50 bp, synthetic construct
  source 1..50: mol_type = other DNA
caccacggtaaaagtgcgcgccgcagctctcgcgaaagagcccggacacg

SEQ ID NO: 493 - DNA, 50 bp, synthetic construct
  source 1..50: mol_type = other DNA
aacgttttacgaagattcggtttaaaaccgtagtaggcaagtgcctctag

SEQ ID NO: 494 - DNA, 50 bp, synthetic construct
  source 1..50: mol_type = other DNA
cacacggggtgcaatctcactgggacatataatatcgtccccgtagatgc

SEQ ID NO: 495 - DNA, 50 bp, synthetic construct
  source 1..50: mol_type = other DNA
ctatggttccggcgttaccaaaatggatttgggtcgctttgactattgcc

SEQ ID NO: 496 - DNA, 50 bp, synthetic construct
  source 1..50: mol_type = other DNA
cagaatatcatggactctagctcaaatgtgaacccatttcccattgtgga

SEQ ID NO: 497 - DNA, 50 bp, synthetic construct
  source 1..50: mol_type = other DNA
aaatagttcccatcgtatcgtctcgccatctacgattccgtagtgtgagc

SEQ ID NO: 498 - DNA, 50 bp, synthetic construct
  source 1..50: mol_type = other DNA
ggatacgatcgagatatgaatatagctctggtgggagaaaactccacacc

SEQ ID NO: 499 - DNA, 50 bp, synthetic construct
  source 1..50: mol_type = other DNA
aggcgatcggagatggaatcggatgcagacgataagtctatcgtcgcaag

SEQ ID NO: 500 - DNA, 50 bp, synthetic construct
  source 1..50: mol_type = other DNA
cgaaccatctacgctgccctgctgagccagacgctggttgatcgattgat

SEQ ID NO: 501 - DNA, 50 bp, synthetic construct
  source 1..50: mol_type = other DNA
cattcaggtctataccaacggatttgagccggcgtctgatgaaagcaccg

SEQ ID NO: 502 - DNA, 40 bp, synthetic construct
  source 1..40: mol_type = other DNA
acccctttctggaggtacatattcatatcaggctccttac

SEQ ID NO: 503 - DNA, 40 bp, synthetic construct
  source 1..40: mol_type = other DNA
aggcagcccgatctattttattattcttcggaactgtaaa

SEQ ID NO: 504 - DNA, 48 bp, synthetic construct
  source 1..48: mol_type = other DNA
acatcacttgtcctcttttgaggaacaagttttcttgtcctgagtaga

SEQ ID NO: 505 - DNA, 48 bp, synthetic construct
  source 1..48: mol_type = other DNA
agaactcaaatcctcttttgaggaacaagttttcttgtctatcggcct

SEQ ID NO: 506 - DNA, 48 bp, synthetic construct
  source 1..48: mol_type = other DNA
tgctggtaattcctcttttgaggaacaagttttcttgtatccagaaca

SEQ ID NO: 507 - DNA, 48 bp, synthetic construct
  source 1..48: mol_type = other DNA
atattaccgctcctcttttgaggaacaagttttcttgtcagccattgc

SEQ ID NO: 508 - DNA, 48 bp, synthetic construct
  source 1..48: mol_type = other DNA
aacaggaaaatcctcttttgaggaacaagttttcttgtacgctcatgg

SEQ ID NO: 509 - DNA, 48 bp, synthetic construct
  source 1..48: mol_type = other DNA
aaatacctactcctcttttgaggaacaagttttcttgtattttgacgc

SEQ ID NO: 510 - DNA, 48 bp, synthetic construct
  source 1..48: mol_type = other DNA
tcaatcgtcttcctcttttgaggaacaagttttcttgtgaaatggatt

SEQ ID NO: 511 - DNA, 48 bp, synthetic construct
  source 1..48: mol_type = other DNA
atttacattgtcctcttttgaggaacaagttttcttgtgcagattcac

SEQ ID NO: 512 - DNA, 30 bp, synthetic construct
  source 1..30: mol_type = other DNA
cagtcacacgaccagtaataaaagggacat

SEQ ID NO: 513 - DNA, 44 bp, synthetic construct
  source 1..44: mol_type = other DNA
ttacctgagcaaaagaagatgatgaaacaaacatcaagaaaaca

SEQ ID NO: 514 - DNA, 48 bp, synthetic construct
  source 1..48: mol_type = other DNA
aaattaattatcctcttttgaggaacaagttttcttgtcatttaacaa

SEQ ID NO: 515 - DNA, 48 bp, synthetic construct
  source 1..48: mol_type = other DNA
tttcatttgatcctcttttgaggaacaagttttcttgtattacctttt

SEQ ID NO: 516 - DNA, 48 bp, synthetic construct
  source 1..48: mol_type = other DNA
ttaatggaaatcctcttttgaggaacaagttttcttgtcagtacataa

SEQ ID NO: 517 - DNA, 48 bp, synthetic construct
  source 1..48: mol_type = other DNA
atcaatatattcctcttttgaggaacaagttttcttgtgtgagtgaat

SEQ ID NO: 518 - DNA, 48 bp, synthetic construct
  source 1..48: mol_type = other DNA
aaccttgctttcctcttttgaggaacaagttttcttgtctgtaaatcg

SEQ ID NO: 519 - DNA, 48 bp, synthetic construct
  source 1..48: mol_type = other DNA
tcgctattaatcctcttttgaggaacaagttttcttgtttaattttcc

SEQ ID NO: 520 - DNA, 48 bp, synthetic construct
  source 1..48: mol_type = other DNA
cttagaatcctcctcttttgaggaacaagttttcttgtttgaaaacat

SEQ ID NO: 521 - DNA, 48 bp, synthetic construct
  source 1..48: mol_type = other DNA
agcgatagcttcctcttttgaggaacaagttttcttgttagattaaga

SEQ ID NO: 522 - DNA, 24 bp, synthetic construct
  source 1..24: mol_type = other DNA
cgctgagaagagtcaatagtgaat

SEQ ID NO: 523 - DNA, 50 bp, synthetic construct
  source 1..50: mol_type = other DNA
tgaatcttaccaacgctaacgagcgtctttccagagcctaatttgccagt

SEQ ID NO: 524 - DNA, 48 bp, synthetic construct
  source 1..48: mol_type = other DNA
tacaaaataatcctcttttgaggaacaagttttcttgtacagccatat

SEQ ID NO: 525 - DNA, 48 bp, synthetic construct
  source 1..48: mol_type = other DNA
tatttatccctcctcttttgaggaacaagttttcttgtaatccaaata

SEQ ID NO: 526 - DNA, 48 bp, synthetic construct
  source 1..48: mol_type = other DNA
agaaacgatttcctcttttgaggaacaagttttcttgtttttgtttaa

SEQ ID NO: 527 - DNA, 48 bp, synthetic construct
  source 1..48: mol_type = other DNA
cgtcaaaaattcctcttttgaggaacaagttttcttgtgaaaatagca

SEQ ID NO: 528 - DNA, 48 bp, synthetic construct
  source 1..48: mol_type = other DNA
gcctttacagtcctcttttgaggaacaagttttcttgtagagaataac

SEQ ID NO: 529 - DNA, 48 bp, synthetic construct
  source 1..48: mol_type = other DNA
ataaaaacagtcctcttttgaggaacaagttttcttgtggaagcgcat

SEQ ID NO: 530 - DNA, 48 bp, synthetic construct
  source 1..48: mol_type = other DNA
tagacgggagtcctcttttgaggaacaagttttcttgtaattaactga

SEQ ID NO: 531 - DNA, 48 bp, synthetic construct
  source 1..48: mol_type = other DNA
acaccctgaatcctcttttgaggaacaagttttcttgtcaaagtcaga

SEQ ID NO: 532 - DNA, 56 bp, synthetic construct
  source 1..56: mol_type = other DNA
gggtaattgagcgctaatatcagagagataacccacaagaattgagttaagcccaa

SEQ ID NO: 533 - DNA, 56 bp, synthetic construct
  source 1..56: mol_type = other DNA
tcacaaacaaataaatcctcattaaagccagaatggaaagcgcagtctctgaattt

SEQ ID NO: 534 - DNA, 48 bp, synthetic construct
  source 1..48: mol_type = other DNA
accgttccagtcctcttttgaggaacaagttttcttgttaagcgtcat

SEQ ID NO: 535 - DNA, 48 bp, synthetic construct
  source 1..48: mol_type = other DNA
acatggcttttcctcttttgaggaacaagttttcttgttgatgataca

SEQ ID NO: 536 - DNA, 48 bp, synthetic construct
  source 1..48: mol_type = other DNA
ggagtgtacttcctcttttgaggaacaagttttcttgtggtaataagt

SEQ ID NO: 537 - DNA, 48 bp, synthetic construct
  source 1..48: mol_type = other DNA
tttaacggggtcctcttttgaggaacaagttttcttgttcagtgcctt

SEQ ID NO: 538 - DNA, 48 bp, synthetic construct
  source 1..48: mol_type = other DNA
gagtaacagttcctcttttgaggaacaagttttcttgtgcccgtataa

SEQ ID NO: 539 - DNA, 48 bp, synthetic construct
  source 1..48: mol_type = other DNA
acagttaatgtcctcttttgaggaacaagttttcttgtccccctgcct

SEQ ID NO: 540 - DNA, 48 bp, synthetic construct
  source 1..48: mol_type = other DNA
atttcggaactcctcttttgaggaacaagttttcttgtctattattct

SEQ ID NO: 541 - DNA, 48 bp, synthetic construct
  source 1..48: mol_type = other DNA
gaaacatgaatcctcttttgaggaacaagttttcttgtagtattaaga

SEQ ID NO: 542 - DNA, 50 bp, synthetic construct
  source 1..50: mol_type = other DNA
ggctgagactcctcaagagaaggattaggattagcggggttttgctcagt

SEQ ID NO: 543 - DNA, 24 bp, synthetic construct
  source 1..24: mol_type = other DNA
caaagtacaacggagatttgtatc

SEQ ID NO: 544 - DNA, 48 bp, synthetic construct
  source 1..48: mol_type = other DNA
atcgcctgattcctcttttgaggaacaagttttcttgtaaattgtgtc

SEQ ID NO: 545 - DNA, 48 bp, synthetic construct
  source 1..48: mol_type = other DNA
gaaatccgcgtcctcttttgaggaacaagttttcttgtacctgctcca

SEQ ID NO: 546 - DNA, 48 bp, synthetic construct
  source 1..48: mol_type = other DNA
tgttacttagtcctcttttgaggaacaagttttcttgtccggaacgag

SEQ ID NO: 547 - DNA, 48 bp, synthetic construct
  source 1..48: mol_type = other DNA
gcgcagacggtcctcttttgaggaacaagttttcttgttcaatcataa

SEQ ID NO: 548 - DNA, 48 bp, synthetic construct
  source 1..48: mol_type = other DNA
gggaaccgaatcctcttttgaggaacaagttttcttgtctgaccaact

SEQ ID NO: 549 - DNA, 48 bp, synthetic construct
  source 1..48: mol_type = other DNA
ttgaaagaggtcctcttttgaggaacaagttttcttgtacagatgaac

SEQ ID NO: 550 - DNA, 48 bp, synthetic construct
  source 1..48: mol_type = other DNA
ggtgtacagatcctcttttgaggaacaagttttcttgtccaggcgcat

SEQ ID NO: 551 - DNA, 48 bp, synthetic construct
  source 1..48: mol_type = other DNA
aggctggctgtcctcttttgaggaacaagttttcttgtaccttcatca

SEQ ID NO: 552 - DNA, 44 bp, synthetic construct
  source 1..44: mol_type = other DNA
agagtaatcttgacaagaaccggatattcattacccaaatcaac

SEQ ID NO: 553 - DNA, 30 bp, synthetic construct
  source 1..30: mol_type = other DNA
acagttgattcccaattctgcgaacgagta

SEQ ID NO: 554 - DNA, 48 bp, synthetic construct
  source 1..48: mol_type = other DNA
gatttagttttcctcttttgaggaacaagttttcttgtgaccattaga

SEQ ID NO: 555 - DNA, 48 bp, synthetic construct
  source 1..48: mol_type = other DNA
tacatttcgctcctcttttgaggaacaagttttcttgtaaatggtcaa

SEQ ID NO: 556 - DNA, 48 bp, synthetic construct
  source 1..48: mol_type = other DNA
taacctgttttcctcttttgaggaacaagttttcttgtagctatattt

SEQ ID NO: 557 - DNA, 48 bp, synthetic construct
  source 1..48: mol_type = other DNA
tcatttggggtcctcttttgaggaacaagttttcttgtcgcgagctga

SEQ ID NO: 558 - DNA, 48 bp, synthetic construct
  source 1..48: mol_type = other DNA
aaaggtggcatcctcttttgaggaacaagttttcttgttcaattctac

SEQ ID NO: 559 - DNA, 48 bp, synthetic construct
  source 1..48: mol_type = other DNA
taatagtagttcctcttttgaggaacaagttttcttgtagcattaaca

SEQ ID NO: 560 - DNA, 48 bp, synthetic construct
  source 1..48: mol_type = other DNA
tccaataaattcctcttttgaggaacaagttttcttgtcatacaggca

SEQ ID NO: 561 - DNA, 48 bp, synthetic construct
  source 1..48: mol_type = other DNA
aggcaaagaatcctcttttgaggaacaagttttcttgtttagcaaaat

SEQ ID NO: 562 - DNA, 25 bp, synthetic construct
  source 1..25: mol_type = other DNA
tgggtggtaactagccaagcagcta

SEQ ID NO: 563 - DNA, 32 bp, synthetic construct
  source 1..32: mol_type = other DNA
gttaccaaatcgggagaatcccgggtcctctc

SEQ ID NO: 564 - DNA, 40 bp, synthetic construct
  source 1..40: mol_type = other DNA
tttagggggaggtccctgggccgaagcccgcccacctttc

SEQ ID NO: 565 - DNA, 40 bp, synthetic construct
  source 1..40: mol_type = other DNA
ggtggagccggaccgctttcgcacccgtgctctttcgagc

SEQ ID NO: 566 - DNA, 40 bp, synthetic construct
  source 1..40: mol_type = other DNA
acacccaccccgtttacgggggtccctcggtcagctaccg

SEQ ID NO: 567 - DNA, 40 bp, synthetic construct
  source 1..40: mol_type = other DNA
aggagagctcgctggcccacactcctgagggaatgtggga

SEQ ID NO: 568 - DNA, 40 bp, synthetic construct
  source 1..40: mol_type = other DNA
accggcgttagccactccgaagtgcgtataacgcgcacgc

SEQ ID NO: 569 - DNA, 40 bp, synthetic construct
  source 1..40: mol_type = other DNA
cggcggacttcatgctgtcggtgatttcacctccagtatg

SEQ ID NO: 570 - DNA, 40 bp, synthetic construct
  source 1..40: mol_type = other DNA
gaaccacgctatgtagcgaccactgtcgtgcttttcgctg

SEQ ID NO: 571 - DNA, 40 bp, synthetic construct
  source 1..40: mol_type = other DNA
aagaacttgcgttctcgagcgatacgagcaagacggaaac

SEQ ID NO: 572 - DNA, 40 bp, synthetic construct
  source 1..40: mol_type = other DNA
ccgaggtacgggtatccgcgagcagccgcccgtacggagt

SEQ ID NO: 573 - DNA, 40 bp, synthetic construct
  source 1..40: mol_type = other DNA
cttggtgtataccgagactgccgtaggcgggctgactacg

SEQ ID NO: 574 - DNA, 40 bp, synthetic construct
  source 1..40: mol_type = other DNA
tagtagtcggcagcgaggtccgtcccaccgaagaacatcg

SEQ ID NO: 575 - DNA, 40 bp, synthetic construct
  source 1..40: mol_type = other DNA
aaggcacctgggaggagagccgtacccacaccttatagag

SEQ ID NO: 576 - DNA, 40 bp, synthetic construct
  source 1..40: mol_type = other DNA
gcgtggatctgacatacctccgacaactccccaaccccgt

SEQ ID NO: 577 - DNA, 40 bp, synthetic construct
  source 1..40: mol_type = other DNA
agccgatttaatatcagcatcagggcgaagagattgtcaa

SEQ ID NO: 578 - DNA, 40 bp, synthetic construct
  source 1..40: mol_type = other DNA
caggtttcttgatgtaaaacggtttgacatcgacaccacg

SEQ ID NO: 579 - DNA, 40 bp, synthetic construct
  source 1..40: mol_type = other DNA
gtaaaagtgcgcgccgcagctctcgcgaaagagcccggac

SEQ ID NO: 580 - DNA, 40 bp, synthetic construct
  source 1..40: mol_type = other DNA
acgaacgttttacgaagattcggtttaaaaccgtagtagg

SEQ ID NO: 581 - DNA, 40 bp, synthetic construct
  source 1..40: mol_type = other DNA
caagtgcctctagcacacggggtgcaatctcactgggaca

SEQ ID NO: 582 - DNA, 40 bp, synthetic construct
  source 1..40: mol_type = other DNA
tataatatcgtccccgtagatgcctatggttccggcgtta

SEQ ID NO: 583 - DNA, 40 bp, synthetic construct
  source 1..40: mol_type = other DNA
ccaaaatggatttgggtcgctttgactattgcccagaata

SEQ ID NO: 584 - DNA, 25 bp, synthetic construct
  source 1..25: mol_type = other DNA
tcatggactctagctcaaatgtgaa

SEQ ID NO: 585 - DNA, 25 bp, synthetic construct
  source 1..25: mol_type = other DNA
cccatttcccattgtggaaaatagt

SEQ ID NO: 586 - DNA, 25 bp, synthetic construct
  source 1..25: mol_type = other DNA
tcccatcgtatcgtctcgccatcta

SEQ ID NO: 587 - DNA, 40 bp, synthetic construct
  source 1..40: mol_type = other DNA
cgattccgtagtgtgagcggatacgatcgagatatgaata

SEQ ID NO: 588 - DNA, 40 bp, synthetic construct
  source 1..40: mol_type = other DNA
tagctctggtgggagaaaactccacaccaggcgatcggag

SEQ ID NO: 589 - DNA, 40 bp, synthetic construct
  source 1..40: mol_type = other DNA
atggaatcggatgcagacgataagtctatcgtcgcaagcg

SEQ ID NO: 590 - DNA, 40 bp, synthetic construct
  source 1..40: mol_type = other DNA
aaccatctacgctgccctgctgagccagacgctggttgat

SEQ ID NO: 591 - DNA, 40 bp, synthetic construct
  source 1..40: mol_type = other DNA
cgattgatcattcaggtctataccaacggatttgagccgg

SEQ ID NO: 592 - DNA, 40 bp, synthetic construct
  source 1..40: mol_type = other DNA
cgtctgatgaaagcaccgacccctttctggaggtacatat

SEQ ID NO: 593 - DNA, 40 bp, synthetic construct
  source 1..40: mol_type = other DNA
tcatatcaggctccttacaggcagcccgatctattttatt

SEQ ID NO: 594 - DNA, 40 bp, synthetic construct
  source 1..40: mol_type = other DNA
attcttcggaactgtaaacactccgttccctacaacgagc

SEQ ID NO: 595 - DNA, 40 bp, synthetic construct
  source 1..40: mol_type = other DNA
ctaaattcatatgactcgttatagcggaccgcgtgtctga

SEQ ID NO: 596 - DNA, 40 bp, synthetic construct
  source 1..40: mol_type = other DNA
tccacggcgcacattggtctcggaccaatagagccgctct

SEQ ID NO: 597 - DNA, 40 bp, synthetic construct
  source 1..40: mol_type = other DNA
cagagcgcggggggtaacggttgcttgttcagcgaacttc

SEQ ID NO: 598 - DNA, 40 bp, synthetic construct
  source 1..40: mol_type = other DNA
ttgtaaggcgctgcatcctgcaacttgtgccccataggag

SEQ ID NO: 599 - DNA, 40 bp, synthetic construct
  source 1..40: mol_type = other DNA
caccgttggagaacgtgcattgcccaaacaacgacgatcg

SEQ ID NO: 600 - DNA, 40 bp, synthetic construct
  source 1..40: mol_type = other DNA
gtagccagagaggaggttgccaataaggctacggatgctg

SEQ ID NO: 601 - DNA, 40 bp, synthetic construct
  source 1..40: mol_type = other DNA
gtttgtaaaacatccggatcccatgacaaggatttgtcat

SEQ ID NO: 602 - DNA, 40 bp, synthetic construct
  source 1..40: mol_type = other DNA
gtaagaaaccttctctatttatctgaccgcgatcaccatt

SEQ ID NO: 603 - DNA, 40 bp, synthetic construct
  source 1..40: mol_type = other DNA
cgcctcccgtagcttagcgatagctaaggtacgacgggtc

SEQ ID NO: 604 - DNA, 40 bp, synthetic construct
  source 1..40: mol_type = other DNA
gcctcgtcattaccagaacctaaggtcggatgctttgtga

SEQ ID NO: 605 - DNA, 40 bp, synthetic construct
  source 1..40: mol_type = other DNA
gcaattcgtcccttaagtaagcaattgctgtaaagtcgtc

SEQ ID NO: 606 - DNA, 40 bp, synthetic construct
  source 1..40: mol_type = other DNA
actgtgcggatcaccgcttccagtagcgacagaagcaatt

SEQ ID NO: 607 - DNA, 40 bp, synthetic construct
  source 1..40: mol_type = other DNA
gattggtaaatttcgagagaaagatcgcgaggaagatcaa

SEQ ID NO: 608 - DNA, 25 bp, synthetic construct
  source 1..25: mol_type = other DNA
tacataaagagttgaacttctttgt

SEQ ID NO: 609 - DNA, 25 bp, synthetic construct
  source 1..25: mol_type = other DNA
tgtcttcgacatgggtaatcctcat

SEQ ID NO: 610 - DNA, 40 bp, synthetic construct
  source 1..40: mol_type = other DNA
gtttgaatggccggcgtctattagtagatgccggagtttg

SEQ ID NO: 611 - DNA, 40 bp, synthetic construct
  source 1..40: mol_type = other DNA
ctgcgattgctgagggaatcgggtttccatcttttaggag

SEQ ID NO: 612 - DNA, 40 bp, synthetic construct
  source 1..40: mol_type = other DNA
accttgcattgccttaacaataagctcgcagtcggaattc

SEQ ID NO: 613 - DNA, 40 bp, synthetic construct
  source 1..40: mol_type = other DNA
gtagcgaaaattggaatggttagttccatatttaagtacg

SEQ ID NO: 614 - DNA, 40 bp, synthetic construct
  source 1..40: mol_type = other DNA
aacgccatgcggctacaggaagctctacaccaccaacagt

SEQ ID NO: 615 - DNA, 40 bp, synthetic construct
  source 1..40: mol_type = other DNA
ctgggttgccactttaggcacctcgactttgatggtgtat

SEQ ID NO: 616 - DNA, 40 bp, synthetic construct
  source 1..40: mol_type = other DNA
ttgcgattctgcgcagagctctgacgaacgctacaggtta

SEQ ID NO: 617 - DNA, 40 bp, synthetic construct
  source 1..40: mol_type = other DNA
ctttgtaagcctgtgaacgcgagttagagctgatccattc

SEQ ID NO: 618 - DNA, 40 bp, synthetic construct
  source 1..40: mol_type = other DNA
agcgaccccgttagcgaagttgcttggggcgacagtcacg

SEQ ID NO: 619 - DNA, 40 bp, synthetic construct
  source 1..40: mol_type = other DNA
tcgccagttccgccattgtcgacgagaacgaactgagtaa

SEQ ID NO: 620 - DNA, 40 bp, synthetic construct
  source 1..40: mol_type = other DNA
agttagaagccatgcttcaaactccggttgagggctctat

SEQ ID NO: 621 - DNA, 40 bp, synthetic construct
  source 1..40: mol_type = other DNA
ctagagagccgttgcctgattaatgctaacgcatctaagg

SEQ ID NO: 622 - DNA, 40 bp, synthetic construct
  source 1..40: mol_type = other DNA
tatggaccatcgagaaaggagactttacgtacgcgccagt

SEQ ID NO: 623 - DNA, 40 bp, synthetic construct
  source 1..40: mol_type = other DNA
tgttggccatacggattgtacccctcgatgcatggctgag

SEQ ID NO: 624 - DNA, 40 bp, synthetic construct
  source 1..40: mol_type = other DNA
atttgggccttagcagtgccctgtctctccacagtccacc

SEQ ID NO: 625 - DNA, 40 bp, synthetic construct
  source 1..40: mol_type = other DNA
cgtagggagcgtcaacgcttatgatggactcacccgttat

SEQ ID NO: 626 - DNA, 40 bp, synthetic construct
  source 1..40: mol_type = other DNA
tacgtcagtaactgttcctgacatgtaggagcatcccacg

SEQ ID NO: 627 - DNA, 40 bp, synthetic construct
  source 1..40: mol_type = other DNA
ggggccgtaaggccctcgagcatgttacctacaggtagga

SEQ ID NO: 628 - DNA, 40 bp, synthetic construct
  source 1..40: mol_type = other DNA
gccagtcgacaacgaatgagaaaggcaccttttcccacac

SEQ ID NO: 629 - DNA, 40 bp, synthetic construct
  source 1..40: mol_type = other DNA
tatacctagtgggttcaagatacctagagacgacaaccat

SEQ ID NO: 630 - DNA, 40 bp, synthetic construct
  source 1..40: mol_type = other DNA
gccaaacgtgcatcgtttatgtaaaaccatatcacgatac

SEQ ID NO: 631 - DNA, 25 bp, synthetic construct
  source 1..25: mol_type = other DNA
gtcgcgatatgttgcacgttgtctg

SEQ ID NO: 632 - DNA, 25 bp, synthetic construct
  source 1..25: mol_type = other DNA
gaagtttgcagctggatacgacaga

SEQ ID NO: 633 - DNA, 40 bp, synthetic construct
  source 1..40: mol_type = other DNA
cggccatctaacttgatgttagtaccgacctgacgtacgg

SEQ ID NO: 634 - DNA, 40 bp, synthetic construct
  source 1..40: mol_type = other DNA
ctctcataggaagaaactcttgaaggtgaaccttcgtaag

SEQ ID NO: 635 - DNA, 40 bp, synthetic construct
  source 1..40: mol_type = other DNA
catctcatatgcaccctggatatcactcattagtggtaac

SEQ ID NO: 636 - DNA, 40 bp, synthetic construct
  source 1..40: mol_type = other DNA
caaccgaactgcaactccaaccacctgccggccacgtgtt

SEQ ID NO: 637 - DNA, 40 bp, synthetic construct
  source 1..40: mol_type = other DNA
ttgatcgaaactttcgatcttcgtttagggcaaggtagcg

SEQ ID NO: 638 - DNA, 40 bp, synthetic construct
  source 1..40: mol_type = other DNA
gagcgcctggcgccaattaccgcgacgagcggcagtgtac

SEQ ID NO: 639 - DNA, 40 bp, synthetic construct
  source 1..40: mol_type = other DNA
gccttcacgagcgcaatggtttgcgtcgcgagttgtgagg

SEQ ID NO: 640 - DNA, 40 bp, synthetic construct
  source 1..40: mol_type = other DNA
ctgtcgacctggcctctgctaaagcaacaccaaggttaaa

SEQ ID NO: 641 - DNA, 40 bp, synthetic construct
  source 1..40: mol_type = other DNA
attaccctgggtgaccttttgcaggacttcggtcgacgcc

SEQ ID NO: 642 - DNA, 40 bp, synthetic construct
  source 1..40: mol_type = other DNA
cggttcgcaacgttctgcggcacttcgatgtaagtcaagt

SEQ ID NO: 643 - DNA, 40 bp, synthetic construct
  source 1..40: mol_type = other DNA
tttggcttacagggaagaggctgtagcaggagcgtgcgtc

SEQ ID NO: 644 - DNA, 40 bp, synthetic construct
  source 1..40: mol_type = other DNA
gagggagaagccgaaaccggctttctcctcgtacgggcga

SEQ ID NO: 645 - DNA, 40 bp, synthetic construct
  source 1..40: mol_type = other DNA
ccccacgatgacccacttcgcttgtaggcaccttgatcta

SEQ ID NO: 646 - DNA, 40 bp, synthetic construct
  source 1..40: mol_type = other DNA
tcgatgtgacacttaacgccccccgtgaatacggagaggg

SEQ ID NO: 647 - DNA, 40 bp, synthetic construct
  source 1..40: mol_type = other DNA
gtagtgccactgtttcgttttggccccagtcgagttaaaa

SEQ ID NO: 648 - DNA, 40 bp, synthetic construct
  source 1..40: mol_type = other DNA
cgaccgggagtccagttcgaacgatattttaaagagaatg

SEQ ID NO: 649 - DNA, 40 bp, synthetic construct
  source 1..40: mol_type = other DNA
agttatcttcagtctcaccgtccgcgtaaacgcgaacgga

SEQ ID NO: 650 - DNA, 40 bp, synthetic construct
  source 1..40: mol_type = other DNA
ggggacgaaggtctcgttctccctatcaagggtactaaaa

SEQ ID NO: 651 - DNA, 40 bp, synthetic construct
  source 1..40: mol_type = other DNA
gctcgcacaggtcaaacctcctaggaatggaattccggct

SEQ ID NO: 652 - DNA, 40 bp, synthetic construct
  source 1..40: mol_type = other DNA
acctacagcgatagccatggtagcgtctcgctaaagacat

SEQ ID NO: 653 - DNA, 32 bp, synthetic construct
  source 1..32: mol_type = other DNA
taaaaatggcattagctcgacaggaagttgag

SEQ ID NO: 654 - DNA, 25 bp, synthetic construct
  source 1..25: mol_type = other DNA
caggaccccgaaaggggtcccaccc

SEQ ID NO: 655 - DNA, 45 bp, synthetic construct
  source 1..45: mol_type = other DNA
gagatggaatcggatgcagatgggtggtaactagccaagcagcta

SEQ ID NO: 656 - DNA, 48 bp, synthetic construct
  source 1..48: mol_type = other DNA
tcatggactctagctcaaatgtgaatttggatatcactcattagtggt

SEQ ID NO: 657 - DNA, 48 bp, synthetic construct
  source 1..48: mol_type = other DNA
tacataaagagttgaacttctttgttttggatatcactcattagtggt

SEQ ID NO: 658 - DNA, 48 bp, synthetic construct
  source 1..48: mol_type = other DNA
gtcgcgatatgttgcacgttgtctgtttggatatcactcattagtggt

SEQ ID NO: 659 - DNA, 48 bp, synthetic construct
  source 1..48: mol_type = other DNA
gaagtttgcagctggatacgacagatttggatatcactcattagtggt

SEQ ID NO: 660 - DNA, 45 bp, synthetic construct
  source 1..45: mol_type = other DNA
caggaccccgaaaggggtcccaccccctgatatgaatatgtacct

SEQ ID NO: 661 - DNA, 45 bp, synthetic construct
  source 1..45: mol_type = other DNA
tctgcatccgattccatctctgggtggtaactagccaagcagcta

SEQ ID NO: 662 - DNA, 48 bp, synthetic construct; identical to SEQ ID NO: 656
  source 1..48: mol_type = other DNA

SEQ ID NO: 663 - DNA, 48 bp, synthetic construct
  source 1..48: mol_type = other DNA
cccatttcccattgtggaaaatagttttggatatcactcattagtggt

SEQ ID NO: 664 - DNA, 48 bp, synthetic construct
  source 1..48: mol_type = other DNA
tcccatcgtatcgtctcgccatctatttggatatcactcattagtggt

SEQ ID NO: 665 - DNA, 48 bp, synthetic construct; identical to SEQ ID NO: 657
  source 1..48: mol_type = other DNA

SEQ ID NO: 666 - DNA, 48 bp, synthetic construct; identical to SEQ ID NO: 658
  source 1..48: mol_type = other DNA

SEQ ID NO: 667 - DNA, 48 bp, synthetic construct; identical to SEQ ID NO: 659
  source 1..48: mol_type = other DNA

SEQ ID NO: 668 - DNA, 45 bp, synthetic construct; identical to SEQ ID NO: 660
  source 1..45: mol_type = other DNA

SEQ ID NO: 669 - DNA, 45 bp, synthetic construct; identical to SEQ ID NO: 661
  source 1..45: mol_type = other DNA

SEQ ID NO: 670 - DNA, 48 bp, synthetic construct; identical to SEQ ID NO: 656
  source 1..48: mol_type = other DNA

SEQ ID NO: 671 - DNA, 48 bp, synthetic construct; identical to SEQ ID NO: 663
  source 1..48: mol_type = other DNA

SEQ ID NO: 672 - DNA, 48 bp, synthetic construct; identical to SEQ ID NO: 664
  source 1..48: mol_type = other DNA

SEQ ID NO: 673 - DNA, 48 bp, synthetic construct; identical to SEQ ID NO: 657
  source 1..48: mol_type = other DNA

SEQ ID NO: 674 - DNA, 48 bp, synthetic construct
  source 1..48: mol_type = other DNA
tgtcttcgacatgggtaatcctcattttggatatcactcattagtggt

SEQ ID NO: 675 - DNA, 48 bp, synthetic construct; identical to SEQ ID NO: 658
  source 1..48: mol_type = other DNA

SEQ ID NO: 676 - DNA, 45 bp, synthetic construct
  source 1..45: mol_type = other DNA
caggaccccgaaaggggtcccacccaggtacatattcatatcagg

SEQ ID NO: 677 - DNA, 45 bp, synthetic construct; identical to SEQ ID NO: 661
  source 1..45: mol_type = other DNA

SEQ ID NO: 678 - DNA, 48 bp, synthetic construct; identical to SEQ ID NO: 504
  source 1..48: mol_type = other DNA

SEQ ID NO: 679 - DNA, 48 bp, synthetic construct; identical to SEQ ID NO: 505
  source 1..48: mol_type = other DNA

SEQ ID NO: 680 - DNA, 48 bp, synthetic construct; identical to SEQ ID NO: 506
  source 1..48: mol_type = other DNA

SEQ ID NO: 681 - DNA, 48 bp, synthetic construct; identical to SEQ ID NO: 507
  source 1..48: mol_type = other DNA

SEQ ID NO: 682 - DNA, 48 bp, synthetic construct; identical to SEQ ID NO: 508
  source 1..48: mol_type = other DNA

SEQ ID NO: 683 - DNA, 48 bp, synthetic construct; identical to SEQ ID NO: 509
  source 1..48: mol_type = other DNA

SEQ ID NO: 684 - DNA, 48 bp, synthetic construct; identical to SEQ ID NO: 510
  source 1..48: mol_type = other DNA

SEQ ID NO: 685 - DNA, 48 bp, synthetic construct; identical to SEQ ID NO: 511
  source 1..48: mol_type = other DNA

SEQ ID NO: 686 - DNA, 30 bp, synthetic construct; identical to SEQ ID NO: 512
  source 1..30: mol_type = other DNA

SEQ ID NO: 687 - DNA, 44 bp, synthetic construct; identical to SEQ ID NO: 513
  source 1..44: mol_type = other DNA

SEQ ID NO: 688 - DNA, 48 bp, synthetic construct; identical to SEQ ID NO: 514
  source 1..48: mol_type = other DNA

SEQ ID NO: 689 - DNA, 48 bp, synthetic construct; identical to SEQ ID NO: 515
  source 1..48: mol_type = other DNA

SEQ ID NO: 690 - DNA, 48 bp, synthetic construct; identical to SEQ ID NO: 516
  source 1..48: mol_type = other DNA

SEQ ID NO: 691 - DNA, 48 bp, synthetic construct; identical to SEQ ID NO: 517
  source 1..48: mol_type = other DNA

SEQ ID NO: 692 - DNA, 48 bp, synthetic construct; identical to SEQ ID NO: 518
  source 1..48: mol_type = other DNA

SEQ ID NO: 693 - DNA, 48 bp, synthetic construct; identical to SEQ ID NO: 519
  source 1..48: mol_type = other DNA

SEQ ID NO: 694 - DNA, 48 bp, synthetic construct; identical to SEQ ID NO: 520
  source 1..48: mol_type = other DNA

SEQ ID NO: 695 - DNA, 48 bp, synthetic construct; identical to SEQ ID NO: 521
  source 1..48: mol_type = other DNA

SEQ ID NO: 696 - DNA, 24 bp, synthetic construct
  source 1..24: mol_type = other DNA
cgctgagaagagtcaatagtgaat

SEQ ID NO: 697 - DNA, 50 bp, synthetic construct; identical to SEQ ID NO: 523
  source 1..50: mol_type = other DNA

SEQ ID NO: 698 - DNA, 48 bp, synthetic construct; identical to SEQ ID NO: 524
  source 1..48: mol_type = other DNA

SEQ ID NO: 699 - DNA, 48 bp, synthetic construct; identical to SEQ ID NO: 525
  source 1..48: mol_type = other DNA

SEQ ID NO: 700 - DNA, 48 bp, synthetic construct; identical to SEQ ID NO: 526
  source 1..48: mol_type = other DNA

SEQ ID NO: 701 - DNA, 48 bp, synthetic construct; identical to SEQ ID NO: 527
  source 1..48: mol_type = other DNA

SEQ ID NO: 702 - DNA, 48 bp, synthetic construct; identical to SEQ ID NO: 528
  source 1..48: mol_type = other DNA

SEQ ID NO: 703 - DNA, 48 bp, synthetic construct; identical to SEQ ID NO: 529
  source 1..48: mol_type = other DNA

SEQ ID NO: 704 - DNA, 48 bp, synthetic construct; identical to SEQ ID NO: 530
  source 1..48: mol_type = other DNA

SEQ ID NO: 705 - DNA, 48 bp, synthetic construct; identical to SEQ ID NO: 531
  source 1..48: mol_type = other DNA

SEQ ID NO: 706 - DNA, 56 bp, synthetic construct; identical to SEQ ID NO: 532
  source 1..56: mol_type = other DNA

SEQ ID NO: 707 - DNA, 38 bp, synthetic construct
  source 1..38: mol_type = other DNA
tattctcatgggtcactgaggctttttattttgagcac

SEQ ID NO: 708 - DNA, 38 bp, synthetic construct
  source 1..38: mol_type = other DNA
aaaaccaccggggatctagcctgtggccaccccggaga

SEQ ID NO: 709 - DNA, 38 bp, synthetic construct
  source 1..38: mol_type = other DNA
tgacacgaggctcacatgactctagacacttggtggaa

SEQ ID NO: 710 - DNA, 38 bp, synthetic construct
  source 1..38: mol_type = other DNA
agtgaggcgagaaaaacaatgacttgggccaattacac

SEQ ID NO: 711 - DNA, 38 bp, synthetic construct
  source 1..38: mol_type = other DNA
gactgcaaagctagagctgccaacagggctccagggag

SEQ ID NO: 712 - DNA, 38 bp, synthetic construct
  source 1..38: mol_type = other DNA
cttggcttctgtagaagttctaaggaagcggtacgaac

SEQ ID NO: 713 - DNA, 38 bp, synthetic construct
  source 1..38: mol_type = other DNA
tccacggcggtggggcgctaactagcagggacccctgc

SEQ ID NO: 714 - DNA, 38 bp, synthetic construct
  source 1..38: mol_type = other DNA
aagtgttggtcgggggcctcgagctgcctgagctgaca

SEQ ID NO: 715 - DNA, 38 bp, synthetic construct
  source 1..38: mol_type = other DNA
cgaggggaggggtctgtgtagccaacaggtgaccgaag

SEQ ID NO: 716 - DNA, 38 bp, synthetic construct
  source 1..38: mol_type = other DNA
ggcttgcctgcccacagcttacttggccaaggggtttc

SEQ ID NO: 717 - DNA, 38 bp, synthetic construct
  source 1..38: mol_type = other DNA
tgaagttcctgccggcaaacttagccttgctgcccagc

SEQ ID NO: 718 - DNA, 38 bp, synthetic construct
  source 1..38: mol_type = other DNA
tcctcttcaattctgaggagctggttgtacttggccaa

SEQ ID NO: 719 - DNA, 38 bp, synthetic construct
  source 1..38: mol_type = other DNA
gcgctcagatcggcaaggggcaccagtcttgatctgcc

SEQ ID NO: 720 - DNA, 38 bp, synthetic construct
  source 1..38: mol_type = other DNA
cagtgcacagccccacaaccaggtcagcgatgaaggta

SEQ ID NO: 721 - DNA, 34 bp, synthetic construct
  source 1..34: mol_type = other DNA
tcttcagtctcccccgaacgatgagacaccatga

SEQ ID NO: 722 - DNA, 48 bp, synthetic construct
  source 1..48: mol_type = other DNA
cgccccaacctcctcttttgaggaacaagttttcttgtattggcctgg

SEQ ID NO: 723 - DNA, 48 bp, synthetic construct
  source 1..48: mol_type = other DNA
gccagcttgctcctcttttgaggaacaagttttcttgtacgcctgaag

SEQ ID NO: 724 - DNA, 48 bp, synthetic construct
  source 1..48: mol_type = other DNA
agactcggtctcctcttttgaggaacaagttttcttgtacggagccaa

SEQ ID NO: 725 - DNA, 48 bp, synthetic construct
  source 1..48: mol_type = other DNA
tctggttgactcctcttttgaggaacaagttttcttgttttgagcagg

SEQ ID NO: 726 - DNA, 48 bp, synthetic construct
  source 1..48: mol_type = other DNA
aggcagttgctcctcttttgaggaacaagttttcttgtaggacttctc

SEQ ID NO: 727 - DNA, 48 bp, synthetic construct
  source 1..48: mol_type = other DNA
gttcacggcctcctcttttgaggaacaagttttcttgtttggcgatcc

SEQ ID NO: 728 - DNA, 38 bp, synthetic construct
  source 1..38: mol_type = other DNA
tctttgggttggtcactgtgagatcatcccccactacc

SEQ ID NO: 729 - DNA, 38 bp, synthetic construct
  source 1..38: mol_type = other DNA
tggattcctgcactggctgtgaacttctgccaagctcc

SEQ ID NO: 730 - DNA, 38 bp, synthetic construct
  source 1..38: mol_type = other DNA
ccagtcatcctggtcaaagggatcttcgatagacacca

SEQ ID NO: 731 - DNA, 38 bp, synthetic construct
  source 1..38: mol_type = other DNA
ctgggtagtccttgatgaaggacttgtacaggtcagcc

SEQ ID NO: 732 - DNA, 38 bp, synthetic construct
  source 1..38: mol_type = other DNA
agctggtcaggcgagatgtacctgctggggtcatcggg

SEQ ID NO: 733 - DNA, 38 bp, synthetic construct
  source 1..38: mol_type = other DNA
agacttgaagtccaggtcatacttcccagacctgaaga

SEQ ID NO: 734 - DNA, 50 bp, synthetic construct
  source 1..50: mol_type = other DNA
actcggaggccgctacgtccatgccgatgaccaccttatcagtgtagcca

SEQ ID NO: 735 - DNA, 48 bp, synthetic construct
  source 1..48: mol_type = other DNA
gctttcccaatcctcttttgaggaacaagttttcttgttagcagtctt

SEQ ID NO: 736 - DNA, 48 bp, synthetic construct
  source 1..48: mol_type = other DNA
cagcagctcctcctcttttgaggaacaagttttcttgtaggccttctt

SEQ ID NO: 737 - DNA, 48 bp, synthetic construct
  source 1..48: mol_type = other DNA
tattctccagtcctcttttgaggaacaagttttcttgtgatgttggga

SEQ ID NO: 738 - DNA, 48 bp, synthetic construct
  source 1..48: mol_type = other DNA
gcaaacccgctcctcttttgaggaacaagttttcttgtcttcatcccc

SEQ ID NO: 739 - DNA, 48 bp, synthetic construct
  source 1..48: mol_type = other DNA
cacattggtgtcctcttttgaggaacaagttttcttgtgcatctttcc

SEQ ID NO: 740 - DNA, 48 bp, synthetic construct
  source 1..48: mol_type = other DNA
catatttctctcctcttttgaggaacaagttttcttgtcttgatgaca

SEQ ID NO: 741 - DNA, 38 bp, synthetic construct
  source 1..38: mol_type = other DNA
ttcttcaggttgtggtaaacctctgctccaatgcgcat

SEQ ID NO: 742 - DNA, 38 bp, synthetic construct
  source 1..38: mol_type = other DNA
ggcttccctgaagtttgctgcaccgactgggaggatca

SEQ ID NO: 743 - DNA, 38 bp, synthetic construct
  source 1..38: mol_type = other DNA
tgaactcctgcatggccagcttgttgccagcatgagaa

SEQ ID NO: 744 - DNA, 38 bp, synthetic construct
  source 1..38: mol_type = other DNA
ccgccattgatgacattgaacgccgggactggcaggat

SEQ ID NO: 745 - DNA, 38 bp, synthetic construct
  source 1..38: mol_type = other DNA
gacttcagagttgccagccaagtcagcgatgtggcggt

SEQ ID NO: 746 - DNA, 38 bp, synthetic construct
  source 1..38: mol_type = other DNA
acagggggacccccttctcaacggcaccagctttgcag

SEQ ID NO: 747 - DNA, 50 bp, synthetic construct
  source 1..50: mol_type = other DNA
acggcaagggacacccccagaatggcgttcgcaccaaacttagatttatt

SEQ ID NO: 748 - DNA, 48 bp, synthetic construct
  source 1..48: mol_type = other DNA
ttctgttccatcctcttttgaggaacaagttttcttgttccatctcga

SEQ ID NO: 749 - DNA, 48 bp, synthetic construct
  source 1..48: mol_type = other DNA
tcatcagttttcctcttttgaggaacaagttttcttgtgtcaatcttc

SEQ ID NO: 750 - DNA, 48 bp, synthetic construct
  source 1..48: mol_type = other DNA
tcttgttctgtcctcttttgaggaacaagttttcttgttgacgttcag

SEQ ID NO: 751 - DNA, 48 bp, synthetic construct
  source 1..48: mol_type = other DNA
tttcttgctatcctcttttgaggaacaagttttcttgtaccagggcag

SEQ ID NO: 752 - DNA, 48 bp, synthetic construct
  source 1..48: mol_type = other DNA
gcgcaatagttcctcttttgaggaacaagttttcttgttttattgatg

SEQ ID NO: 753 - DNA, 48 bp, synthetic construct
  source 1..48: mol_type = other DNA
tgctcaacagtcctcttttgaggaacaagttttcttgtcctttgagac

SEQ ID NO: 754 - DNA, 38 bp, synthetic construct
  source 1..38: mol_type = other DNA
acctggaaggaacaatcaaatcaaattactgacattaa

SEQ ID NO: 755 - DNA, 38 bp, synthetic construct
  source 1..38: mol_type = other DNA
ctttaaaacaggcttgagcagcattgttagagagaacc

SEQ ID NO: 756 - DNA, 38 bp, synthetic construct
  source 1..38: mol_type = other DNA
actgttgaggccgacgcggaagcccaccaagggacctt

SEQ ID NO: 757 - DNA, 38 bp, synthetic construct
  source 1..38: mol_type = other DNA
ctgtgggacctcttccccctctcccctttccccccaga

SEQ ID NO: 758 - DNA, 38 bp, synthetic construct
  source 1..38: mol_type = other DNA
atcggaggactttccggcccagtctgagtgctcctacc

SEQ ID NO: 759 - DNA, 38 bp, synthetic construct
  source 1..38: mol_type = other DNA
taggaccccagagggttagagccacacaaaacagcagc

SEQ ID NO: 760 - DNA, 38 bp, synthetic construct
  source 1..38: mol_type = other DNA
tggctcagaacgatctgactggaggttagtttgcaaga

SEQ ID NO: 761 - DNA, 38 bp, synthetic construct
  source 1..38: mol_type = other DNA
ccatgcactggaaacgccaactgcagactaaaggctgt

SEQ ID NO: 762 - DNA, 38 bp, synthetic construct
  source 1..38: mol_type = other DNA
tcaatcagttacctgagtgcacagggctggtgccagga

SEQ ID NO: 763 - DNA, 38 bp, synthetic construct
  source 1..38: mol_type = other DNA
actcattaatatacttaatgggtctggagacgcctgcc

SEQ ID NO: 764 - DNA, 38 bp, synthetic construct
  source 1..38: mol_type = other DNA
cagcagaggatgaagaaggaaaaatgagagagactcag

SEQ ID NO: 765 - DNA, 38 bp, synthetic construct
  source 1..38: mol_type = other DNA
aagagaaccggtgattaaggactgcgagtgagagacag

SEQ ID NO: 766 - DNA, 38 bp, synthetic construct
  source 1..38: mol_type = other DNA
tgagggggcaggaaagcagaggagagcatttcaggggc

SEQ ID NO: 767 - DNA, 38 bp, synthetic construct
  source 1..38: mol_type = other DNA
catgcgttcaatcttccacaaatgcttacttactgagc

SEQ ID NO: 768 - DNA, 38 bp, synthetic construct
  source 1..38: mol_type = other DNA
actcattaccatcaaggcacagcatcatcctgctccgg

SEQ ID NO: 769 - DNA, 38 bp, synthetic construct
  source 1..38: mol_type = other DNA
ctaagtccccacgtacgccattaaacaacggtcaaatg

SEQ ID NO: 770 - DNA, 38 bp, synthetic construct
  source 1..38: mol_type = other DNA
gtaacatgttcgtgtgtgtagagtgcttcttacagcat

SEQ ID NO: 771 - DNA, 38 bp, synthetic construct
  source 1..38: mol_type = other DNA
taaagcgggactgaacacccagcaccaccactgcgaca

SEQ ID NO: 772 - DNA, 38 bp, synthetic construct
  source 1..38: mol_type = other DNA
ccagcccagtgtttcacaacctgatttcatcattgtcc

SEQ ID NO: 773 - DNA, 38 bp, synthetic construct
  source 1..38: mol_type = other DNA
cctccctgcagcctttttggagttttccgggtgcccca

SEQ ID NO: 774 - DNA, 38 bp, synthetic construct
  source 1..38: mol_type = other DNA
ctgagaatgcgtgatctagccctatgtgcttttctgta

SEQ ID NO: 775 - DNA, 38 bp, synthetic construct
  source 1..38: mol_type = other DNA
atttggccacatgttctatctctagaaaggtcaatgtt

SEQ ID NO: 776 - DNA, 26 bp, synthetic construct
  source 1..26: mol_type = other DNA
gcacacacactaagtcaatttgaata

SEQ ID NO: 777 - DNA, 48 bp, synthetic construct
  source 1..48: mol_type = other DNA
cagccagaggtcctcttttgaggaacaagttttcttgtctagctctgc

SEQ ID NO: 778 - DNA, 48 bp, synthetic construct
  source 1..48: mol_type = other DNA
cacagagcagtcctcttttgaggaacaagttttcttgtggctcctatg

SEQ ID NO: 779 - DNA, 48 bp, synthetic construct
  source 1..48: mol_type = other DNA
agtcagtcaatcctcttttgaggaacaagttttcttgtcaggaaaatc

SEQ ID NO: 780 - DNA, 48 bp, synthetic construct
  source 1..48: mol_type = other DNA
acactaaatgtcctcttttgaggaacaagttttcttgtacacacctgc

SEQ ID NO: 781 - DNA, 48 bp, synthetic construct
  source 1..48: mol_type = other DNA
agctcaccattcctcttttgaggaacaagttttcttgttcagggcagg

SEQ ID NO: 782 - DNA, 48 bp, synthetic construct
  source 1..48: mol_type = other DNA
tcattggttatcctcttttgaggaacaagttttcttgtgaccttccct

SEQ ID NO: 783 - DNA, 47 bp, synthetic construct
  source 1..47: mol_type = other DNA
ggatcctttctagcccgtgaagaggctgatgatctctggagggccca

SEQ ID NO: 784 - DNA, 47 bp, synthetic construct
  source 1..47: mol_type = other DNA
cagaaccctcctgggagatggggaaacttttctttagttaacaggcc

SEQ ID NO: 785 - DNA, 47 bp, synthetic construct
  source 1..47: mol_type = other DNA
ctgttctcatgcttgggttcctgtcctaaccgtgaaggagtatttcg

SEQ ID NO: 786 - DNA, 47 bp, synthetic construct
  source 1..47: mol_type = other DNA
cagattgggaacaaactgcaatagaagcaagcattggaactctcgac

SEQ ID NO: 787 - DNA, 47 bp, synthetic construct
  source 1..47: mol_type = other DNA
ccagagcatgcaggctcgggaaccccggaatccacacaccaactttc

SEQ ID NO: 788 - DNA, 43 bp, synthetic construct
  source 1..43: mol_type = other DNA
ctgtccacaaggtggtgcactgcttccatcaacatcatgggtc

SEQ ID NO: 789 - DNA, 48 bp, synthetic construct
  source 1..48: mol_type = other DNA
acagcaggtttcctcttttgaggaacaagttttcttgttacctgccat

SEQ ID NO: 790 - DNA, 48 bp, synthetic construct
  source 1..48: mol_type = other DNA
aaacctgcaatcctcttttgaggaacaagttttcttgtgtgcaagtgc

SEQ ID NO: 791 - DNA, 48 bp, synthetic construct
  source 1..48: mol_type = other DNA
cacccagagatcctcttttgaggaacaagttttcttgtggacaggact

SEQ ID NO: 792 - DNA, 48 bp, synthetic construct
  source 1..48: mol_type = other DNA
gggcaaggcctcctcttttgaggaacaagttttcttgtaggaatgggg

SEQ ID NO: 793 - DNA, 48 bp, synthetic construct
  source 1..48: mol_type = other DNA
ctggaagcagtcctcttttgaggaacaagttttcttgtggaggccatg

SEQ ID NO: 794 - DNA, 48 bp, synthetic construct
  source 1..48: mol_type = other DNA
ggctgtgggttcctcttttgaggaacaagttttcttgttctaaggctt

SEQ ID NO: 795 - DNA, 50 bp, synthetic construct
  source 1..50: mol_type = other DNA
acccttccccatatagcgagtcttatcattgtcccggagctctagggcct

SEQ ID NO: 796 - DNA, 50 bp, synthetic construct
  source 1..50: mol_type = other DNA
catagataccagttgaagcaccactgggcacagcagctctgaagagacct

SEQ ID NO: 797 - DNA, 50 bp, synthetic construct
  source 1..50: mol_type = other DNA
tttgaggtgaagagatcaacctcaacagtgggattcccgcgagagtcaaa

SEQ ID NO: 798 - DNA, 50 bp, synthetic construct
  source 1..50: mol_type = other DNA
gatctccctggcatggatcttgagaatagacatggtgaacttctagccac

SEQ ID NO: 799 - DNA, 50 bp, synthetic construct
  source 1..50: mol_type = other DNA
tgggtctcgtcgcctaggagaggaagcggagggtgctgcagacaccgagg

SEQ ID NO: 800 - DNA, 49 bp, synthetic construct
  source 1..49: mol_type = other DNA
tgaacgtaaagccggcgagatctccgtgctccgggtacccacagatact

SEQ ID NO: 801 - DNA, 28 bp, synthetic construct
  source 1..28: mol_type = other DNA
gagtgcacagggctggtgccaggaactc

SEQ ID NO: 802 - DNA, 28 bp, synthetic construct
  source 1..28: mol_type = other DNA
attaatatacttaatgggtctggagacg

SEQ ID NO: 803 - DNA, 38 bp, synthetic construct
  source 1..38: mol_type = other DNA
ttttttttttttttttcaaagttttcaaaacagtatat

SEQ ID NO: 804 - DNA, 38 bp, synthetic construct
  source 1..38: mol_type = other DNA
tttattttacaatagcaaccaactccccagtttgtttc

SEQ ID NO: 805 - DNA, 38 bp, synthetic construct
  source 1..38: mol_type = other DNA
aattgtgacatctagatggcttaagattactttctggt

SEQ ID NO: 806 - DNA, 38 bp, synthetic construct
  source 1..38: mol_type = other DNA
ggtcacccatgctgaacaatatttttcaatcttccaaa

SEQ ID NO: 807 - DNA, 38 bp, synthetic construct
  source 1..38: mol_type = other DNA
cagcaaagactcaaaagagattctgcatttcacatcag

SEQ ID NO: 808 - DNA, 38 bp, synthetic construct
  source 1..38: mol_type = other DNA
ttcacaagttcaagagtcttccatttatcttagctttt

SEQ ID NO: 809 - DNA, 38 bp, synthetic construct
  source 1..38: mol_type = other DNA
ggaataaattatctttgaggtagaaggacaatgacgaa

SEQ ID NO: 810 - DNA, 38 bp, synthetic construct
  source 1..38: mol_type = other DNA
gccacttaattccttgtgtctgcataaaagcagattta

SEQ ID NO: 811 - DNA, 38 bp, synthetic construct
  source 1..38: mol_type = other DNA
ttcatcacaacttcatttatgtgaataaagcagatgat

SEQ ID NO: 812 - DNA, 38 bp, synthetic construct
  source 1..38: mol_type = other DNA
gataaaatgttctcttattcttgtttaatcagtagtgg

SEQ ID NO: 813 - DNA, 48 bp, synthetic construct
  source 1..48: mol_type = other DNA
tagtgatgcctcctcttttgaggaacaagttttcttgtagaaactgtg

SEQ ID NO: 814 - DNA, 48 bp, synthetic construct
  source 1..48: mol_type = other DNA
aaaggaaggctcctcttttgaggaacaagttttcttgtttttagttac

SEQ ID NO: 815 - DNA, 48 bp, synthetic construct
  source 1..48: mol_type = other DNA
tttcttcttttcctcttttgaggaacaagttttcttgtccattttcca

SEQ ID NO: 816 - DNA, 48 bp, synthetic construct
  source 1..48: mol_type = other DNA
ataatccatttcctcttttgaggaacaagttttcttgtccccatcccc

SEQ ID NO: 817 - DNA, 48 bp, synthetic construct
  source 1..48: mol_type = other DNA
agctgaagaatcctcttttgaggaacaagttttcttgtaggggtgtta

SEQ ID NO: 818 - DNA, 48 bp, synthetic construct
  source 1..48: mol_type = other DNA
ctgagtccagtcctcttttgaggaacaagttttcttgtctgataccac

SEQ ID NO: 819 - DNA, 38 bp, synthetic construct
  source 1..38: mol_type = other DNA
acattgaaaggtaaacattaatatttcaaatctgatgt

SEQ ID NO: 820 - DNA, 38 bp, synthetic construct
  source 1..38: mol_type = other DNA
ctaactaaaaatgtacagaatgaaaactagaaaatttc

SEQ ID NO: 821 - DNA, 38 bp, synthetic construct
  source 1..38: mol_type = other DNA
aaccccagattatcttcaaccttgctccctccaccaat

SEQ ID NO: 822 - DNA, 38 bp, synthetic construct
  source 1..38: mol_type = other DNA
catactttgacatttatctatttccttctccacttatg

SEQ ID NO: 823 - DNA, 38 bp, synthetic construct
  source 1..38: mol_type = other DNA
gatgtaattggcttgctatagaaactacagttcagatg

SEQ ID NO: 824 - DNA, 38 bp, synthetic construct
  source 1..38: mol_type = other DNA
ctttgaatgtatgaactacaatgaacaataaagtcctc

SEQ ID NO: 825 - DNA, 25 bp, synthetic construct
  source 1..25: mol_type = other DNA
ttcttttgaagcatattttggcttc

SEQ ID NO: 826 - DNA, 25 bp, synthetic construct
  source 1..25: mol_type = other DNA
agctttaagataatcttatgacaag

SEQ ID NO: 827 - DNA, 48 bp, synthetic construct
  source 1..48: mol_type = other DNA
aagggtcacatcctcttttgaggaacaagttttcttgtctgattcact

SEQ ID NO: 828 - DNA, 48 bp, synthetic construct
  source 1..48: mol_type = other DNA
taataaattctcctcttttgaggaacaagttttcttgtcattcttacc

SEQ ID NO: 829 - DNA, 48 bp, synthetic construct
  source 1..48: mol_type = other DNA
taacacaaggtcctcttttgaggaacaagttttcttgttttagttgat

SEQ ID NO: 830 - DNA, 48 bp, synthetic construct
  source 1..48: mol_type = other DNA
aagcacttggtcctcttttgaggaacaagttttcttgtacaaaaataa

SEQ ID NO: 831 - DNA, 48 bp, synthetic construct
  source 1..48: mol_type = other DNA
tacttttcaatcctcttttgaggaacaagttttcttgtaaatgtaaag

SEQ ID NO: 832 - DNA, 48 bp, synthetic construct
  source 1..48: mol_type = other DNA
caaactagtgtcctcttttgaggaacaagttttcttgtaggacaaagg

SEQ ID NO: 833 - DNA, 38 bp, synthetic construct
  source 1..38: mol_type = other DNA
attttgtcctcatctcaacaatgatcagctattggaac

SEQ ID NO: 834 - DNA, 38 bp, synthetic construct
  source 1..38: mol_type = other DNA
tgcatgaaactgaacaatttaaacctggagctggtaat

SEQ ID NO: 835 - DNA, 38 bp, synthetic construct
  source 1..38: mol_type = other DNA
gttcttaagaccaattcagaacaaaggcaggttgccct

SEQ ID NO: 836 - DNA, 38 bp, synthetic construct
  source 1..38: mol_type = other DNA
taaaacaggtttgaccttttccttcactcttcctcctg

SEQ ID NO: 837 - DNA, 38 bp, synthetic construct
  source 1..38: mol_type = other DNA
tcccaccctctgtgagtgatttaaaaacggaaaaggtc

SEQ ID NO: 838 - DNA, 38 bp, synthetic construct
  source 1..38: mol_type = other DNA
aaagcccagccaggcctacatttagagaaattttaaaa

SEQ ID NO: 839 - DNA, 25 bp, synthetic construct
  source 1..25: mol_type = other DNA
aaatttttcctttcaattttggcca

SEQ ID NO: 840 - DNA, 25 bp, synthetic construct
  source 1..25: mol_type = other DNA
attatttccaatttttattttattc

SEQ ID NO: 841 - DNA, 48 bp, synthetic construct
  source 1..48: mol_type = other DNA
ttaaaacttatcctcttttgaggaacaagttttcttgtagcatggtaa

SEQ ID NO: 842 - DNA, 48 bp, synthetic construct
  source 1..48: mol_type = other DNA
aatgttaagctcctcttttgaggaacaagttttcttgttgttttcatc

SEQ ID NO: 843 - DNA, 48 bp, synthetic construct
  source 1..48: mol_type = other DNA
cactgatatttcctcttttgaggaacaagttttcttgtattctactat

SEQ ID NO: 844 - DNA, 48 bp, synthetic construct
  source 1..48: mol_type = other DNA
aaaaagcccttcctcttttgaggaacaagttttcttgttcttgagcag

SEQ ID NO: 845 - DNA, 48 bp, synthetic construct
  source 1..48: mol_type = other DNA
atttgatgattcctcttttgaggaacaagttttcttgtaaaaaggaga

SEQ ID NO: 846 - DNA, 48 bp, synthetic construct
  source 1..48: mol_type = other DNA
acatttctaatcctcttttgaggaacaagttttcttgtggtataatta

SEQ ID NO: 847 - DNA, 38 bp, synthetic construct
  source 1..38: mol_type = other DNA
gagaagctgttgcatgagaaatcatgtctccatctcca

SEQ ID NO: 848 - DNA, 38 bp, synthetic construct
  source 1..38: mol_type = other DNA
ttttgctatgcgttatctgaggattgtttctgaaagag

SEQ ID NO: 849 - DNA, 38 bp, synthetic construct
  source 1..38: mol_type = other DNA
atctatttaggcagtgtatgtatgtgtcagcatgtaaa

SEQ ID NO: 850 - DNA, 38 bp, synthetic construct
  source 1..38: mol_type = other DNA
aagtaaagaactgaaaatagacaaaccttgtaaatgca

SEQ ID NO: 851 - DNA, 38 bp, synthetic construct
  source 1..38: mol_type = other DNA
cttcaaaaccaattgtggctcaagtgtaggtggttccc

SEQ ID NO: 852 - DNA, 38 bp, synthetic construct
  source 1..38: mol_type = other DNA
caaggctggtaccaatgagactggggtttgggaattag

SEQ ID NO: 853 - DNA, 25 bp, synthetic construct
  source 1..25: mol_type = other DNA
ttggtcatcatccctcctgctgccc

SEQ ID NO: 854 - DNA, 25 bp, synthetic construct
  source 1..25: mol_type = other DNA
agcagtggtcagtcatttttcatga

SEQ ID NO: 855 - DNA, 48 bp, synthetic construct
  source 1..48: mol_type = other DNA
gggatggacttcctcttttgaggaacaagttttcttgtaggaaaatga

SEQ ID NO: 856 - DNA, 48 bp, synthetic construct
  source 1..48: mol_type = other DNA
agtgtctatttcctcttttgaggaacaagttttcttgtataaggagag

SEQ ID NO: 857 - DNA, 48 bp, synthetic construct
  source 1..48: mol_type = other DNA
ttcctgtaactcctcttttgaggaacaagttttcttgtttaaggagca

SEQ ID NO: 858 - DNA, 48 bp, synthetic construct
  source 1..48: mol_type = other DNA
agctcactactcctcttttgaggaacaagttttcttgtaaaacccagc

SEQ ID NO: 859 - DNA, 48 bp, synthetic construct
  source 1..48: mol_type = other DNA
tacctgcttatcctcttttgaggaacaagttttcttgttcgtagtggc

SEQ ID NO: 860 - DNA, 48 bp, synthetic construct
  source 1..48: mol_type = other DNA
cagagtggtatcctcttttgaggaacaagttttcttgtgaagagatac

SEQ ID NO: 861 - DNA, 38 bp, synthetic construct
  source 1..38: mol_type = other DNA
ggagtaggaattaaaccacacaatgttatttagggact

SEQ ID NO: 862 - DNA, 38 bp, synthetic construct
  source 1..38: mol_type = other DNA
aagccatgcccctaacaagaaaacaagccaaaaggaaa

SEQ ID NO: 863 - DNA, 38 bp, synthetic construct
  source 1..38: mol_type = other DNA
gtattaggcattctctgggaaggcatgcatttttttcc

SEQ ID NO: 864 - DNA, 38 bp, synthetic construct
  source 1..38: mol_type = other DNA
catgtctctggggccaaaaaccttataccaagtaccta

SEQ ID NO: 865 - DNA, 38 bp, synthetic construct
  source 1..38: mol_type = other DNA
ttggcacccgaatatatttgtagaatgaatgaatacat

SEQ ID NO: 866 - DNA, 38 bp, synthetic construct
  source 1..38: mol_type = other DNA
gaaaaaaaataaacagtaacctttctcctatattctac

SEQ ID NO: 867 - DNA, 25 bp, synthetic construct
  source 1..25: mol_type = other DNA
tttccaagccaattaataagcaagt

SEQ ID NO: 868 - DNA, 25 bp, synthetic construct
  source 1..25: mol_type = other DNA
gtcttttcgtcatgattttttttgt

SEQ ID NO: 869 - DNA, 48 bp, synthetic construct
  source 1..48: mol_type = other DNA
tttctgtttatcctcttttgaggaacaagttttcttgtggatttaaca

SEQ ID NO: 870 - DNA, 48 bp, synthetic construct
  source 1..48: mol_type = other DNA
aaatggttgatcctcttttgaggaacaagttttcttgtgataacagtc

SEQ ID NO: 871 - DNA, 48 bp, synthetic construct
  source 1..48: mol_type = other DNA
acttctgttttcctcttttgaggaacaagttttcttgtgatgaagagt

SEQ ID NO: 872 - DNA, 48 bp, synthetic construct
  source 1..48: mol_type = other DNA
atcacttcattcctcttttgaggaacaagttttcttgttccatttttg

SEQ ID NO: 873 - DNA, 48 bp, synthetic construct
  source 1..48: mol_type = other DNA
tgtttttgtttcctcttttgaggaacaagttttcttgtgcatctccaa

SEQ ID NO: 874 - DNA, 48 bp, synthetic construct
  source 1..48: mol_type = other DNA
gtcagaataatcctcttttgaggaacaagttttcttgtatgccttttg

SEQ ID NO: 875 - DNA, 38 bp, synthetic construct
  source 1..38: mol_type = other DNA
gagcagatatatttcatttagcatttagtatcatcttc

SEQ ID NO: 876 - DNA, 38 bp, synthetic construct
  source 1..38: mol_type = other DNA
atcaatactcgtatgaacgaaaaaataaaaagccctct

SEQ ID NO: 877 - DNA, 38 bp, synthetic construct
  source 1..38: mol_type = other DNA
cttattcccactctacaacgcatgtcaaaggtgatctg

SEQ ID NO: 878 - DNA, 38 bp, synthetic construct
  source 1..38: mol_type = other DNA
tttagtttttccttagtatcgaacatatcacagctact

SEQ ID NO: 879 - DNA, 38 bp, synthetic construct
  source 1..38: mol_type = other DNA
caatgaagtttcttctcaactaaagaaacacagtctct

SEQ ID NO: 880 - DNA, 38 bp, synthetic construct
  source 1..38: mol_type = other DNA
tagagaatttgttcctgtgtttccaccataagataaat

SEQ ID NO: 881 - DNA, 25 bp, synthetic construct
  source 1..25: mol_type = other DNA
gagataaagtgcactttggtttacg

SEQ ID NO: 882 - DNA, 25 bp, synthetic construct
  source 1..25: mol_type = other DNA
tctttcaagcacttaagaacagcac

SEQ ID NO: 883 - DNA, 48 bp, synthetic construct
  source 1..48: mol_type = other DNA
tttattcagttcctcttttgaggaacaagttttcttgttttaataaat

SEQ ID NO: 884 - DNA, 48 bp, synthetic construct
  source 1..48: mol_type = other DNA
ttttactaaatcctcttttgaggaacaagttttcttgtttggtaaatg

SEQ ID NO: 885 - DNA, 48 bp, synthetic construct
  source 1..48: mol_type = other DNA
caaaagaatctcctcttttgaggaacaagttttcttgtttttttttat

SEQ ID NO: 886 - DNA, 48 bp, synthetic construct
  source 1..48: mol_type = other DNA
tgctcatatgtcctcttttgaggaacaagttttcttgttcttcctgtc

SEQ ID NO: 887 - DNA, 48 bp, synthetic construct
  source 1..48: mol_type = other DNA
tctaagccagtcctcttttgaggaacaagttttcttgtaccaatgagc

SEQ ID NO: 888 - DNA, 48 bp, synthetic construct
  source 1..48: mol_type = other DNA
aaataccttttcctcttttgaggaacaagttttcttgtaaaactagtt

SEQ ID NO: 889 - DNA, 38 bp, synthetic construct
  source 1..38: mol_type = other DNA
gttacatcttgaaccatttaactgtaataaaagcagaa

SEQ ID NO: 890 - DNA, 38 bp, synthetic construct
  source 1..38: mol_type = other DNA
tgtttagttaatgaattaaagaacaaaccctgagccct

SEQ ID NO: 891 - DNA, 38 bp, synthetic construct
  source 1..38: mol_type = other DNA
tttatcagtctcctggctttaaactaagccaatgagga

SEQ ID NO: 892 - DNA, 38 bp, synthetic construct
  source 1..38: mol_type = other DNA
agtgatttgggggattcctgaaactaggaaaaatgtct

SEQ ID NO: 893 - DNA, 38 bp, synthetic construct
  source 1..38: mol_type = other DNA
ctttattttgaaagaaacttgcatttttctttcttttt

SEQ ID NO: 894 - DNA, 38 bp, synthetic construct
  source 1..38: mol_type = other DNA
ttttttttttttaatctcaaaggcaattgagtgggtct

SEQ ID NO: 895 - DNA, 38 bp, synthetic construct
  source 1..38: mol_type = other DNA
tctgggccagacctatttaatttacgaaacatagtacc

SEQ ID NO: 896 - DNA, 38 bp, synthetic construct
  source 1..38: mol_type = other DNA
ttgcagagaataggcattgaaatattatttaaacaatc

SEQ ID NO: 897 - DNA, 38 bp, synthetic construct
  source 1..38: mol_type = other DNA
aaaccaaagatgttcttctatcttcagctgtcagtgat

SEQ ID NO: 898 - DNA, 38 bp, synthetic construct
  source 1..38: mol_type = other DNA
ctaatgccctcatctctcttatcctcaggacccagaat

SEQ ID NO: 899 - DNA, 38 bp, synthetic construct
  source 1..38: mol_type = other DNA
tgggtggtaactagccaagcagctagttaccaaatcgg

SEQ ID NO: 900 - DNA, 38 bp, synthetic construct
  source 1..38: mol_type = other DNA
gagaatcccgggtcctctctttagggggaggtccctgg

SEQ ID NO: 901 - DNA, 38 bp, synthetic construct
  source 1..38: mol_type = other DNA
gccgaagcccgcccacctttcggtggagccggaccgct

SEQ ID NO: 902 - DNA, 38 bp, synthetic construct
  source 1..38: mol_type = other DNA
ttcgcacccgtgctctttcgagcacacccaccccgttt

SEQ ID NO: 903 - DNA, 38 bp, synthetic construct
  source 1..38: mol_type = other DNA
acgggggtccctcggtcagctaccgaggagagctcgct

SEQ ID NO: 904 - DNA, 38 bp, synthetic construct
  source 1..38: mol_type = other DNA
ggcccacactcctgagggaatgtgggaaccggcgttag

SEQ ID NO: 905 - DNA, 38 bp, synthetic construct
  source 1..38: mol_type = other DNA
ccactccgaagtgcgtataacgcgcacgccggcggact

SEQ ID NO: 906 - DNA, 38 bp, synthetic construct
  source 1..38: mol_type = other DNA
tcatgctgtcggtgatttcacctccagtatggaaccac

SEQ ID NO: 907 - DNA, 38 bp, synthetic construct
  source 1..38: mol_type = other DNA
gctatgtagcgaccactgtcgtgcttttcgctgaagaa

SEQ ID NO: 908 - DNA, 25 bp, synthetic construct
  source 1..25: mol_type = other DNA
cttgcgttctcgagcgatacgagca

SEQ ID NO: 909 - DNA, 26 bp, synthetic construct
  source 1..26: mol_type = other DNA
agacggaaacccgaggtacgggtatc

SEQ ID NO: 910 - DNA, 50 bp, synthetic construct
  source 1..50: mol_type = other DNA
cgcgagcagccgcccgtacggagtctagaggcgtggatctgacatacctc

SEQ ID NO: 911 - DNA, 26 bp, synthetic construct
  source 1..26: mol_type = other DNA
cgacaactccccaaccccgtagccga

SEQ ID NO: 912 - DNA, 25 bp, synthetic construct
  source 1..25: mol_type = other DNA
tttaatatcagcatcagggcgaaga

SEQ ID NO: 913 - DNA, 38 bp, synthetic construct
  source 1..38: mol_type = other DNA
gattgtcaacaggtttcttgatgtaaaacggtttgaca

SEQ ID NO: 914 - DNA, 38 bp, synthetic construct
  source 1..38: mol_type = other DNA
tcgacaccacggtaaaagtgcgcgccgcagctctcgcg

SEQ ID NO: 915 - DNA, 38 bp, synthetic construct
  source 1..38: mol_type = other DNA
aaagagcccggacacgaacgttttacgaagattcggtt

SEQ ID NO: 916 - DNA, 38 bp, synthetic construct
  source 1..38: mol_type = other DNA
taaaaccgtagtaggcaagtgcctctagcacacggggt

SEQ ID NO: 917 - DNA, 38 bp, synthetic construct
  source 1..38: mol_type = other DNA
gcaatctcactgggacatataatatcgtccccgtagat

SEQ ID NO: 918 - DNA, 38 bp, synthetic construct
  source 1..38: mol_type = other DNA
gcctatggttccggcgttaccaaaatggatttgggtcg

SEQ ID NO: 919 - DNA, 38 bp, synthetic construct
  source 1..38: mol_type = other DNA
ctttgactattgcccagaatatcatggactctagctca

SEQ ID NO: 920 - DNA, 38 bp, synthetic construct
  source 1..38: mol_type = other DNA
aatgtgaacccatttcccattgtggaaaatagttccca

SEQ ID NO: 921 - DNA, 38 bp, synthetic construct
  source 1..38: mol_type = other DNA
tcgtatcgtctcgccatctacgattccgtagtgtgagc

SEQ ID NO: 922 - DNA, 38 bp, synthetic construct
  source 1..38: mol_type = other DNA
ggatacgatcgagatatgaatatagctctggtgggaga

SEQ ID NO: 923 - DNA, 38 bp, synthetic construct
  source 1..38: mol_type = other DNA
aaactccacaccaggcgatcggagatggaatcggatgc

SEQ ID NO: 924 - DNA, 38 bp, synthetic construct
  source 1..38: mol_type = other DNA
agacgataagtctatcgtcgcaagcgaaccatctacgc

SEQ ID NO: 925 - DNA, 38 bp, synthetic construct
  source 1..38: mol_type = other DNA
tgccctgctgagccagacgctggttgatcgattgatca

SEQ ID NO: 926 - DNA, 38 bp, synthetic construct
  source 1..38: mol_type = other DNA
ttcaggtctataccaacggatttgagccggcgtctgat

SEQ ID NO: 927 - DNA, 38 bp, synthetic construct
  source 1..38: mol_type = other DNA
gaaagcaccgacccctttctggaggtacatattcatat

SEQ ID NO: 928 - DNA, 25 bp, synthetic construct
  source 1..25: mol_type = other DNA
caggctccttacaggcagcccgatc

SEQ ID NO: 929 - DNA, 26 bp, synthetic construct
  source 1..26: mol_type = other DNA
tattttattattcttcggaactgtaa

SEQ ID NO: 930 - DNA, 50 bp, synthetic construct
  source 1..50: mol_type = other DNA
acactccgttccctacaacgagcctaaattcatatgactcgttatagcgg

SEQ ID NO: 931 - DNA, 25 bp, synthetic construct
  source 1..25: mol_type = other DNA
accgcgtgtctgaacggatgctggt

SEQ ID NO: 932 - DNA, 26 bp, synthetic construct
  source 1..26: mol_type = other DNA
ttgtaaaacatccggatcccatgaca

SEQ ID NO: 933 - DNA, 38 bp, synthetic construct
  source 1..38: mol_type = other DNA
aggatttgtcatgtaagaaaccttctctatttatctga

SEQ ID NO: 934 - DNA, 38 bp, synthetic construct
  source 1..38: mol_type = other DNA
ccgcgatcaccattcgcctcccgtagcttagcgatagc

SEQ ID NO: 935 - DNA, 38 bp, synthetic construct
  source 1..38: mol_type = other DNA
taaggtacgacgggtcgcctcgtcattaccagaaccta

SEQ ID NO: 936 - DNA, 38 bp, synthetic construct
  source 1..38: mol_type = other DNA
aggtcggatgctttgtgagcaattcgtcccttaagtaa

SEQ ID NO: 937 - DNA, 38 bp, synthetic construct
  source 1..38: mol_type = other DNA
gcaattgctgtaaagtcgtcactgtgcggatcaccgct

SEQ ID NO: 938 - DNA, 38 bp, synthetic construct
  source 1..38: mol_type = other DNA
tccagtagcgacagaagcaattgattggtaaatttcga

SEQ ID NO: 939 - DNA, 38 bp, synthetic construct
  source 1..38: mol_type = other DNA
gagaaagatcgcgaggaagatcaatacataaagagttg

SEQ ID NO: 940 - DNA, 38 bp, synthetic construct
  source 1..38: mol_type = other DNA
aacttctttgttgtcttcgacatgggtaatcctcatgt

SEQ ID NO: 941 - DNA, 38 bp, synthetic construct
  source 1..38: mol_type = other DNA
ttgaatggccggcgtctattagtagatgccggagtttg

SEQ ID NO: 942 - DNA, 38 bp, synthetic construct
  source 1..38: mol_type = other DNA
ctgcgattgctgagggaatcgggtttccatcttttagg

SEQ ID NO: 943 - DNA, 38 bp, synthetic construct
  source 1..38: mol_type = other DNA
agaccttgcattgccttaacaataagctcgcagtcgga

SEQ ID NO: 944 - DNA, 38 bp, synthetic construct
  source 1..38: mol_type = other DNA
attcgtagcgaaaattggaatggttagttccatattta

SEQ ID NO: 945 - DNA, 38 bp, synthetic construct
  source 1..38: mol_type = other DNA
agtacgaacgccatgcggctacaggaagctctacacca

SEQ ID NO: 946 - DNA, 38 bp, synthetic construct
  source 1..38: mol_type = other DNA
ccaacagtctgggttgccactttaggcacctcgacttt

SEQ ID NO: 947 - DNA, 38 bp, synthetic construct
  source 1..38: mol_type = other DNA
gatggtgtatttgcgattctgcgcagagctctgacgaa

SEQ ID NO: 948 - DNA, 38 bp, synthetic construct
  source 1..38: mol_type = other DNA
cgctacaggttactttgtaagcctgtgaacgcgagtta

SEQ ID NO: 949 - DNA, 38 bp, synthetic construct
  source 1..38: mol_type = other DNA
gagctgatccattcagcgaccccgttagcgaagttgct

SEQ ID NO: 950 - DNA, 38 bp, synthetic construct
  source 1..38: mol_type = other DNA
tggggcgacagtcacgtcgccagttccgccattgtcga

SEQ ID NO: 951 - DNA, 38 bp, synthetic construct
  source 1..38: mol_type = other DNA
cgagaacgaactgagtaaagttagaagccatgcttcaa

SEQ ID NO: 952 - DNA, 38 bp, synthetic construct
  source 1..38: mol_type = other DNA
actccggttgagggctctatctagagagccgttgcctg

SEQ ID NO: 953 - DNA, 38 bp, synthetic construct
  source 1..38: mol_type = other DNA
attaatgctaacgcatctaaggtatggaccatcgagaa

SEQ ID NO: 954 - DNA, 38 bp, synthetic construct
  source 1..38: mol_type = other DNA
aggagactttacgtacgcgccagttgttggccatacgg

SEQ ID NO: 955 - DNA, 38 bp, synthetic construct
  source 1..38: mol_type = other DNA
attgtacccctcgatgcatggctgagatttgggcctta

SEQ ID NO: 956 - DNA, 38 bp, synthetic construct
  source 1..38: mol_type = other DNA
gcagtgccctgtctctccacagtccacccgtagggagc

SEQ ID NO: 957 - DNA, 38 bp, synthetic construct
  source 1..38: mol_type = other DNA
gtcaacgcttatgatggactcacccgttattacgtcag

SEQ ID NO: 958 - DNA, 38 bp, synthetic construct
  source 1..38: mol_type = other DNA
taactgttcctgacatgtaggagcatcccacgggggcc

SEQ ID NO: 959 - DNA, 38 bp, synthetic construct
  source 1..38: mol_type = other DNA
gtaaggccctcgagcatgttacctacaggtaggagcca

SEQ ID NO: 960 - DNA, 38 bp, synthetic construct
  source 1..38: mol_type = other DNA
gtcgacaacgaatgagaaaggcaccttttcccacacta

SEQ ID NO: 961 - DNA, 38 bp, synthetic construct
  source 1..38: mol_type = other DNA
tacctagtgggttcaagatacctagagacgacaaccat

SEQ ID NO: 962 - DNA, 38 bp, synthetic construct
  source 1..38: mol_type = other DNA
gccaaacgtgcatcgtttatgtaaaaccatatcacgat

SEQ ID NO: 963 - DNA, 25 bp, synthetic construct
  source 1..25: mol_type = other DNA
acgtcgcgatatgttgcacgttgtc

SEQ ID NO: 964 - DNA, 26 bp, synthetic construct
  source 1..26: mol_type = other DNA
tggaagtttgcagctggatacgacag

SEQ ID NO: 965 - DNA, 50 bp, synthetic construct
  source 1..50: mol_type = other DNA
acggccatctaacttgatgttagtagcaacgttctgcggcacttcgatgt

SEQ ID NO: 966 - DNA, 25 bp, synthetic construct
  source 1..25: mol_type = other DNA
aagtcaagttttggcttacagggaa

SEQ ID NO: 967 - DNA, 26 bp, synthetic construct
  source 1..26: mol_type = other DNA
gaggctgtagcaggagcgtgcgtcga

SEQ ID NO: 968 - DNA, 38 bp, synthetic construct
  source 1..38: mol_type = other DNA
gggagaagccgaaaccggctttctcctcgtacgggcga

SEQ ID NO: 969 - DNA, 38 bp, synthetic construct
  source 1..38: mol_type = other DNA
ccccacgatgacccacttcgcttgtaggcaccttgatc

SEQ ID NO: 970 - DNA, 38 bp, synthetic construct
  source 1..38: mol_type = other DNA
tatcgatgtgacacttaacgccccccgtgaatacggag

SEQ ID NO: 971 - DNA, 38 bp, synthetic construct
  source 1..38: mol_type = other DNA
aggggtagtgccactgtttcgttttggccccagtcgag

SEQ ID NO: 972 - DNA, 38 bp, synthetic construct
  source 1..38: mol_type = other DNA
ttaaaacgaccgggagtccagttcgaacgatattttaa

SEQ ID NO: 973 - DNA, 38 bp, synthetic construct
  source 1..38: mol_type = other DNA
agagaatgagttatcttcagtctcaccgtccgcgtaaa

SEQ ID NO: 974 - DNA, 38 bp, synthetic construct
  source 1..38: mol_type = other DNA
cgcgaacggaggggacgaaggtctcgttctccctatca

SEQ ID NO: 975 - DNA, 38 bp, synthetic construct
  source 1..38: mol_type = other DNA
agggtactaaaagctcgcacaggtcaaacctcctagga

SEQ ID NO: 976 - DNA, 38 bp, synthetic construct
  source 1..38: mol_type = other DNA
atggaattccggctacctacagcgatagccatggtagc

SEQ ID NO: 977 - DNA, 38 bp, synthetic construct
  source 1..38: mol_type = other DNA
gtctcgctaaagacattaaaaatggcattagctcgaca

SEQ ID NO: 978 - DNA, 35 bp, synthetic construct
  source 1..35: mol_type = other DNA
ggaagttgagcaggaccccgaaaggggtcccaccc

SEQ ID NO: 979 - DNA, 38 bp, synthetic construct; identical to SEQ ID NO: 265
  source 1..38: mol_type = other DNA

SEQ ID NO: 980 - DNA, 38 bp, synthetic construct; identical to SEQ ID NO: 266
  source 1..38: mol_type = other DNA

SEQ ID NO: 981 - DNA, 38 bp, synthetic construct; identical to SEQ ID NO: 267
  source 1..38: mol_type = other DNA

SEQ ID NO: 982 - DNA, 38 bp, synthetic construct; identical to SEQ ID NO: 268
  source 1..38: mol_type = other DNA

SEQ ID NO: 983 - DNA, 38 bp, synthetic construct; identical to SEQ ID NO: 269
  source 1..38: mol_type = other DNA

SEQ ID NO: 984 - DNA, 38 bp, synthetic construct; identical to SEQ ID NO: 270
  source 1..38: mol_type = other DNA

SEQ ID NO: 985 - DNA, 38 bp, synthetic construct; identical to SEQ ID NO: 271
  source 1..38: mol_type = other DNA

SEQ ID NO: 986 - DNA, 38 bp, synthetic construct; identical to SEQ ID NO: 272
  source 1..38: mol_type = other DNA

SEQ ID NO: 987 - DNA, 38 bp, synthetic construct; identical to SEQ ID NO: 273
  source 1..38: mol_type = other DNA

SEQ ID NO: 988 - DNA, 40 bp, synthetic construct; identical to SEQ ID NO: 603
  source 1..40: mol_type = other DNA

SEQ ID NO: 989 - DNA, 40 bp, synthetic construct; identical to SEQ ID NO: 604
  source 1..40: mol_type = other DNA

SEQ ID NO: 990 - DNA, 40 bp, synthetic construct; identical to SEQ ID NO: 605
  source 1..40: mol_type = other DNA

SEQ ID NO: 991 - DNA, 32 bp, synthetic construct; identical to SEQ ID NO: 280
  source 1..32: mol_type = other DNA

SEQ ID NO: 992 - DNA, 38 bp, synthetic construct; identical to SEQ ID NO: 281
  source 1..38: mol_type = other DNA

SEQ ID NO: 993 - DNA, 38 bp, synthetic construct; identical to SEQ ID NO: 282
  source 1..38: mol_type = other DNA

SEQ ID NO: 994 - DNA, 38 bp, synthetic construct; identical to SEQ ID NO: 283
  source 1..38: mol_type = other DNA

SEQ ID NO: 995 - DNA, 38 bp, synthetic construct; identical to SEQ ID NO: 284
  source 1..38: mol_type = other DNA

SEQ ID NO: 996 - DNA, 38 bp, synthetic construct; identical to SEQ ID NO: 285
  source 1..38: mol_type = other DNA

SEQ ID NO: 997 - DNA, 38 bp, synthetic construct; identical to SEQ ID NO: 286
  source 1..38: mol_type = other DNA

SEQ ID NO: 998 - DNA, 38 bp, synthetic construct; identical to SEQ ID NO: 287
  source 1..38: mol_type = other DNA

SEQ ID NO: 999 - DNA, 38 bp, synthetic construct; identical to SEQ ID NO: 288
  source 1..38: mol_type = other DNA

SEQ ID NO: 1000 - DNA, 38 bp, synthetic construct; identical to SEQ ID NO: 289
  source 1..38: mol_type = other DNA

SEQ ID NO: 1001 - DNA, 38 bp, synthetic construct; identical to SEQ ID NO: 290
  source 1..38: mol_type = other DNA

SEQ ID NO: 1002 - DNA, 38 bp, synthetic construct; identical to SEQ ID NO: 291
  source 1..38: mol_type = other DNA

SEQ ID NO: 1003 - DNA, 38 bp, synthetic construct; identical to SEQ ID NO: 292
  source 1..38: mol_type = other DNA

SEQ ID NO: 1004 - DNA, 40 bp, synthetic construct
  source 1..40: mol_type = other DNA
cgtcgccagttccgccattgtcgacgagaacgaactgagt

SEQ ID NO: 1005 - DNA, 40 bp, synthetic construct
  source 1..40: mol_type = other DNA
aaagttagaagccatgcttcaaactccggttgagggctct

SEQ ID NO: 1006 - DNA, 40 bp, synthetic construct
  source 1..40: mol_type = other DNA
atctagagagccgttgcctgattaatgctaacgcatctaa

SEQ ID NO: 1007 - DNA, 32 bp, synthetic construct; identical to SEQ ID NO: 299
  source 1..32: mol_type = other DNA

SEQ ID NO: 1008 - DNA, 38 bp, synthetic construct; identical to SEQ ID NO: 300
  source 1..38: mol_type = other DNA

SEQ ID NO: 1009 - DNA, 38 bp, synthetic construct; identical to SEQ ID NO: 301
  source 1..38: mol_type = other DNA

SEQ ID NO: 1010 - DNA, 38 bp, synthetic construct; identical to SEQ ID NO: 302
  source 1..38: mol_type = other DNA

SEQ ID NO: 1011 - DNA, 38 bp, synthetic construct; identical to SEQ ID NO: 303
  source 1..38: mol_type = other DNA

SEQ ID NO: 1012 - DNA, 38 bp, synthetic construct; identical to SEQ ID NO: 304
  source 1..38: mol_type = other DNA

SEQ ID NO: 1013 - DNA, 38 bp, synthetic construct; identical to SEQ ID NO: 305
  source 1..38: mol_type = other DNA

SEQ ID NO: 1014 - DNA, 38 bp, synthetic construct; identical to SEQ ID NO: 306
  source 1..38: mol_type = other DNA

SEQ ID NO: 1015 - DNA, 38 bp, synthetic construct; identical to SEQ ID NO: 307
  source 1..38: mol_type = other DNA

SEQ ID NO: 1016 - DNA, 38 bp, synthetic construct; identical to SEQ ID NO: 308
  source 1..38: mol_type = other DNA

SEQ ID NO: 1017 - DNA, 40 bp, synthetic construct
  source 1..40: mol_type = other DNA
tgcacgttgtctggaagtttgcagctggatacgacagacg

SEQ ID NO: 1018 - DNA, 40 bp, synthetic construct
  source 1..40: mol_type = other DNA
gccatctaacttgatgttagtaccgacctgacgtacggct

SEQ ID NO: 1019 - DNA, 40 bp, synthetic construct
  source 1..40: mol_type = other DNA
ctcataggaagaaactcttgaaggtgaaccttcgtaagca

SEQ ID NO: 1020 - DNA, 32 bp, synthetic construct; identical to SEQ ID NO: 315
  source 1..32: mol_type = other DNA

SEQ ID NO: 1021 - DNA, 38 bp, synthetic construct; identical to SEQ ID NO: 316
  source 1..38: mol_type = other DNA

SEQ ID NO: 1022 - DNA, 38 bp, synthetic construct; identical to SEQ ID NO: 317
  source 1..38: mol_type = other DNA

SEQ ID NO: 1023 - DNA, 38 bp, synthetic construct; identical to SEQ ID NO: 318
  source 1..38: mol_type = other DNA

SEQ ID NO: 1024 - DNA, 38 bp, synthetic construct; identical to SEQ ID NO: 319
  source 1..38: mol_type = other DNA

SEQ ID NO: 1025 - DNA, 38 bp, synthetic construct; identical to SEQ ID NO: 320
  source 1..38: mol_type = other DNA

SEQ ID NO: 1026 - DNA, 38 bp, synthetic construct; identical to SEQ ID NO: 321
  source 1..38: mol_type = other DNA

SEQ ID NO: 1027 - DNA, 38 bp, synthetic construct; identical to SEQ ID NO: 322
  source 1..38: mol_type = other DNA

SEQ ID NO: 1028 - DNA, 38 bp, synthetic construct; identical to SEQ ID NO: 323
  source 1..38: mol_type = other DNA

SEQ ID NO: 1029 - DNA, 38 bp, synthetic construct; identical to SEQ ID NO: 324
  source 1..38: mol_type = other DNA

SEQ ID NO: 1030 - DNA, 3569 bp, Bacteriophage MS2
  source 1..3569: mol_type = genomic DNA
gggtgggacccctttcggggtcctgctcaacttcctgtcgagctaatgccatttttaatgtctttagcgagacgctaccatggctatcgctgtaggtagccggaattccattcctaggaggtttgacctgtgcgagcttttagtacccttgatagggagaacgagaccttcgtcccctccgttcgcgtttacgcggacggtgagactgaagataactcattctctttaaaatatcgttcgaactggactcccggtcgttttaactcgactggggccaaaacgaaacagtggcactacccctctccgtattcacggggggcgttaagtgtcacatcgatagatcaaggtgcctacaagcgaagtgggtcatcgtggggtcgcccgtacgaggagaaagccggtttcggcttctccctcgacgcacgctcctgctacagcctcttccctgtaagccaaaacttgacttacatcgaagtgccgcagaacgttgcgaaccgggcgtcgaccgaagtcctgcaaaaggtcacccagggtaattttaaccttggtgttgctttagcagaggccaggtcgacagcctcacaactcgcgacgcaaaccattgcgctcgtgaaggcgtacactgccgctcgtcgcggtaattggcgccaggcgctccgctaccttgccctaaacgaagatcgaaagtttcgatcaaaacacgtggccggcaggtggttggagttgcagttcggttggttaccactaatgagtgatatccagggtgcatatgagatgcttacgaaggttcaccttcaagagtttcttcctatgagagccgtacgtcaggtcggtactaacatcaagttagatggccgtctgtcgtatccagctgcaaacttccagacaacgtgcaacatatcgcgacgtatcgtgatatggttttacataaacgatgcacgtttggcatggttgtcgtctctaggtatcttgaacccactaggtatagtgtgggaaaaggtgcctttctcattcgttgtcgactggctcctacctgtaggtaacatgctcgagggccttacggcccccgtgggatgctcctacatgtcaggaacagttactgacgtaataacgggtgagtccatcataagcgttgacgctccctacgggtggactgtggagagacagggcactgctaaggcccaaatctcagccatgcatcgaggggtacaatccgtatggccaacaactggcgcgtacgtaaagtctcctttctcgatggtccataccttagatgcgttagcattaatcaggcaacggctctctagatagagccctcaaccggagtttgaagcatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccattcaaacatgaggattacccatgtcgaagacaacaaagaagttcaactctttatgtattgatcttcctcgcgatctttctctcgaaatttaccaatcaattgcttctgtcgctactggaagcggtgatccgcacagtgacgactttacagcaattgcttacttaagggacgaattgctcacaaagcatccgaccttaggttctggtaatgacgaggcgacccgtcgtaccttagctatcgctaagctacgggaggcgaatggtgatcgcggtcagataaatagagaaggtttcttacatgacaaatccttgtcatgggatccggatgttttacaaaccagcatccgtagccttattggcaacctcctctctggctaccgatcgtcgttgtttgggcaatgcacgttctccaacggtgctcctatggggcacaagttgcaggatgcagcgccttacaagaagttcgctgaacaagcaaccgttaccccccgcgctctgagagcggctctattggtccgagaccaatgtgcgccgtggatcagacacgcggtccgctataacgagtcatatgaatttaggctcgttgtagggaacggagtgtttacagttccgaagaataataaaatagatcgggctgcctgtaaggagcctgatatgaatatgtacctccagaaaggggtcggtgctttcatcagacgccggctcaaatccgttggtatagacctgaatgatcaatcgatcaaccagcgtctggctcagcagggcagcgtagatggttcgcttgcgacgatagacttatcgtctgcatccgattccatctccgatcgcctggtgtggagttttctcccaccagagctatattcatatctcgatcgtatccgctcacactacggaatcgtagatggcgagacgatacgatgggaactattttccacaatgggaaatgggttcacatttgagctagagtccatgatattctgggcaatagtcaaagcgacccaaatccattttggtaacgccggaaccataggcatctacggggacgatattatatgtcccagtgagattgcaccccgtgtgctagaggcacttgcctactacggttttaaaccgaatcttcgtaaaacgttcgtgtccgggctctttcgcgagagctgcggcgcgcacttttaccgtggtgtcgatgtcaaaccgttttacatcaagaaacctgttgacaatctcttcgccctgatgctgatattaaatcggctacggggttggggagttgtcggaggtatgtcagatccacgcctctataaggtgtgggtacggctctcctcccaggtgccttcgatgttcttcggtgggacggacctcgctgccgactactacgtagtcagcccgcctacggcagtctcggtatacaccaagactccgtacgggcggctgctcgcggatacccgtacctcgggtttccgtcttgctcgtatcgctcgagaacgcaagttcttcagcgaaaagcacgacagtggtcgctacatagcgtggttccatactggaggtgaaatcaccgacagcatgaagtccgccggcgtgcgcgttatacgcacttcggagtggctaacgccggttcccacattccctcaggagtgtgggccagcgagctctcctcggtagctgaccgagggacccccgtaaacggggtgggtgtgctcgaaagagcacgggtgcgaaagcggtccggctccaccgaaaggtgggcgggcttcggcccagggacctccccctaaagagaggacccgggattctcccgatttggtaactagctgcttggctagttaccaccca

SEQ ID NO: 1031 - RNA, 3569 nt, Bacteriophage MS2
  source 1..3569: mol_type = genomic RNA
gggtgggacccctttcggggtcctgctcaacttcctgtcgagctaatgccatttttaatgtctttagcgagacgctaccatggctatcgctgtaggtagccggaattccattcctaggaggtttgacctgtgcgagcttttagtacccttgatagggagaacgagaccttcgtcccctccgttcgcgtttacgcggacggtgagactgaagataactcattctctttaaaatatcgttcgaactggactcccggtcgttttaactcgactggggccaaaacgaaacagtggcactacccctctccgtattcacggggggcgttaagtgtcacatcgatagatcaaggtgcctacaagcgaagtgggtcatcgtggggtcgcccgtacgaggagaaagccggtttcggcttctccctcgacgcacgctcctgctacagcctcttccctgtaagccaaaacttgacttacatcgaagtgccgcagaacgttgcgaaccgggcgtcgaccgaagtcctgcaaaaggtcacccagggtaattttaaccttggtgttgctttagcagaggccaggtcgacagcctcacaactcgcgacgcaaaccattgcgctcgtgaaggcgtacactgccgctcgtcgcggtaattggcgccaggcgctccgctaccttgccctaaacgaagatcgaaagtttcgatcaaaacacgtggccggcaggtggttggagttgcagttcggttggttaccactaatgagtgatatccagggtgcatatgagatgcttacgaaggttcaccttcaagagtttcttcctatgagagccgtacgtcaggtcggtactaacatcaagttagatggccgtctgtcgtatccagctgcaaacttccagacaacgtgcaacatatcgcgacgtatcgtgatatggttttacataaacgatgcacgtttggcatggttgtcgtctctaggtatcttgaacccactaggtatagtgtgggaaaaggtgcctttctcattcgttgtcgactggctcctacctgtaggtaacatgctcgagggccttacggcccccgtgggatgctcctacatgtcaggaacagttactgacgtaataacgggtgagtccatcataagcgttgacgctccctacgggtggactgtggagagacagggcactgctaaggcccaaatctcagccatgcatcgaggggtacaatccgtatggccaacaactggcgcgtacgtaaagtctcctttctcgatggtccataccttagatgcgttagcattaatcaggcaacggctctctagatagagccctcaaccggagtttgaagcatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccattcaaacatgaggattacccatgtcgaagacaacaaagaagttcaactctttatgtattgatcttcctcgcgatctttctctcgaaatttaccaatcaattgcttctgtcgctactggaagcggtgatccgcacagtgacgactttacagcaattgcttacttaagggacgaattgctcacaaagcatccgaccttaggttctggtaatgacgaggcgacccgtcgtaccttagctatcgctaagctacgggaggcgaatggtgatcgcggtcagataaatagagaaggtttcttacatgacaaatccttgtcatgggatccggatgttttacaaaccagcatccgtagccttattggcaacctcctctctggctaccgatcgtcgttgtttgggcaatgcacgttctccaacggtgctcctatggggcacaagttgcaggatgcagcgccttacaagaagttcgctgaacaagcaaccgttaccccccgcgctctgagagcggctctattggtccgagaccaatgtgcgccgtggatcagacacgcggtccgctataacgagtcatatgaatttaggctcgttgtagggaacggagtgtttacagttccgaagaataataaaatagatcgggctgcctgtaaggagcctgatatgaatatgtacctccagaaaggggtcggtgctttcatcagacgccggctcaaatccgttggtatagacctgaatgatcaatcgatcaaccagcgtctggctcagcagggcagcgtagatggttcgcttgcgacgatagacttatcgtctgcatccgattccatctccgatcgcctggtgtggagttttctcccaccagagctatattcatatctcgatcgtatccgctcacactacggaatcgtagatggcgagacgatacgatgggaactattttccacaatgggaaatgggttcacatttgagctagagtccatgatattctgggcaatagtcaaagcgacccaaatccattttggtaacgccggaaccataggcatctacggggacgatattatatgtcccagtgagattgcaccccgtgtgctagaggcacttgcctactacggttttaaaccgaatcttcgtaaaacgttcgtgtccgggctctttcgcgagagctgcggcgcgcacttttaccgtggtgtcgatgtcaaaccgttttacatcaagaaacctgttgacaatctcttcgccctgatgctgatattaaatcggctacggggttggggagttgtcggaggtatgtcagatccacgcctctataaggtgtgggtacggctctcctcccaggtgccttcgatgttcttcggtgggacggacctcgctgccgactactacgtagtcagcccgcctacggcagtctcggtatacaccaagactccgtacgggcggctgctcgcggatacccgtacctcgggtttccgtcttgctcgtatcgctcgagaacgcaagttcttcagcgaaaagcacgacagtggtcgctacatagcgtggttccatactggaggtgaaatcaccgacagcatgaagtccgccggcgtgcgcgttatacgcacttcggagtggctaacgccggttcccacattccctcaggagtgtgggccagcgagctctcctcggtagctgaccgagggacccccgtaaacggggtgggtgtgctcgaaagagcacgggtgcgaaagcggtccggctccaccgaaaggtgggcgggcttcggcccagggacctccccctaaagagaggacccgggattctcccgatttggtaactagctgcttggctagttaccaccca